(19) United States
(12) Patent Application Publication
(10) Pub. No.: US 2023/0066830 A1
(43) Pub. Date: Mar. 2, 2023

(54) METHODS AND COMPOSITIONS FOR INDUCING APOPTOSIS IN CANCER STEM CELLS

(71) Applicant: VIRGINIA COMMONWEALTH UNIVERSITY, Richmond, VA (US)

(72) Inventors: Paul B. FISHER, Richmond, VA (US); Sarmistha TALUKDAR, Richmond, VA (US); Swadesh K. DAS, Richmond, VA (US); Anjan PRADHAN, Richmond, VA (US); Luni EMDAD, Richmond, VA (US)

(21) Appl. No.: 17/787,483

(22) PCT Filed: Dec. 18, 2020
(86) PCT No.: PCT/US20/65790

§ 371 Date: Jun. 20, 2022

Related U.S. Application Data
(60) Provisional application No. 62/951,340, filed on Dec. 20, 2019.
(60) Provisional application No. 63/066,857, filed on Aug. 18, 2020.

(51) Int. Cl. A61K 31/519; C12N 15/113; A61K 31/337; A61K 31/439; A61K 31/165; A61K 31/18; A61K 31/4353; A61K 38/15; A61K 31/336; A61K 31/167; A61K 31/4406

(52) U.S. Cl. CPC A61K 31/519; C12N 15/113; A61K 31/337; A61K 31/439; A61K 31/165; A61K 31/18; A61K 31/4353; A61K 38/15; A61K 31/336; A61K 31/167; A61K 31/4406; C12N 2310/14; C12N 2310/531; C12N 2320/31

(57) ABSTRACT

Provided herein are pharmaceutical compositions and methods useful for treating cancer and preventing cancer metastasis, particularly in cancers that have increased MDA-9/Syntenin expression.

### CROSS REFERENCE TO RELATED APPLICATIONS

This application claims priority to U.S. Provisional Application No. 63/066,857, filed on Aug. 18, 2020, and U.S. Provisional Application No. 62/951,340, filed on Dec. 20, 2019, which are hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. CA097318, CA 168517, and CA016059, awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 052893-507001WO_SEQUENCE_LISTING_ST25, created on Dec. 11, 2020, which is 3,276 bytes in size is hereby incorporated by reference.

### BACKGROUND

Described herein, inter alia, are compositions for the treatment of cancer, including small molecule inhibitors capable of treating or preventing cancer invasion, attachment and/or metastasis, for example difficult to treat tumors such as glioblastoma.

Cancer (malignant neoplasia) is a disease involving unregulated cell growth. In cancer, cells divide and grow uncontrollably, forming malignant tumors, which may invade nearby parts of the body. The cancer may also spread to more distant parts of the body through the lymphatic system or bloodstream (metastasis). Metastasis is a complex series of steps in which cancer cells leave the primary tumor site, migrate and colonize to distant sites or organs of the body via the bloodstream or the lymphatic system. Cancer is usually treated with one or a combination of chemotherapy, radiation therapy and/or surgery. While treatment methods have advanced significantly, the outcomes for particular cancers are still not optimal, current treatments often have very harsh side effects, and if the cancer is not detected early, the chances of survival are greatly reduced. Invasive, metastatic cancer is particularly difficult to treat.

Glioblastoma multiforme (GBM) is an especially intractable tumor despite therapeutic advances principally because of its invasive properties. Radiation is a staple in modem therapeutic regimens. However, when glioblastoma multiforme (GBM) cells are irradiated (a common mode of therapy after tumor debulking) the invasive ability of GBM is increased, and cells surviving radiation become even more aggressive and invasive.

There is a need in the art for additional agents to treat cancer. In particular, there is a need for additional agents that prevent cancer invasion, attachment and/or metastasis, and that prevent or attenuate the increase in invasive ability of cancer cells after exposure to irradiation. Described herein are solutions to these and other problems in the art.

### BRIEF SUMMARY

In an aspect, the present disclosure provides a method of treating cancer in a subject in need thereof. In embodiments, the method includes administering to the subject a combined effective amount of (a) an agent that inhibits MDA-9, and (b) an anti-cancer agent, wherein the anti-cancer agent is a mitotic inhibitor or a histone deacetylase (HDAC) inhibitor.

In an aspect, the present disclosure provides a pharmaceutical composition including (a) an agent that inhibits MDA-9, and (b) an anti-cancer agent, wherein the anti-cancer agent is a mitotic inhibitor or a histone deacetylase (HDAC) inhibitor.

In an aspect, the present disclosure provides uses of a pharmaceutical composition disclosed herein for the treatment of cancer.

In an aspect, the present disclosure provides kits including one or more pharmaceutical compositions disclosed herein.

### BRIEF DESCRIPTION OF THE DRAWINGS

FIGS. 1A-1E present data showing that mda-9 expression correlated with sternness markers. FIG. 1A is a bar graph showing the expression of mda-9 in normal prostate and prostate cancer stem cells. FIG. 1B is a bar graph showing the expression of Nanog. and Oct4 in mda-9 overexpressing normal prostate stem cells (bars within a group as indicated in the legend, from left to right). FIG. 1C shows confocal images showing the size of RWPE-1 cells in parental and mda-9 overexpressing prostaspheres. FIG. 1D is a bar graph showing graphical depiction of the spheroid size in RWPE-1 prostaspheres in parental and mda-9 overexpressing cells. FIG. 1E is a bar graph showing the effect of mda-9 overexpression on normal prostate stem cell populations (bars within a group as indicated in the legend, from left to right). In FIGS. 1A, 1B, 1D and 1E * p<0.05, ** p<0.01, using the Student's t-test and ANOVA. In FIG. 1C, the scale bars represent 5 μm.

FIGS. 2A-2D present data showing that MDA-9 regulated survival and tumorigenic potential in prostate cancer stem cells (PCSCs). FIG. 2A is a bar graph showing the effect of mda-9 knock down (kd) in promoting PCSCs cell death analyzed by Annexin V assays. FIG. 2B is a graph showing the effect of mda-9 kd on PCSC tumorigenicity (n=10). FIG. 2C is a bar graph showing the effect of intra-tumoral mda-9 kd on tumorigenicity (n=10). FIG. 2D is a bar graph showing PCSC populations (percentage of CD44⁺133⁺ cells) in tumors following intra-tumoral mda-9 kd. In FIGS. 2A, 2B, and 2D, the bars represent SEM. ** p<0.01 using the Student's t-test and ANOVA.

FIGS. 3A-3D present data showing that mda-9 regulated chemoresistance in PCSCs. FIG. 3A shows fluorescence microscopy pictures showing the effect of mda-9 kd on PCSC sensitivity to Docetaxel assessed by Live/Dead assay: live cells are green, dead cells are red. FIG. 3B is a bargraph showing the effect of mda-9 kd on PCSC sensitivity to Docetaxel (5 and 10 nM), assessed by MTT assay. FIG. 3C shows the effect of mda-9 overexpression on PCSC sensitivity to Docetaxel assessed by caspase activity (bars within a group as indicated in the legend, from left to right). FIG. 3D is a bar graph showing the effect of intra-tumoral mda-9 kd on Docetaxel sensitivity measured by tumor weight (n=10). In FIGS. 3B, 3C and 3D, the bars represent SEM. * p<0.05, ** p<0.01, using the Student's t-test and ANOVA.

FIGS. 4A-4D present data showing that STAT3 activation is downstream of mda-9 and it regulates PCSC chemoresistance. FIG. 4A shows image analysis of shcon or mda-9 kd (shmda-9) PCSCs overexpressing constitutively active (CA) STAT3 or treated with STAT3 inhibitor STATTIC, of DU-145 treated with or without Docetaxel. FIG. 4B shows image analysis of shcon or mda-9 kd (shmda-9) PCSCs overexpressing constitutively active (CA) STAT3 or treated with STAT3 inhibitor STATTIC of DU-145 treated with or without TSA. FIG. 4C shows image analysis of shcon or mda-9 kd (shmda-9) PCSCs overexpressing constitutively active (CA) STAT3 or treated with STAT3 inhibitor STATTIC of PC3-ML treated with or without Docetaxel. FIG. 4D shows image analysis of shcon or mda-9 kd (shmda-9) PCSCs overexpressing constitutively active (CA) STAT3 or treated with STAT3 inhibitor STATTIC of PC3-ML treated with or without TSA. In FIGS. 4A-4D, live cells are green, dead cells are red.

FIGS. 5A-5E present data showing that mda-9 regulated MDR1 expression in PCSCs. FIG. 5A is a bar graph showing RT-PCR analysis to study the effect of mda-9 kd on ABC family gene expression in DU-145 PCSCs (bottom portion of each bar showing results for shcon, top portion of each bar showing results for shmda-9). FIG. 5B is a bar graph showing RT-PCR analysis to study the effect of mda-9 kd on ABC family gene expression in PC3-ML PCSCs (bottom portion of each bar showing results for shcon, top portion of each bar showing results for shmda-9). FIG. 5C is a picture of a Western blot showing the effect of mda-9 kd on MDR1 protein expression in DU-145 and PC3-ML PCSCs. FIG. 5D is a bar graph showing the effect of mda-9 kd on MDR1 mRNA expression in DU-145 and PC3-ML PCSCs (bars within a group as indicated in the legend, from left to right). FIG. 5E shows microscopy pictures showing the expression of MDR1, STAT3 and P-STAT3 in control, mda-9 kd, PDZ1i and Docetaxel treated tumors analyzed by IHC (n=8). In FIGS. 5A-5D, the bars represent SEM. * p<0.05, ** p<0.01, using the Student's t-test and ANOVA.

FIGS. 6A-6C present data showing that mda-9 regulated MDR1 expression through STAT3 activation. FIG. 6A is a bar graphs showing the effect of MDR1 overexpression on mda-9 kd mediated caspase activity. The bars represent SEM. * p<0.05, using the Student's t-test and ANOVA. FIG. 6B shows the effect of CA-STAT3 overexpression on MDR1 protein expression in DU-145 and PC3-ML PCSCs by Western blotting and as quantified in arbitrary units (pairs of bars represent results for DU-145 (left) and PC3-ML (right)). FIG. 6C shows flow cytometry plots showing the effect of CA-STAT3 overexpression on MDR1 protein expression in ARCaP-M PCSCs.

FIG. 7 is a schematic representation of potential routes by which MDA-9 mediates regulation of chemoresistance, in PCSCs. Without wishing to be bound by theory, MDA-9 potentially regulates chemoresistance through IGF-1R/p44/42/STAT3/MDR1 and C-myc/MDR1 axis, and stem regulatory genes OCT4, Nanog and SOX-2 through STAT3.

FIGS. 8A-8B illustrate the effect of mda-9 overexpression on PCSCs. FIG. 8A shows cell sorting plots presenting the effect of overexpression of mda-9 in prostate cancer stem cells and non-stem cancer cells on stem populations as assayed by cell surface stemness markers. FIG. 8B shows bar graphs presenting the effect of overexpression of mda-9 in prostate cancer stem cells and non-stem cancer cells on stem regulatory molecule (NANOG, OCT4 and SOX2) expression (pairs of bars from left to right as indicated in the legend from top to bottom).

FIGS. 9A-9D illustrate the effect of kd of mda-9 on PCSCs. FIG. 9A is a bar graph showing that kd of mda-9 enhanced PCSC cell death. FIG. 9B is a bar graph showing PCSC sensitivity to docetaxel and TSA treatment in vitro and effect of mda-9 overexpression on PCSC sensitivity to chemotherapeutics (bars within a group as indicated in the legend, from left to right). FIG. 9C is a bar graph showing the effect of PDZ1i (pharmacological inhibitor of MDA-9) on docetaxel sensitivity of tumors measured by tumor weight. FIG. 9D is a bar graph showing the effect of PDZ1i (pharmacological inhibitor of MDA-9) on docetaxel sensitivity of tumors analyzed by histopathology. In FIGS. 2A-2C, bars represent SEM. *P<0.05, using student t-test and ANOVA.

FIGS. 10A-10C illustrate MDA-9-mediated STAT3 regulation, and its downstream effects in PCSCs. FIG. 10A is a bar graph showing that STAT3 signaling through MDA-9 regulated chemosensitivity to the HDAC inhibitor TSA. FIG. 10B is a bar graph showing that STAT3 activation by MDA-9 regulated stem regulatory molecules including OCT4, SOX2 and NANOG. FIG. 10C is a bar graph showing image analysis data showing the role of STAT3 in MDA-9-based chemoresistance to Docetaxel and TSA in DU-145 and PC3-ML PCSCs. In FIG. 10A-10C, bars represent SEM. *P<0.05, using student t-test and ANOVA. NS depicts results that are not statistically significant. All bars within a group are as indicated in the respective legend, from left to right.

FIGS. 11A-11B present data related to MDR1 expression. FIG. 11A is a bar graph showing an Image J analysis of the Western blots shown in FIG. 5C to quantify MDR1 expression (bars within a group as indicated in the legend, from left to right). FIG. 11B expression of mda-9 in shcontrol, mda-9 kd and mda-9 kd PCSCs overexpressing MDR1.

FIGS. 12A-12B present data showing that MDA-9 regulated chemoresistance in PCSCs though the cmyc/MDR1 axis. FIG. 12A shows Live/Dead analysis of PCSC sensitivity to Docetaxel after cmyci, Docetaxel, mda-9 overexpression treatment alone and in combination, by fluorescence microscopy. In the microscopy pictures, live cells are green, dead cells are red. In each group of three images, from left to right, the images show live staining, dead staining, and a merge. FIG. 12B shows cell sorting plots showing the effect of cmyc inhibition on MDR1 expression.

FIG. 13 shows fluorescence microscopy pictures showing that mda-9 regulated PCSC survival through the NOTCH1 pathway, via peptide blocking studies to elucidate the effect of Notch1 blocking peptide (NBP) on PCSC viability. In the microscopy pictures, live cells are green, dead cells are red.

### DETAILED DESCRIPTION

MDA-9 is a scaffold protein that plays a key role in tumor progression and metastasis in cancer. MDA-9 can effect tumor progression and metastasis through protein-protein interactions. For example, in breast cancer MDA-9 interacts with TGFβ1 to facilitate epithelial mesenchymal transition (EMT), a key step in the processes of metastatsis. MDA-9 protein-protein interactions can occur through binding of a MDA-9 PDZ domain (i.e., PDZ1 domain) to a downstream target (e.g., TGFβ1, c-Src, FAK, STAT3, IGF-R1, etc.) in the MDA-9 signaling pathway.

There are an estimated ˜150 PDZ domain-containing proteins that are involved in cancer and associated with important physiological processes in transformed cells. Targeting the PDZ domain to develop specific and effective small molecule inhibitors has historically proven difficult. However, as described herein, specific and effective PDZ1 domain binders which target the PDZ1 domain of MDA-9/Syntenin, thereby inhibiting MDA-9 protein-protein interactions, have been developed for use in cancer treatment.

### I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. The bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through an atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through an atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through an atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

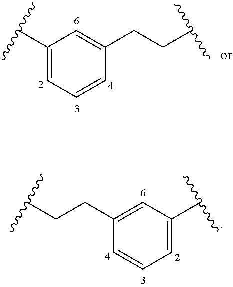 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group” or “substituent” as used herein, means a group selected from the following moieties:
(A) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀ alkyl, C₁-C₁₂ alkyl, C₁-C₈ alkyl, C₁-C₆ alkyl, C₁-C₄ alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂C₁, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀ alkyl, C₁-C₁₂alkyl, C₁-C₈ alkyl, C₁-C₆alkyl, C₁-C₄alkyl, or C₁-C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃-C₁₀ cycloalkyl, C₃-C₈ cycloalkyl, C₃-C₆ cycloalkyl, C₄-C₆ cycloalkyl, or C₅-C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆-C₁₂ aryl, C₆-C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo,
      halogen, —CCl₃, —CBr₃, —CF₃, —CI₃, —CHCl₂, —CHBr₂,
      —CHF₂, —CHI₂, —CH₂C₁, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound is a chemical species set forth herein, for example in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on a R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g. R^{1.1}, R^{2.1}, R^{3.1}, R^{4.1}, R^{5.1} . . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g. R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g. R^{1.3}, R^{2.3}, R^{3.3}, R^{4.3}3, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g. when R^{WW.1} is R^{WW.2} substituted alkyl, examples of groups so formed include but are not limited to itself optionally substituted by 1 or more R^{WW.2}, which R^{WW.2} is optionally substituted by one or more R^{WW.3}. By way of example when R^{WW.1} is alkyl, groups that could be formed, include but are not limited to:

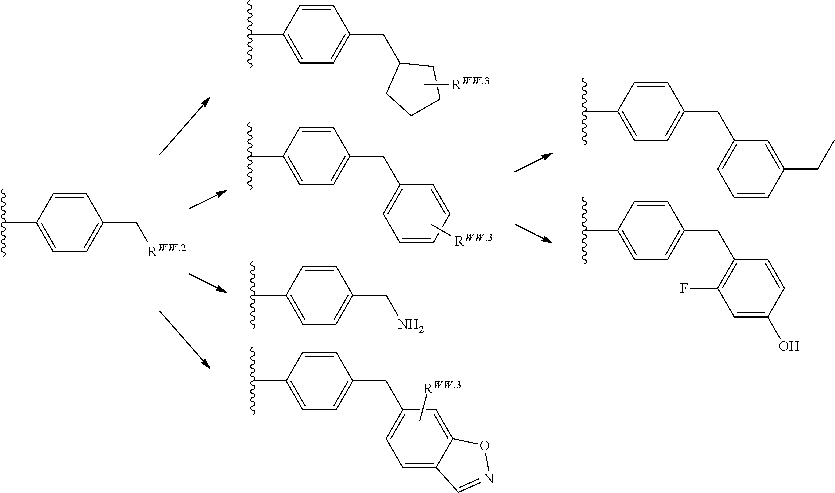

R^{WW.1} is independently oxo,

halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —O H, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo,
halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo,

halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo,
halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo,

halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo,

halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —C N, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo,
halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo,

halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo,
halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo,

halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo,

halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g. 1, 2, 3, 1A, 2A, 3A, 1B, 2B, 3B etc.). R^{WW.1}, R^{WW.2}, and R^{WW.3}, are as defined above.

In the event that any L linker group recited in a claim or chemical formula description set forth herein (i.e. an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂NH—, —NHSO₂—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B etc.). R^{LWW.1}, as well as R^{LWW.2} and R^{LWW.3}, are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. For example, formula I may be written as

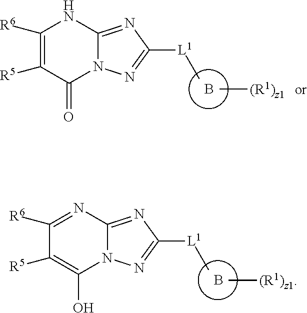

Additionally, any formula or compound described herein may be written as either isomeric (e.g. tautomeric) form.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein may be bound, for example, by covalent bond, linker (e.g. a first linker of second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “phosphorothioate nucleic acid” refers to a nucleic acid in which one or more internucleotide linkages are through a phosphorothioate moiety (thiophosphate) moiety. The phosphorothioate moiety may be a monothiophosphate (—P(O)₃(S)³⁻—) or a dithiophosphate (—P(O)₂(S)₂³⁻—). In embodiments of all the aspects provided herein, the phosphorothioate moiety is a monothiophosphate (—P(O)₃(S)³⁻—). That is, in embodiments of all the aspects provided herein, the phosphorothioate nucleic acid is a monothiophosphate nucleic acid. In embodiments, one or more of the nucleosides of a phosphorothioate nucleic acid are linked through a phosphorothioate moiety (e.g. monothiophosphate) moiety, and the remaining nucleosides are linked through a phosphodiester moiety (—P(O)₄³⁻—). In embodiments, one or more of the nucleosides of a phosphorothioate nucleic acid are linked through a phosphorothioate moiety (e.g. monothiophosphate) moiety, and the remaining nucleosides are linked through a methylphosphonate linkage. In embodiments, all the nucleosides of a phosphorothioate nucleic acid are linked through a phosphorothioate moiety (e.g. a monothiophosphate) moiety.

Phosphorothioate oligonucleotides (phosphorothioate nucleic acids) are typically from about 5, 6, 7, 8, 9, 10, 12, 15, 25, 30, 40, 50 or more nucleotides in length, up to about 100 nucleotides in length. Phosphorothioate nucleic acids may also be longer in lengths, e.g., 200, 300, 500, 1000, 2000, 3000, 5000, 7000, 10,000, etc. As described above, in certain embodiments. the phosphorothioate nucleic acids herein contain one or more phosphodiester bonds. In other embodiments, the phosphorothioate nucleic acids include alternate backbones (e.g., mimics or analogs of phosphodiesters as known in the art, such as, boranophosphate, methylphosphonate, phosphoramidate, or O-methylphosphoroamidite linkages (see Eckstein, Oligonucleotides and Analogues: A Practical Approach, Oxford University Press). The phosphorothioate nucleic acids may also include one or more nucleic acid analog monomers known in the art, such as, peptide nucleic acid monomer or polymer, locked nucleic acid monomer or polymer, morpholino monomer or polymer, glycol nucleic acid monomer or polymer, or threose nucleic acid monomer or polymer. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. Phosphorothioate nucleic acids and phosphorothioate polymer backbones can be linear or branched. For example, the branched nucleic acids are repetitively branched to form higher ordered structures such as dendrimers and the like.

As used herein, a “phosphorothioate polymer backbone” is a chemical polymer with at least two phosphorothioate linkages (e.g. monothiophosphate) (e.g. linking together sugar subunits, cyclic subunits or alkyl subunits). The phosphorothioate polymer backbone may be a phosphorothioate sugar polymer, which is a phosphorothioate nucleic acid in which one or more (or all) of the chain of pentose sugars lack the bases (nucleobases) normally present in a nucleic acid. The phosphorothioate polymer backbone can include two or more phosphorothioate linkages. The phosphorothioate polymer backbone can include 5, 6, 7, 8, 9, 10, 12, 15, 25, 30, 40, 50 or more linkages and can contain up to about 100 phosphorothioate linkages. Phosphorothioate polymer backbones may also contain a larger number of linkages, e.g., 200, 300, 500, 1000, 2000, 3000, 5000, 7000, 10,000, and the like.

The phosphorothioate nucleic acids and phophorothioate polymer backbones may be partially or completely phosphorothioated. For example, 50% or more of the internucleotide linkages of a phosphorothioate nucleic acid can be phosphorothioate linkages. Optionally, 5%, 10%, 15%, 20%, 25%, 30%, 35%, 40%, 45%, 50%, 55%, 60%, 65%, 70%, 75%, 80%, 85%, 90%, 95%, or 99% of the internucleotide linkages of a phosphorothioate nucleic acid are phosphorothioate linkages. Optionally, 50%, 55%, 60%, 65%, 70%, 75%, 80%, 85%, 90%, 95%, or 99% of the internucleotide linkages of a phosphorothioate nucleic acid are phosphorothioate linkages. Optionally, 75%, 80%, 85%, 90%, 95%, or 99% of the internucleotide linkages of a phosphorothioate nucleic acid are phosphorothioate linkages. Optionally, 90%, 95%, or 99% of the internucleotide linkages of a phosphorothioate nucleic acid are phosphorothioate linkages. In embodiments, the remaining internucleotide linkages are phosphodiester linkages. In embodiments, the remaining internucleotide linkages are methylphosphonate linkages. Optionally, 100% of the internucleotide linkages of the phosphorothioate nucleic acids are phosphorothioate linkages. Similarly, 5%, 10%, 15%, 20%, 25%, 30%, 35%, 40%, 45%, 50%, 55%, 60%, 65%, 70%, 75%, 80%, 85%, 90%, 95%, or 99%, of the intersugar linkages in a phosphorothioate polymer backbone can be phosphorothioate linkages. Optionally, 50%, 55%, 60%, 65%, 70%, 75%, 80%, 85%, 90%, 95%, or 99%, of the intersugar linkages in a phosphorothioate polymer backbone can be phosphorothioate linkages. Optionally, 75%, 80%, 85%, 90%, 95%, or 99%, of the intersugar linkages in a phosphorothioate polymer backbone can be phosphorothioate linkages. Optionally, 90%, 95%, or 99%, of the intersugar linkages in a phosphorothioate polymer backbone can be phosphorothioate linkages. In embodiments, the remaining internucleotide linkages are phosphodiester linkages. In embodiments, the remaining internucleotide linkages are methylphosphonate linkages. Optionally, 100% of the intersugar linkages of the phosphorothioate polymer backbone are phosphorothioate linkages.

Optionally, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the internucleotide linkages of a phosphorothioate nucleic acid are phosphorothioate linkages. Optionally, about 50%, about 55%, about 60%, about 65%, about 70%, about 75%, about 80%, about 85%, about 90%, about 95%, or about 99% of the internucleotide linkages of a phosphorothioate nucleic acid are phosphorothioate linkages. Optionally, about 75%, about 80%, about 85%, about 90%, about 95%, or about 99% of the internucleotide linkages of a phosphorothioate nucleic acid are phosphorothioate linkages. Optionally, about 90%, about 95%, or about 99% of the internucleotide linkages of a phosphorothioate nucleic acid are phosphorothioate linkages. In embodiments, the remaining internucleotide linkages are phosphodiester linkages. In embodiments, the remaining internucleotide linkages are methylphosphonate linkages. Optionally, about 100% of the internucleotide linkages of the phosphorothioate nucleic acids are phosphorothioate linkages. Similarly, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%, of the intersugar linkages in a phosphorothioate polymer backbone can be phosphorothioate linkages. Optionally, about 50%, about 55%, about 60%, about 65%, about 70%, about 75%, about 80%, about 85%, about 90%, about 95%, or about 99%, of the intersugar linkages in a phosphorothioate polymer backbone can be phosphorothioate linkages. Optionally, about 75%, about 80%, about 85%, about 90%, about 95%, or about 99%, of the intersugar linkages in a phosphorothioate polymer backbone can be phosphorothioate linkages. Optionally, about 90%, about 95%, or about 99%, of the intersugar linkages in a phosphorothioate polymer backbone can be phosphorothioate linkages. In embodiments, the remaining internucleotide linkages are phosphodiester linkages. In embodiments, the remaining internucleotide linkages are methylphosphonate linkages. Optionally, about 100% of the intersugar linkages of the phosphorothioate polymer backbone are phosphorothioate linkages.

The term “aptamer” as provided herein refers to oligonucleotides (e.g. short oligonucleotides or deoxyribonucleotides), that bind (e.g. with high affinity and specificity) to proteins, peptides, and small molecules. Aptamers may have secondary or tertiary structure and, thus, may be able to fold into diverse and intricate molecular structures. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified. Aptamers exhibit many desirable properties for targeted drug delivery, such as ease of selection and synthesis, high binding affinity and specificity, low immunogenicity, and versatile synthetic accessibility. To date, a variety of anti-cancer agents (e.g. chemotherapy drugs, toxins, and siRNAs) have been successfully delivered to cancer cells in vitro using apatmers.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., microRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see. e.g., NCBI web site http://www.ncbi.nlninih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlnmnih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to, for example, glutamine at position 110 of a human MDA-9 protein when the selected residue occupies the same essential spatial or other structural relationship as a glutamine at position 110 in human MDA-9 protein. In some embodiments, where a selected protein is aligned for maximum homology with the human MDA-9 protein, the position in the aligned selected protein aligning with glutamine 110 is said to correspond to glutamine 110. Instead of a primary sequence alignment, a three dimensional structural alignment can also be used, e.g., where the structure of the selected protein is aligned for maximum correspondence with the human MDA-9 protein and the overall structures compared. In this case, an amino acid that occupies the same essential position as glutamine 110 in the structural model is said to correspond to the glutamine 110 residue.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) or light chain variable region and variable heavy chain (VH) or heavy chain variable region refer to these light and heavy chain regions, respectively. The terms variable light chain (VL) and light chain variable region as referred to herein may be used interchangeably. The terms variable heavy chain (VH) and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody region provided herein, wherein the binding site is not the peptide binding site.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or target polypeptide (e.g, PDZ1 domain).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a PDZ1 domain binder (e.g., small molecule, antibody, aptamer, ligand, or compound as described herein and a polypeptide provided herein (e.g., MDA-9 (e.g, PDZ1 domain)). In embodiments, contacting includes, for example, allowing a PDZ1 domain binder as described herein, including embodiments thereof, to interact with a PDZ1 domain of a MDA-9 protein.

As defined herein, the term “inhibition”, “inhibit”, “inhibiting” and the like means negatively affecting (e.g. decreasing) the activity of a target. In embodiments, the target is a protein (e.g., a histone deacetylase, or MDA-9), protein domain, or protein-binder (e.g., a PDZ1 domain binder). In embodiments, the target is a process, such as mitosis. Where the target is a protein, an inhibitor negatively affects the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the binder. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

A “PDZ1 domain binder” is a compound that negatively affects (e.g. decreases) the activity or function of MDA-9 relative to the activity or function of MDA-9 in the absence of the PDZ1 domain binder by binding to the PDZ1 domain of the MDA-9 protein. A decrease in MDA-9 activity may result in changes in the signaling pathway downstream of MDA-9. For example, MDA-9 inhibition by a PDZ1 domain binder may decrease activation, activity, expression, or stability of TGFβ1, p38 MAPK, NF-κB, STAT3, IGF-R1, AEG-1, JNK, EGFR, AKT, phohoinositide 3-kinase, FAK, and/or c-Src. In embodiments, an MDA-9 inhibitor is a small molecule inhibitor. In embodiments, inhibition is achieved at the level of gene expression, such as by targeting a gene for knockout, inhibiting gene transcription, targeting a gene transcript for degradation, or block translation of an RNA transcript of a target gene (e.g., mda-9). Non-limiting examples of compounds that inhibit gene expression include small interfering RNA (siRNA), micro RNA (miRNA), and short hairpin RNA (shRNA).

In embodiments, an inhibitor is an inhibitor of mitosis, also referred to as a “mitotic inhibitor.” In general, a mitotic inhibitor is an agent that slows or blocks the ability of a cell to complete mitosis, or reduces the fraction of a given population of cells that is able to complete mitosis. Mitotic inhibitors include anti-microtubule agents, such as taxanes, epothelones, and halichondrins. In general, an “anti-microtubule agent” is an agent that inhibits normal microtubule function, such as by inhibiting microtubule polymerization or depolymerization. Non-limiting examples of mitotic inhibitors include ixabepilone, vincristine, auristatins, dolastatins, maytansinoids, and plant alkaloids. A variety of such compounds are available. See, for example, the mitotic inhibitors disclosed in US20170326252.

A “siRNA”, “small interfering RNA”, “small RNA”, or “RNAi” as provided herein refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi refers to a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. Non-limiting examples of siRNAs include ribozymes, RNA decoys, short hairpin RNAs (shRNA), micro RNAs (miRNA) and small nucleolar RNAs (snoRNA).

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of a MDA-9 protein with a compound as described herein (e.g., PDZ1 domain binder) may reduce the level of a product of the MDA-9 catalyzed reaction or the level of a downstream derivatives of the product or binding may reduce the interactions between MDA-9 or an MDA-9 reaction product and downstream effectors or signaling pathway components, resulting in changes in cell growth, proliferation, metastasis, or survival.

As defined herein, the term “activation”, “activate”, “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

The terms “inhibitor,” “repressor” or “antagonist” or “downregulator” interchangeably refer to a substance capable of detectably decreasing the expression or activity of a given gene or protein, or reducing the activity of a target process (e.g., mitosis, in the case of a mitotic inhibitor, or microtubule polymerization or depolymerization in the case of an anti-microtubule agent). The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The terms “MDA-9,” “Syntenin,” “MDA-9/Syntenin,” or “SDCBP” refer to a protein (including homologs, isoforms, and functional fragments thereof) with MDA-9 activity. The term includes any recombinant or naturally-occurring form of MDA-9 variants thereof that maintain MDA-9 activity (e.g. within at least 30%, 40%, 50%, 60%, 70%, 80%, 90%, 95%, or 100% activity compared to wildtype MDA-9). In embodiments, the MDA-9 protein encoded by the SDCBP gene has the amino acid sequence set forth in or corresponding to Entrez 6386, UniProt 000560, or RefSeq (protein) NP_001007068.1. In embodiments, the SDCBP gene has the nucleic acid sequence set forth in RefSeq (mRNA) NM_001007067.1. In embodiments, the amino acid sequence or nucleic acid sequence is the sequence known at the time of filing of the present application. In embodiments, the MDA-9 protein includes the sequence of SEQ ID NO: 1.

The term “PDZ domain” as referred to herein refers to a common structural domain typically including 80-90 amino acids. PDZ domains are common to scaffold and signaling proteins and play an important role in signal transduction complexes. PDZ domains of a protein can facilitate protein-protein interactions by binding target proteins. MDA-9 proteins include two tandem PDZ domains, PDZ1 and PDZ2, respectively, separated by a sequence of amino acids linking the two domains. The amino acid sequence linking the PDZ1 and PDZ2 domains is referred to herein as an “interface region”. In embodiments, the amino acid sequence of PDZ1 includes the sequence of SEQ ID NO: 1. In embodiments, the amino acid sequence of PDZ1 is the sequence of SEQ ID NO: 1.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a compound or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, or neuroblastoma).

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of cancer. In embodiments, “treating” refers to treatment of infectious disease. In embodiments, “treating” refers to treatment of neurodegenerative disease. In embodiments, “treating” refers to treatment of inflammatory disease.

In embodiments, treatment or treating includes (1) inhibiting a disease in a subject or patient experiencing or displaying the pathology or symptomatology of cancer (e.g., arresting further development of the pathology and/or symptomatology), (2) ameliorating a disease in a subject or patient that is experiencing or displaying the pathology or symptomatology of cancer (e.g., reversing the pathology and/or symptomatology), and/or (3) effecting any measurable decrease in a disease in a subject or patient that is experiencing or displaying the pathology or symptomatology of cancer. In embodiments, the subject treated as described herein may also fully recover from cancer and may become cancer-free as a result of the present methods.

In embodiments, treating cancer refers to at least ameliorating and/or decreasing and/or eradicating aspects of the disease such as the following: the size of a tumor may be lessened and/or the tumor may be completely destroyed; remnants of a tumor (e.g. after surgery) may be lessened and/or destroyed; the growth of a tumor may be prevented and/or the growth rate may be slowed; the metastatic potential of a tumor may be decreased or eliminated; cancer cells may be sensitized to radiation therapy, etc. For example, when cancer cells are exposed to a compound or drug described herein prior to, during or after radiation therapy, they are more susceptible to killing by radiation, e.g. at least about 25% more of the cancer cells die without dividing, and typically at least about 50, 75 or even 100% of the cells die without dividing, compared to the number that die when exposed to radiation alone. In addition, in embodiments, select changes which typically occur in cancer cells when exposed to radiation are decreased or eliminated when a compound or drug described herein is administered to a subject receiving radiotherapy. For example, cancer cells frequently exhibit an increased ability to grow, divide, and/or metastasize after radiation therapy, and administration of the present drugs (e.g., compound described herein) attenuates or eliminates this ability. In embodiments, the treatment of cancer metastasis includes the treatment of at least one of invasion, migration, and angiogenesis.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” For example, for a given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist or inhibitor (e.g., PDZ1 domain binder) required to disrupt the function of an enzyme or protein (e.g., MDA-9) relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a pharmaceutical composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The pharmaceutical compositions of the present disclosure can be delivered transdermally, by a topical route, or formulated as applicator sticks, solutions, suspensions, emulsions, gels, creams, ointments, pastes, jellies, paints, powders, and aerosols.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

In embodiments, a patient who is treated by the compounds described herein, including embodiments thereof (e.g., as pure drugs, salts, or prodrugs), and methods disclosed herein suffers from cancer. Examples of cancer that may be so treated include but are not limited to: adrenal cortical cancer, anal cancer, bile duct cancer (e.g. peripheral cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, glioma, glioblastoma, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), urothelial cell cancer, vaginal cancer, vulvar cancer, and uterine cancer (e.g. uterine leiomyosarcoma). Generally, the cancer is characterized by the presence of at least one solid tumor.

The term “associated” or “associated with” in the context of a substance or substance activity or function associated with a disease (e.g., cancer (e.g. melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, or neuroblastoma) means that the disease (e.g. melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, or neuroblastoma) is caused by (in whole or in part), or a symptom of the disease is caused by (in whole or in part) the substance or substance activity or function.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The compounds and pharmaceutical compositions described herein can be used in combination with one another, with other active agents known to be useful in treating a cancer such as anti-cancer agents. A pharmaceutical composition may comprise one or more active agents (e.g., an agent that inhibits MDA-9 and an anti-cancer agent), and a pharmaceutically acceptable excipient. In embodiments, combinations of active agents are provided as a single dosage form. In embodiments, agents used in combination are formulated in separate dosage forms.

Examples of anti-cancer agents include, but are not limited to, radiation, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bi; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfim; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), Cryptophycin 52 (i.e. LY-355703), Vitilevuamide, Tubulysin A, Canadensol, Centaureidin (i.e. NSC-106969), Oncocidin A1 (i.e. BTO-956 and DIME), Fijianolide B, Laulimalide, Narcosine (also known as NSC-5366), Nascapine, Vanadocene acetylacetonate, T-138026 (Tularik), Monsatrol, lnanocine (i.e. NSC-698666), Eleutherobins (such as Desmethyleleutherobin, Desaetyleleutherobin, lsoeleutherobin A, and Z-Eleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), 5-FU, MCL-1 inhibitor, sorafenib, imatinib, sunitinib, dasatinib, or the like. In embodiments, the compounds and pharmaceutical compositions herein may be used in combination with adjunctive agents that may not be effective alone, but may contribute to the efficacy of the active agent in treating cancer.

“Chemotherapeutic” or “chemotherapeutic agent” is used in accordance with its plain ordinary meaning and refers to a chemical composition or compound having antineoplastic properties or the ability to inhibit the growth or proliferation of cells.

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis. In embodiments, the inflammatory disease is asthma. In embodiments, the disease associated with elevated expression of mda-9 is an inflammatory disease.

As used herein, the term “neurodegenerative disorder” or “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The term “infection” or “infectious disease” refers to a disease or condition that can be caused by organisms such as a bacterium, virus, fungi or any other pathogenic microbial agents. In embodiments, the infectious diseases is a viral infection (e.g., HIV, SARS, HPV, influenza), or bacterial colonization in the human gastrointestinal tract (e.g., pathenogenic bacterial colonization). In embodiments, the infectious disease is associated with elevated expression of mda-9. In embodiments, the infectious disease is characterized by the presence of virus shedding (e.g., HIV viral shedding or Herpes viral shedding). In embodiments, the infectious disease is a bacterial infection. In embodiments, the infectious disease is a gram-positive bacterial infection. In embodiments, the infectious disease is a Staphylococcus aureus infection. In embodiments, the infectious disease is Gram-positive or Gram-negative bacterial infection. In embodiments, the infectious disease is an infection associated with S. aureus, E. facium, E. faecalis, K. pneumonoiaea, H. influenzaea, or P. aeruginosa. In embodiments, the infectious disease is a S. aureus, E. facium, E. faecalis, K. pneumonoiaea, H. influenzaea, or P. aeruginosa infection.

### II. Compounds

In embodiments, the present disclosure provides pharmaceutical compositions comprising (a) an agent that inhibits MDA-9, and (b) an anti-cancer agent, wherein the anti-cancer agent is a mitotic inhibitor or a histone deacetylase (HDAC) inhibitor.

In embodiments, the agent that inhibits MDA-9 is represented by a chemical structure disclosed herein. In embodiments the agent that inhibits MDA-9 is a compound that inhibits MDA-9, or pharmaceutically acceptable salt thereof, having the formula:

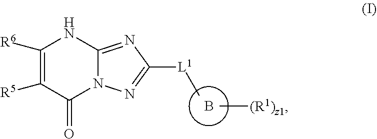

or a tautomer thereof.

Ring B is a cycloalkyl, heterocycloalkyl, aryl, or heteroaryl.

R¹ is independently oxo, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

L¹ is a bond, —S(O)₂—, —N(R³)—, —O—, —S—, —C(O)—, —C(O)N(R³)—, —N(R³)C(O)—, —N(R³)C(O)NH—, —NHC(O)N(R³)—, —S(O)₂N(R³)—, —N(R³)S(O)₂—, —C(O)S(O)₂N(R³)—, —N(R³)S(O)₂C(O)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R³ is independently hydrogen, —CX³₃, —CHX³₂, —CH₂X³, —CN, —C(O)R^{3C}, —C(O)OR^{3C}, —C(O)NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is independently hydrogen,

halogen, —CX⁵₃, —CHX⁵₂, —CH₂X⁵, —OCX⁵₃, —OCH₂X⁵, —OCHX⁵₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ is independently hydrogen,

halogen, —CX⁶₃, —CHX⁶₂, —CH₂X⁶, —OCX⁶₃, —OCH₂X⁶, —OCHX⁶₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ and R⁶ substituents may optionally be joined to form a substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{3A}, R^{3B}, and R^{3C} are independently

hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A} and R^{1B} substituents bonded to the same nitrogen atom may optionally be joined to form a substituted or unsubstituted heterocycloalkyl or substituted or unsubstituted heteroaryl. R^{3A} and R^{3B} substituents bonded to the same nitrogen atom may optionally be joined to form a substituted or unsubstituted heterocycloalkyl or substituted or unsubstituted heteroaryl.

X, X¹, X³, X⁵, and X⁶ are independently —F, —Cl, —Br, or —I. The symbol n1 is an integer from 0 to 4. The symbols m1 and v1 are independently 1 or 2. The symbol z1 is an integer from 0 to 5.

In embodiments, Ring B is a (C₃-C₁₀) cycloalkyl, a 3 to 10 membered heterocycloalkyl, a (C₆-C₁₀) aryl, or a 5 to 10 membered heteroaryl. In embodiments, Ring B is a cycloalkyl (e.g., C₃-C₈ cycloalkyl, C₃-C₆ cycloalkyl, or C₅-C₆ cycloalkyl). In embodiments, Ring B is a C₃-C₈ cycloalkyl. In embodiments, Ring B is a C₃-C₆ cycloalkyl. In embodiments, Ring B is a C₅-C₆ cycloalkyl. In embodiments, Ring B is a C₆ cycloalkyl. In embodiments, Ring B is a C₅ cycloalkyl. In embodiments, Ring B is a (C₆-C₁₀) aryl. In embodiments, Ring B is a 5 to 10 membered heteroaryl. In embodiments, Ring B is a 5 membered heteroaryl. In embodiments, Ring B is a 6 membered heteroaryl. In embodiments, Ring B is a 9 membered heteroaryl. In embodiments, Ring B is a 10 membered heteroaryl. In embodiments, Ring B is phenyl. In embodiments, Ring B is naphthyl. In embodiments, Ring B is aziridinyl, oxiranyl, thiiranyl, azetidinyl, oxetanyl, thietanyl, pyrrolidinyl, pyrrolyl, imidazolyl, imidazolinyl, pyrazolinyl, tetrahydrofuranyl, thiolanyl, piperidinyl, piperazinyl, pyranyl, morpholinyl, 1,4-dioxanyl, tetrahydro-2H-pyranyl, thianyl, or dithianyl. In embodiments, Ring B is a phenyl, thiofuranyl, imidazolyl, pyrazolyl, triazolyl, tetrazolyl, furanyl, oxazolyl, isooxazolyl, oxadiazolyl, oxatriazolyl, thienyl, thiazolyl, isothiazolyl, pyridinyl, pyrazinyl, pyrimidinyl, pyridazinyl, or triazinyl (e.g., 1,3,5-triazinyl, 1,2,3-triazinyl, or 1,2,4-triazinyl). In embodiments, Ring B is indolyl, benzimidazolyl, indazolyl, benzotriazolyl, pyrrolopyrimidinyl, purinyl, indolizinyl, pyrrolopyriazinyl, pyrrolopyrimidinyl, imidazopyridazinyl, imidazopyridinyl, imidazopyrimidinyl, cinnolinyl, quinazolinyl, quinoxalinyl, phthalazinyl, pyridopyrazinyl, pteridinyl, pyrazolopyridinyl, quinolinyl, isoquinolinyl, naphthyridinyl, or carbazolyl. In embodiments, Ring B is

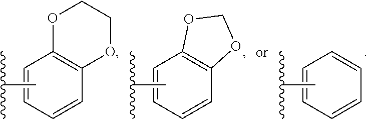

In embodiments, R⁵ is hydrogen, halogen, unsubstituted methyl, unsubstituted ethyl, unsubstituted propyl (e.g., unsubstituted n-propyl or unsubstituted isopropyl). In embodiments, R⁵ is a substituted or unsubstituted C₁-C₆ alkyl. In embodiments, R⁵ is substituted (e.g., substituted with a substituent group, a size-limited substituent group, or lower substituent group) or unsubstituted alkyl. In embodiments, R⁵ is substituted (e.g., substituted with a substituent group, a size-limited substituent group, or lower substituent group) alkyl. In embodiments, R⁵ is unsubstituted alkyl. In embodiments, R⁵ is substituted or unsubstituted alkyl (e.g., C₁-C₈, C₁-C₆, C₁-C₄, or C₁-C₂). In embodiments, R⁵ is substituted alkyl (e.g., C₁-C₈, C₁-C₆, C₁-C₄, or C₁-C₂). In embodiments, R⁵ is unsubstituted alkyl (e.g., C₁-C₈, C₁-C₆, C₁-C₄, or C₁-C₂).

In embodiments, R⁵ is independently hydrogen,

halogen, —CX⁵₃, —CHX⁵₂, —CH₂X⁵, —OCX⁵₃, —OCH₂X⁵, —OCHX⁵₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is independently unsubstituted methyl, unsubstituted ethyl, unsubstituted isopropyl, or unsubstituted tert-butyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted n-propyl. In embodiments, R⁵ is independently unsubstituted isopropyl. In embodiments, R³ is independently unsubstituted butyl. In embodiments, R³ is independently unsubstituted n-butyl. In embodiments, R⁵ is independently unsubstituted isobutyl. In embodiments, R⁵ is independently unsubstituted tert-butyl. In embodiments, R⁵ is independently unsubstituted pentyl. In embodiments, R⁵ is independently unsubstituted hexyl. In embodiments, R⁵ is independently unsubstituted heptyl. In embodiments, R⁵ is independently unsubstituted octyl.

In embodiments, R⁵ is substituted (e.g., substituted with a substituent group, a size-limited substituent group, or lower substituent group) or unsubstituted heteroalkyl. In embodiments, R⁵ is substituted (e.g., substituted with a substituent group, a size-limited substituent group, or lower substituent group) heteroalkyl. In embodiments, R⁵ is unsubstituted heteroalkyl. In embodiments, R⁵ is substituted or unsubstituted heteroalkyl (e.g., 2 to 8 membered, 2 to 6 membered, 4 to 6 membered, 2 to 3 membered, or 4 to 5 membered). In embodiments, R⁵ is substituted heteroalkyl (e.g., 2 to 8 membered, 2 to 6 membered, 4 to 6 membered, 2 to 3 membered, or 4 to 5 membered). In embodiments, R⁵ is an unsubstituted heteroalkyl (e.g., 2 to 8 membered, 2 to 6 membered, 4 to 6 membered, 2 to 3 membered, or 4 to 5 membered).

In embodiments, R⁶ is independently hydrogen,

halogen, —CX⁶₃, —CHX⁶₂, —CH₂X⁶, —OCX⁶₃, —OCH₂X⁶, —OCHX⁶₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, substituted (e.g., substituted with at least one substituent group, size-limited substituent group, or lower substituent group) or unsubstituted alkyl (e.g., C₁-C₈, C₁-C₆, C₁-C₄, or C₁-C₂), substituted (e.g., substituted with at least one substituent group, size-limited substituent group, or lower substituent group) or unsubstituted heteroalkyl (e.g., 2 to 8 membered, 2 to 6 membered, 4 to 6 membered, 2 to 3 membered, or 4 to 5 membered), substituted (e.g., substituted with at least one substituent group, size-limited substituent group, or lower substituent group) or unsubstituted cycloalkyl (e.g., C₃-C₈, C₃-C₆, C₄-C₆, or C₅-C₆), substituted (e.g., substituted with at least one substituent group, size-limited substituent group, or lower substituent group) or unsubstituted heterocycloalkyl (e.g., 3 to 8 membered, 3 to 6 membered, 4 to 6 membered, 4 to 5 membered, or 5 to 6 membered), substituted (e.g., substituted with at least one substituent group, size-limited substituent group, or lower substituent group) or unsubstituted aryl (e.g., C₆-C₁₀ or phenyl), or substituted (e.g., substituted with at least one substituent group, size-limited substituent group, or lower substituent group) or unsubstituted heteroaryl (e.g., 5 to 10 membered, 5 to 9 membered, or 5 to 6 membered).

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁶ is hydrogen, halogen, unsubstituted methyl, unsubstituted ethyl, unsubstituted propyl (e.g., unsubstituted n-propyl or unsubstituted isopropyl). In embodiments, R⁶ is a substituted or unsubstituted C₁-C₆ alkyl. In embodiments, R⁶ is substituted (e.g., substituted with a substituent group, a size-limited substituent group, or lower substituent group) or unsubstituted alkyl. In embodiments, R⁶ is substituted (e.g., substituted with a substituent group, a size-limited substituent group, or lower substituent group) alkyl. In embodiments, R⁶ is unsubstituted alkyl. In embodiments, R⁶ is substituted or unsubstituted alkyl (e.g., C₁-C₈, C₁-C₆, C₁-C₄, or C₁-C₂). In embodiments, R⁶ is substituted alkyl (e.g., C₁-C₈, C₁-C₆, C₁-C₄, or C₁-C₂). In embodiments, R⁶ is unsubstituted alkyl (e.g., C₁-C₈, C₁-C₆, C₁-C₄, or C₁-C₂).

In embodiments, R⁶ is substituted (e.g., substituted with a substituent group, a size-limited substituent group, or lower substituent group) or unsubstituted heteroalkyl. In embodiments, R⁶ is substituted (e.g., substituted with a substituent group, a size-limited substituent group, or lower substituent group) heteroalkyl. In embodiments, R⁶ is unsubstituted heteroalkyl. In embodiments, R⁶ is substituted or unsubstituted heteroalkyl (e.g., 2 to 8 membered, 2 to 6 membered, 4 to 6 membered, 2 to 3 membered, or 4 to 5 membered). In embodiments, R⁶ is substituted heteroalkyl (e.g., 2 to 8 membered, 2 to 6 membered, 4 to 6 membered, 2 to 3 membered, or 4 to 5 membered). In embodiments, R⁶ is an unsubstituted heteroalkyl (e.g., 2 to 8 membered, 2 to 6 membered, 4 to 6 membered, 2 to 3 membered, or 4 to 5 membered).

In embodiments, R⁶ is independently unsubstituted methyl, unsubstituted ethyl, unsubstituted isopropyl, or unsubstituted tert-butyl. In embodiments, R⁶ is independently unsubstituted methyl. In embodiments, R⁶ is independently unsubstituted ethyl. In embodiments, R⁶ is independently unsubstituted propyl. In embodiments, R⁶ is independently unsubstituted n-propyl. In embodiments, R⁶ is independently unsubstituted isopropyl. In embodiments, R⁶ is independently unsubstituted butyl. In embodiments, R⁶ is independently unsubstituted n-butyl. In embodiments, R⁶ is independently unsubstituted isobutyl. In embodiments, R⁶ is independently unsubstituted tert-butyl. In embodiments, R⁶ is independently unsubstituted pentyl. In embodiments, R⁶ is independently unsubstituted hexyl. In embodiments, R⁶ is independently unsubstituted heptyl. In embodiments, R⁶ is independently unsubstituted octyl.

In embodiments, R⁵ and R⁶ are joined to form a substituted or unsubstituted cyclopropyl, substituted or unsubstituted cyclobutyl, substituted or unsubstituted cyclopentyl, or substituted or unsubstituted cyclohexyl. In embodiments, R⁵ and R⁶ are joined to form a substituted cyclopropyl, substituted cyclobutyl, substituted cyclopentyl, or substituted cyclohexyl. In embodiments, R⁵ and R⁶ are joined to form an unsubstituted cyclopropyl, unsubstituted cyclobutyl, an unsubstituted cyclopentyl, or an unsubstituted cyclohexyl. In embodiments, R⁵ and R⁶ are joined to form a substituted cyclopropyl, substituted cyclopentyl, or substituted cyclohexyl. In embodiments, R⁵ and R⁶ are joined to form an unsubstituted cyclopropyl, an unsubstituted cyclopentyl, or an unsubstituted cyclohexyl.

In embodiments, R⁵ and R⁶ are joined to form a substituted or unsubstituted cycloalkyl (e.g., C₃-C₈, C₃-C₆, C₄-C₆, or C₅-C₆). In embodiments, R⁵ and R⁶ are joined to form a substituted (e.g., substituted with a substituent group, a size-limited substituent group, or lower substituent group) or unsubstituted cycloalkyl (e.g., C₃-C₈, C₃-C₆, C₄-C₆, or C₅-C₆). In embodiments, R⁵ and R⁶ are joined to form a substituted (e.g., substituted with a substituent group, a size-limited substituent group, or lower substituent group) cycloalkyl (e.g., C₃-C₈, C₃-C₆, C₄-C₆, or C₅-C₆). In embodiments, R⁵ and R⁶ are joined to form an unsubstituted cycloalkyl (e.g., C₃-C₈, C₃-C₆, C₄-C₆, or C₅-C₆).

In embodiments, R⁵ and R⁶ are joined to form a substituted (e.g., substituted with a substituent group, a size-limited substituent group, or lower substituent group) or unsubstituted heterocycloalkyl (e.g., 3 to 8 membered, 3 to 6 membered, 4 to 6 membered, 4 to 5 membered, or 5 to 6 membered). In embodiments, R⁵ and R⁶ are joined to form a substituted (e.g., substituted with a substituent group, a size-limited substituent group, or lower substituent group) heterocycloalkyl (e.g., 3 to 8 membered, 3 to 6 membered, 4 to 6 membered, 4 to 5 membered, or 5 to 6 membered). In embodiments, R⁵ and R⁶ are joined to form an unsubstituted heterocycloalkyl (e.g., 3 to 8 membered, 3 to 6 membered, 4 to 6 membered, 4 to 5 membered, or 5 to 6 membered).

In embodiments, R⁵ and R⁶ are joined to form a substituted or unsubstituted cyclopropenyl, substituted or unsubstituted cyclobutenyl, substituted or unsubstituted cyclopentenyl, or substituted or unsubstituted cyclohexenyl. In embodiments, R⁵ and R⁶ are joined to form a substituted cyclopropenyl, substituted cyclobutenyl, substituted cyclopentenyl, or substituted cyclohexenyl. In embodiments, R⁵ and R⁶ are joined to form an unsubstituted cyclopropenyl, unsubstituted cyclobutenyl, an unsubstituted cyclopentenyl, or an unsubstituted cyclohexenyl. In embodiments, R⁵ and R⁶ are joined to form a substituted cyclopropenyl, substituted cyclopentenyl, or substituted cyclohexenyl. In embodiments, R⁵ and R⁶ are joined to form an unsubstituted cyclopropenyl, an unsubstituted cyclopentenyl, or an unsubstituted cyclohexenyl.

In embodiments, R⁵ and R⁶ are joined to form a substituted or unsubstituted cycloalkenyl (e.g., C₃-C₈, C₃-C₆, C₄-C₆, or C₅-C₆). In embodiments, R⁵ and R⁶ are joined to form a substituted (e.g., substituted with a substituent group, a size-limited substituent group, or lower substituent group) or unsubstituted cycloalkenyl (e.g., C₃-C₈, C₃-C₆, C₄-C₆, or C₅-C₆). In embodiments, R⁵ and R⁶ are joined to form a substituted (e.g., substituted with a substituent group, a size-limited substituent group, or lower substituent group) cycloalkenyl (e.g., C₃-C₈, C₃-C₆, C₄-C₆, or C₅-C₆). In embodiments, R⁵ and R⁶ are joined to form an unsubstituted cycloalkenyl (e.g., C₃-C₈, C₃-C₆, C₄-C₆, or C₅-C₆).

In embodiments, R⁵ and R⁶ are joined to form a substituted (e.g., substituted with a substituent group, a size-limited substituent group, or lower substituent group) or unsubstituted heterocycloalkenyl (e.g., 3 to 8 membered, 3 to 6 membered, 4 to 6 membered, 4 to 5 membered, or 5 to 6 membered). In embodiments, R⁵ and R⁶ are joined to form a substituted (e.g., substituted with a substituent group, a size-limited substituent group, or lower substituent group) heterocycloalkenyl (e.g., 3 to 8 membered, 3 to 6 membered, 4 to 6 membered, 4 to 5 membered, or 5 to 6 membered). In embodiments, R⁵ and R⁶ are joined to form an unsubstituted heterocycloalkenyl (e.g., 3 to 8 membered, 3 to 6 membered, 4 to 6 membered, 4 to 5 membered, or 5 to 6 membered).

In embodiments, two adjacent R¹ substituents are joined to form a substituted or unsubstituted (C₃-C₁₀) cycloalkyl, substituted or unsubstituted 3 to 10 membered heterocycloalkyl, substituted or unsubstituted (C₆-C₁₀) aryl, or substituted or unsubstituted 5 to 10 membered heteroaryl.

In embodiments, two adjacent R¹ substituents are joined to form a substituted or unsubstituted aziridinyl, substituted or unsubstituted oxiranyl, substituted or unsubstituted thiiranyl, substituted or unsubstituted azetidinyl, substituted or unsubstituted oxetanyl, substituted or unsubstituted thietanyl, substituted or unsubstituted pyrrolidinyl, substituted or unsubstituted pyrrolyl, substituted or unsubstituted imidazolyl, substituted or unsubstituted imidazolinyl, substituted or unsubstituted pyrazolinyl, substituted or unsubstituted tetrahydrofuranyl, substituted or unsubstituted thiolanyl, substituted or unsubstituted piperidinyl, substituted or unsubstituted piperazinyl, substituted or unsubstituted pyranyl, substituted or unsubstituted morpholinyl, substituted or unsubstituted 1,4-dioxanyl, substituted or unsubstituted tetrahydro-2H-pyranyl, substituted or unsubstituted thianyl, or substituted or unsubstituted dithianyl. In embodiments, two adjacent R¹ substituents are joined to form a substituted aziridinyl, substituted oxiranyl, substituted thiiranyl, substituted azetidinyl, substituted oxetanyl, substituted thietanyl, substituted pyrrolidinyl, substituted pyrrolyl, substituted imidazolyl, substituted imidazolinyl, substituted pyrazolinyl, substituted tetrahydrofuranyl, substituted thiolanyl, substituted piperidinyl, substituted piperazinyl, substituted pyranyl, substituted morpholinyl, substituted 1,4-dioxanyl, substituted tetrahydro-2H-pyranyl, substituted thianyl, or substituted dithianyl. In embodiments, two adjacent R¹ substituents are joined to form an unsubstituted aziridinyl, unsubstituted oxiranyl, unsubstituted thiiranyl, unsubstituted azetidinyl, unsubstituted oxetanyl, unsubstituted thietanyl, unsubstituted pyrrolidinyl, unsubstituted pyrrolyl, unsubstituted imidazolyl, unsubstituted imidazolinyl, unsubstituted pyrazolinyl, unsubstituted tetrahydrofuranyl, unsubstituted thiolanyl, unsubstituted piperidinyl, unsubstituted piperazinyl, unsubstituted pyranyl, unsubstituted morpholinyl, unsubstituted 1,4-dioxanyl, unsubstituted tetrahydro-2H-pyranyl, unsubstituted thianyl, or unsubstituted dithianyl.

In an aspect is provided a compound (agent) that inhibits MDA-9, or a pharmaceutically acceptable salt thereof, having the formula:

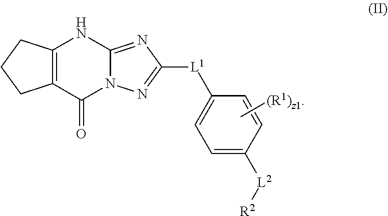

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)^{m1}, —NR^{1A}R^{1B}, —C (O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C (O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹ is a

bond, —S(O)₂—, —N(R³)—, —O—, —S—, —C(O)—, —C(O)N(R³)—, —N(R³)C(O)—, —N(R³)C(O)NH—, —NHC(O)N(R³)—, —S(O)₂N(R³)—, N(R³)S(O)₂—, —C(O)S(O)₂N(R³)—, —N(R³)S(O)₂C(O)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R³ is independently hydrogen, —CX³₃, —CHX³₂, —CH₂X³, —CN, —C(O)R^{3C}, —C(O)OR^{3C}, —C(O)NR^{3A}R^{3B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is a

bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —S(O)₂N(R⁴)—, N(R⁴)S(O)₂—, —C(O)S(O)₂N(R⁴)—, —N(R⁴)S(O)₂C(O)—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R⁴ is independently hydrogen, —CX⁴₃, —CHX⁴₂, —CH₂X⁴, —CN, —C(O)R^{4C}, —C(O)OR^{4C}, —C(O)NR^{4A}R^{4B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{3A}, R^{3B}, R^{3C}, R^{4A}, R^{4B}, and R^{4C} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A} and R^{1B} substituents bonded to the same nitrogen atom may optionally be joined to form a substituted or unsubstituted heterocycloalkyl or substituted or unsubstituted heteroaryl. R^{2A} and R^{2B} substituents bonded to the same nitrogen atom may optionally be joined to form a substituted or unsubstituted heterocycloalkyl or substituted or unsubstituted heteroaryl. R^{3A} and R^{3B} substituents bonded to the same nitrogen atom may optionally be joined to form a substituted or unsubstituted heterocycloalkyl or substituted or unsubstituted heteroaryl. R^{4A} and R^{3B} substituents bonded to the same nitrogen atom may optionally be joined to form a substituted or unsubstituted heterocycloalkyl or substituted or unsubstituted heteroaryl.

X, X¹, X², X³, and X⁴ are independently —F, —Cl, —Br, or —I. The symbols n1 and n2 are independently an integer from 0 to 4. The symbols m1, m2, v1, and v2 are independently 1 or 2. The symbol z1 is an integer from 0 to 4.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C (O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R¹ is independently

halogen, —CX¹³, —CN, —OH, —NH₂, —SH, —OCX¹₃, —OCHX¹₂, —OCH₂X¹, —CHX¹₂, —CH₂X¹, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹ is independently
halogen, —CX¹₃, —CN, —OR^{2D}, —NH₂, —SH, —OCX¹₃, —OCHX¹₂, —OCH₂X¹, —CHX¹₂, —CH₂X¹, unsubstituted C₁-C₄ alkyl, or unsubstituted 2 to 4 membered heteroalkyl.

In embodiments, R¹ is independently halogen, —CX¹₃, —CHX¹₂, —CH₂X¹, —OCX¹₃, —OR^{2D}, —CN, —NR^{1A}R^{1B}, substituted or unsubstituted alkyl or two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. In embodiments, R¹ is independently halogen, —CX¹₃, —CHX¹₂, —CH₂X¹, —OCX¹₃, —OR^{2D}, —CN, —NR^{1A}R^{1B}, or substituted or unsubstituted alkyl.

In embodiments, R¹ is independently,

halogen, —CX¹₃, —CHX¹₂, —CH₂X¹, —OCX¹₃, —OCH₂X¹, —OCHX¹₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²-substituted or unsubstituted alkyl (e.g., C₁-C₈, C₁-C₆, C₁-C₄, or C₁-C₂), R²⁰-substituted or unsubstituted heteroalkyl (e.g., 2 to 8 membered, 2 to 6 membered, 4 to 6 membered, 2 to 3 membered, or 4 to 5 membered), R²⁰-substituted or unsubstituted cycloalkyl (e.g., C₃-C₈, C₃-C₆, C₄-C₆, or C₅-C₆), R²⁰-substituted or unsubstituted heterocycloalkyl (e.g., 3 to 8 membered, 3 to 6 membered, 4 to 6 membered, 4 to 5 membered, or 5 to 6 membered), R²⁰-substituted or unsubstituted aryl (e.g., C₆-C₁₀ or phenyl), or R²⁰-substituted or unsubstituted heteroaryl (e.g., 5 to 10 membered, 5 to 9 membered, or 5 to 6 membered). In embodiments, R¹ is independently hydrogen,
halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X¹ is independently —F, —Cl, —Br, or —I.

In embodiments, R¹ is independently hydrogen, halogen, —NR^{1A}R^{1B}, —OR^{1D}, or substituted or unsubstituted heteroaryl. In embodiments, R¹ is independently substituted or unsubstituted aryl. In embodiments, R¹ is independently substituted or unsubstituted phenyl. In embodiments, R¹ is independently hydrogen. In embodiments, R¹ is independently halogen. In embodiments, R¹ is independently —NR^{1A}R^{1B}. In embodiments, R¹ is independently —OR^{1D}. In embodiments, R¹ is independently substituted or unsubstituted heteroaryl. In embodiments, R¹ is independently substituted heteroaryl. In embodiments, R¹ is independently substituted 5 to 6 membered heteroaryl. In embodiments, R¹ is independently —NH₂. In embodiments, R¹ is independently —OH. In embodiments, R¹ is independently —Cl. In embodiments, R¹ is independently —F. In embodiments, R¹ is independently halogen.

In embodiments, R¹ is independently R²⁰-substituted or unsubstituted alkyl or R²⁰-substituted or unsubstituted heteroalkyl. In embodiments, R¹ is independently R²⁰-substituted methyl. In embodiments, R¹ is independently R²⁰-substituted ethyl. In embodiments, R¹ is independently unsubstituted methyl. In embodiments, R¹ is independently unsubstituted ethyl. In embodiments, R¹ is independently halogen, —OR^{2D}, or —CH₃. In embodiments, R¹ is independently halogen. In embodiments, R¹ is independently —CH₃. In embodiments, R¹ is independently —OR^{2D}. In embodiments, R¹ is independently —OH.

In embodiments, R¹ is independently R²⁰-substituted or unsubstituted alkyl (e.g., C₁-C₈ alkyl, C₁-C₆ alkyl, or C₁-C₄ alkyl). In embodiments, R¹ is independently R²⁰-substituted alkyl (e.g., C₁-C₈ alkyl, C₁-C₆ alkyl, or C₁-C₄ alkyl). In embodiments, R¹ is independently an unsubstituted alkyl (e.g., C₁-C₈ alkyl, C₁-C₆ alkyl, or C₁-C₄ alkyl). In embodiments, R¹ is independently R²⁰-substituted or unsubstituted heteroalkyl (e.g., 2 to 8 membered heteroalkyl, 2 to 6 membered heteroalkyl, or 2 to 4 membered heteroalkyl). In embodiments, R¹ is independently R²⁰-substituted heteroalkyl (e.g., 2 to 8 membered heteroalkyl, 2 to 6 membered heteroalkyl, or 2 to 4 membered heteroalkyl). In embodiments, R¹ is independently an unsubstituted heteroalkyl (e.g., 2 to 8 membered heteroalkyl, 2 to 6 membered heteroalkyl, or 2 to 4 membered heteroalkyl). In embodiments, R¹ is independently R²⁰-substituted or unsubstituted cycloalkyl (e.g., C₃-C₈ cycloalkyl, C₃-C₆ cycloalkyl, or C₅-C₆ cycloalkyl). In embodiments, R¹ is independently R²⁰-substituted cycloalkyl (e.g., C₃-C₈ cycloalkyl, C₃-C₆ cycloalkyl, or C₅-C₆ cycloalkyl). In embodiments, R¹ is independently an unsubstituted cycloalkyl (e.g., C₃-C₈ cycloalkyl, C₃-C₆ cycloalkyl, or C₅-C₆ cycloalkyl). In embodiments, R¹ is independently R²⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is independently R²⁰-substituted heterocycloalkyl (e.g., 3 to 8 membered heterocycloalkyl, 3 to 6 membered heterocycloalkyl, or 5 to 6 membered heterocycloalkyl). In embodiments, R¹ is independently an unsubstituted heterocycloalkyl (e.g., 3 to 8 membered heterocycloalkyl, 3 to 6 membered heterocycloalkyl, or 5 to 6 membered heterocycloalkyl). In embodiments, R¹ is independently R²⁰-substituted or unsubstituted aryl (e.g., C₆-C₁₀ aryl, C₁₀ aryl, or phenyl). In embodiments, R¹ is independently R²⁰-substituted aryl (e.g., C₆-C₁₀ aryl, C₁₀ aryl, or phenyl). In embodiments, R¹ is independently an unsubstituted aryl (e.g., C₆-C₁₀ aryl, C₁₀ aryl, or phenyl). In embodiments, R¹ is independently R²⁰-substituted or unsubstituted heteroaryl (e.g., 5 to 10 membered heteroaryl, 5 to 9 membered heteroaryl, or 5 to 6 membered heteroaryl). In embodiments, R¹ is independently R²⁰-substituted heteroaryl (e.g., 5 to 10 membered heteroaryl, 5 to 9 membered heteroaryl, or 5 to 6 membered heteroaryl). In embodiments, R¹ is independently an unsubstituted heteroaryl (e.g., 5 to 10 membered heteroaryl, 5 to 9 membered heteroaryl, or 5 to 6 membered heteroaryl).

In embodiments, R¹ is independently R²⁰-substituted or unsubstituted methyl. In embodiments, R¹ is independently R²⁰-substituted or unsubstituted C₂ alkyl. In embodiments, R¹ is independently R²⁰-substituted or unsubstituted C₃ alkyl. In embodiments, R¹ is independently R²⁰-substituted or unsubstituted C₄ alkyl. In embodiments, R¹ is independently R²⁰-substituted or unsubstituted C₅ alkyl. In embodiments, R¹ is independently R²⁰-substituted or unsubstituted C₆ alkyl. In embodiments, R¹ is independently R²⁰-substituted or unsubstituted C₇ alkyl. In embodiments, R¹ is independently R²⁰-substituted or unsubstituted C₈ alkyl. In embodiments, R¹ is independently R²⁰-substituted methyl. In embodiments, R¹ is independently R²⁰-substituted C₂ alkyl. In embodiments, R¹ is independently R²⁰-substituted C₃ alkyl. In embodiments, R¹ is independently R²⁰-substituted C₄ alkyl. In embodiments, R¹ is independently R²⁰-substituted C₅ alkyl. In embodiments, R¹ is independently R²⁰-substituted C₆ alkyl. In embodiments, R¹ is independently R²⁰-substituted C₇ alkyl. In embodiments, R¹ is independently R²⁰-substituted C₅ alkyl. In embodiments, R¹ is independently an unsubstituted methyl. In embodiments, R¹ is independently an unsubstituted C₂ alkyl. In embodiments, R¹ is independently an unsubstituted C₃ alkyl. In embodiments, R¹ is independently an unsubstituted C₄ alkyl. In embodiments, R¹ is independently an unsubstituted C₅ alkyl. In embodiments, R¹ is independently an unsubstituted C₆ alkyl. In embodiments, R¹ is independently an unsubstituted C₇ alkyl. In embodiments, R¹ is independently an unsubstituted C₈ alkyl.

R²⁰ is independently oxo,

halogen, —CX²⁰₃, —CHX²⁰₂, —CH₂X²⁰, —OCX²⁰₃, —OCH₂X²⁰, —OCHX²⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²¹-substituted or unsubstituted alkyl (e.g., C₁-C₈, C₁-C₆, C₁-C₄, or C₁-C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₀ or phenyl), or R²¹-substituted or unsubstituted heteroaryl (e.g., 5 to 10 membered, 5 to 9 membered, or 5 to 6 membered). In embodiments, R²⁰ is independently oxo,
halogen, —CX²⁰₃, —CHX²⁰₂, —CH₂X²⁰, —OCX²⁰₃, —OCH₂X²⁰, —OCHX²⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁰ is independently —F, —Cl, —Br, or —I.

In embodiments, R²⁰ is independently R²¹-substituted or unsubstituted alkyl or R²¹-substituted or unsubstituted heteroalkyl. In embodiments, R²⁰ is independently R²¹-substituted methyl. In embodiments, R²⁰ is independently R²¹-substituted ethyl. In embodiments, R²⁰ is independently unsubstituted methyl. In embodiments, R² is independently unsubstituted ethyl.

R²¹ is independently oxo,

halogen, —CX²¹₃, —CHX²¹₂, —CH₂X²¹, —OCX²¹³, —OCH₂X²¹, —OCHX²¹², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, Ru-substituted or unsubstituted alkyl (e.g., C₁-C₈, C₁-C₆, C₁-C₄, or C₁-C₂), Ru-substituted or unsubstituted heteroalkyl (e.g., 2 to 8 membered, 2 to 6 membered, 4 to 6 membered, 2 to 3 membered, or 4 to 5 membered), R²²-substituted or unsubstituted cycloalkyl (e.g., C₃-C₈, C₃-C₆, C₄-C₆, or C₅-C₆), R²²-substituted or unsubstituted heterocycloalkyl (e.g., 3 to 8 membered, 3 to 6 membered, 4 to 6 membered, 4 to 5 membered, or 5 to 6 membered), R²²-substituted or unsubstituted aryl (e.g., C₆-C₁₀ or phenyl), or R²²-substituted or unsubstituted heteroaryl (e.g., 5 to 10 membered, 5 to 9 membered, or 5 to 6 membered). In embodiments, R²¹ is independently oxo,
halogen, —CX²¹₃, —CHX²¹₂, —CH₂X²¹, —OCX²¹₃, —OCH₂X²¹, —OCHX²¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²¹ is independently —F, —Cl, —Br, or —I.

In embodiments, R²¹ is independently R²²-substituted or unsubstituted alkyl or R²²-substituted or unsubstituted heteroalkyl. In embodiments, R²¹ is independently R²²-substituted methyl. In embodiments, R²¹ is independently R²²-substituted ethyl. In embodiments, R²¹ is independently unsubstituted methyl. In embodiments, R²¹ is independently unsubstituted ethyl.

R² is independently oxo,

halogen, —CX²²₃, —CHX²²₂, —CH₂X²², —OCX²²₃, —OCH₂X²², —OCHX²²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²² is independently —F, —Cl, —Br, or —I.

In embodiments, R^{1A} is independently substituted or unsubstituted alkyl (e.g., C₁-C₈, C₁-C₆, C₁-C₄, or C₁-C₂). In embodiments, R^{1A} is independently substituted alkyl (e.g., C₁-C₈, C₁-C₆, C₁-C₄, or C₁-C₂). In embodiments, R^{1A} is independently unsubstituted alkyl (e.g., C₁-C₈, C₁-C₆, C₁-C₄, or C₁-C₂). In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted isopropyl. In embodiments, R^{1A} is independently unsubstituted tert-butyl. In embodiments, R^{1A} is independently substituted or unsubstituted heteroalkyl (e.g., 2 to 8 membered, 2 to 6 membered, 4 to 6 membered, 2 to 3 membered, or 4 to 5 membered). In embodiments, R^{1A} is independently substituted heteroalkyl (e.g., 2 to 8 membered, 2 to 6 membered, 4 to 6 membered, 2 to 3 membered, or 4 to 5 membered). In embodiments, R^{1A} is independently unsubstituted heteroalkyl (e.g., 2 to 8 membered, 2 to 6 membered, 4 to 6 membered, 2 to 3 membered, or 4 to 5 membered). In embodiments, R^{1A} is independently substituted or unsubstituted cycloalkyl (e.g., C₃-C₈, C₃-C₆, C₄-C₆, or C₅-C₆). In embodiments, R^{1A} is independently substituted cycloalkyl (e.g., C₃-C₈, C₃-C₆, C₄-C₆, or C₅-C₆). In embodiments, R^{1A} is independently unsubstituted cycloalkyl (e.g., C₃-C₈, C₃-C₆, C₄-C₆, or C₅-C₆). In embodiments, R^{1A} is independently substituted or unsubstituted heterocycloalkyl (e.g., 3 to 8 membered, 3 to 6 membered, 4 to 6 membered, 4 to 5 membered, or 5 to 6 membered). In embodiments, R^{1A} is independently substituted heterocycloalkyl (e.g., 3 to 8 membered, 3 to 6 membered, 4 to 6 membered, 4 to 5 membered, or 5 to 6 membered). In embodiments, R^{1A} is independently unsubstituted heterocycloalkyl (e.g., 3 to 8 membered, 3 to 6 membered, 4 to 6 membered, 4 to 5 membered, or 5 to 6 membered). In embodiments, R^{1A} is independently substituted or unsubstituted aryl (e.g., C₆-C₁₀ or phenyl). In embodiments, R^{1A} is independently substituted aryl (e.g., C₆-C₁₀ or phenyl). In embodiments, R^{1A} is independently unsubstituted aryl (e.g., C₆-C₁₀ or phenyl). In embodiments, R^{1A} is independently substituted or unsubstituted heteroaryl (e.g., 5 to 10 membered, 5 to 9 membered, or 5 to 6 membered). In embodiments, R^{1A} is independently substituted heteroaryl (e.g., 5 to 10 membered, 5 to 9 membered, or 5 to 6 membered). In embodiments, R^{1A} is independently unsubstituted heteroaryl (e.g., 5 to 10 membered, 5 to 9 membered, or 5 to 6 membered).

In embodiments, R^{1B} is independently substituted or unsubstituted alkyl (e.g., C₁-C₈, C₁-C₆, C₁-C₄, or C₁-C₂). In embodiments, R^{1B} is independently substituted alkyl (e.g., C₁-C₈, C₁-C₆, C₁-C₄, or C₁-C₂). In embodiments, R^{1B} is independently unsubstituted alkyl (e.g., C₁-C₈, C₁-C₆, C₁-C₄, or C₁-C₂). In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted isopropyl. In embodiments, R^{1B} is independently unsubstituted tert-butyl. In embodiments, R^{1B} is independently substituted or unsubstituted heteroalkyl (e.g., 2 to 8 membered, 2 to 6 membered, 4 to 6 membered, 2 to 3 membered, or 4 to 5 membered). In embodiments, R^{1B} is independently substituted heteroalkyl (e.g., 2 to 8 membered, 2 to 6 membered, 4 to 6 membered, 2 to 3 membered, or 4 to 5 membered). In embodiments, R^{1B} is independently unsubstituted heteroalkyl (e.g., 2 to 8 membered, 2 to 6 membered, 4 to 6 membered, 2 to 3 membered, or 4 to 5 membered). In embodiments, R^{1B} is independently substituted or unsubstituted cycloalkyl (e.g., C₃-C₈, C₃-C₆, C₄-C₆, or C₅-C₆). In embodiments, R^{1B} is independently substituted cycloalkyl (e.g., C₃-C₈, C₃-C₆, C₄-C₆, or C₅-C₆). In embodiments, R^{1B} is independently unsubstituted cycloalkyl (e.g., C₃-C₈, C₃-C₆, C₄-C₆, or C₅-C₆). In embodiments, R^{1B} is independently substituted or unsubstituted heterocycloalkyl (e.g., 3 to 8 membered, 3 to 6 membered, 4 to 6 membered, 4 to 5 membered, or 5 to 6 membered). In embodiments, R^{1B} is independently substituted heterocycloalkyl (e.g., 3 to 8 membered, 3 to 6 membered, 4 to 6 membered, 4 to 5 membered, or 5 to 6 membered). In embodiments, R^{1B} is independently unsubstituted heterocycloalkyl (e.g., 3 to 8 membered, 3 to 6 membered, 4 to 6 membered, 4 to 5 membered, or 5 to 6 membered). In embodiments, R^{1B} is independently substituted or unsubstituted aryl (e.g., C₆-C₁₀ or phenyl). In embodiments, R^{1B} is independently substituted aryl (e.g., C₆-C₁₀ or phenyl). In embodiments, R^{1B} is independently unsubstituted aryl (e.g., C₆-C₁₀ or phenyl). In embodiments, R^{1B} is independently substituted or unsubstituted heteroaryl (e.g., 5 to 10 membered, 5 to 9 membered, or 5 to 6 membered). In embodiments, R^{1B} is independently substituted heteroaryl (e.g., 5 to 10 membered, 5 to 9 membered, or 5 to 6 membered). In embodiments, R^{1B} is independently unsubstituted heteroaryl (e.g., 5 to 10 membered, 5 to 9 membered, or 5 to 6 membered).

In embodiments, R^{1A} and R^{1B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{1A} and R^{1B} substituents bonded to the same nitrogen atom may be joined to form a substituted or unsubstituted heteroaryl (e.g., 5 to 10 membered, 5 to 9 membered, or 5 to 6 membered). In embodiments, R^{1A} and R^{1B} substituents bonded to the same nitrogen atom may be joined to form a substituted heteroaryl (e.g., 5 to 10 membered, 5 to 9 membered, or 5 to 6 membered). In embodiments, R^{1A} and R^{1B} substituents bonded to the same nitrogen atom may be joined to form an unsubstituted heteroaryl (e.g., 5 to 10 membered, 5 to 9 membered, or 5 to 6 membered).

In embodiments, R^{1C} is independently substituted or unsubstituted alkyl (e.g., C₁-C₈, C₁-C₆, C₁-C₄, or C₁-C₂). In embodiments, R^{1C} is independently substituted alkyl (e.g., C₁-C₈, C₁-C₆, C₁-C₄, or C₁-C₂). In embodiments, R^{1C} is independently unsubstituted alkyl (e.g., C₁-C₈, C₁-C₆, C₁-C₄, or C₁-C₂). In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted isopropyl. In embodiments, R^{1C} is independently unsubstituted tert-butyl. In embodiments, R^{1C} is independently substituted or unsubstituted heteroalkyl (e.g., 2 to 8 membered, 2 to 6 membered, 4 to 6 membered, 2 to 3 membered, or 4 to 5 membered). In embodiments, R^{1C} is independently substituted heteroalkyl (e.g., 2 to 8 membered, 2 to 6 membered, 4 to 6 membered, 2 to 3 membered, or 4 to 5 membered). In embodiments, R^{1C} is independently unsubstituted heteroalkyl (e.g., 2 to 8 membered, 2 to 6 membered, 4 to 6 membered, 2 to 3 membered, or 4 to 5 membered). In embodiments, R^{1C} is independently substituted or unsubstituted cycloalkyl (e.g., C₃-C₈, C₃-C₆, C₄-C₆, or C₅-C₆). In embodiments, R^{1C} is independently substituted cycloalkyl (e.g., C₃-C₈, C₃-C₆, C₄-C₆, or C₅-C₆). In embodiments, R^{1C} is independently unsubstituted cycloalkyl (e.g., C₃-C₈, C₃-C₆, C₄-C₆, or C₅-C₆). In embodiments, R^{1C} is independently substituted or unsubstituted heterocycloalkyl (e.g., 3 to 8 membered, 3 to 6 membered, 4 to 6 membered, 4 to 5 membered, or 5 to 6 membered). In embodiments, R^{1C} is independently substituted heterocycloalkyl (e.g., 3 to 8 membered, 3 to 6 membered, 4 to 6 membered, 4 to 5 membered, or 5 to 6 membered). In embodiments, R^{1C} is independently unsubstituted heterocycloalkyl (e.g., 3 to 8 membered, 3 to 6 membered, 4 to 6 membered, 4 to 5 membered, or 5 to 6 membered). In embodiments, R^{1C} is independently substituted or unsubstituted aryl (e.g., C₆-C₁₀ or phenyl). In embodiments, R^{1C} is independently substituted aryl (e.g., C₆-C₁₀ or phenyl). In embodiments, R^{1C} is independently unsubstituted aryl (e.g., C₆-C₁₀ or phenyl). In embodiments, R^{1C} is independently substituted or unsubstituted heteroaryl (e.g., 5 to 10 membered, 5 to 9 membered, or 5 to 6 membered). In embodiments, R^{1C} is independently substituted heteroaryl (e.g., 5 to 10 membered, 5 to 9 membered, or 5 to 6 membered). In embodiments, R^{1C} is independently unsubstituted heteroaryl (e.g., 5 to 10 membered, 5 to 9 membered, or 5 to 6 membered).

In embodiments, R^{1D} is independently substituted or unsubstituted alkyl (e.g., C₁-C₈, C₁-C₆, C₁-C₄, or C₁-C₂). In embodiments, R^{1D} is independently substituted alkyl (e.g., C₁-C₈, C₁-C₆, C₁-C₄, or C₁-C₂). In embodiments, R^{1D} is independently unsubstituted alkyl (e.g., C₁-C₈, C₁-C₆, C₁-C₄, or C₁-C₂). In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted isopropyl. In embodiments, R^{1D} is independently unsubstituted tert-butyl. In embodiments, R^{1D} is independently substituted or unsubstituted heteroalkyl (e.g., 2 to 8 membered, 2 to 6 membered, 4 to 6 membered, 2 to 3 membered, or 4 to 5 membered). In embodiments, R^{1D} is independently substituted heteroalkyl (e.g., 2 to 8 membered, 2 to 6 membered, 4 to 6 membered, 2 to 3 membered, or 4 to 5 membered). In embodiments, R^{1D} is independently unsubstituted heteroalkyl (e.g., 2 to 8 membered, 2 to 6 membered, 4 to 6 membered, 2 to 3 membered, or 4 to 5 membered). In embodiments, R^{1D} is independently substituted or unsubstituted cycloalkyl (e.g., C₃-C₈, C₃-C₆, C₄-C₆, or C₅-C₆). In embodiments, R^{1D} is independently substituted cycloalkyl (e.g., C₃-C₈, C₃-C₆, C₄-C₆, or C₅-C₆). In embodiments, R^{1D} is independently unsubstituted cycloalkyl (e.g., C₃-C₈, C₃-C₆, C₄-C₆, or C₅-C₆). In embodiments, R^{1D} is independently substituted or unsubstituted heterocycloalkyl (e.g., 3 to 8 membered, 3 to 6 membered, 4 to 6 membered, 4 to 5 membered, or 5 to 6 membered). In embodiments, R^{1D} is independently substituted heterocycloalkyl (e.g., 3 to 8 membered, 3 to 6 membered, 4 to 6 membered, 4 to 5 membered, or 5 to 6 membered). In embodiments, R^{1D} is independently unsubstituted heterocycloalkyl (e.g., 3 to 8 membered, 3 to 6 membered, 4 to 6 membered, 4 to 5 membered, or 5 to 6 membered). In embodiments, R^{1D} is independently substituted or unsubstituted aryl (e.g., C₆-C₁₀ or phenyl). In embodiments, R^{1D} is independently substituted aryl (e.g., C₆-C₁₀ or phenyl). In embodiments, R^{1D} is independently unsubstituted aryl (e.g., C₆-C₁₀ or phenyl). In embodiments, R^{1D} is independently substituted or unsubstituted heteroaryl (e.g., 5 to 10 membered, 5 to 9 membered, or 5 to 6 membered). In embodiments, R^{1D} is independently substituted heteroaryl (e.g., 5 to 10 membered, 5 to 9 membered, or 5 to 6 membered). In embodiments, R^{1D} is independently unsubstituted heteroaryl (e.g., 5 to 10 membered, 5 to 9 membered, or 5 to 6 membered).

In embodiments, R^{1A} is independently

hydrogen, —CX^{1A}₃, —CHX^{1A}₂, —CH₂X^{1A}, —CN, —COOH, —CONH₂, R^{20A}-substituted or unsubstituted alkyl (e.g., C₁-C₈, C₁-C₆, C₁-C₄, or C₁-C₂), R^{20A}-substituted or unsubstituted heteroalkyl (e.g., 2 to 8 membered, 2 to 6 membered, 4 to 6 membered, 2 to 3 membered, or 4 to 5 membered), R^{20A}-substituted or unsubstituted cycloalkyl (e.g., C₃-C₈, C₃-C₆, C₄-C₆, or C₅-C₆), R^{20A}-substituted or unsubstituted heterocycloalkyl (e.g., 3 to 8 membered, 3 to 6 membered, 4 to 6 membered, 4 to 5 membered, or 5 to 6 membered), R^{20A}-substituted or unsubstituted aryl (e.g., C₆-C₁₀ or phenyl), or R^{20A}-substituted or unsubstituted heteroaryl (e.g., 5 to 10 membered, 5 to 9 membered, or 5 to 6 membered). In embodiments, R^{1A} is independently
hydrogen, —CX^{1A}₃, —CHX^{1A}₂, —CH₂X^{1A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{1A} is independently —F, —Cl, —Br, or —I.

In embodiments, R^{1A} is independently hydrogen. In embodiments, R^{1A} is independently unsubstituted methyl. In embodiments, R^{1A} is independently unsubstituted ethyl.

In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered) or R^{20A}-substituted or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form a R^{20A}-substituted or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form an unsubstituted heteroaryl (e.g., 5 to 10 membered, 5 to 9 membered, or 5 to 6 membered).

R^{20A} is independently oxo,

halogen, —CX^{20A}₃, —CHX^{20A}₂, —CH₂X^{20A}, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{21A}-substituted or unsubstituted alkyl (e.g., C₁-C₈, C₁-C₆, C₁-C₄, or C₁-C₂), R^{21A}-substituted or unsubstituted heteroalkyl (e.g., 2 to 8 membered, 2 to 6 membered, 4 to 6 membered, 2 to 3 membered, or 4 to 5 membered), R^{21A}-substituted or unsubstituted cycloalkyl (e.g., C₃-C₈, C₃-C₆, C₄-C₆, or C₅-C₆), R^{21A}-substituted or unsubstituted heterocycloalkyl (e.g., 3 to 8 membered, 3 to 6 membered, 4 to 6 membered, 4 to 5 membered, or 5 to 6 membered), R^{21A}-substituted or unsubstituted aryl (e.g., C₆-C₁₀ or phenyl), or R^{21A}-substituted or unsubstituted heteroaryl (e.g., 5 to 10 membered, 5 to 9 membered, or 5 to 6 membered). In embodiments, R^{20A} is independently oxo,
halogen, —CX^{20A}₃, —CHX^{20A}₂, —CH₂X^{20A}, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{20A} is independently —F, —Cl, —Br, or —I.

In embodiments, R^{20A} is independently unsubstituted methyl. In embodiments, R^{20A} is independently unsubstituted ethyl.

R^{21A} is independently oxo,

halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{22A}-substituted or unsubstituted alkyl (e.g., C₁-C₈, C₁-C₆, C₁-C₄, or C₁-C₂), R^{22A}-substituted or unsubstituted heteroalkyl (e.g., 2 to 8 membered, 2 to 6 membered, 4 to 6 membered, 2 to 3 membered, or 4 to 5 membered), R^{22A}-substituted or unsubstituted cycloalkyl (e.g., C₃-C₈, C₃-C₆, C₄-C₆, or C₅-C₆), R^{22A}-substituted or unsubstituted heterocycloalkyl (e.g., 3 to 8 membered, 3 to 6 membered, 4 to 6 membered, 4 to 5 membered, or 5 to 6 membered), R^{22A}-substituted or unsubstituted aryl (e.g., C₆-C₁₀ or phenyl), or R^{22A}-substituted or unsubstituted heteroaryl (e.g., 5 to 10 membered, 5 to 9 membered, or 5 to 6 membered). In embodiments, R^{21A} is independently oxo,
halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{21A} is independently —F, —Cl, —Br, or —I.

In embodiments, R^{21A} is independently unsubstituted methyl. In embodiments, R^{21A} is independently unsubstituted ethyl.

R^{22A} is independently oxo,

halogen, —CX^{22A}₃, —CHX^{22A}₂, —CH₂X^{22A}, —OCX^{22A}₃, —OCH₂X^{22A}, —OCHX^{22A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{22A} is independently —F, —Cl, —Br, or —I.

In embodiments, R^{22A} is independently unsubstituted methyl. In embodiments, R^{22A} is independently unsubstituted ethyl.

In embodiments, R^{1B} is independently

hydrogen, —CX^{1B}₃, —CHX^{1B}₂, —CH₂X^{1B}, —CN, —COOH, —CONH₂, R^{20B}-substituted or unsubstituted alkyl (e.g., C₁-C₈, C₁-C₆, C₁-C₄, or C₁-C₂), R^{20B}-substituted or unsubstituted heteroalkyl (e.g., 2 to 8 membered, 2 to 6 membered, 4 to 6 membered, 2 to 3 membered, or 4 to 5 membered), R^{20B}-substituted or unsubstituted cycloalkyl (e.g., C₃-C₈, C₃-C₆, C₄-C₆, or C₅-C₆), R^{20B}-substituted or unsubstituted heterocycloalkyl (e.g., 3 to 8 membered, 3 to 6 membered, 4 to 6 membered, 4 to 5 membered, or 5 to 6 membered), R^{20B}-substituted or unsubstituted aryl (e.g., C₆-C₁₀ or phenyl), or R^{20B}-substituted or unsubstituted heteroaryl (e.g., 5 to 10 membered, 5 to 9 membered, or 5 to 6 membered). In embodiments, R^{1B} is independently
hydrogen, —CX^{1B}₃, —CHX^{1B}₂, —CH₂X^{1B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{1B} is independently —F, —Cl, —Br, or —I.

In embodiments, R^{1B} is independently hydrogen. In embodiments, R^{1B} is independently unsubstituted methyl. In embodiments, R^{1B} is independently unsubstituted ethyl.

In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered) or R^{20B}-substituted or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form a R^{20B}-substituted or unsubstituted heteroaryl (e.g., 5 to 10 membered, 5 to 9 membered, or 5 to 6 membered). In embodiments, R^{1A} and R^{1B} substituents bonded to the same nitrogen atom may optionally be joined to form an unsubstituted heteroaryl (e.g., 5 to 10 membered, 5 to 9 membered, or 5 to 6 membered).

R^{20B} is independently oxo,

halogen, —CX^{20B}₃, —CHX^{20B}₂, —CH₂X^{20B}, —OCX^{20B}₃, —OCH₂X^{20B}, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{21B}-substituted or unsubstituted alkyl (e.g., C₁-C₈, C₁-C₆, C₁-C₄, or C₁-C₂), R^{21B}-substituted or unsubstituted heteroalkyl (e.g., 2 to 8 membered, 2 to 6 membered, 4 to 6 membered, 2 to 3 membered, or 4 to 5 membered), R^{21B}-substituted or unsubstituted cycloalkyl (e.g., C₃-C₈, C₃-C₆, C₄-C₆, or C₅-C₆), R^{21B}-substituted or unsubstituted heterocycloalkyl (e.g., 3 to 8 membered, 3 to 6 membered, 4 to 6 membered, 4 to 5 membered, or 5 to 6 membered), R^{21B}-substituted or unsubstituted aryl (e.g., C₆-C₁₀ or phenyl), or R^{21B}-substituted or unsubstituted heteroaryl (e.g., 5 to 10 membered, 5 to 9 membered, or 5 to 6 membered). In embodiments, R^{20B} is independently oxo,
halogen, —CX^{20B}₃, —CHX^{20B}₂, —CH₂X^{20B}, —OCX^{20B}₃, —OCH₂X^{20B}, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{20B} is independently —F, —Cl, —Br, or —I.

In embodiments, R^{20B} is independently unsubstituted methyl. In embodiments, R^{20B} is independently unsubstituted ethyl.

R^{21B} is independently oxo,

halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{22B}-substituted or unsubstituted alkyl (e.g., C₁-C₈, C₁-C₆, C₁-C₄, or C₁-C₂), R^{22B}-substituted or unsubstituted heteroalkyl (e.g., 2 to 8 membered, 2 to 6 membered, 4 to 6 membered, 2 to 3 membered, or 4 to 5 membered), R^{22B}-substituted or unsubstituted cycloalkyl (e.g., C₃-C₈, C₃-C₆, C₄-C₆, or C₅-C₆), R^{22B}-substituted or unsubstituted heterocycloalkyl (e.g., 3 to 8 membered, 3 to 6 membered, 4 to 6 membered, 4 to 5 membered, or 5 to 6 membered), R^{22B}-substituted or unsubstituted aryl (e.g., C₆-C₁₀ or phenyl), or R^{22B}-substituted or unsubstituted heteroaryl (e.g., 5 to 10 membered, 5 to 9 membered, or 5 to 6 membered). In embodiments, R^{21B} is independently oxo,
halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{21B} is independently —F, —Cl, —Br, or —I.

In embodiments, R^{21B} is independently unsubstituted methyl. In embodiments, R^{21B} is independently unsubstituted ethyl.

R^{22B} is independently oxo,

halogen, —CX^{22B}₃, —CHX^{22B}₂, —CH₂X^{22B}, —OCX^{22B}₃, —OCH₂X^{22B}, —OCHX^{22B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2B} is independently —F, —Cl, —Br, or —I.

In embodiments, R^{22B} is independently unsubstituted methyl. In embodiments, R^{22B} is independently unsubstituted ethyl.

In embodiments, R^{1C} is independently

hydrogen, —CX^{1C}₃, —CHX^{1C}₂, —CH₂X^{1C}, —CN, —COOH, —CONH₂, R^{20C}-substituted or unsubstituted alkyl (e.g., C₁-C₈, C₁-C₆, C₁-C₄, or C₁-C₂), R^{20C}-substituted or unsubstituted heteroalkyl (e.g., 2 to 8 membered, 2 to 6 membered, 4 to 6 membered, 2 to 3 membered, or 4 to 5 membered), R^{20C}-substituted or unsubstituted cycloalkyl (e.g., C₃-C₈, C₃-C₆, C₄-C₆, or C₅-C₆), R^{20C}-substituted or unsubstituted heterocycloalkyl (e.g., 3 to 8 membered, 3 to 6 membered, 4 to 6 membered, 4 to 5 membered, or 5 to 6 membered), R^{20C}-substituted or unsubstituted aryl (e.g., C₆-C₁₀ or phenyl), or R^{20C}-substituted or unsubstituted heteroaryl (e.g., 5 to 10 membered, 5 to 9 membered, or 5 to 6 membered). In embodiments, R^{1C} is independently
hydrogen, —CX^{1C}₃, —CHX^{1C}₂, —CH₂X^{1C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{1C} is independently —F, —Cl, —Br, or —I.

In embodiments, R^{1C} is independently hydrogen. In embodiments, R^{1C} is independently unsubstituted methyl. In embodiments, R^{1C} is independently unsubstituted ethyl.

R^{20C} is independently oxo,

halogen, —CX^{20C}₃, —CHX^{20C}₂, —CH₂X^{20C}, —OCX^{20C}₃, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{21C}-substituted or unsubstituted alkyl (e.g., C₁-C₈, C₁-C₆, C₁-C₄, or C₁-C₂), R^{21C}-substituted or unsubstituted heteroalkyl (e.g., 2 to 8 membered, 2 to 6 membered, 4 to 6 membered, 2 to 3 membered, or 4 to 5 membered), R^{21C}-substituted or unsubstituted cycloalkyl (e.g., C₃-C₈, C₃-C₆, C₄-C₆, or C₅-C₆), R^{21C}-substituted or unsubstituted heterocycloalkyl (e.g., 3 to 8 membered, 3 to 6 membered, 4 to 6 membered, 4 to 5 membered, or 5 to 6 membered), R^{21C}-substituted or unsubstituted aryl (e.g., C₆-C₁₀ or phenyl), or R^{21C}-substituted or unsubstituted heteroaryl (e.g., 5 to 10 membered, 5 to 9 membered, or 5 to 6 membered). In embodiments, R^{20C} is independently oxo,
halogen, —CX^{20C}₃, —CHX^{20C}₂, —CH₂X^{20C}, —OCX^{20C}₃, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{20C} is independently —F, —Cl, —Br, or —I.

In embodiments, R^{20C} is independently unsubstituted methyl. In embodiments, R^{20C} is independently unsubstituted ethyl.

R^{21C} is independently oxo,

halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{22C}-substituted or unsubstituted alkyl (e.g., C₁-C₈, C₁-C₆, C₁-C₄, or C₁-C₂), R^{22C}-substituted or unsubstituted heteroalkyl (e.g., 2 to 8 membered, 2 to 6 membered, 4 to 6 membered, 2 to 3 membered, or 4 to 5 membered), R^{22C}-substituted or unsubstituted cycloalkyl (e.g., C₃-C₈, C₃-C₆, C₄-C₆, or C₅-C₆), R^{22C}-substituted or unsubstituted heterocycloalkyl (e.g., 3 to 8 membered, 3 to 6 membered, 4 to 6 membered, 4 to 5 membered, or 5 to 6 membered), R^{22C}-substituted or unsubstituted aryl (e.g., C₆-C₁₀ or phenyl), or R^{22C}-substituted or unsubstituted heteroaryl (e.g., 5 to 10 membered, 5 to 9 membered, or 5 to 6 membered). In embodiments, R^{21C} is independently oxo,
halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{21C} is independently —F, —Cl, —Br, or —I.

In embodiments, R^{21C} is independently unsubstituted methyl. In embodiments, R^{21C} is independently unsubstituted ethyl.

R^{22C} is independently oxo,

halogen, —CX^{22C}₃, —CHX^{22C}₂, —CH₂X^{22C}, —OCX^{22C}₃, —OCH₂X^{22C}, —OCHX^{22C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{22C} is independently —F, —Cl, —Br, or —I.

In embodiments, R^{22C} is independently unsubstituted methyl. In embodiments, R^{22C} is independently unsubstituted ethyl.

In embodiments, R^{1D} is independently

hydrogen, —CX^{1D}₃, —CHX^{1D}₂, —CH₂X^{1D}, —CN, —COOH, —CONH₂, R^{20D}-substituted or unsubstituted alkyl (e.g., C₁-C₈, C₁-C₆, C₁-C₄, or C₁-C₂), R^{20D}-substituted or unsubstituted heteroalkyl (e.g., 2 to 8 membered, 2 to 6 membered, 4 to 6 membered, 2 to 3 membered, or 4 to 5 membered), R^{20D}-substituted or unsubstituted cycloalkyl (e.g., C₃-C₈, C₃-C₆, C₄-C₆, or C₅-C₆), R^{20D}-substituted or unsubstituted heterocycloalkyl (e.g., 3 to 8 membered, 3 to 6 membered, 4 to 6 membered, 4 to 5 membered, or 5 to 6 membered), R^{20D}-substituted or unsubstituted aryl (e.g., C₆-C₁₀ or phenyl), or R^{20D}-substituted or unsubstituted heteroaryl (e.g., 5 to 10 membered, 5 to 9 membered, or 5 to 6 membered). In embodiments, R^{1D} is independently
hydrogen, —CX^{1D}₃, —CHX^{1D}₂, —CH₂X^{1D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{1D} is independently —F, —Cl, —Br, or —I.

In embodiments, R^{1D} is independently hydrogen. In embodiments, R^{1D} is independently unsubstituted methyl. In embodiments, R^{1D} is independently unsubstituted ethyl.

R^{20D} is independently oxo,

halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{21D}-substituted or unsubstituted alkyl (e.g., C₁-C₈, C₁-C₆, C₁-C₄, or C₁-C₂), R^{21D}-substituted or unsubstituted heteroalkyl (e.g., 2 to 8 membered, 2 to 6 membered, 4 to 6 membered, 2 to 3 membered, or 4 to 5 membered), R^{21D}-substituted or unsubstituted cycloalkyl (e.g., C₃-C₈, C₃-C₆, C₄-C₆, or C₅-C₆), R^{21D}-substituted or unsubstituted heterocycloalkyl (e.g., 3 to 8 membered, 3 to 6 membered, 4 to 6 membered, 4 to 5 membered, or 5 to 6 membered), R^{21D}-substituted or unsubstituted aryl (e.g., C₆-C₁₀ or phenyl), or R^{21D}-substituted or unsubstituted heteroaryl (e.g., 5 to 10 membered, 5 to 9 membered, or 5 to 6 membered). In embodiments, R^{20D} is independently oxo,
halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{20D} is independently —F, —Cl, —Br, or —I.

In embodiments, R^{20D} is independently unsubstituted methyl. In embodiments, R^{20D} is independently unsubstituted ethyl.

R^{21D} is independently oxo,

halogen, —CX^{21D}₃, —CHX^{21D}₂, —CH₂X^{21D}, OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{22D}-substituted or unsubstituted alkyl (e.g., C₁-C₈, C₁-C₆, C₁-C₄, or C₁-C₂), R^{22D}-substituted or unsubstituted heteroalkyl (e.g., 2 to 8 membered, 2 to 6 membered, 4 to 6 membered, 2 to 3 membered, or 4 to 5 membered), R^{22D}-substituted or unsubstituted cycloalkyl (e.g., C₃-C₈, C₃-C₆, C₄-C₆, or C₅-C₆), R^{22D}-substituted or unsubstituted heterocycloalkyl (e.g., 3 to 8 membered, 3 to 6 membered, 4 to 6 membered, 4 to 5 membered, or 5 to 6 membered), R^{22D}-substituted or unsubstituted aryl (e.g., C₆-C₁₀ or phenyl), or R^{22D}-substituted or unsubstituted heteroaryl (e.g., 5 to 10 membered, 5 to 9 membered, or 5 to 6 membered). In embodiments, R^{21D} is independently oxo,
halogen, —CX^{21D}₃, —CHX^{21D}₂, —CH₂X^{21D}, OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{21D} is independently —F, —Cl, —Br, or —I.

In embodiments, R^{21D} is independently unsubstituted methyl. In embodiments, R^{21D} is independently unsubstituted ethyl.

R^{22D} is independently oxo,

halogen, —CX^{22D}₃, —CHX^{22D}₂, —CH₂X^{22D}, —OCX^{22D}₃, —OCH₂X^{22D}, —OCHX^{22D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{22D} is independently —F, —Cl, —Br, or —I.

In embodiments, R^{22D} is independently unsubstituted methyl. In embodiments, R^{22D} is independently unsubstituted ethyl.

In embodiments, R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C (O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R² is independently hydrogen,

halogen, —CX²₃, —CN, —OH, —NH₂, —SH, —OCX²₃, —OCHX²₂, —OCH₂X², —CHX²₂, —CH₂X², substituted or unsubstituted C₁-C₄ alkyl, or substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R² is independently hydrogen,
halogen, —CX²₃, —CN, —OH, —NH₂, —SH, —OCX²₃, —OCHX²₂, —OCH₂X², —CHX²₂, —CH₂X², unsubstituted C₁-C₄ alkyl, or unsubstituted 2 to 4 membered heteroalkyl.

In embodiments, R² is independently hydrogen, halogen, —CX²₃, —CHX²₂, —CH₂X², —OCX²₃, —OCH₂X², —OCHX²₂, —NR^{2A}R^{2B}, —C(O)R^{2C}, —C(O)—OR^{2C}, —C(O)NR^{2A}R^{2B}, —OR^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen,

halogen, —NR^{2A}R^{2B}, —C(O)R^{2C}, —C(O)—OR^{2C}, —C(O)NR^{2A}R^{2B}, —OR^{2D}, —NR^{2A}C(O)R^{2C}, —NR^{2A}C(O) OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently hydrogen, halogen, —NR^{2A}R^{2B}, —OR^{2D}, or substituted or unsubstituted heteroaryl. In embodiments, R² is substituted or unsubstituted aryl. In embodiments, R² is substituted or unsubstituted phenyl. In embodiments, R² is hydrogen. In embodiments, R² is halogen. In embodiments, R² is —NR^{2A}R^{2B}. In embodiments, R² is —OR^{2D}. In embodiments, R² is substituted or unsubstituted heteroaryl. In embodiments, R² is substituted heteroaryl. In embodiments, R² is substituted 5 to 6 membered heteroaryl. In embodiments, R² is —NH₂. In embodiments, R² is —OH. In embodiments, R² is —Cl. In embodiments, R² is —F. In embodiments, R² is halogen.

In embodiments, R² is substituted or unsubstituted (C₃-C₁₀) cycloalkyl, substituted or unsubstituted 3 to 10 membered heterocycloalkyl, substituted or unsubstituted (C₆-C₁₀) aryl, or substituted or unsubstituted 5 to 10 membered heteroaryl. In embodiments, R² is a substituted or unsubstituted heteroaryl. In embodiments, R² is a substituted or unsubstituted 5 to 6 membered heteroaryl. In embodiments, R² is a substituted or unsubstituted 5 membered heteroaryl.

In embodiments, R² is a substituted or unsubstituted (C₃-C₁₀) cycloalkyl, a substituted or unsubstituted 3 to 10 membered heterocycloalkyl, a substituted or unsubstituted (C₆-C₁₀) aryl, or a substituted or unsubstituted 5 to 10 membered heteroaryl. In embodiments, R² is a substituted or unsubstituted cycloalkyl (e.g., C₃-C₈ cycloalkyl, C₃-C₆ cycloalkyl, or C₅-C₆ cycloalkyl). In embodiments, R² is a substituted or unsubstituted C₃-C₈ cycloalkyl. In embodiments, R² is a substituted or unsubstituted C₃-C₆ cycloalkyl. In embodiments, R² is a substituted or unsubstituted C₅-C₆ cycloalkyl. In embodiments, R² is a substituted or unsubstituted C₆ cycloalkyl. In embodiments, R² is a substituted or unsubstituted C₅ cycloalkyl. In embodiments, R² is a substituted or unsubstituted (C₆-C₁₀) aryl. In embodiments, R² is substituted or unsubstituted phenyl. In embodiments, R² is substituted or unsubstituted naphthyl. In embodiments, R² is substituted or unsubstituted aziridinyl, substituted or unsubstituted oxiranyl, substituted or unsubstituted thiiranyl, substituted or unsubstituted azetidinyl, substituted or unsubstituted oxetanyl, substituted or unsubstituted thietanyl, substituted or unsubstituted pyrrolidinyl, substituted or unsubstituted pyrrolyl, substituted or unsubstituted imidazolyl, substituted or unsubstituted imidazolinyl, substituted or unsubstituted pyrazolinyl, substituted or unsubstituted tetrahydrofuranyl, substituted or unsubstituted thiolanyl, substituted or unsubstituted piperidinyl, substituted or unsubstituted piperazinyl, substituted or unsubstituted pyranyl, substituted or unsubstituted morpholinyl, substituted or unsubstituted 1,4-dioxanyl, substituted or unsubstituted tetrahydro-2H-pyranyl, substituted or unsubstituted thianyl, or substituted or unsubstituted dithianyl. In embodiments, R² is substituted or unsubstituted phenyl, substituted or unsubstituted thiofuranyl, substituted or unsubstituted imidazolyl, substituted or unsubstituted pyrazolyl, substituted or unsubstituted triazolyl, substituted or unsubstituted tetrazolyl, substituted or unsubstituted furanyl, substituted or unsubstituted oxazolyl, substituted or unsubstituted isooxazolyl, substituted or unsubstituted oxadiazolyl, substituted or unsubstituted oxatriazolyl, substituted or unsubstituted thienyl, substituted or unsubstituted thiazolyl, substituted or unsubstituted isothiazolyl, substituted or unsubstituted pyridinyl, substituted or unsubstituted pyrazinyl, substituted or unsubstituted pyrimidinyl, substituted or unsubstituted pyridazinyl, or substituted or unsubstituted triazinyl (e.g., 1,3,5-triazinyl, 1,2,3-triazinyl, or 1,2,4-triazinyl). In embodiments, R² is substituted or unsubstituted indolyl, substituted or unsubstituted benzimidazolyl, substituted or unsubstituted indazolyl, substituted or unsubstituted benzotriazolyl, substituted or unsubstituted pyrrolopyrimidinyl, substituted or unsubstituted purinyl, substituted or unsubstituted indolizinyl, substituted or unsubstituted pyrrolopyriazinyl, substituted or unsubstituted pyrrolopyrimidinyl, substituted or unsubstituted imidazopyridazinyl, substituted or unsubstituted imidazopyridinyl, substituted or unsubstituted imidazopyrimidinyl, substituted or unsubstituted cinnolinyl, substituted or unsubstituted quinazolinyl, substituted or unsubstituted quinoxalinyl, substituted or unsubstituted phthalazinyl, substituted or unsubstituted pyridopyrazinyl, substituted or unsubstituted pteridinyl, substituted or unsubstituted pyrazolopyridinyl, substituted or unsubstituted quinolinyl, substituted or unsubstituted isoquinolinyl, substituted or unsubstituted naphthyridinyl, or substituted or unsubstituted carbazolyl. In embodiments, R² is substituted aziridinyl, substituted oxiranyl, substituted thiiranyl, substituted azetidinyl, substituted oxetanyl, substituted thietanyl, substituted pyrrolidinyl, substituted pyrrolyl, substituted imidazolyl, substituted imidazolinyl, substituted pyrazolinyl, substituted tetrahydrofuranyl, substituted thiolanyl, substituted piperidinyl, substituted piperazinyl, substituted pyranyl, substituted morpholinyl, substituted 1,4-dioxanyl, substituted tetrahydro-2H-pyranyl, substituted thianyl, or substituted dithianyl. In embodiments, R² is substituted phenyl, substituted thiofuranyl, substituted imidazolyl, substituted pyrazolyl, substituted triazolyl, substituted tetrazolyl, substituted furanyl, substituted oxazolyl, substituted isooxazolyl, substituted oxadiazolyl, substituted oxatriazolyl, substituted thienyl, substituted thiazolyl, substituted isothiazolyl, substituted pyridinyl, substituted pyrazinyl, substituted pyrimidinyl, substituted pyridazinyl, or substituted triazinyl (e.g., 1,3,5-triazinyl, 1,2,3-triazinyl, or 1,2,4-triazinyl). In embodiments, R² is substituted indolyl, substituted benzimidazolyl, substituted indazolyl, substituted benzotriazolyl, substituted pyrrolopyrimidinyl, substituted purinyl, substituted indolizinyl, substituted pyrrolopyriazinyl, substituted pyrrolopyrimidinyl, substituted imidazopyridazinyl, substituted imidazopyridinyl, substituted imidazopyrimidinyl, substituted cinnolinyl, substituted quinazolinyl, substituted quinoxalinyl, substituted phthalazinyl, substituted pyridopyrazinyl, substituted pteridinyl, substituted pyrazolopyridinyl, substituted quinolinyl, substituted isoquinolinyl, substituted naphthyridinyl, or substituted carbazolyl. In embodiments, R² is unsubstituted aziridinyl, unsubstituted oxiranyl, unsubstituted thiiranyl, unsubstituted azetidinyl, unsubstituted oxetanyl, unsubstituted thietanyl, unsubstituted pyrrolidinyl, unsubstituted pyrrolyl, unsubstituted imidazolyl, unsubstituted imidazolinyl, unsubstituted pyrazolinyl, unsubstituted tetrahydrofuranyl, unsubstituted thiolanyl, unsubstituted piperidinyl, unsubstituted piperazinyl, unsubstituted pyranyl, unsubstituted morpholinyl, unsubstituted 1,4-dioxanyl, unsubstituted tetrahydro-2H-pyranyl, unsubstituted thianyl, or unsubstituted dithianyl. In embodiments, R² is unsubstituted phenyl, unsubstituted thiofuranyl, unsubstituted imidazolyl, unsubstituted pyrazolyl, unsubstituted triazolyl, unsubstituted tetrazolyl, unsubstituted furanyl, unsubstituted oxazolyl, unsubstituted isooxazolyl, unsubstituted oxadiazolyl, unsubstituted oxatriazolyl, unsubstituted thienyl, unsubstituted thiazolyl, unsubstituted isothiazolyl, unsubstituted pyridinyl, unsubstituted pyrazinyl, unsubstituted pyrimidinyl, unsubstituted pyridazinyl, or unsubstituted triazinyl (e.g., 1,3,5-triazinyl, 1,2,3-triazinyl, or 1,2,4-triazinyl). In embodiments, R² is unsubstituted indolyl, unsubstituted benzimidazolyl, unsubstituted indazolyl, unsubstituted benzotriazolyl, unsubstituted pyrrolopyrimidinyl, unsubstituted purinyl, unsubstituted indolizinyl, unsubstituted pyrrolopyriazinyl, unsubstituted pyrrolopyrimidinyl, unsubstituted imidazopyridazinyl, unsubstituted imidazopyridinyl, unsubstituted imidazopyrimidinyl, unsubstituted cinnolinyl, unsubstituted quinazolinyl, unsubstituted quinoxalinyl, unsubstituted phthalazinyl, unsubstituted pyridopyrazinyl, unsubstituted pteridinyl, unsubstituted pyrazolopyridinyl, unsubstituted quinolinyl, unsubstituted isoquinolinyl, unsubstituted naphthyridinyl, or unsubstituted carbazolyl.

In embodiments, —(R²)—(R²³)_{z23} is:

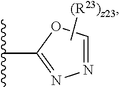

wherein R²³ and z23 are as described herein including embodiments.

In embodiments, —(R²)—(R²³)_{z23} is:

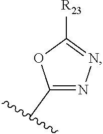

wherein R²³ is as described herein, including embodiments.

In embodiments, R² is R²³-substituted phenyl. In embodiments, R² is R²³-substituted 5 to 6 membered heteroaryl. R²³ is independently halogen, —CX²³₃, —CHX²³₂, —CH₂X²³, —OCX²³₃, —OCH₂X²³, —OCHX²³₂, —CN, —SOₙ₂₃R^{100D}, —SOᵥ₂₃NR^{100A}R^{100B}, —NHC(O)NR^{100A}R^{100B}, —N(O)ₘ₂₃, —NR^{100A}R^{100B}, —C(O)R^{100C}, —C(O)—OR^{100C}, —C(O)NR^{100A}R^{100B}, —OR^{100D}, —NR^{100A}SO₂R^{100D}, —NR^{100A} C(O)R^{100C}, —NR^{100A}C(O)OR^{100C}, —NR^{100A}OR^{100C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R² substituents may optionally be joined to form a substituted or unsubstituted cycloalkyl, substituted or unsubstituted heterocycloalkyl, substituted or unsubstituted aryl, or substituted or unsubstituted heteroaryl. R^{100A}, R^{100B}, R^{100C}, and R^{100D} are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{100A} and R^{100B} substituents bonded to the same nitrogen atom may optionally be joined to form a substituted or unsubstituted heterocycloalkyl or substituted or unsubstituted heteroaryl; X and X²³ are independently —F, —Cl, —Br, or —I. The symbol n23 is independently an integer from 0 to 4. The symbols m23 and v23 are independently 1 or 2.

In embodiments, R^{2A} is independently substituted or unsubstituted alkyl (e.g., C₁-C₈, C₁-C₆, C₁-C₄, or C₁-C₂). In embodiments, R^{2A} is independently substituted alkyl (e.g., C₁-C₈, C₁-C₆, C₁-C₄, or C₁-C₂). In embodiments, R^{2A} is independently unsubstituted alkyl (e.g., C₁-C₈, C₁-C₆, C₁-C₄, or C₁-C₂). In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted isopropyl. In embodiments, R^{2A} is independently unsubstituted tert-butyl. In embodiments, R^{2A} is independently substituted or unsubstituted heteroalkyl (e.g., 2 to 8 membered, 2 to 6 membered, 4 to 6 membered, 2 to 3 membered, or 4 to 5 membered). In embodiments, R^{2A} is independently substituted heteroalkyl (e.g., 2 to 8 membered, 2 to 6 membered, 4 to 6 membered, 2 to 3 membered, or 4 to 5 membered). In embodiments, R^{2A} is independently unsubstituted heteroalkyl (e.g., 2 to 8 membered, 2 to 6 membered, 4 to 6 membered, 2 to 3 membered, or 4 to 5 membered). In embodiments, R^{2A} is independently substituted or unsubstituted cycloalkyl (e.g., C₃-C₈, C₃-C₆, C₄-C₆, or C₅-C₆). In embodiments, R^{2A} is independently substituted cycloalkyl (e.g., C₃-C₈, C₃-C₆, C₄-C₆, or C₅-C₆). In embodiments, R^{2A} is independently unsubstituted cycloalkyl (e.g., C₃-C₈, C₃-C₆, C₄-C₆, or C₅-C₆). In embodiments, R^{2A} is independently substituted or unsubstituted heterocycloalkyl (e.g., 3 to 8 membered, 3 to 6 membered, 4 to 6 membered, 4 to 5 membered, or 5 to 6 membered). In embodiments, R^{2A} is independently substituted heterocycloalkyl (e.g., 3 to 8 membered, 3 to 6 membered, 4 to 6 membered, 4 to 5 membered, or 5 to 6 membered). In embodiments, R² is independently unsubstituted heterocycloalkyl (e.g., 3 to 8 membered, 3 to 6 membered, 4 to 6 membered, 4 to 5 membered, or 5 to 6 membered). In embodiments, R^{2A} is independently substituted or unsubstituted aryl (e.g., C₆-C₁₀ or phenyl). In embodiments, R^{2A} is independently substituted aryl (e.g., C₆-C₁₀ or phenyl). In embodiments, R^{2A} is independently unsubstituted aryl (e.g., C₆-C₁₀ or phenyl). In embodiments, R^{2A} is independently substituted or unsubstituted heteroaryl (e.g., 5 to 10 membered, 5 to 9 membered, or 5 to 6 membered).

In embodiments, R^{2A} is independently substituted heteroaryl (e.g., 5 to 10 membered, 5 to 9 membered, or 5 to 6 membered). In embodiments, R^{2A} is independently unsubstituted heteroaryl (e.g., 5 to 10 membered, 5 to 9 membered, or 5 to 6 membered). [0289] In embodiments, R^{2A} is substituted or unsubstituted aryl (e.g., C₆-C₁₀ aryl, C₁₀ aryl, or phenyl). In embodiments, R^{2A} is substituted aryl (e.g., C₆-C₁₀ aryl, C₁₀ aryl, or phenyl). In embodiments, R^{2A} is an unsubstituted aryl (e.g., C₆-C₁₀ aryl, C₁₀ aryl, or phenyl).

In embodiments, R^{2B} is independently substituted or unsubstituted alkyl (e.g., C₁-C₈, C₁-C₆, C₁-C₄, or C₁-C₂). In embodiments, R^{2B} is independently substituted alkyl (e.g., C₁-C₈, C₁-C₆, C₁-C₄, or C₁-C₂). In embodiments, R^{2B} is independently unsubstituted alkyl (e.g., C₁-C₈, C₁-C₆, C₁-C₄, or C₁-C₂). In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted isopropyl. In embodiments, R^{2B} is independently unsubstituted tert-butyl. In embodiments, R^{2B} is independently substituted or unsubstituted heteroalkyl (e.g., 2 to 8 membered, 2 to 6 membered, 4 to 6 membered, 2 to 3 membered, or 4 to 5 membered). In embodiments, R^{2B} is independently substituted heteroalkyl (e.g., 2 to 8 membered, 2 to 6 membered, 4 to 6 membered, 2 to 3 membered, or 4 to 5 membered). In embodiments, R^{2B} is independently unsubstituted heteroalkyl (e.g., 2 to 8 membered, 2 to 6 membered, 4 to 6 membered, 2 to 3 membered, or 4 to 5 membered). In embodiments, R^{2B} is independently substituted or unsubstituted cycloalkyl (e.g., C₃-C₈, C₃-C₆, C₄-C₆, or C₅-C₆). In embodiments, R^{2B} is independently substituted cycloalkyl (e.g., C₃-C₈, C₃-C₆, C₄-C₆, or C₅-C₆). In embodiments, R^{2B} is independently unsubstituted cycloalkyl (e.g., C₃-C₈, C₃-C₆, C₄-C₆, or C₅-C₆). In embodiments, R^{2B} is independently substituted or unsubstituted heterocycloalkyl (e.g., 3 to 8 membered, 3 to 6 membered, 4 to 6 membered, 4 to 5 membered, or 5 to 6 membered). In embodiments, R^{2B} is independently substituted heterocycloalkyl (e.g., 3 to 8 membered, 3 to 6 membered, 4 to 6 membered, 4 to 5 membered, or 5 to 6 membered). In embodiments, R^{2B} is independently unsubstituted heterocycloalkyl (e.g., 3 to 8 membered, 3 to 6 membered, 4 to 6 membered, 4 to 5 membered, or 5 to 6 membered). In embodiments, R^{2B} is independently substituted or unsubstituted aryl (e.g., C₆-C₁₀ or phenyl). In embodiments, R^{2B} is independently substituted aryl (e.g., C₆-C₁₀ or phenyl). In embodiments, R^{Z}B is independently unsubstituted aryl (e.g., C₆-C₁₀ or phenyl). In embodiments, R^{2B} is independently substituted or unsubstituted heteroaryl (e.g., 5 to 10 membered, 5 to 9 membered, or 5 to 6 membered). In embodiments, R^{2B} is independently substituted heteroaryl (e.g., 5 to 10 membered, 5 to 9 membered, or 5 to 6 membered). In embodiments, R^{2B} is independently unsubstituted heteroaryl (e.g., 5 to 10 membered, 5 to 9 membered, or 5 to 6 membered).

In embodiments, R^{2A} and R^{2B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{2A} and R^{2B} substituents bonded to the same nitrogen atom may be joined to form a substituted or unsubstituted heteroaryl (e.g., 5 to 10 membered, 5 to 9 membered, or 5 to 6 membered). In embodiments, R^{2A} and R^{2B} substituents bonded to the same nitrogen atom may be joined to form a substituted heteroaryl (e.g., 5 to 10 membered, 5 to 9 membered, or 5 to 6 membered). In embodiments, R^{2A} and R^{2B} substituents bonded to the same nitrogen atom may be joined to form an unsubstituted heteroaryl (e.g., 5 to 10 membered, 5 to 9 membered, or 5 to 6 membered).

In embodiments, R^{2C} is independently substituted or unsubstituted alkyl (e.g., C₁-C₈, C₁-C₆, C₁-C₄, or C₁-C₂). In embodiments, R^{2C} is independently substituted alkyl (e.g., C₁-C₈, C₁-C₆, C₁-C₄, or C₁-C₂). In embodiments, R^{2C} is independently unsubstituted alkyl (e.g., C₁-C₈, C₁-C₆, C₁-C₄, or C₁-C₂). In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted isopropyl. In embodiments, R^{2C} is independently unsubstituted tert-butyl. In embodiments, R^{2C} is independently substituted or unsubstituted heteroalkyl (e.g., 2 to 8 membered, 2 to 6 membered, 4 to 6 membered, 2 to 3 membered, or 4 to 5 membered). In embodiments, R^{2C} is independently substituted heteroalkyl (e.g., 2 to 8 membered, 2 to 6 membered, 4 to 6 membered, 2 to 3 membered, or 4 to 5 membered). In embodiments, R² is independently unsubstituted heteroalkyl (e.g., 2 to 8 membered, 2 to 6 membered, 4 to 6 membered, 2 to 3 membered, or 4 to 5 membered). In embodiments, R^{2C} is independently substituted or unsubstituted cycloalkyl (e.g., C₃-C₈, C₃-C₆, C₄-C₆, or C₅-C₆). In embodiments, R^{2C} is independently substituted cycloalkyl (e.g., C₃-C₈, C₃-C₆, C₄-C₆, or C₅-C₆). In embodiments, R^{2C} is independently unsubstituted cycloalkyl (e.g., C₃-C₈, C₃-C₆, C₄-C₆, or C₅-C₆). In embodiments, R² is independently substituted or unsubstituted heterocycloalkyl (e.g., 3 to 8 membered, 3 to 6 membered, 4 to 6 membered, 4 to 5 membered, or 5 to 6 membered). In embodiments, R^{2C} is independently substituted heterocycloalkyl (e.g., 3 to 8 membered, 3 to 6 membered, 4 to 6 membered, 4 to 5 membered, or 5 to 6 membered). In embodiments, R^{2C} is independently unsubstituted heterocycloalkyl (e.g., 3 to 8 membered, 3 to 6 membered, 4 to 6 membered, 4 to 5 membered, or 5 to 6 membered). In embodiments, R^{2C} is independently substituted or unsubstituted aryl (e.g., C₆-C₁₀ or phenyl). In embodiments, R^{2C} is independently substituted aryl (e.g., C₆-C₁₀ or phenyl). In embodiments, R^{2C} is independently unsubstituted aryl (e.g., C₆-C₁₀ or phenyl). In embodiments, R^{2C} is independently substituted or unsubstituted heteroaryl (e.g., 5 to 10 membered, 5 to 9 membered, or 5 to 6 membered). In embodiments, R^{2C} is independently substituted heteroaryl (e.g., 5 to 10 membered, 5 to 9 membered, or 5 to 6 membered). In embodiments, R^{2C} is independently unsubstituted heteroaryl (e.g., 5 to 10 membered, 5 to 9 membered, or 5 to 6 membered).

In embodiments, R^{2D} is independently substituted or unsubstituted alkyl (e.g., C₁-C₈, C₁-C₆, C₁-C₄, or C₁-C₂). In embodiments, R^{2D} is independently substituted alkyl (e.g., C₁-C₈, C₁-C₆, C₁-C₄, or C₁-C₂). In embodiments, R^{2D} is independently unsubstituted alkyl (e.g., C₁-C₈, C₁-C₆, C₁-C₄, or C₁-C₂). In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted isopropyl. In embodiments, R^{2D} is independently unsubstituted tert-butyl. In embodiments, R^{2D} is independently substituted or unsubstituted heteroalkyl (e.g., 2 to 8 membered, 2 to 6 membered, 4 to 6 membered, 2 to 3 membered, or 4 to 5 membered). In embodiments, R^{2D} is independently substituted heteroalkyl (e.g., 2 to 8 membered, 2 to 6 membered, 4 to 6 membered, 2 to 3 membered, or 4 to 5 membered). In embodiments, R^{2D} is independently unsubstituted heteroalkyl (e.g., 2 to 8 membered, 2 to 6 membered, 4 to 6 membered, 2 to 3 membered, or 4 to 5 membered). In embodiments, R^{2D} is independently substituted or unsubstituted cycloalkyl (e.g., C₃-C₈, C₃-C₆, C₄-C₆, or C₅-C₆). In embodiments, R^{2D} is independently substituted cycloalkyl (e.g., C₃-C₈, C₃-C₆, C₄-C₆, or C₅-C₆). In embodiments, R^{2D} is independently unsubstituted cycloalkyl (e.g., C₃-C₈, C₃-C₆, C₄-C₆, or C₅-C₆). In embodiments, R^{2D} is independently substituted or unsubstituted heterocycloalkyl (e.g., 3 to 8 membered, 3 to 6 membered, 4 to 6 membered, 4 to 5 membered, or 5 to 6 membered). In embodiments, R^{2D} is independently substituted heterocycloalkyl (e.g., 3 to 8 membered, 3 to 6 membered, 4 to 6 membered, 4 to 5 membered, or 5 to 6 membered). In embodiments, R^{2D} is independently unsubstituted heterocycloalkyl (e.g., 3 to 8 membered, 3 to 6 membered, 4 to 6 membered, 4 to 5 membered, or 5 to 6 membered). In embodiments, R^{2D} is independently substituted or unsubstituted aryl (e.g., C₆-C₁₀ or phenyl). In embodiments, R^{2D} is independently substituted aryl (e.g., C₆-C₁₀ or phenyl). In embodiments, R^{2D} is independently unsubstituted aryl (e.g., C₆-C₁₀ or phenyl). In embodiments, R^{2D} is independently substituted or unsubstituted heteroaryl (e.g., 5 to 10 membered, 5 to 9 membered, or 5 to 6 membered). In embodiments, R^{2D} is independently substituted heteroaryl (e.g., 5 to 10 membered, 5 to 9 membered, or 5 to 6 membered). In embodiments, R^{2D} is independently unsubstituted heteroaryl (e.g., 5 to 10 membered, 5 to 9 membered, or 5 to 6 membered).

In embodiments, R² is independently hydrogen,

halogen, —CX²₃, —CHX²₂, —CH₂X², —OCX²₃, —OCH₂X², —OCHX²₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²³-substituted or unsubstituted alkyl (e.g., C₁-C₈, C₁-C₆, C₁-C₄, or C₁-C₂), R²³-substituted or unsubstituted heteroalkyl (e.g., 2 to 8 membered, 2 to 6 membered, 4 to 6 membered, 2 to 3 membered, or 4 to 5 membered), R²³-substituted or unsubstituted cycloalkyl (e.g., C₃-C₈, C₃-C₆, C₄-C₆, or C₅-C₆), R²³-substituted or unsubstituted heterocycloalkyl (e.g., 3 to 8 membered, 3 to 6 membered, 4 to 6 membered, 4 to 5 membered, or 5 to 6 membered), R²³-substituted or unsubstituted aryl (e.g., C₆-C₁₀ or phenyl), or R²³-substituted or unsubstituted heteroaryl (e.g., 5 to 10 membered, 5 to 9 membered, or 5 to 6 membered). In embodiments, R² is independently hydrogen,
halogen, —CX²₃, —CHX²₂, —CH₂X², —OCX²₃, —OCH₂X², —OCHX²₂, —CN, —OH, —NH₂, —COOH, —CO NH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X² is independently —F, —Cl, —Br, or —I.

In embodiments, R² is independently hydrogen. In embodiments, R² is independently R²³-substituted methyl. In embodiments, R² is independently R²³-substituted ethyl. In embodiments, R² is independently unsubstituted methyl. In embodiments, R² is independently unsubstituted ethyl.

R²³ is independently oxo,

halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²⁴-substituted or unsubstituted alkyl (e.g., C₁-C₈, C₁-C₆, C₁-C₄, or C₁-C₂), Ru-substituted or unsubstituted heteroalkyl (e.g., 2 to 8 membered, 2 to 6 membered, 4 to 6 membered, 2 to 3 membered, or 4 to 5 membered), R²⁴-substituted or unsubstituted cycloalkyl (e.g., C₃-C₈, C₃-C₆, C₄-C₆, or C₅-C₆), R²⁴-substituted or unsubstituted heterocycloalkyl (e.g., 3 to 8 membered, 3 to 6 membered, 4 to 6 membered, 4 to 5 membered, or 5 to 6 membered), R²⁴-substituted or unsubstituted aryl (e.g., C₆-C₁₀ or phenyl), or R²⁴-substituted or unsubstituted heteroaryl (e.g., 5 to 10 membered, 5 to 9 membered, or 5 to 6 membered). In embodiments, R²³ is independently oxo,
halogen, —CX²³₃, —CHX²³₂, —CH₂X²³, —OCX²³₃, —OCH₂X²³, —OCHX²³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²³ is independently —F, —Cl, —Br, or —I.

In embodiments, R²³ is independently halogen or —CX²³₃. In embodiments, R²³ is independently unsubstituted methyl. In embodiments, R²³ is independently unsubstituted ethyl. In embodiments, R²³ is independently hydrogen. In embodiments, R²³ is independently unsubstituted phenyl. In embodiments, R²³ is independently unsubstituted thiophenyl. In embodiments, R²³ is independently R²⁴-substituted aryl. In embodiments, R²³ is independently unsubstituted thienyl. In embodiments, R²³ is independently substituted phenyl. In embodiments, R²³ is independently R²⁴-substituted phenyl.

R²⁴ is independently oxo,

halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²⁵-substituted or unsubstituted alkyl (e.g., C₁-C₈, C₁-C₆, C₁-C₄, or C₁-C₂), R²⁵-substituted or unsubstituted heteroalkyl (e.g., 2 to 8 membered, 2 to 6 membered, 4 to 6 membered, 2 to 3 membered, or 4 to 5 membered), R²⁵-substituted or unsubstituted cycloalkyl (e.g., C₃-C₈, C₃-C₆, C₄-C₆, or C₅-C₆), R²⁵-substituted or unsubstituted heterocycloalkyl (e.g., 3 to 8 membered, 3 to 6 membered, 4 to 6 membered, 4 to 5 membered, or 5 to 6 membered), R²⁵-substituted or unsubstituted aryl (e.g., C₆-C₁₀ or phenyl), or R²-substituted or unsubstituted heteroaryl (e.g., 5 to 10 membered, 5 to 9 membered, or 5 to 6 membered). In embodiments, R²⁴ is independently oxo,
halogen, —CX²⁴₃, —CHX²⁴₂, —CH₂X²⁴, —OCX²⁴₃, —OCH₂X²⁴, —OCHX²⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁴ is independently —F, —Cl, —Br, or —I.

In embodiments, R²⁴ is independently hydrogen. In embodiments, R²⁴ is independently —OCH₃. In embodiments, R²⁴ is independently halogen. In embodiments, R²⁴ is independently —CF₃. In embodiments, R²⁴ is independently —Br. In embodiments, R²⁴ is independently —I. In embodiments, R²⁴ is independently unsubstituted 2 to 4 membered heteroalkyl. In embodiments, R²⁴ is independently unsubstituted C₁-C₄ alkoxy.

R²⁵ is independently oxo,

halogen, —CX²⁵₃, —CHX²⁵₂, —CH₂X²⁵, —OCX²⁵₃, —OCH₂X²⁵, —OCHX²⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁵ is independently —F, —Cl, —Br, or —I.

In embodiments, R²⁵ is independently unsubstituted methyl. In embodiments, R²⁵ is independently unsubstituted ethyl.

In embodiments, R^{2A} is independently

hydrogen, —CX^{2A}₃, —CHX^{2A}₂, —CH₂X^{2A}, —CN, —COOH, —CONH₂, R^{23A}-substituted or unsubstituted alkyl (e.g., C₁-C₈, C₁-C₆, C₁-C₄, or C₁-C₂), R^{23A}-substituted or unsubstituted heteroalkyl (e.g., 2 to 8 membered, 2 to 6 membered, 4 to 6 membered, 2 to 3 membered, or 4 to 5 membered), R^{23A}-substituted or unsubstituted cycloalkyl (e.g., C₃-C₈, C₃-C₆, C₄-C₆, or C₅-C₆), R^{23A}-substituted or unsubstituted heterocycloalkyl (e.g., 3 to 8 membered, 3 to 6 membered, 4 to 6 membered, 4 to 5 membered, or 5 to 6 membered), R^{23A}-substituted or unsubstituted aryl (e.g., C₆-C₁₀ or phenyl), or R^{23A}-substituted or unsubstituted heteroaryl (e.g., 5 to 10 membered, 5 to 9 membered, or 5 to 6 membered). In embodiments, R^{2A} is independently
hydrogen, —CX^{2A}₃, —CHX^{2A}₂, —CH₂X^{2A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2A} is independently —F, —Cl, —Br, or —I.

In embodiments, R^{2A} is independently hydrogen. In embodiments, R^{2A} is independently unsubstituted methyl. In embodiments, R^{2A} is independently unsubstituted ethyl.

In embodiments, R^{2A} and R^{2B} substituents bonded to the same nitrogen atom may optionally be joined to form a R^{23A}-substituted or unsubstituted heterocycloalkyl (e.g., 3 to 8 membered, 3 to 6 membered, 4 to 6 membered, 4 to 5 membered, or 5 to 6 membered) or R^{23A}-substituted or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form a R^{23A}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form a R^{23A}-substituted or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form an unsubstituted heteroaryl (e.g., 5 to 10 membered, 5 to 9 membered, or 5 to 6 membered).

R^{23A} is independently oxo,

halogen, —CX^{23A}₃, —CHX^{23A}₂, —CH₂X^{23A}, —OCX^{23A}₃, —OCH₂X^{23A}, —OCHX^{23A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{24A}-substituted or unsubstituted alkyl (e.g., C₁-C₈, C₁-C₆, C₁-C₄, or C₁-C₂), R^{24A}-substituted or unsubstituted heteroalkyl (e.g., 2 to 8 membered, 2 to 6 membered, 4 to 6 membered, 2 to 3 membered, or 4 to 5 membered), R^{24A}-substituted or unsubstituted cycloalkyl (e.g., C₃-C₈, C₃-C₆, C₄-C₆, or C₅-C₆), R^{24A}-substituted or unsubstituted heterocycloalkyl (e.g., 3 to 8 membered, 3 to 6 membered, 4 to 6 membered, 4 to 5 membered, or 5 to 6 membered), R^{24A}-substituted or unsubstituted aryl (e.g., C₆-C₁₀ or phenyl), or R^{24A}-substituted or unsubstituted heteroaryl (e.g., 5 to 10 membered, 5 to 9 membered, or 5 to 6 membered). In embodiments, R^{23A} is independently oxo,
halogen, —CX^{23A}₃, —CHX^{23A}₂, —CH₂X^{23A}, —OCX^{23A}₃, —OCH₂X^{23A}, —OCHX^{23A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{23A} is independently —F, —Cl, —Br, or —I.

In embodiments, R^{23A} is independently unsubstituted methyl. In embodiments, R^{23A} is independently unsubstituted ethyl.

R^{24A} is independently oxo,

halogen, —CX^{24A}₃, —CHX^{24A}₂, —CH₂X^{24A}, —OCX^{24A}₃, —OCH₂X^{24A}, —OCHX^{24A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{25A}-substituted or unsubstituted alkyl (e.g., C₁-C₈, C₁-C₆, C₁-C₄, or C₁-C₂), R^{25A}-substituted or unsubstituted heteroalkyl (e.g., 2 to 8 membered, 2 to 6 membered, 4 to 6 membered, 2 to 3 membered, or 4 to 5 membered), R^{25A}-substituted or unsubstituted cycloalkyl (e.g., C₃-C₈, C₃-C₆, C₄-C₆, or C₅-C₆), R^{25A}-substituted or unsubstituted heterocycloalkyl (e.g., 3 to 8 membered, 3 to 6 membered, 4 to 6 membered, 4 to 5 membered, or 5 to 6 membered), R^{25A}-substituted or unsubstituted aryl (e.g., C₆-C₁₀ or phenyl), or R^{25A}-substituted or unsubstituted heteroaryl (e.g., 5 to 10 membered, 5 to 9 membered, or 5 to 6 membered). In embodiments, R^{24A} is independently oxo,
halogen, —CX^{24A}₃, —CHX^{24A}₂, —CH₂X^{24A}, —OCX^{24A}₃, —OCH₂X^{24A}, —OCHX^{24A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{24A} is independently —F, —Cl, —Br, or —I.

R^{25A} is independently oxo,

halogen, —CX^{25A}₃, —CHX^{25A}₂, —CH₂X^{25A}, —OCX^{25A}₃, —OCH₂X^{25A}, —OCHX^{25A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{25A} is independently —F, —Cl, —Br, or —I.

In embodiments, R^{2B} is independently

hydrogen, —CX^{2B}₃, —CHX^{2B}₂, —CH₂X^{B}, —CN, —COOH, —CONH₂, R^{23B}-substituted or unsubstituted alkyl (e.g., C₁-C₈, C₁-C₆, C₁-C₄, or C₁-C₂), R^{23B}-substituted or unsubstituted heteroalkyl (e.g., 2 to 8 membered, 2 to 6 membered, 4 to 6 membered, 2 to 3 membered, or 4 to 5 membered), R^{23B}-substituted or unsubstituted cycloalkyl (e.g., C₃-C₈, C₃-C₆, C₄-C₆, or C₅-C₆), R^{23B}-substituted or unsubstituted heterocycloalkyl (e.g., 3 to 8 membered, 3 to 6 membered, 4 to 6 membered, 4 to 5 membered, or 5 to 6 membered), R^{23B}-substituted or unsubstituted aryl (e.g., C₆-C₁₀ or phenyl), or R^{23B}-substituted or unsubstituted heteroaryl (e.g., 5 to 10 membered, 5 to 9 membered, or 5 to 6 membered). In embodiments, R^{2B} is independently
hydrogen, —CX^{2B}₃, —CHX^{2B}₂, —CH₂X^{2B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2B} is independently —F, —Cl, —Br, or —I.

In embodiments, R^{2B} is independently hydrogen. In embodiments, R^{2B} is independently unsubstituted methyl. In embodiments, R^{2B} is independently unsubstituted ethyl.

In embodiments, R^{2A} and R^{2B} substituents bonded to the same nitrogen atom may optionally be joined to form a R^{23B}-substituted or unsubstituted heterocycloalkyl (e.g., 3 to 8 membered, 3 to 6 membered, 4 to 6 membered, 4 to 5 membered, or 5 to 6 membered) or R^{23B}-substituted or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form a R^{23B}-substituted or unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{2A} and R^{2B} substituents bonded to the same nitrogen atom may optionally be joined to form a R^{23B}-substituted or unsubstituted heteroaryl (e.g., 5 to 10 membered, 5 to 9 membered, or 5 to 6 membered). In embodiments, R^{2A} and R^{2B} substituents bonded to the same nitrogen atom may optionally be joined to form an unsubstituted heteroaryl (e.g., 5 to 10 membered, 5 to 9 membered, or 5 to 6 membered).

R^{23B} is independently oxo,

halogen, —CX^{23B}₃, —CHX^{23B}₂, —CH₂X^{23B}, —OCX^{23B}₃, —OCH₂X^{23B}, —OCHX^{23B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{24B}-substituted or unsubstituted alkyl (e.g., C₁-C₈, C₁-C₆, C₁-C₄, or C₁-C₂), R^{24B}-substituted or unsubstituted heteroalkyl (e.g., 2 to 8 membered, 2 to 6 membered, 4 to 6 membered, 2 to 3 membered, or 4 to 5 membered), R^{24B}-substituted or unsubstituted cycloalkyl (e.g., C₃-C₈, C₃-C₆, C₄-C₆, or C₅-C₆), R^{24B}-substituted or unsubstituted heterocycloalkyl (e.g., 3 to 8 membered, 3 to 6 membered, 4 to 6 membered, 4 to 5 membered, or 5 to 6 membered), R^{24B}-substituted or unsubstituted aryl (e.g., C₆-C₁₀ or phenyl), or R^{24B}-substituted or unsubstituted heteroaryl (e.g., 5 to 10 membered, 5 to 9 membered, or 5 to 6 membered). In embodiments, R^{23B} is independently oxo,
halogen, —CX^{23B}₃, —CHX^{23B}₂, —CH₂X^{23B}, —OCX^{23B}₃, —OCH₂X^{23B}, —OCHX^{23B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{23B} is independently —F, —Cl, —Br, or —I.

In embodiments, R^{23B} is independently unsubstituted methyl. In embodiments, R^{23B} is independently unsubstituted ethyl.

R^{24B} is independently oxo,

halogen, —CX^{24B}₃, —CHX^{24B}₂, —CH₂X^{24B}, —OCX^{24B}₃, —OCH₂X^{24B}, —OCHX^{24B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{25B}-substituted or unsubstituted alkyl (e.g., C₁-C₈, C₁-C₆, C₁-C₄, or C₁-C₂), R^{25B}-substituted or unsubstituted heteroalkyl (e.g., 2 to 8 membered, 2 to 6 membered, 4 to 6 membered, 2 to 3 membered, or 4 to 5 membered), R^{25B}-substituted or unsubstituted cycloalkyl (e.g., C₃-C₈, C₃-C₆, C₄-C₆, or C₅-C₆), R^{25B}-substituted or unsubstituted heterocycloalkyl (e.g., 3 to 8 membered, 3 to 6 membered, 4 to 6 membered, 4 to 5 membered, or 5 to 6 membered), R^{25B}-substituted or unsubstituted aryl (e.g., C₆-C₁₀ or phenyl), or R^{25B}-substituted or unsubstituted heteroaryl (e.g., 5 to 10 membered, 5 to 9 membered, or 5 to 6 membered). In embodiments, R^{24B} is independently oxo,
halogen, —CX^{24B}₃, —CHX^{24B}₂, —CH₂X^{24B}, —OCX^{24B}₃, —OCH₂X^{24B}, —OCHX^{24B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{24B} is independently —F, —Cl, —Br, or —I.

R^{25B} is independently oxo,

halogen, —CX^{25B}₃, —CHX^{25B}₂, —CH₂X^{25B}, —OCX^{25B}₃, —OCH₂X^{25B}, —OCHX^{25B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{25B} is independently —F, —Cl, —Br, or —I.

In embodiments, R^{2C} is independently

hydrogen, —CX^{2C}₃, —CHX^{2C}₂, —CH₂X^{2C}, —CN, —COOH, —CONH₂, R^{23C}-substituted or unsubstituted alkyl (e.g., C₁-C₈, C₁-C₆, C₁-C₄, or C₁-C₂), R^{23C}-substituted or unsubstituted heteroalkyl (e.g., 2 to 8 membered, 2 to 6 membered, 4 to 6 membered, 2 to 3 membered, or 4 to 5 membered), R^{23C}-substituted or unsubstituted cycloalkyl (e.g., C₃-C₈, C₃-C₆, C₄-C₆, or C₅-C₆), R^{23C}-substituted or unsubstituted heterocycloalkyl (e.g., 3 to 8 membered, 3 to 6 membered, 4 to 6 membered, 4 to 5 membered, or 5 to 6 membered), R^{23C}-substituted or unsubstituted aryl (e.g., C₆-C₁₀ or phenyl), or R^{23C}-substituted or unsubstituted heteroaryl (e.g., 5 to 10 membered, 5 to 9 membered, or 5 to 6 membered). In embodiments, R^{2C} is independently
hydrogen, —CX^{2C}₃, —CHX^{2C}₂, —CH₂X^{2C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2C} is independently —F, —Cl, —Br, or —I.

In embodiments, R^{2C} is independently hydrogen. In embodiments, R^{2C} is independently unsubstituted methyl. In embodiments, R^{2C} is independently unsubstituted ethyl.

R^{23C} is independently oxo,

halogen, —CX^{23C}₃, —CHX^{23C}₂, —CH₂X^{23C}, —OCX^{23C}₃, —OCH₂X^{23C}, —OCHX^{23C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{24C}-substituted or unsubstituted alkyl (e.g., C₁-C₈, C₁-C₆, C₁-C₄, or C₁-C₂), R^{24C}-substituted or unsubstituted heteroalkyl (e.g., 2 to 8 membered, 2 to 6 membered, 4 to 6 membered, 2 to 3 membered, or 4 to 5 membered), R^{24C}-substituted or unsubstituted cycloalkyl (e.g., C₃-C₈, C₃-C₆, C₄-C₆, or C₅-C₆), R^{24C}-substituted or unsubstituted heterocycloalkyl (e.g., 3 to 8 membered, 3 to 6 membered, 4 to 6 membered, 4 to 5 membered, or 5 to 6 membered), R^{24C}-substituted or unsubstituted aryl (e.g., C₆-C₁₀ or phenyl), or R^{24C}-substituted or unsubstituted heteroaryl (e.g., 5 to 10 membered, 5 to 9 membered, or 5 to 6 membered). In embodiments, R^{23C} is independently oxo,
halogen, —CX^{23C}₃, —CHX^{23C}₂, —CH₂X^{23C}, —OCX^{23C}₃, —OCH₂X^{23C}, —OCHX^{23C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{23C} is independently —F, —Cl, —Br, or —I.

In embodiments, R^{23C} is independently unsubstituted methyl. In embodiments, R^{23C} is independently unsubstituted ethyl.

R^{24C} is independently oxo,

halogen, —CX^{24C}₃, —CHX^{24C}₂, —CH₂X^{24C}, —OCX^{24C}₃, —OCH₂X^{24C}, —OCHX^{24C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{25C}-substituted or unsubstituted alkyl (e.g., C₁-C₈, C₁-C₆, C₁-C₄, or C₁-C₂), R^{25C}-substituted or unsubstituted heteroalkyl (e.g., 2 to 8 membered, 2 to 6 membered, 4 to 6 membered, 2 to 3 membered, or 4 to 5 membered), R^{25C}-substituted or unsubstituted cycloalkyl (e.g., C₃-C₈, C₃-C₆, C₄-C₆, or C₅-C₆), R^{25C}-substituted or unsubstituted heterocycloalkyl (e.g., 3 to 8 membered, 3 to 6 membered, 4 to 6 membered, 4 to 5 membered, or 5 to 6 membered), R^{25C}-substituted or unsubstituted aryl (e.g., C₆-C₁₀ or phenyl), or R^{25C}-substituted or unsubstituted heteroaryl (e.g., 5 to 10 membered, 5 to 9 membered, or 5 to 6 membered). In embodiments, R^{24C} is independently oxo,
halogen, —CX^{24C}₃, —CHX^{24C}₂, —CH₂X^{24C}, —OCX^{24C}₃, —OCH₂X^{24C}, —OCHX^{24C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{24C} is independently —F, —Cl, —Br, or —I.

R^{25C} is independently oxo,

halogen, —CX^{25C}₃, —CHX^{25C}₂, —CH₂X^{25C}, —OCX^{25C}₃, —OCH₂X^{25C}, —OCHX^{25C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{25C} is independently —F, —Cl, —Br, or —I.

In embodiments, R^{2D} is independently

hydrogen, —CX^{2D}₃, —CHX^{2D}₂, —CH₂X^{2D}, —CN, —COOH, —CONH₂, R^{23D}-substituted or unsubstituted alkyl (e.g., C₁-C₈, C₁-C₆, C₁-C₄, or C₁-C₂), R^{23D}-substituted or unsubstituted heteroalkyl (e.g., 2 to 8 membered, 2 to 6 membered, 4 to 6 membered, 2 to 3 membered, or 4 to 5 membered), R^{23D}-substituted or unsubstituted cycloalkyl (e.g., C₃-C₈, C₃-C₆, C₄-C₆, or C₅-C₆), R^{23D}-substituted or unsubstituted heterocycloalkyl (e.g., 3 to 8 membered, 3 to 6 membered, 4 to 6 membered, 4 to 5 membered, or 5 to 6 membered), R^{23D}-substituted or unsubstituted aryl (e.g., C₆-C₁₀ or phenyl), or R^{23D}-substituted or unsubstituted heteroaryl (e.g., 5 to 10 membered, 5 to 9 membered, or 5 to 6 membered). In embodiments, R^{2D} is independently
hydrogen, —CX^{2D}₃, —CHX^{2D}₂, —CH₂X^{2D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{2D} is independently —F, —Cl, —Br, or —I.

In embodiments, R^{2D} is independently hydrogen. In embodiments, R^{2D} is independently unsubstituted methyl. In embodiments, R^{2D} is independently unsubstituted ethyl.

R^{23D} is independently oxo,

halogen, —CX^{23D}₃, —CHX^{23D}₂, —CH₂X^{23D}, —OCX^{23D}₃, —OCH₂X^{23D}, —OCHX^{23D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{24D}-substituted or unsubstituted alkyl (e.g., C₁-C₈, C₁-C₆, C₁-C₄, or C₁-C₂), R^{4D}-substituted or unsubstituted heteroalkyl (e.g., 2 to 8 membered, 2 to 6 membered, 4 to 6 membered, 2 to 3 membered, or 4 to 5 membered), R^{24D}-substituted or unsubstituted cycloalkyl (e.g., C₃-C₈, C₃-C₆, C₄-C₆, or C₅-C₆), R^{24D}-substituted or unsubstituted heterocycloalkyl (e.g., 3 to 8 membered, 3 to 6 membered, 4 to 6 membered, 4 to 5 membered, or 5 to 6 membered), R^{24D}-substituted or unsubstituted aryl (e.g., C₆-C₁₀ or phenyl), or R^{24D}-substituted or unsubstituted heteroaryl (e.g., 5 to 10 membered, 5 to 9 membered, or 5 to 6 membered). In embodiments, R^{23D} is independently oxo,
halogen, —CX^{23D}₃, —CHX^{23D}₂, —CH₂X^{23D}, —OCX^{23D}₃, —OCH₂X^{23D}, —OCHX^{23D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{23D} is independently —F, —Cl, —Br, or —I.

In embodiments, R^{23D} is independently unsubstituted methyl. In embodiments, R^{23D} is independently unsubstituted ethyl.

R^{24D} is independently oxo,

halogen, —CX^{24D}₃, —CHX^{24D}₂, —CH₂X^{24D}, —OCX^{24D}₃, —OCH₂X^{24D}, —OCHX^{24D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{25D}-substituted or unsubstituted alkyl (e.g., C₁-C₈, C₁-C₆, C₁-C₄, or C₁-C₂), R^{25D}-substituted or unsubstituted heteroalkyl (e.g., 2 to 8 membered, 2 to 6 membered, 4 to 6 membered, 2 to 3 membered, or 4 to 5 membered), R^{25D}-substituted or unsubstituted cycloalkyl (e.g., C₃-C₈, C₃-C₆, C₄-C₆, or C₅-C₆), R^{25D}-substituted or unsubstituted heterocycloalkyl (e.g., 3 to 8 membered, 3 to 6 membered, 4 to 6 membered, 4 to 5 membered, or 5 to 6 membered), R^{25D}-substituted or unsubstituted aryl (e.g., C₆-C₁₀ or phenyl), or R^{25D}-substituted or unsubstituted heteroaryl (e.g., 5 to 10 membered, 5 to 9 membered, or 5 to 6 membered). In embodiments, R^{24D} is independently oxo,
halogen, —CX^{24D}₃, —CHX^{24D}₂, —CH₂X^{24D}, —OCX^{24D}₃, —OCH₂X^{24D}, —OCHX^{24D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{24D} is independently —F, —Cl, —Br, or —I.

R^{25D} is independently oxo,

halogen, —CX^{25D}₃, —CHX^{25D}₂, —CH₂X^{25D}, —OCX^{25D}₃, —OCH₂X^{25D}, —OCHX^{25D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{25D} is independently —F, —Cl, —Br, or —I.

In embodiments, R³ is independently hydrogen, —CX³₃, —CHX³₂, —CH₂X³, —CN, —C(O)R^{3C}, —C(O)OR^{3C}, —C(O)NR^{3A}R^{3B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R³ is independently hydrogen, —CX³₃, —CHX³₂, —CH₂X³, —CN, —C(O)R^{3C}, —C(O)OR^{3C}, —C(O)NR^{3A}R^{3B}, Substituted or unsubstituted C₁-C₄ alkyl, or substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R³ is independently hydrogen, —CX³₃, —CHX³₂, —CH₂X³, —CN, —C(O)R^{3C}, —C(O)OR^{3C}, —C(O)NR^{3A}R^{3B}, unsubstituted C₁-C₄ alkyl, or unsubstituted 2 to 4 membered heteroalkyl.

In embodiments, R^{3A} is independently substituted or unsubstituted alkyl (e.g., C₁-C₈, C₁-C₆, C₁-C₄, or C₁-C₂). In embodiments, R^{3A} is independently substituted alkyl (e.g., C₁-C₈, C₁-C₆, C₁-C₄, or C₁-C₂). In embodiments, R^{3A} is independently unsubstituted alkyl (e.g., C₁-C₈, C₁-C₆, C₁-C₄, or C₁-C₂). In embodiments, R^{3A} is independently unsubstituted methyl. In embodiments, R^{3A} is independently unsubstituted ethyl. In embodiments, R^{3A} is independently unsubstituted propyl. In embodiments, R^{3A} is independently unsubstituted isopropyl. In embodiments, R^{3A} is independently unsubstituted tert-butyl. In embodiments, R^{3A} is independently substituted or unsubstituted heteroalkyl (e.g., 2 to 8 membered, 2 to 6 membered, 4 to 6 membered, 2 to 3 membered, or 4 to 5 membered). In embodiments, R^{3A} is independently substituted heteroalkyl (e.g., 2 to 8 membered, 2 to 6 membered, 4 to 6 membered, 2 to 3 membered, or 4 to 5 membered). In embodiments, R^{3A} is independently unsubstituted heteroalkyl (e.g., 2 to 8 membered, 2 to 6 membered, 4 to 6 membered, 2 to 3 membered, or 4 to 5 membered). In embodiments, R^{3A} is independently substituted or unsubstituted cycloalkyl (e.g., C₃-C₈, C₃-C₆, C₄-C₆, or C₅-C₆). In embodiments, R^{3A} is independently substituted cycloalkyl (e.g., C₃-C₈, C₃-C₆, C₄-C₆, or C₅-C₆). In embodiments, R^{3A} is independently unsubstituted cycloalkyl (e.g., C₃-C₈, C₃-C₆, C₄-C₆, or C₅-C₆). In embodiments, R^{3A} is independently substituted or unsubstituted heterocycloalkyl (e.g., 3 to 8 membered, 3 to 6 membered, 4 to 6 membered, 4 to 5 membered, or 5 to 6 membered). In embodiments, R^{3A} is independently substituted heterocycloalkyl (e.g., 3 to 8 membered, 3 to 6 membered, 4 to 6 membered, 4 to 5 membered, or 5 to 6 membered). In embodiments, R^{3A} is independently unsubstituted heterocycloalkyl (e.g., 3 to 8 membered, 3 to 6 membered, 4 to 6 membered, 4 to 5 membered, or 5 to 6 membered). In embodiments, R^{3A} is independently substituted or unsubstituted aryl (e.g., C₆-C₁₀ or phenyl). In embodiments, R^{3A} is independently substituted aryl (e.g., C₆-C₁₀ or phenyl). In embodiments, R^{3A} is independently unsubstituted aryl (e.g., C₆-C₁₀ or phenyl). In embodiments, R^{3A} is independently substituted or unsubstituted heteroaryl (e.g., 5 to 10 membered, 5 to 9 membered, or 5 to 6 membered). In embodiments, R^{3A} is independently substituted heteroaryl (e.g., 5 to 10 membered, 5 to 9 membered, or 5 to 6 membered). In embodiments, R^{3A} is independently unsubstituted heteroaryl (e.g., 5 to 10 membered, 5 to 9 membered, or 5 to 6 membered).

In embodiments, R^{3B} is independently substituted or unsubstituted alkyl (e.g., C₁-C₈, C₁-C₆, C₁-C₄, or C₁-C₂). In embodiments, R^{3B} is independently substituted alkyl (e.g., C₁-C₈, C₁-C₆, C₁-C₄, or C₁-C₂). In embodiments, R^{3B} is independently unsubstituted alkyl (e.g., C₁-C₈, C₁-C₆, C₁-C₄, or C₁-C₂). In embodiments, R^{3B} is independently unsubstituted methyl. In embodiments, R^{3B} is independently unsubstituted ethyl. In embodiments, R^{3B} is independently unsubstituted propyl. In embodiments, R^{3B} is independently unsubstituted isopropyl. In embodiments, R^{3B} is independently unsubstituted tert-butyl. In embodiments, R^{3B} is independently substituted or unsubstituted heteroalkyl (e.g., 2 to 8 membered, 2 to 6 membered, 4 to 6 membered, 2 to 3 membered, or 4 to 5 membered). In embodiments, R^{3B} is independently substituted heteroalkyl (e.g., 2 to 8 membered, 2 to 6 membered, 4 to 6 membered, 2 to 3 membered, or 4 to 5 membered). In embodiments, R^{3B} is independently unsubstituted heteroalkyl (e.g., 2 to 8 membered, 2 to 6 membered, 4 to 6 membered, 2 to 3 membered, or 4 to 5 membered). In embodiments, R^{3B} is independently substituted or unsubstituted cycloalkyl (e.g., C₃-C₈, C₃-C₆, C₄-C₆, or C₅-C₆). In embodiments, R^{3B} is independently substituted cycloalkyl (e.g., C₃-C₈, C₃-C₆, C₄-C₆, or C₅-C₆). In embodiments, R^{3B} is independently unsubstituted cycloalkyl (e.g., C₃-C₈, C₃-C₆, C₄-C₆, or C₅-C₆). In embodiments, R^{3B} is independently substituted or unsubstituted heterocycloalkyl (e.g., 3 to 8 membered, 3 to 6 membered, 4 to 6 membered, 4 to 5 membered, or 5 to 6 membered). In embodiments, R^{3B} is independently substituted heterocycloalkyl (e.g., 3 to 8 membered, 3 to 6 membered, 4 to 6 membered, 4 to 5 membered, or 5 to 6 membered). In embodiments, R^{3B} is independently unsubstituted heterocycloalkyl (e.g., 3 to 8 membered, 3 to 6 membered, 4 to 6 membered, 4 to 5 membered, or 5 to 6 membered). In embodiments, R^{3B} is independently substituted or unsubstituted aryl (e.g., C₆-C₁₀ or phenyl). In embodiments, R^{3B} is independently substituted aryl (e.g., C₆-C₁₀ or phenyl). In embodiments, R^{3B} is independently unsubstituted aryl (e.g., C₆-C₁₀ or phenyl). In embodiments, R^{3B} is independently substituted or unsubstituted heteroaryl (e.g., 5 to 10 membered, 5 to 9 membered, or 5 to 6 membered). In embodiments, R^{3B} is independently substituted heteroaryl (e.g., 5 to 10 membered, 5 to 9 membered, or 5 to 6 membered). In embodiments, R^{3B} is independently unsubstituted heteroaryl (e.g., 5 to 10 membered, 5 to 9 membered, or 5 to 6 membered).

In embodiments, R^{3A} and R^{3B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{3A} and R^{3B} substituents bonded to the same nitrogen atom may be joined to form a substituted or unsubstituted heteroaryl (e.g., 5 to 10 membered, 5 to 9 membered, or 5 to 6 membered). In embodiments, R^{3A} and R^{3B} substituents bonded to the same nitrogen atom may be joined to form a substituted heteroaryl (e.g., 5 to 10 membered, 5 to 9 membered, or 5 to 6 membered). In embodiments, R^{3A} and R^{3B} substituents bonded to the same nitrogen atom may be joined to form an unsubstituted heteroaryl (e.g., 5 to 10 membered, 5 to 9 membered, or 5 to 6 membered).

In embodiments, R^{3C} is independently substituted or unsubstituted alkyl (e.g., C₁-C₈, C₁-C₆, C₁-C₄, or C₁-C₂). In embodiments, R^{3C} is independently substituted alkyl (e.g., C₁-C₈, C₁-C₆, C₁-C₄, or C₁-C₂). In embodiments, R^{3C} is independently unsubstituted alkyl (e.g., C₁-C₈, C₁-C₆, C₁-C₄, or C₁-C₂). In embodiments, R^{3C} is independently unsubstituted methyl. In embodiments, R^{3C} is independently unsubstituted ethyl. In embodiments, R^{3C} is independently unsubstituted propyl. In embodiments, R^{3C} is independently unsubstituted isopropyl. In embodiments, R^{3C} is independently unsubstituted tert-butyl. In embodiments, R^{3C} is independently substituted or unsubstituted heteroalkyl (e.g., 2 to 8 membered, 2 to 6 membered, 4 to 6 membered, 2 to 3 membered, or 4 to 5 membered). In embodiments, R^{3C} is independently substituted heteroalkyl (e.g., 2 to 8 membered, 2 to 6 membered, 4 to 6 membered, 2 to 3 membered, or 4 to 5 membered). In embodiments, R³ is independently unsubstituted heteroalkyl (e.g., 2 to 8 membered, 2 to 6 membered, 4 to 6 membered, 2 to 3 membered, or 4 to 5 membered). In embodiments, R^{3C} is independently substituted or unsubstituted cycloalkyl (e.g., C₃-C₈, C₃-C₆, C₄-C₆, or C₅-C₆). In embodiments, R^{3C} is independently substituted cycloalkyl (e.g., C₃-C₈, C₃-C₆, C₄-C₆, or C₅-C₆). In embodiments, R^{3C} is independently unsubstituted cycloalkyl (e.g., C₃-C₈, C₃-C₆, C₄-C₆, or C₅-C₆). In embodiments, R^{3C} is independently substituted or unsubstituted heterocycloalkyl (e.g., 3 to 8 membered, 3 to 6 membered, 4 to 6 membered, 4 to 5 membered, or 5 to 6 membered). In embodiments, R^{3C} is independently substituted heterocycloalkyl (e.g., 3 to 8 membered, 3 to 6 membered, 4 to 6 membered, 4 to 5 membered, or 5 to 6 membered). In embodiments, R^{3C} is independently unsubstituted heterocycloalkyl (e.g., 3 to 8 membered, 3 to 6 membered, 4 to 6 membered, 4 to 5 membered, or 5 to 6 membered). In embodiments, R^{3C} is independently substituted or unsubstituted aryl (e.g., C₆-C₁₀ or phenyl). In embodiments, R^{3C} is independently substituted aryl (e.g., C₆-C₁₀ or phenyl). In embodiments, R^{3C} is independently unsubstituted aryl (e.g., C₆-C₁₀ or phenyl). In embodiments, R^{3C} is independently substituted or unsubstituted heteroaryl (e.g., 5 to 10 membered, 5 to 9 membered, or 5 to 6 membered). In embodiments, R^{3C} is independently substituted heteroaryl (e.g., 5 to 10 membered, 5 to 9 membered, or 5 to 6 membered). In embodiments, R^{3C} is independently unsubstituted heteroaryl (e.g., 5 to 10 membered, 5 to 9 membered, or 5 to 6 membered).

In embodiments, R³ is independently hydrogen, —CX³₃, —CHX³₂, —CH₂X³, —CN, C(O)R^{3C}, —C(O)OR^{3C}, —C(O)NR^{3A}R^{3B}, R⁶-substituted or unsubstituted alkyl (e.g., C₁-C₈, C₁-C₆, C₁-C₄, or C₁-C₂), R²-substituted or unsubstituted heteroalkyl (e.g., 2 to 8 membered, 2 to 6 membered, 4 to 6 membered, 2 to 3 membered, or 4 to 5 membered), R²⁶-substituted or unsubstituted cycloalkyl (e.g., C₃-C₈, C₃-C₆, C₄-C₆, or C₅-C₆), R²⁶-substituted or unsubstituted heterocycloalkyl (e.g., 3 to 8 membered, 3 to 6 membered, 4 to 6 membered, 4 to 5 membered, or 5 to 6 membered), R²⁶-substituted or unsubstituted aryl (e.g., C₆-C₁₀ or phenyl), or R²⁶-substituted or unsubstituted heteroaryl (e.g., 5 to 10 membered, 5 to 9 membered, or 5 to 6 membered). In embodiments, R³ is independently

hydrogen, —CX³₃, —CHX³₂, —CH₂X³, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³ is independently —F, —Cl, —Br, or —I.

In embodiments, R³ is independently hydrogen. In embodiments, R³ is independently unsubstituted methyl. In embodiments, R³ is independently unsubstituted ethyl.

R² is independently oxo,

halogen, —CX²⁶₃, —CHX²⁶₂, —CH₂X²⁶, —OCX²⁶₃, —OCH₂X²⁶, —OCHX²⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²⁷-substituted or unsubstituted alkyl (e.g., C₁-C₈, C₁-C₆, C₁-C₄, or C₁-C₂), R²⁷-substituted or unsubstituted heteroalkyl (e.g., 2 to 8 membered, 2 to 6 membered, 4 to 6 membered, 2 to 3 membered, or 4 to 5 membered), R²⁷-substituted or unsubstituted cycloalkyl (e.g., C₃-C₈, C₃-C₆, C₄-C₆, or C₅-C₆), R²⁷-substituted or unsubstituted heterocycloalkyl (e.g., 3 to 8 membered, 3 to 6 membered, 4 to 6 membered, 4 to 5 membered, or 5 to 6 membered), R²⁷-substituted or unsubstituted aryl (e.g., C₆-C₁₀ or phenyl), or R²⁷-substituted or unsubstituted heteroaryl (e.g., 5 to 10 membered, 5 to 9 membered, or 5 to 6 membered). In embodiments, R² is independently oxo,
halogen, —CX²⁶₃, —CHX²⁶₂, —CH₂X²⁶, —OCX²⁶₃, —OCH₂X²⁶, —OCHX²⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁶ is independently —F, —Cl, —Br, or —I.

In embodiments, R²⁶ is independently unsubstituted methyl. In embodiments, R²⁶ is independently unsubstituted ethyl.

R²⁷ is independently oxo,

halogen, —CX²⁷₃, —CHX²⁷₂, —CH₂X²⁷, —OCX²⁷₃, —OCH₂X²⁷, —OCHX²⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R²⁸-substituted or unsubstituted alkyl (e.g., C₁-C₈, C₁-C₆, C₁-C₄, or C₁-C₂), R²-substituted or unsubstituted heteroalkyl (e.g., 2 to 8 membered, 2 to 6 membered, 4 to 6 membered, 2 to 3 membered, or 4 to 5 membered), R²⁸-substituted or unsubstituted cycloalkyl (e.g., C₃-C₈, C₃-C₆, C₄-C₆, or C₅-C₆), R²⁸-substituted or unsubstituted heterocycloalkyl (e.g., 3 to 8 membered, 3 to 6 membered, 4 to 6 membered, 4 to 5 membered, or 5 to 6 membered), R²⁸-substituted or unsubstituted aryl (e.g., C₆-C₁₀ or phenyl), or R²⁸-substituted or unsubstituted heteroaryl (e.g., 5 to 10 membered, 5 to 9 membered, or 5 to 6 membered). In embodiments, R²⁷ is independently oxo,
halogen, —CX²⁷₃, —CHX²⁷₂, —CH₂X²⁷, —OCX²⁷₃, —OCH₂X²⁷, —OCHX²⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁷ is independently —F, —Cl, —Br, or —I. In embodiments, R² is independently unsubstituted methyl. In embodiments, R² is independently unsubstituted ethyl.

R²⁸ is independently oxo,

halogen, —CX²⁸₃, —CHX²⁸₂, —CH₂X²⁸, —OCX²⁸₃, —OCH₂X²⁸, —OCHX²⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁸ is independently —F, —Cl, —Br, or —I.

In embodiments, R²⁸ is independently unsubstituted methyl. In embodiments, R³ is independently unsubstituted ethyl.

In embodiments, R^{3A} is independently

hydrogen, —CX^{3A}₃, —CHX^{3A}₂, —CH₂X^{3A}, —CN, —COOH, —CONH₂, R^{26A}-substituted or unsubstituted alkyl (e.g., C₁-C₈, C₁-C₆, C₁-C₄, or C₁-C₂), R^{26A}-substituted or unsubstituted heteroalkyl (e.g., 2 to 8 membered, 2 to 6 membered, 4 to 6 membered, 2 to 3 membered, or 4 to 5 membered), R^{26A}-substituted or unsubstituted cycloalkyl (e.g., C₃-C₈, C₃-C₆, C₄-C₆, or C₅-C₆), R^{26A}-substituted or unsubstituted heterocycloalkyl (e.g., 3 to 8 membered, 3 to 6 membered, 4 to 6 membered, 4 to 5 membered, or 5 to 6 membered), R^{26A}-substituted or unsubstituted aryl (e.g., C₆-C₁₀ or phenyl), or R^{26A}-substituted or unsubstituted heteroaryl (e.g., 5 to 10 membered, 5 to 9 membered, or 5 to 6 membered). In embodiments, R^{3A} is independently
hydrogen, —CX^{3A}₃, —CHX^{3A}₂, —CH₂X^{3A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{3A} is independently —F, —Cl, —Br, or —I.

In embodiments, R^{3A} is independently hydrogen. In embodiments, R^{3A} is independently unsubstituted methyl. In embodiments, R^{3A} is independently unsubstituted ethyl.

In embodiments, R^{3A} and R^{3B} substituents bonded to the same nitrogen atom may optionally be joined to form a R^{26A}-substituted or unsubstituted heterocycloalkyl (e.g., 3 to 8 membered, 3 to 6 membered, 4 to 6 membered, 4 to 5 membered, or 5 to 6 membered) or R^{26A}-substituted or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form a R^{26A}-substituted or unsubstituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may optionally be joined to form a R^{26A}-substituted or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form an unsubstituted heteroaryl (e.g., 5 to 10 membered, 5 to 9 membered, or 5 to 6 membered).

R^{26A} is independently oxo,

halogen, —CX^{26A}₃, —CHX^{26A}₂, —CH₂X^{26A}, —OCX^{26A}₃, —OCH₂X^{26A}, —OCHX^{26A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{27A}-substituted or unsubstituted alkyl (e.g., C₁-C₈, C₁-C₆, C₁-C₄, or C₁-C₂), R^{27A}-substituted or unsubstituted heteroalkyl (e.g., 2 to 8 membered, 2 to 6 membered, 4 to 6 membered, 2 to 3 membered, or 4 to 5 membered), R^{27A}-substituted or unsubstituted cycloalkyl (e.g., C₃-C₈, C₃-C₆, C₄-C₆, or C₅-C₆), R^{27A}-substituted or unsubstituted heterocycloalkyl (e.g., 3 to 8 membered, 3 to 6 membered, 4 to 6 membered, 4 to 5 membered, or 5 to 6 membered), R^{27A}-substituted or unsubstituted aryl (e.g., C₆-C₁₀ or phenyl), R^{27A}-substituted or unsubstituted heteroaryl (e.g., 5 to 10 membered, 5 to 9 membered, or 5 to 6 membered). In embodiments, R^{26A} is independently oxo,
halogen, —CX^{26A}₃, —CHX^{26A}₂, —CH₂X^{26A}, —OCX^{26A}₃, —OCH₂X^{26A}, —OCHX^{26A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), unsubstituted heteroaryl (e.g., 5 to 10 membered, 5 to 9 membered, or 5 to 6 membered). X^{26A} is independently —F, —Cl, —Br, or —I.

In embodiments, R^{26A} is independently unsubstituted methyl. In embodiments, R^{26A} is independently unsubstituted ethyl.

R^{27A} is independently oxo,

halogen, —CX^{27A}₃, —CHX^{27A}₂, —CH₂X^{27A}, —OCX^{27A}₃, —OCH₂X^{27A}, —OCHX^{27A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{28A}-substituted or unsubstituted alkyl (e.g., C₁-C₈, C₁-C₆, C₁-C₄, or C₁-C₂), R^{28A}-substituted or unsubstituted heteroalkyl (e.g., 2 to 8 membered, 2 to 6 membered, 4 to 6 membered, 2 to 3 membered, or 4 to 5 membered), R^{28A}-substituted or unsubstituted cycloalkyl (e.g., C₃-C₈, C₃-C₆, C₄-C₆, or C₅-C₆), R^{28A}-substituted or unsubstituted heterocycloalkyl (e.g., 3 to 8 membered, 3 to 6 membered, 4 to 6 membered, 4 to 5 membered, or 5 to 6 membered), R^{28A}-substituted or unsubstituted aryl (e.g., C₆-C₁₀ or phenyl), or R^{28A}-substituted or unsubstituted heteroaryl (e.g., 5 to 10 membered, 5 to 9 membered, or 5 to 6 membered). In embodiments, R^{27A} is independently oxo,
halogen, —CX^{27A}₃, —CHX^{27A}₂, —CH₂X^{27A}, —OCX^{27A}₃, —OCH₂X^{27A}, —OCHX^{27A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{27A} is independently —F, —Cl, —Br, or —I.

R^{2A} is independently oxo,

halogen, —CX^{28A}₃, —CHX^{28A}₂, —CH₂X^{28A}, —OCX^{28A}₃, —OCH₂X^{28A}, —OCHX^{28A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{28A} is independently —F, —Cl, —Br, or —I.

In embodiments, R^{3B} is independently

hydrogen, —CX^{3B}₃, —CHX^{3B}₂, —CH₂X^{3B}, —CN, —COOH, —CONH₂, R^{26B}-substituted or unsubstituted alkyl (e.g., C₁-C₈, C₁-C₆, C₁-C₄, or C₁-C₂), R^{26B}-substituted or unsubstituted heteroalkyl (e.g., 2 to 8 membered, 2 to 6 membered, 4 to 6 membered, 2 to 3 membered, or 4 to 5 membered), R^{26B}-substituted or unsubstituted cycloalkyl (e.g., C₃-C₈, C₃-C₆, C₄-C₆, or C₅-C₆), R^{26B}-substituted or unsubstituted heterocycloalkyl (e.g., 3 to 8 membered, 3 to 6 membered, 4 to 6 membered, 4 to 5 membered, or 5 to 6 membered), R^{26B}-substituted or unsubstituted aryl (e.g., C₆-C₁₀ or phenyl), or R^{26B}-substituted or unsubstituted heteroaryl (e.g., 5 to 10 membered, 5 to 9 membered, or 5 to 6 membered). In embodiments, R^{3B} is independently
hydrogen, —CX^{3B}₃, —CHX^{3B}₂, —CH₂X^{3B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{3B} is independently —F, —Cl, —Br, or —I.

In embodiments, R^{3B} is independently hydrogen. In embodiments, R^{3B} is independently unsubstituted methyl. In embodiments, R^{3B} is independently unsubstituted ethyl.

In embodiments, R^{3A} and R^{3B} substituents bonded to the same nitrogen atom may optionally be joined to form a R^{26B}-substituted or unsubstituted heterocycloalkyl (e.g., 3 to 8 membered, 3 to 6 membered, 4 to 6 membered, 4 to 5 membered, or 5 to 6 membered) or R^{26B}-substituted or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form a R^{26B}-substituted or unsubstituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{3A} and R^{3B} substituents bonded to the same nitrogen atom may optionally be joined to form a R^{26B}-substituted or unsubstituted heteroaryl (e.g., 5 to 10 membered, 5 to 9 membered, or 5 to 6 membered). In embodiments, R^{3A} and R^{3B} substituents bonded to the same nitrogen atom may optionally be joined to form an unsubstituted heteroaryl (e.g., 5 to 10 membered, 5 to 9 membered, or 5 to 6 membered).

R^{26B} is independently oxo,

halogen, —CX^{26B}₃, —CHX^{26B}₂, —CH₂X^{26B}, —OCX^{26B}₃, —OCH₂X^{26B}, —OCHX^{26B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{27B}-substituted or unsubstituted alkyl (e.g., C₁-C₈, C₁-C₆, C₁-C₄, or C₁-C₂), R^{27B}-substituted or unsubstituted heteroalkyl (e.g., 2 to 8 membered, 2 to 6 membered, 4 to 6 membered, 2 to 3 membered, or 4 to 5 membered), R^{27B}-substituted or unsubstituted cycloalkyl (e.g., C₃-C₈, C₃-C₆, C₄-C₆, or C₅-C₆), R^{27B}-substituted or unsubstituted heterocycloalkyl (e.g., 3 to 8 membered, 3 to 6 membered, 4 to 6 membered, 4 to 5 membered, or 5 to 6 membered), R^{27B}-substituted or unsubstituted aryl (e.g., C₆-C₁₀ or phenyl), R^{27B}-substituted or unsubstituted heteroaryl (e.g., 5 to 10 membered, 5 to 9 membered, or 5 to 6 membered). In embodiments, R^{26B} is independently oxo,
halogen, —CX^{26B}₃, —CHX^{26B}₂, —CH₂X^{26B}, —OCX^{26B}₃, —OCH₂X^{26B}, —OCHX^{26B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), unsubstituted heteroaryl (e.g., 5 to 10 membered, 5 to 9 membered, or 5 to 6 membered). X^{26B} is independently —F, —Cl, —Br, or —I.

In embodiments, R^{26B} is independently unsubstituted methyl. In embodiments, R^{26B} is independently unsubstituted ethyl.

R^{27B} is independently oxo,

halogen, —CX^{27B}₃, —CHX^{27B}₂, —CH₂X^{27B}, —OCX^{27B}₃, —OCH₂X^{27B}, —OCHX^{27B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{28B}-substituted or unsubstituted alkyl (e.g., C₁-C₈, C₁-C₆, C₁-C₄, or C₁-C₂), R^{28B}-substituted or unsubstituted heteroalkyl (e.g., 2 to 8 membered, 2 to 6 membered, 4 to 6 membered, 2 to 3 membered, or 4 to 5 membered), R^{28B}-substituted or unsubstituted cycloalkyl (e.g., C₃-C₈, C₃-C₆, C₄-C₆, or C₅-C₆), R²-substituted or unsubstituted heterocycloalkyl (e.g., 3 to 8 membered, 3 to 6 membered, 4 to 6 membered, 4 to 5 membered, or 5 to 6 membered), R^{28B}-substituted or unsubstituted aryl (e.g., C₆-C₁₀ or phenyl), or R^{28B}-substituted or unsubstituted heteroaryl (e.g., 5 to 10 membered, 5 to 9 membered, or 5 to 6 membered). In embodiments, R^{27B} is independently oxo,

halogen, —CX^{27B}₃, —CHX^{27B}₂, —CH₂X^{27B}, —OCX^{27B}₃, —OCH₂X^{27B}, —OCHX^{27B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{27B} is independently —F, —Cl, —Br, or —I.

R^{28B} is independently oxo,

halogen, —CX^{28B}₃, —CHX^{28B}₂, —CH₂X^{28B}, —OCX^{28B}₃, —OCH₂X^{28B}, —OCHX^{28B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{28B} is independently —F, —Cl, —Br, or —I.

In embodiments, R^{3C} is independently

hydrogen, —CX^{3C}₃, —CHX^{3C}₂, —CH₂X^{3C}, —CN, —COOH, —CONH₂, R^{26C}-substituted or unsubstituted alkyl (e.g., C₁-C₈, C₁-C₆, C₁-C₄, or C₁-C₂), R²⁶-substituted or unsubstituted heteroalkyl (e.g., 2 to 8 membered, 2 to 6 membered, 4 to 6 membered, 2 to 3 membered, or 4 to 5 membered), R^{26C}-substituted or unsubstituted cycloalkyl (e.g., C₃-C₈, C₃-C₆, C₄-C₆, or C₅-C₆), R^{26C}-substituted or unsubstituted heterocycloalkyl (e.g., 3 to 8 membered, 3 to 6 membered, 4 to 6 membered, 4 to 5 membered, or 5 to 6 membered), R^{26C}-substituted or unsubstituted aryl (e.g., C₆-C₁₀ or phenyl), or R^{26C}-substituted or unsubstituted heteroaryl (e.g., 5 to 10 membered, 5 to 9 membered, or 5 to 6 membered). In embodiments, R^{3C} is independently
hydrogen, —CX^{3C}₃, —CHX^{3C}₂, —CH₂X^{3C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{3C} is independently —F, —Cl, —Br, or —I.

In embodiments, R^{3C} is independently hydrogen. In embodiments, R^{3C} is independently unsubstituted methyl. In embodiments, R^{3C} is independently unsubstituted ethyl.

R^{26C} is independently oxo,

halogen, —CX^{26C}₃, —CHX^{26C}₂, —CH₂X^{26C}, —OCX^{26C}₃, —OCH₂X^{26C}, —OCHX^{26C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{27C}-substituted unsubstituted alkyl (e.g., C₁-C₈, C₁-C₆, C₁-C₄, or C₁-C₂), R^{27C}-substituted unsubstituted heteroalkyl (e.g., 2 to 8 membered, 2 to 6 membered, 4 to 6 membered, 2 to 3 membered, or 4 to 5 membered), R^{27C}-substituted unsubstituted cycloalkyl (e.g., C₃-C₈, C₃-C₆, C₄-C₆, or C₅-C₆), R^{27C}-substituted unsubstituted heterocycloalkyl (e.g., 3 to 8 membered, 3 to 6 membered, 4 to 6 membered, 4 to 5 membered, or 5 to 6 membered), R^{27C}-substituted unsubstituted aryl (e.g., C₆-C₁₀ or phenyl), R^{27C}-substituted or unsubstituted heteroaryl (e.g., 5 to 10 membered, 5 to 9 membered, or 5 to 6 membered). In embodiments, R^{26C} is independently oxo,
halogen, —CX^{26C}₃, —CHX^{26C}₂, —CH₂X^{26C}, —OCX^{26C}₃, —OCH₂X^{26C}, —OCHX^{26C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), unsubstituted heteroaryl (e.g., 5 to 10 membered, 5 to 9 membered, or 5 to 6 membered). X^{26C} is independently —F, —Cl, —Br, or —I.

In embodiments, R^{26C} is independently unsubstituted methyl. In embodiments, R^{26C} is independently unsubstituted ethyl.

R^{27C} is independently oxo,

halogen, —CX^{27C}₃, —CHX^{27C}₂, —CH₂X^{27C}, —OCX^{27C}₃, —OCH₂X^{27C}, —OCHX^{27C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{28C}-substituted or unsubstituted alkyl (e.g., C₁-C₈, C₁-C₆, C₁-C₄, or C₁-C₂), R^{28C}-substituted or unsubstituted heteroalkyl (e.g., 2 to 8 membered, 2 to 6 membered, 4 to 6 membered, 2 to 3 membered, or 4 to 5 membered), R^{28C}-substituted or unsubstituted cycloalkyl (e.g., C₃-C₈, C₃-C₆, C₄-C₆, or C₅-C₆), R^{28C}-substituted or unsubstituted heterocycloalkyl (e.g., 3 to 8 membered, 3 to 6 membered, 4 to 6 membered, 4 to 5 membered, or 5 to 6 membered), R^{28C}-substituted or unsubstituted aryl (e.g., C₆-C₁₀ or phenyl), or R^{28C}-substituted or unsubstituted heteroaryl (e.g., 5 to 10 membered, 5 to 9 membered, or 5 to 6 membered). In embodiments, R^{27C} is independently oxo,
halogen, —CX^{27C}₃, —CHX^{27C}₂, —CH₂X^{27C}, —OCX^{27C}₃, —OCH₂X^{27C}, —OCHX^{27C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{27C} is independently —F, —Cl, —Br, or —I.

R^{28C} is independently oxo,

halogen, —CX^{28C}₃, —CHX^{28C}₂, —CH₂X^{28C}, —OCX^{28C}₃, —OCH₂X^{28C}, —OCHX^{28C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{28C} is independently —F, —Cl, —Br, or —I.

In embodiments, R⁴ is independently hydrogen, —CX⁴₃, —CHX⁴₂, —CH₂X⁴, —CN, —C(O)R^{4C}, —C(O)OR^{4C}, —C(O)NR^{4A}R^{4B}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is independently hydrogen,

halogen, —CX⁴₃, —CN, —CHX⁴₂, —CH₂X⁴, —COOH, —CONH₂, substituted or unsubstituted C₁-C₄ alkyl, or substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is independently hydrogen, halogen, —CX⁴₃, —CN, —CHX⁴₂, —CH₂X⁴, unsubstituted C₁-C₄ alkyl, or unsubstituted 2 to 4 membered heteroalkyl.

In embodiments, R^{4A} is independently substituted or unsubstituted alkyl (e.g., C₁-C₈, C₁-C₆, C₁-C₄, or C₁-C₂). In embodiments, R^{4A} is independently substituted alkyl (e.g., C₁-C₈, C₁-C₆, C₁-C₄, or C₁-C₂). In embodiments, R^{4A} is independently unsubstituted alkyl (e.g., C₁-C₈, C₁-C₆, C₁-C₄, or C₁-C₂). In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted isopropyl. In embodiments, R^{4A} is independently unsubstituted tert-butyl. In embodiments, R^{4A} is independently substituted or unsubstituted heteroalkyl (e.g., 2 to 8 membered, 2 to 6 membered, 4 to 6 membered, 2 to 3 membered, or 4 to 5 membered). In embodiments, R^{4A} is independently substituted heteroalkyl (e.g., 2 to 8 membered, 2 to 6 membered, 4 to 6 membered, 2 to 3 membered, or 4 to 5 membered). In embodiments, R^{4A} is independently unsubstituted heteroalkyl (e.g., 2 to 8 membered, 2 to 6 membered, 4 to 6 membered, 2 to 3 membered, or 4 to 5 membered). In embodiments, R^{4A} is independently substituted or unsubstituted cycloalkyl (e.g., C₃-C₈, C₃-C₆, C₄-C₆, or C₅-C₆). In embodiments, R^{4A} is independently substituted cycloalkyl (e.g., C₃-C₈, C₃-C₆, C₄-C₆, or C₅-C₆). In embodiments, R^{4A} is independently unsubstituted cycloalkyl (e.g., C₃-C₈, C₃-C₆, C₄-C₆, or C₅-C₆). In embodiments, R^{4A} is independently substituted or unsubstituted heterocycloalkyl (e.g., 3 to 8 membered, 3 to 6 membered, 4 to 6 membered, 4 to 5 membered, or 5 to 6 membered). In embodiments, R^{A} is independently substituted heterocycloalkyl (e.g., 3 to 8 membered, 3 to 6 membered, 4 to 6 membered, 4 to 5 membered, or 5 to 6 membered). In embodiments, R^{4A} is independently unsubstituted heterocycloalkyl (e.g., 3 to 8 membered, 3 to 6 membered, 4 to 6 membered, 4 to 5 membered, or 5 to 6 membered). In embodiments, R^{4A} is independently substituted or unsubstituted aryl (e.g., C₆-C₁₀ or phenyl). In embodiments, R^{4A} is independently substituted aryl (e.g., C₆-C₁₀ or phenyl). In embodiments, R^{4A} is independently unsubstituted aryl (e.g., C₆-C₁₀ or phenyl). In embodiments, R^{4A} is independently substituted or unsubstituted heteroaryl (e.g., 5 to 10 membered, 5 to 9 membered, or 5 to 6 membered). In embodiments, R^{4A} is independently substituted heteroaryl (e.g., 5 to 10 membered, 5 to 9 membered, or 5 to 6 membered). In embodiments, R^{4A} is independently unsubstituted heteroaryl (e.g., 5 to 10 membered, 5 to 9 membered, or 5 to 6 membered).

In embodiments, R^{4B} is independently substituted or unsubstituted alkyl (e.g., C₁-C₈, C₁-C₆, C₁-C₄, or C₁-C₂). In embodiments, R^{4B} is independently substituted alkyl (e.g., C₁-C₈, C₁-C₆, C₁-C₄, or C₁-C₂). In embodiments, R^{4B} is independently unsubstituted alkyl (e.g., C₁-C₈, C₁-C₆, C₁-C₄, or C₁-C₂). In embodiments, R^{4B} is independently unsubstituted methyl. In embodiments, R^{4B} is independently unsubstituted ethyl. In embodiments, R^{4B} is independently unsubstituted propyl. In embodiments, R^{4B} is independently unsubstituted isopropyl. In embodiments, R^{4B} is independently unsubstituted tert-butyl. In embodiments, R^{4B} is independently substituted or unsubstituted heteroalkyl (e.g., 2 to 8 membered, 2 to 6 membered, 4 to 6 membered, 2 to 3 membered, or 4 to 5 membered). In embodiments, R^{4B} is independently substituted heteroalkyl (e.g., 2 to 8 membered, 2 to 6 membered, 4 to 6 membered, 2 to 3 membered, or 4 to 5 membered). In embodiments, R^{4B} is independently unsubstituted heteroalkyl (e.g., 2 to 8 membered, 2 to 6 membered, 4 to 6 membered, 2 to 3 membered, or 4 to 5 membered). In embodiments, R^{4B} is independently substituted or unsubstituted cycloalkyl (e.g., C₃-C₈, C₃-C₆, C₄-C₆, or C₅-C₆). In embodiments, R^{4B} is independently substituted cycloalkyl (e.g., C₃-C₈, C₃-C₆, C₄-C₆, or C₅-C₆). In embodiments, R^{4B} is independently unsubstituted cycloalkyl (e.g., C₃-C₈, C₃-C₆, C₄-C₆, or C₅-C₆). In embodiments, R^{4B} is independently substituted or unsubstituted heterocycloalkyl (e.g., 3 to 8 membered, 3 to 6 membered, 4 to 6 membered, 4 to 5 membered, or 5 to 6 membered). In embodiments, R^{4B} is independently substituted heterocycloalkyl (e.g., 3 to 8 membered, 3 to 6 membered, 4 to 6 membered, 4 to 5 membered, or 5 to 6 membered). In embodiments, R^{4B} is independently unsubstituted heterocycloalkyl (e.g., 3 to 8 membered, 3 to 6 membered, 4 to 6 membered, 4 to 5 membered, or 5 to 6 membered). In embodiments, R^{4B} is independently substituted or unsubstituted aryl (e.g., C₆-C₁₀ or phenyl). In embodiments, R^{4B} is independently substituted aryl (e.g., C₆-C₁₀ or phenyl). In embodiments, R^{4B} is independently unsubstituted aryl (e.g., C₆-C₁₀ or phenyl). In embodiments, R^{4B} is independently substituted or unsubstituted heteroaryl (e.g., 5 to 10 membered, 5 to 9 membered, or 5 to 6 membered). In embodiments, R^{4B} is independently substituted heteroaryl (e.g., 5 to 10 membered, 5 to 9 membered, or 5 to 6 membered). In embodiments, R^{4B} is independently unsubstituted heteroaryl (e.g., 5 to 10 membered, 5 to 9 membered, or 5 to 6 membered).

In embodiments, R^{4A} and R^{4B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{4A} and R^{4B} substituents bonded to the same nitrogen atom may be joined to form a substituted or unsubstituted heteroaryl (e.g., 5 to 10 membered, 5 to 9 membered, or 5 to 6 membered). In embodiments, R^{4A} and R^{4B} substituents bonded to the same nitrogen atom may be joined to form a substituted heteroaryl (e.g., 5 to 10 membered, 5 to 9 membered, or 5 to 6 membered). In embodiments, R^{4A} and R^{4B} substituents bonded to the same nitrogen atom may be joined to form an unsubstituted heteroaryl (e.g., 5 to 10 membered, 5 to 9 membered, or 5 to 6 membered).

In embodiments, R^{4C} is independently substituted or unsubstituted alkyl (e.g., C₁-C₈, C₁-C₆, C₁-C₄, or C₁-C₂). In embodiments, R^{4C} is independently substituted alkyl (e.g., C₁-C₈, C₁-C₆, C₁-C₄, or C₁-C₂). In embodiments, R^{4C} is independently unsubstituted alkyl (e.g., C₁-C₈, C₁-C₆, C₁-C₄, or C₁-C₂). In embodiments, R^{4C} is independently unsubstituted methyl. In embodiments, R^{4C} is independently unsubstituted ethyl. In embodiments, R^{4C} is independently unsubstituted propyl. In embodiments, R^{4C} is independently unsubstituted isopropyl. In embodiments, R^{4C} is independently unsubstituted tert-butyl. In embodiments, R⁴ is independently substituted or unsubstituted heteroalkyl (e.g., 2 to 8 membered, 2 to 6 membered, 4 to 6 membered, 2 to 3 membered, or 4 to 5 membered). In embodiments, R^{4C} is independently substituted heteroalkyl (e.g., 2 to 8 membered, 2 to 6 membered, 4 to 6 membered, 2 to 3 membered, or 4 to 5 membered). In embodiments, R^{4C} is independently unsubstituted heteroalkyl (e.g., 2 to 8 membered, 2 to 6 membered, 4 to 6 membered, 2 to 3 membered, or 4 to 5 membered). In embodiments, R^{4C} is independently substituted or unsubstituted cycloalkyl (e.g., C₃-C₈, C₃-C₆, C₄-C₆, or C₅-C₆). In embodiments, R^{4C} is independently substituted cycloalkyl (e.g., C₃-C₈, C₃-C₆, C₄-C₆, or C₅-C₆). In embodiments, R^{4C} is independently unsubstituted cycloalkyl (e.g., C₃-C₈, C₃-C₆, C₄-C₆, or C₅-C₆). In embodiments, R^{4C} is independently substituted or unsubstituted heterocycloalkyl (e.g., 3 to 8 membered, 3 to 6 membered, 4 to 6 membered, 4 to 5 membered, or 5 to 6 membered). In embodiments, R^{4C} is independently substituted heterocycloalkyl (e.g., 3 to 8 membered, 3 to 6 membered, 4 to 6 membered, 4 to 5 membered, or 5 to 6 membered). In embodiments, R^{4C} is independently unsubstituted heterocycloalkyl (e.g., 3 to 8 membered, 3 to 6 membered, 4 to 6 membered, 4 to 5 membered, or 5 to 6 membered). In embodiments, R^{4C} is independently substituted or unsubstituted aryl (e.g., C₆-C₁₀ or phenyl). In embodiments, R^{4C} is independently substituted aryl (e.g., C₆-C₁₀ or phenyl). In embodiments, R^{4C} is independently unsubstituted aryl (e.g., C₆-C₁₀ or phenyl). In embodiments, R^{4C} is independently substituted or unsubstituted heteroaryl (e.g., 5 to 10 membered, 5 to 9 membered, or 5 to 6 membered). In embodiments, R^{4C} is independently substituted heteroaryl (e.g., 5 to 10 membered, 5 to 9 membered, or 5 to 6 membered). In embodiments, R^{4C} is independently unsubstituted heteroaryl (e.g., 5 to 10 membered, 5 to 9 membered, or 5 to 6 membered).

In embodiments, R⁴ is independently hydrogen, —CX⁴₃, —CHX⁴₂, —CH₂X⁴, —CN, —C(O)R^{4C}, —C(O)OR^{4C}, —C(O)NR^{4A}R^{4B}, R²⁹-substituted or unsubstituted alkyl (e.g., C₁-C₈, C₁-C₆, C₁-C₄, or C₁-C₂), R²-substituted or unsubstituted heteroalkyl (e.g., 2 to 8 membered, 2 to 6 membered, 4 to 6 membered, 2 to 3 membered, or 4 to 5 membered), R²⁹-substituted or unsubstituted cycloalkyl (e.g., C₃-C₈, C₃-C₆, C₄-C₆, or C₅-C₆), R²⁹-substituted or unsubstituted heterocycloalkyl (e.g., 3 to 8 membered, 3 to 6 membered, 4 to 6 membered, 4 to 5 membered, or 5 to 6 membered), R²⁹-substituted or unsubstituted aryl (e.g., C₆-C₁₀ or phenyl), or R²⁹-substituted or unsubstituted heteroaryl (e.g., 5 to 10 membered, 5 to 9 membered, or 5 to 6 membered). In embodiments, R⁴ is independently hydrogen, —CX⁴₃, —CHX⁴₂, —CH₂X⁴, —CN, —C(O)R^{4C}, —C(O)OR^{4C}, —C(O)NR^{4A}R^{4B}, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴ is independently —F, —Cl, —Br, or —I.

In embodiments, R⁴ is independently hydrogen. In embodiments, R⁴ is independently unsubstituted methyl. In embodiments, R⁴ is independently unsubstituted ethyl.

R²⁹ is independently oxo,

halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁰-substituted or unsubstituted alkyl (e.g., C₁-C₈, C₁-C₆, C₁-C₄, or C₁-C₂), R³⁰-substituted or unsubstituted heteroalkyl (e.g., 2 to 8 membered, 2 to 6 membered, 4 to 6 membered, 2 to 3 membered, or 4 to 5 membered), R³⁰-substituted or unsubstituted cycloalkyl (e.g., C₃-C₈, C₃-C₆, C₄-C₆, or C₅-C₆), R³⁰-substituted or unsubstituted heterocycloalkyl (e.g., 3 to 8 membered, 3 to 6 membered, 4 to 6 membered, 4 to 5 membered, or 5 to 6 membered), R³⁰-substituted or unsubstituted aryl (e.g., C₆-C₁₀ or phenyl), or R³⁰-substituted or unsubstituted heteroaryl (e.g., 5 to 10 membered, 5 to 9 membered, or 5 to 6 membered). In embodiments, R²⁹ is independently oxo,
halogen, —CX²⁹₃, —CHX²⁹₂, —CH₂X²⁹, —OCX²⁹₃, —OCH₂X²⁹, —OCHX²⁹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁹ is independently —F, —Cl, —Br, or —I.

In embodiments, R²⁹ is independently unsubstituted methyl. In embodiments, R²⁹ is independently unsubstituted ethyl.

R³⁰ is independently oxo,

halogen, —CX³⁰₃, —CHX³⁰₂, —CH₂X³⁰, —OCX³⁰₂, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered). In embodiments, R³⁰ is independently oxo,
halogen, —CX³⁰₃, —CHX³⁰₂, —CH₂X³⁰, —OCX³⁰₃, —OCH₂X³⁰, —OCHX³⁰₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁰ is independently —F, —Cl, —Br, or —I. In embodiments, R³⁰ is independently unsubstituted methyl. In embodiments, R³⁰ is independently unsubstituted ethyl.

R³¹ is independently oxo,

halogen, —CX³¹₃, —CHX³¹₂, —CH₂X³¹, —OCX³¹₃, —OCH₂X³¹, —OCHX³¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³¹ is independently —F, —Cl, —Br, or —I.

In embodiments, R³¹ is independently unsubstituted methyl. In embodiments, R³¹ is independently unsubstituted ethyl.

In embodiments, R^{4A} is independently

hydrogen, —CX^{4A}₃, —CHX^{4A}₂, —CH₂X^{4A}, —CN, —COOH, —CONH₂, R^{29A}-substituted or unsubstituted alkyl (e.g., C₁-C₈, C₁-C₆, C₁-C₄, or C₁-C₂), R^{29A}-substituted or unsubstituted heteroalkyl (e.g., 2 to 8 membered, 2 to 6 membered, 4 to 6 membered, 2 to 3 membered, or 4 to 5 membered), R^{29A}-substituted or unsubstituted cycloalkyl (e.g., C₃-C₈, C₃-C₆, C₄-C₆, or C₅-C₆), R^{29A}-substituted or unsubstituted heterocycloalkyl (e.g., 3 to 8 membered, 3 to 6 membered, 4 to 6 membered, 4 to 5 membered, or 5 to 6 membered), R^{29A}-substituted or unsubstituted aryl (e.g., C₆-C₁₀ or phenyl), or R^{29A}-substituted or unsubstituted heteroaryl (e.g., 5 to 10 membered, 5 to 9 membered, or 5 to 6 membered). In embodiments, R^{4A} is independently
hydrogen, —CX^{4A}₃, —CHX^{4A}₂, —CH₂X^{4A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4A} is independently —F, —Cl, —Br, or —I. In embodiments, R^{4A} is independently hydrogen. In embodiments, R^{4A} is independently unsubstituted methyl. In embodiments, R^{4A} is independently unsubstituted ethyl.

In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered) or R^{29A}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form a R^{29A}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heteroaryl (e.g., 5 to 10 membered, 5 to 9 membered, or 5 to 6 membered).

R^{29A} is independently oxo,

halogen, —CX^{29A}₃, —CHX^{29A}₂, —CH₂X^{29A}, —OCX^{29A}₃, —OCH₂X^{29A}, —CHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{30A}-substituted or unsubstituted alkyl (e.g., C₁-C₈, C₁-C₆, C₁-C₄, or C₁-C₂), R^{30A}-substituted or unsubstituted heteroalkyl (e.g., 2 to 8 membered, 2 to 6 membered, 4 to 6 membered, 2 to 3 membered, or 4 to 5 membered), R^{30A}-substituted or unsubstituted cycloalkyl (e.g., C₃-C₈, C₃-C₆, C₄-C₆, or C₅-C₆), R^{30A}-substituted or unsubstituted heterocycloalkyl (e.g., 3 to 8 membered, 3 to 6 membered, 4 to 6 membered, 4 to 5 membered, or 5 to 6 membered), R^{30A}-substituted or unsubstituted aryl (e.g., C₆-C₁₀ or phenyl), or R^{30A}-substituted or unsubstituted heteroaryl (e.g., 5 to 10 membered, 5 to 9 membered, or 5 to 6 membered). In embodiments, R^{29A} is independently oxo,
halogen, —CX^{29A}₃, —CHX^{29A}₂, —CH₂X^{29A}, —OCX^{29A}₃, —OCH₂X^{29A}, —OCHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29A} is independently —F, —Cl, —Br, or —I.

In embodiments, R^{29A} is independently unsubstituted methyl. In embodiments, R^{29A} is independently unsubstituted ethyl.

R^{30A} is independently oxo,

halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{31A}-substituted or unsubstituted alkyl (e.g., C₁-C₈, C₁-C₆, C₁-C₄, or C₁-C₂), R^{31A}-substituted or unsubstituted heteroalkyl (e.g., 2 to 8 membered, 2 to 6 membered, 4 to 6 membered, 2 to 3 membered, or 4 to 5 membered), R^{31A}-substituted or unsubstituted cycloalkyl (e.g., C₃-C₈, C₃-C₆, C₄-C₆, or C₅-C₆), R^{31A}-substituted or unsubstituted heterocycloalkyl (e.g., 3 to 8 membered, 3 to 6 membered, 4 to 6 membered, 4 to 5 membered, or 5 to 6 membered), R^{31A}-substituted or unsubstituted aryl (e.g., C₆-C₁₀ or phenyl), or R^{31A}-substituted or unsubstituted heteroaryl (e.g., 5 to 10 membered, 5 to 9 membered, or 5 to 6 membered). In embodiments, R^{30A} is independently oxo,
halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30A} is independently —F, —Cl, —Br, or —I.

R^{31A} is independently oxo,

halogen, —CX^{31A}₃, —CHX^{31A}₂, —CH₂X^{31A}, —OCX^{31A}₃, —OCH₂X^{31A}, —OCHX^{31A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31A} is independently —F, —Cl, —Br, or —I

In embodiments, R^{4B} is independently

hydrogen, —CX^{4B}₃, —CHX^{4B}₂, —CH₂X^{4B}, —CN, —COOH, —CONH₂, R^{29B}-substituted or unsubstituted alkyl (e.g., C₁-C₈, C₁-C₆, C₁-C₄, or C₁-C₂), R^{29B}-substituted or unsubstituted heteroalkyl (e.g., 2 to 8 membered, 2 to 6 membered, 4 to 6 membered, 2 to 3 membered, or 4 to 5 membered), R^{29B}-substituted or unsubstituted cycloalkyl (e.g., C₃-C₈, C₃-C₆, C₄-C₆, or C₅-C₆), R^{29B}-substituted or unsubstituted heterocycloalkyl (e.g., 3 to 8 membered, 3 to 6 membered, 4 to 6 membered, 4 to 5 membered, or 5 to 6 membered), R^{29B}-substituted or unsubstituted aryl (e.g., C₆-C₁₀ or phenyl), or R^{29B}-substituted or unsubstituted heteroaryl (e.g., 5 to 10 membered, 5 to 9 membered, or 5 to 6 membered). In embodiments, R^{4B} is independently
hydrogen, —CX^{4B}₃, —CHX^{4B}₂, —CH₂X^{4B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4B} is independently —F, —Cl, —Br, or —I.

In embodiments, R^{4B} is independently hydrogen. In embodiments, R^{4B} is independently unsubstituted methyl. In embodiments, R^{4B} is independently unsubstituted ethyl.

In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered) or R^{29B}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form a R^{29B}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heteroaryl (e.g., 5 to 10 membered, 5 to 9 membered, or 5 to 6 membered).

R^{29B} is independently oxo,

halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{30B}-substituted or unsubstituted alkyl (e.g., C₁-C₈, C₁-C₆, C₁-C₄, or C₁-C₂), R^{30B}-substituted or unsubstituted heteroalkyl (e.g., 2 to 8 membered, 2 to 6 membered, 4 to 6 membered, 2 to 3 membered, or 4 to 5 membered), R^{30B}-substituted or unsubstituted cycloalkyl (e.g., C₃-C₈, C₃-C₆, C₄-C₆, or C₅-C₆), R^{30B}-substituted or unsubstituted heterocycloalkyl (e.g., 3 to 8 membered, 3 to 6 membered, 4 to 6 membered, 4 to 5 membered, or 5 to 6 membered), R^{30B}-substituted or unsubstituted aryl (e.g., C₆-C₁₀ or phenyl), or R^{30B}-substituted or unsubstituted heteroaryl (e.g., 5 to 10 membered, 5 to 9 membered, or 5 to 6 membered). In embodiments, R^{29B} is independently oxo,
halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29B} is independently —F, —Cl, —Br, or —I.

In embodiments, R^{29B} is independently unsubstituted methyl. In embodiments, R^{29B} is independently unsubstituted ethyl.

R^{30B} is independently oxo,

halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{31B}-substituted or unsubstituted alkyl (e.g., C₁-C₈, C₁-C₆, C₁-C₄, or C₁-C₂), R^{31B}-substituted or unsubstituted heteroalkyl (e.g., 2 to 8 membered, 2 to 6 membered, 4 to 6 membered, 2 to 3 membered, or 4 to 5 membered), R^{31B}-substituted or unsubstituted cycloalkyl (e.g., C₃-C₈, C₃-C₆, C₄-C₆, or C₅-C₆), R^{31B}-substituted or unsubstituted heterocycloalkyl (e.g., 3 to 8 membered, 3 to 6 membered, 4 to 6 membered, 4 to 5 membered, or 5 to 6 membered), R^{31B}-substituted or unsubstituted aryl (e.g., C₆-C₁₀ or phenyl), or R^{31B}-substituted or unsubstituted heteroaryl (e.g., 5 to 10 membered, 5 to 9 membered, or 5 to 6 membered). In embodiments, R^{30B} is independently oxo,
halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30B} is independently —F, —Cl, —Br, or —I.

R^{31B} is independently oxo,

halogen, —CX^{31B}₃, —CHX^{31B}₂, —CH₂X^{31B}, —OCX^{31B}₃, —OCH₂X^{31B}, —OCHX^{31B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31B} is independently —F, —Cl, —Br, or —I

In embodiments, R^{4C} is independently

hydrogen, —CX^{4C}₃, —CHX^{4C}₂, —CH₂X^{4C}, —CN, —COOH, —CONH₂, R^{29C}-substituted or unsubstituted alkyl (e.g., C₁-C₈, C₁-C₆, C₁-C₄, or C₁-C₂), R²⁹-substituted or unsubstituted heteroalkyl (e.g., 2 to 8 membered, 2 to 6 membered, 4 to 6 membered, 2 to 3 membered, or 4 to 5 membered), R^{29C}-substituted or unsubstituted cycloalkyl (e.g., C₃-C₈, C₃-C₆, C₄-C₆, or C₅-C₆), R^{29C}-substituted or unsubstituted heterocycloalkyl (e.g., 3 to 8 membered, 3 to 6 membered, 4 to 6 membered, 4 to 5 membered, or 5 to 6 membered), R^{29C}-substituted or unsubstituted aryl (e.g., C₆-C₁₀ or phenyl), or R^{29C}-substituted or unsubstituted heteroaryl (e.g., 5 to 10 membered, 5 to 9 membered, or 5 to 6 membered). In embodiments, R^{4C} is independently
hydrogen, —CX^{4C}₃, —CHX^{4C}₂, —CH₂X^{4C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4C} is independently —F, —Cl, —Br, or —I. In embodiments, R^{4C} is independently hydrogen. In embodiments, R^{4C} is independently unsubstituted methyl. In embodiments, R^{4C} is independently unsubstituted ethyl.

R^{29C} is independently oxo,

halogen, —CX^{29C}₃, —CHX^{29C}₂, —CH₂X^{29C}, —OCX^{29C}₃, —OCH₂X^{29C}, —OCHX^{29C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{30C}-substituted or unsubstituted alkyl (e.g., C₁-C₈, C₁-C₆, C₁-C₄, or C₁-C₂), R^{30C}-substituted or unsubstituted heteroalkyl (e.g., 2 to 8 membered, 2 to 6 membered, 4 to 6 membered, 2 to 3 membered, or 4 to 5 membered), R^{30C}-substituted or unsubstituted cycloalkyl (e.g., C₃-C₈, C₃-C₆, C₄-C₆, or C₅-C₆), R^{30C}-substituted or unsubstituted heterocycloalkyl (e.g., 3 to 8 membered, 3 to 6 membered, 4 to 6 membered, 4 to 5 membered, or 5 to 6 membered), R^{30C}-substituted or unsubstituted aryl (e.g., C₆-C₁₀ or phenyl), or R^{30C}-substituted or unsubstituted heteroaryl (e.g., 5 to 10 membered, 5 to 9 membered, or 5 to 6 membered). In embodiments, R^{29C} is independently oxo,
halogen, —CX^{29C}₃, —CHX^{29C}₂, —CH₂X^{29C}, —OCX^{29C}₃, —OCH₂X^{29C}, —OCHX^{29C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29C} is independently —F, —Cl, —Br, or —I.

In embodiments, R^{29C} is independently unsubstituted methyl. In embodiments, R^{29C} is independently unsubstituted ethyl.

R^{30C} is independently oxo,

halogen, —CX^{30C}₃, —CHX^{30C}₂, —CH₂X^{30C}, —OCX^{30C}₃, —OCH₂X^{30C}, —OCHX^{30C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{31C}-substituted or unsubstituted alkyl (e.g., C₁-C₈, C₁-C₆, C₁-C₄, or C₁-C₂), R^{31C}-substituted or unsubstituted heteroalkyl (e.g., 2 to 8 membered, 2 to 6 membered, 4 to 6 membered, 2 to 3 membered, or 4 to 5 membered), R^{31C}-substituted or unsubstituted cycloalkyl (e.g., C₃-C₈, C₃-C₆, C₄-C₆, or C₅-C₆), R^{31C}-substituted or unsubstituted heterocycloalkyl (e.g., 3 to 8 membered, 3 to 6 membered, 4 to 6 membered, 4 to 5 membered, or 5 to 6 membered), R^{31C}-substituted or unsubstituted aryl (e.g., C₆-C₁₀ or phenyl), or R^{31C}-substituted or unsubstituted heteroaryl (e.g., 5 to 10 membered, 5 to 9 membered, or 5 to 6 membered). In embodiments, R^{30C} is independently oxo,
halogen, —CX^{30C}₃, —CHX^{30C}₂, —CH₂X^{30C}, —OCX^{30C}₃, —OCH₂X^{30C}, —OCHX^{30C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30C} is independently —F, —Cl, —Br, or —I.

R^{31C} is independently oxo,

halogen, —CX^{31C}₃, —CHX^{31C}₂, —CH₂X^{31C}, —OCX^{31C}₃, —OCH₂X^{31C}, —OCHX^{31C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31C} is independently —F, —Cl, —Br, or —I

L¹ is —O—, —S—, substituted or unsubstituted C₁-C₂ alkylene

(e.g., —CH₂—, —CH₂CH₂—, —C(CH₃)H—, or —CH(CH₃)CH₂—), or substituted or unsubstituted 2 membered heteroalkylene
(e.g., —CH₂O—, —OCH₂—, —CH₂S—, —SCH₂—, —CH₂NH—, —NHCH₂—, —CH(CH₃)O—, —OCH(CH₃)—, —CH(CH₃)S—, —SCH(CH₃)—, —CH(CH₃)NH—, —NHCH(CH₃)—, —CH₂N(CH₃)—, or —N(CH₃)CH₂—). In embodiments, L¹ is —O—, —S—, or substituted or unsubstituted methylene. In embodiments, L¹ is —SCH₂—. In embodiments, L¹ is —O—. In embodiments, L¹ is —S—. In embodiments, L¹ is —CH(CH₃)—.

In embodiments, L¹ is a

bond, —S(O)₂—, —N(R³)—, —O—, —S—, —C(O)—, —C(O)N(R³)—, —N(R³)C(O)—, —N(R³)C(O)NH—, substituted or unsubstituted alkylene, or substituted or unsubstituted heteroalkylene. In embodiments, L¹ is a bond, —C(O)N(R³)—, —N(R³)C(O)—, substituted or unsubstituted alkylene, or substituted or unsubstituted heteroalkylene. In embodiments, L¹ is —C(O)N(R³)—, unsubstituted alkylene, or unsubstituted heteroalkylene. In embodiments, L¹ is —C(O)N(R³)—. In embodiments, L¹ is unsubstituted alkylene. In embodiments, L¹ is unsubstituted heteroalkylene. In embodiments, L¹ is —C(O)NH—.

In embodiments, L¹ is a

bond, —S(O)₂—, —N(R³)—, —O—, —S—, —C(O)—, —C(O)N(R³)—, —N(R³)C(O)—, —N(R³)C(O)NH—, —NHC(O)N(R³)—, —S(O)₂N(R³)—, —N(R³)S(O)₂—, —C(O)S(O)₂N(R³)—, —N(R³)S(O)₂C(O)—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L¹ is

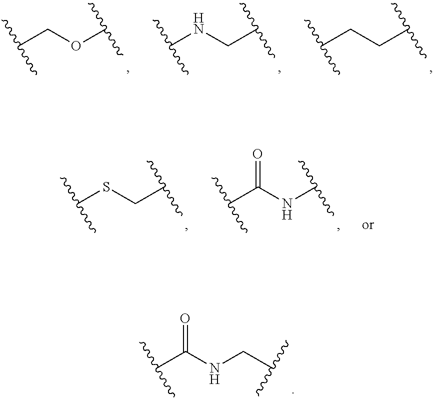

In embodiments, L¹ is

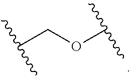 [SMILES: *CO*]

In embodiments L¹ is

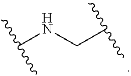 [SMILES: *CN*]

In embodiments, L¹ is

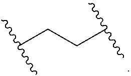 [SMILES: *CC*]

In embodiments, L¹ is

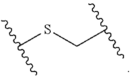 [SMILES: *CS*]

In embodiments, L¹ is

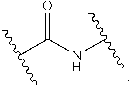 [SMILES: *NC(*)=O]

In embodiments, L¹ is

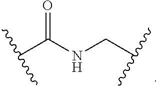 [SMILES: *CNC(*)=O]

In embodiments, L¹ is independently —O—, —S—, R³²-substituted or unsubstituted C₁-C₂ alkylene (e.g., C₁ or C₂) or R³²-substituted or unsubstituted 2 membered heteroalkylene. In embodiments, L¹ is R³²-substituted or unsubstituted alkylene (e.g., C₁-C₈ alkylene, C₁-C₆ alkylene, or C₁-C₄ alkylene), R³²-substituted or unsubstituted heteroalkylene (e.g., 2 to 8 membered heteroalkylene, 2 to 6 membered heteroalkylene, or 2 to 4 membered heteroalkylene), R³²-substituted or unsubstituted cycloalkylene (e.g., C₃-C₈ cycloalkylene, C₃-C₆ cycloalkylene, or C₅-C₆ cycloalkylene), R³²-substituted or unsubstituted heterocycloalkylene (e.g., 3 to 8 membered heterocycloalkylene, 3 to 6 membered heterocycloalkylene, or 5 to 6 membered heterocycloalkylene), R³²-substituted or unsubstituted arylene (e.g., C₆-C₁₀ arylene, C₁₀ arylene, or phenylene), or R³²-substituted or unsubstituted heteroarylene (e.g., 5 to 10 membered heteroarylene, 5 to 9 membered heteroarylene, or 5 to 6 membered heteroarylene). In embodiments, L¹ is independently —O—, —S—, unsubstituted C₁-C₂ alkylene (e.g., C₁ or C₂) or unsubstituted 2 membered heteroalkylene. In embodiments, L¹ is independently unsubstituted methylene. In embodiments, L¹ is independently unsubstituted ethylene. In embodiments, L¹ is substituted 2 membered heteroalkylene. In embodiments, L¹ is substituted 3 membered heteroalkylene. In embodiments, L¹ is substituted 4 membered heteroalkylene. In embodiments, L¹ is an unsubstituted 2 membered heteroalkylene. In embodiments, L¹ is an unsubstituted 3 membered heteroalkylene. In embodiments, L¹ is an unsubstituted 4 membered heteroalkylene. In embodiments, L¹ is —CONHCH₂—.

R³² is independently oxo,

halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). In embodiments, R³² is independently oxo,
halogen, —CX³²₃, —CHX³²₂, —CH₂X³², —OCX³²₃, —OCH₂X³², —OCHX³²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³² is independently —F, —Cl, —Br, or —I.

In embodiments, R³² is independently unsubstituted methyl. In embodiments, R³² is independently unsubstituted ethyl.

R³³ is independently oxo,

halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). In embodiments, R³³ is independently oxo,
halogen, —CX³³₃, —CHX³³₂, —CH₂X³³, —OCX³³₃, —OCH₂X³³, —OCHX³³₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³³ is independently —F, —Cl, —Br, or —I.

In embodiments, R³³ is independently unsubstituted methyl. In embodiments, R³³ is independently unsubstituted ethyl.

R³⁴ is independently oxo,

halogen, —CX³⁴₃, —CHX³⁴₂, —CH₂X³⁴, —OCX³⁴₃, —OCH₂X³⁴, —OCHX³⁴₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I.

In embodiments, R³⁴ is independently unsubstituted methyl. In embodiments, R³⁴ is independently unsubstituted ethyl.

In embodiments, L² is —O—, —S—, substituted or unsubstituted C₁-C₂ alkylene (e.g., —CH₂—, —CH₂CH₂—, —C(CH₃)H—, or —CH(CH₃)CH₂—), or substituted or unsubstituted 2 membered heteroalkylene (e.g., —CH₂O—, —OCH₂—, —CH₂S—, —SCH₂—, —CH₂NH—, —NHCH₂—, —CH(CH₃)O—, —OCH(CH₃)—, —CH(CH₃)S—, —SCH(CH₃)—, —CH(CH₃)NH—, —NHCH(CH₃)—, —CH₂N(CH₃)—, or —N(CH₃)CH₂—). In embodiments, L² is —O—, —S—, or substituted or unsubstituted methylene. In embodiments, L² is —SCH₂—. In embodiments, L² is —O—. In embodiments, L² is —S—. In embodiments, L² is —CH(CH₃)—. In embodiments, L² is a bond, —S(O)₂—, —N(R⁴)—, —O—, —S—, —C(O)—, —C(O)N(R⁴)—, —N(R⁴)C(O)—, —N(R⁴)C(O)NH—, substituted or unsubstituted alkylene, or substituted or unsubstituted heteroalkylene. In embodiments, L² is a bond, —N(R⁴)C(O)—, or substituted heteroalkylene. In embodiments, L² is a bond. In embodiments, L² is —N(R⁴)C(O)—. In embodiments, L² is substituted heteroalkylene.

In embodiments, L² is a

bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —S(O)₂N(R⁴)—, N(R⁴)S(O)₂—, —C(O)S(O)₂N(R⁴)—, —N(R⁴)S(O)₂C(O)—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, L² is a bond. In embodiments, L² is —O—. In embodiments, L² is

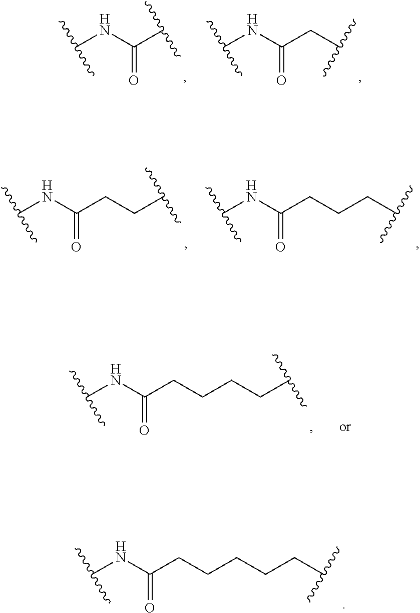 [SMILES: *NC(*)=O ; *CC(=O)N* ; *CCC(=O)N* ; *CCCC(=O)N* ; *CCCCC(=O)N* ; *CCCCCC(=O)N*]

In embodiments, L² is

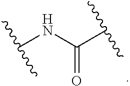 [SMILES: *NC(*)=O]

In embodiments, L² is

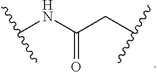 [SMILES: *CC(=O)N*]

In embodiments, L² is

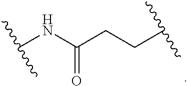 [SMILES: *CCC(=O)N*]

In embodiments, L² is

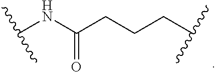 [SMILES: *CCCC(=O)N*]

In embodiments, L² is

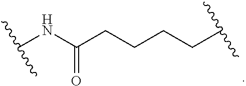 [SMILES: *CCCCC(=O)N*]

In embodiments, L² is

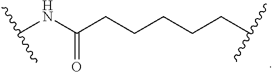 [SMILES: *CCCCCC(=O)N*]

In embodiments, L² is

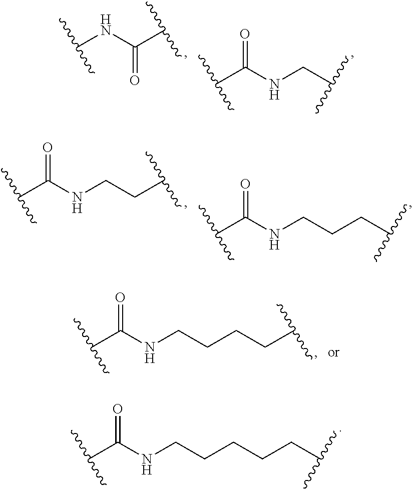 [SMILES: *NC(*)=O ; *CNC(*)=O ; *CCNC(*)=O ; *CCCNC(*)=O ; *CCCCNC(*)=O ; *CCCCCNC(*)=O]

In embodiments, L² is a

bond, —S(O)₂—, —NH—, —O—, —S—, —C(O)—, —C(O)NH—, —NHC(O)—, —NHC(O)NH—, substituted or unsubstituted alkylene, or substituted or unsubstituted heteroalkylene. In embodiments, L² is a bond, —NHC(O)—, or substituted heteroalkylene. In embodiments, L² is a bond. In embodiments, L² is —NHC(O)—. In embodiments, L² is substituted heteroalkylene.

In embodiments, L² is substituted 2 to 6 membered heteroalkylene. In embodiments, L² is substituted 3 to 6 membered heteroalkylene. In embodiments, L² is substituted 3 to 5 membered heteroalkylene. In embodiments, L² is substituted 2 membered heteroalkylene. In embodiments, L² is substituted 3 membered heteroalkylene. In embodiments, L² is substituted 4 membered heteroalkylene. In embodiments, L² is substituted 5 membered heteroalkylene. In embodiments, L² is substituted 6 membered heteroalkylene. In embodiments, L² is —NHC(O)CH₂CH₂—. In embodiments, L² is —NHC(O)CH₂—. In embodiments, L² is a bond.

In embodiments, L² is independently —O—, —S—, R³⁵-substituted or unsubstituted C₁-C₂ alkylene (e.g., C₁ or C₂) or R³⁵-substituted or unsubstituted 2 membered heteroalkylene. In embodiments, L² is R³⁵-substituted or unsubstituted alkylene (e.g., C₁-C₈ alkylene, C₁-C₆ alkylene, or C₁-C₄ alkylene), R³⁵-substituted or unsubstituted heteroalkylene (e.g., 2 to 8 membered heteroalkylene, 2 to 6 membered heteroalkylene, or 2 to 4 membered heteroalkylene), R³⁵-substituted or unsubstituted cycloalkylene (e.g., C₃-C₈ cycloalkylene, C₃-C₆ cycloalkylene, or C₅-C₆ cycloalkylene), R³⁵-substituted or unsubstituted heterocycloalkylene (e.g., 3 to 8 membered heterocycloalkylene, 3 to 6 membered heterocycloalkylene, or 5 to 6 membered heterocycloalkylene), R³⁵-substituted or unsubstituted arylene (e.g., C₆-C₁₀ arylene, C₁₀ arylene, or phenylene), or R³⁵-substituted or unsubstituted heteroarylene (e.g., 5 to 10 membered heteroarylene, 5 to 9 membered heteroarylene, or 5 to 6 membered heteroarylene). In embodiments, L² is independently —O—, —S—, unsubstituted C₁-C₂ alkylene (e.g., C₁ or C₂) or unsubstituted 2 membered heteroalkylene. In embodiments, L² is independently unsubstituted methylene. In embodiments, L² is independently unsubstituted ethylene.

R³⁵ is independently oxo,

halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R⁶-substituted or unsubstituted alkyl (e.g., C₁-C₈, C₁-C₆, C₁-C₄, or C₁-C₂), R⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). In embodiments, R³⁵ is independently oxo,
halogen, —CX³⁵₃, —CHX³⁵₂, —CH₂X³⁵, —OCX³⁵₃, —OCH₂X³⁵, —OCHX³⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁵ is independently —F, —Cl, —Br, or —I.

In embodiments, R³⁵ is independently unsubstituted methyl. In embodiments, R³⁵ is independently unsubstituted ethyl.

R³⁶ is independently oxo,

halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). In embodiments, R³⁶ is independently oxo,
halogen, —CX³⁶₃, —CHX³⁶₂, —CH₂X³⁶, —OCX³⁶₃, —OCH₂X³⁶, —OCHX³⁶₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁶ is independently —F, —Cl, —Br, or —I.

In embodiments, R³⁶ is independently unsubstituted methyl. In embodiments, R³⁶ is independently unsubstituted ethyl.

R³⁷ is independently oxo,

halogen, —CX³⁷₃, —CHX³⁷₂, —CH₂X³⁷, —OCX³⁷₃, —OCH₂X³⁷, —OCHX³⁷₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I.

In embodiments, R³⁷ is independently unsubstituted methyl. In embodiments, R³⁷ is independently unsubstituted ethyl.

In embodiments, the compound as described herein, including embodiments thereof, has the formula:

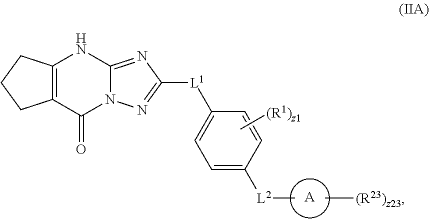

wherein R²³ is as described herein, including embodiments. Ring A is a cycloalkyl, heterocycloalkyl, aryl, or heteroaryl. The symbol z23 is an integer from 0 to 5.

In embodiments, Ring A is (C₃-C₁₀) cycloalkyl, 3 to 10 membered heterocycloalkyl, (C₆-C₁₀) aryl, or 5 to 10 membered heteroaryl. In embodiments, Ring A is a heteroaryl. In embodiments, Ring A is a 5 to 6 membered heteroaryl. In embodiments, Ring A is a 5 membered heteroaryl.

In embodiments, Ring A is a (C₃-C₁₀) cycloalkyl, a 3 to 10 membered heterocycloalkyl, a (C₆-C₁₀) aryl, or a 5 to 10 membered heteroaryl. In embodiments, Ring A is a cycloalkyl (e.g., C₃-C₈ cycloalkyl, C₃-C₆ cycloalkyl, or C₅-C₆ cycloalkyl). In embodiments, Ring A is a C₃-C₈ cycloalkyl. In embodiments, Ring A is a C₃-C₆ cycloalkyl. In embodiments, Ring A is a C₅-C₆ cycloalkyl. In embodiments, Ring A is a C₆ cycloalkyl. In embodiments, Ring A is a C₅ cycloalkyl. In embodiments, Ring A is a (C₆-C₁₀) aryl. In embodiments, Ring A is phenyl. In embodiments, Ring A is naphthyl. In embodiments, Ring A is aziridinyl, oxiranyl, thiiranyl, azetidinyl, oxetanyl, thietanyl, pyrrolidinyl, pyrrolyl, imidazolyl, imidazolinyl, pyrazolinyl, tetrahydrofuranyl, thiolanyl, piperidinyl, piperazinyl, pyranyl, morpholinyl, 1,4-dioxanyl, tetrahydro-2H-pyranyl, thianyl, or dithianyl. In embodiments, Ring A is a phenyl, thiofuranyl, imidazolyl, pyrazolyl, triazolyl, tetrazolyl, furanyl, oxazolyl, isooxazolyl, oxadiazolyl, oxatriazolyl, thienyl, thiazolyl, isothiazolyl, pyridinyl, pyrazinyl, pyrimidinyl, pyridazinyl, or triazinyl (e.g., 1,3,5-triazinyl, 1,2,3-triazinyl, or 1,2,4-triazinyl). In embodiments, Ring A is indolyl, benzimidazolyl, indazolyl, benzotriazolyl, pyrrolopyrimidinyl, purinyl, indolizinyl, pyrrolopyriazinyl, pyrrolopyrimidinyl, imidazopyridazinyl, imidazopyridinyl, imidazopyrimidinyl, cinnolinyl, quinazolinyl, quinoxalinyl, phthalazinyl, pyridopyrazinyl, pteridinyl, pyrazolopyridinyl, quinolinyl, isoquinolinyl, naphthyridinyl, or carbazolyl.

In embodiments, -(Ring A)-(R²³)_{z23} is:

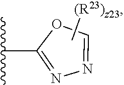

wherein R²³ and z23 are as described herein including embodiments.

In embodiments, -(ring A)-(R²³)_{z23} is:

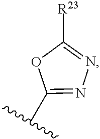

wherein R²³ is as described herein, including embodiments.

In embodiments, R²³ is independently

halogen, —CX²³₃, —C(O)R^{100C}, —C(O)—OR^{100C}, —C(O)NR^{100A}R^{100B}, —OR^{100D}, —NR^{100A}SO₂R^{100D}, —NR^{100A}C(O)R^{100C}, —NR^{100A}C(O)OR^{100C}, —NR^{100A}OR^{100C}, substituted or unsubstituted alkyl, substituted or unsubstituted heteroalkyl, substituted or unsubstituted aryl, or substituted or unsubstituted heteroaryl.

In embodiments, R²³ is independently halogen, —CX²³₃, C(O)R^{100C}, substituted or unsubstituted heterocycloalkyl, substituted or unsubstituted aryl, or substituted or unsubstituted heteroaryl. In embodiments, R²³ is independently substituted or unsubstituted phenyl. In embodiments, R²³ is independently substituted phenyl. In embodiments, R²³ is independently unsubstituted phenyl.

In embodiments, R²³ is independently halogen, —CX²³₃, C(O)R^{100C}, R²⁴-substituted or unsubstituted heterocycloalkyl, R²⁴-substituted or unsubstituted aryl, or R²⁴-substituted or unsubstituted heteroaryl. In embodiments, R²³ is independently R²⁴-substituted or unsubstituted phenyl. In embodiments, R²³ is independently R²⁴-substituted phenyl.

In embodiments, R²³ is independently R²⁴-substituted or unsubstituted cycloalkyl (e.g., C₃-C₈ cycloalkyl, C₃-C₆ cycloalkyl, or C₅-C₆ cycloalkyl). In embodiments, R²³ is independently R²⁴-substituted cycloalkyl (e.g., C₃-C₈ cycloalkyl, C₃-C₆ cycloalkyl, or C₅-C₆ cycloalkyl). In embodiments, R²³ is independently an unsubstituted cycloalkyl (e.g., C₃-C₈ cycloalkyl, C₃-C₆ cycloalkyl, or C₅-C₆ cycloalkyl).

In embodiments, R²³ is independently R²⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R²³ is independently R²⁴-substituted heterocycloalkyl (e.g., 3 to 8 membered heterocycloalkyl, 3 to 6 membered heterocycloalkyl, or 5 to 6 membered heterocycloalkyl). In embodiments, R²³ is independently an unsubstituted heterocycloalkyl (e.g., 3 to 8 membered heterocycloalkyl, 3 to 6 membered heterocycloalkyl, or 5 to 6 membered heterocycloalkyl).

In embodiments, R²³ is independently R²⁴-substituted or unsubstituted aryl (e.g., C₆-C₁₀ aryl, C₁₀ aryl, or phenyl). In embodiments, R²³ is independently R²⁴-substituted aryl (e.g., C₆-C₁₀ aryl, C₁₀ aryl, or phenyl). In embodiments, R²³ is independently an unsubstituted aryl (e.g., C₆-C₁₀ aryl, C₁₀ aryl, or phenyl). In embodiments, R²³ is independently an unsubstituted phenyl. In embodiments, R²³ is a R²⁴-substituted phenyl.

In embodiments, R²³ is independently R²⁴-substituted or unsubstituted heteroaryl (e.g., 5 to 10 membered heteroaryl, 5 to 9 membered heteroaryl, or 5 to 6 membered heteroaryl). In embodiments, R²³ is independently R²⁴-substituted heteroaryl (e.g., 5 to 10 membered heteroaryl, 5 to 9 membered heteroaryl, or 5 to 6 membered heteroaryl). In embodiments, R²³ is independently an unsubstituted heteroaryl (e.g., 5 to 10 membered heteroaryl, 5 to 9 membered heteroaryl, or 5 to 6 membered heteroaryl). In embodiments, R²³ is independently R²⁴-substituted imidazolyl, R²⁴-substituted pyrrolyl, R²⁴-substituted pyrazolyl, R²⁴-substituted triazolyl, R²⁴-substituted tetrazolyl, R²⁴-substituted furanyl, R²⁴-substituted oxazolyl, R²⁴-substituted isooxazolyl, R²⁴-substituted oxadiazolyl, R²⁴-substituted oxatriazolyl, R²⁴-substituted thienyl, R²⁴-substituted thiazolyl, R²⁴-substituted isothiazolyl, R²⁴-substituted pyridinyl, R²⁴-substituted pyrazinyl, R²⁴-substituted pyrimidinyl, R²⁴-substituted pyridazinyl, R²⁴-substituted triazinyl (e.g., R²⁴-substituted 1,3,5-triazinyl, R²⁴-substituted 1,2,3-triazinyl, or R²⁴-substituted 1,2,4-triazinyl). In embodiments, R²³ is independently an unsubstituted imidazolyl, an unsubstituted pyrrolyl, an unsubstituted pyrazolyl, an unsubstituted triazolyl, an unsubstituted tetrazolyl, an unsubstituted furanyl, an unsubstituted oxazolyl, an unsubstituted isooxazolyl, an unsubstituted oxadiazolyl, an unsubstituted oxatriazolyl, an unsubstituted thienyl, an unsubstituted thiazolyl, an unsubstituted isothiazolyl, an unsubstituted pyridinyl, an unsubstituted pyrazinyl, an unsubstituted pyrimidinyl, an unsubstituted pyridazinyl, an unsubstituted triazinyl (e.g., an unsubstituted 1,3,5-triazinyl, an unsubstituted 1,2,3-triazinyl, or an unsubstituted 1,2,4-triazinyl). In embodiments, R²³ is independently an unsubstituted thiofuranyl. In embodiments, R²³ is independently unsubstituted thienyl.

In embodiments, R²⁴ is independently —Br. In embodiments, R²⁴ is —Cl. In embodiments, R²⁴ is independently —I. In embodiments, R²⁴ is independently —F. In embodiments, R²⁴ is independently —OCH₃. In embodiments, R²⁴ is independently —OCH₂CH₃. In embodiments, R²⁴ is independently substituted or unsubstituted 2 to 3 membered heteroalkyl. In embodiments, R²⁴ is independently substituted or unsubstituted 2 to 4 membered heteroalkyl. In embodiments, R²⁴ is independently substituted or unsubstituted 2 to 6 membered heteroalkyl. In embodiments, R²⁴ is independently —CF₃. In embodiments, R²⁴ is independently —CHF₂. In embodiments, R²⁴ is independently —CH₂F. In embodiments, R²⁴ is independently —CCl₃. In embodiments, R²⁴ is independently —CHCl₂. In embodiments, R²⁴ is independently —CH₂Cl. In embodiments, R²⁴ is independently —CBr₃. In embodiments, R²⁴ is independently —CHBr₂. In embodiments, R²⁴ is independently —CH₂Br. In embodiments, R²⁴ is independently —C₁₃. In embodiments, R²⁴ is independently —CHI₂. In embodiments, R²⁴ is independently —CH₂I. In embodiments, R²⁴ is independently —OCF₃. In embodiments, R²⁴ is independently —OCHF₂. In embodiments, R²⁴ is independently —OCH₂F. In embodiments, R²⁴ is independently —OCCl₃. In embodiments, R²⁴ is independently —OCHCl₂. In embodiments, R²⁴ is independently —OCH₂Cl. In embodiments, R²⁴ is independently —OCBr₃. In embodiments, R²⁴ is independently —OCHBr₂. In embodiments, R²⁴ is independently —OCH₂Br. In embodiments, R²⁴ is independently —OCI₃. In embodiments, R²⁴ is independently —OCHI₂. In embodiments, R²⁴ is independently —OCH₂I.

In embodiments, R²⁴ is independently substituted or unsubstituted C₁-C₆ alkyl. In embodiments, R²⁴ is independently substituted or unsubstituted 2 to 6 membered heteroalkyl. In embodiments, R²⁴ is independently substituted or unsubstituted C₃-C₈ cycloalkyl. In embodiments, R²⁴ is independently substituted or unsubstituted 3 to 8 membered heterocycloalkyl. In embodiments, R²⁴ is independently substituted or unsubstituted phenyl. In embodiments, R²⁴ is independently substituted or unsubstituted 5 to 6 membered heteroaryl. In embodiments, R²⁴ is independently halogen. In embodiments, R²⁴ is independently —CX²⁴₃. In embodiments, R²⁴ is independently —CHX²⁴₂. In embodiments, R²⁴ is independently —CH₂X²⁴. In embodiments, R²⁴ is independently —OCX²⁴₃. In embodiments, R²⁴ is independently —OCH₂X²⁴. In embodiments, R²⁴ is independently —OCHX²⁴₂. In embodiments, R²⁴ is independently —OCH₂X²⁴. In embodiments, R²⁴ is independently —CN. In embodiments, R²⁴ is independently —CH₃. In embodiments, R²⁴ is independently —OCH₃.

In embodiments, R²⁴ is independently substituted or unsubstituted alkyl (e.g., C₁-C₈ alkyl, C₁-C₆ alkyl, or C₁-C₄ alkyl). In embodiments, R²⁴ is independently substituted alkyl (e.g., C₁-C₈ alkyl, C₁-C₆ alkyl, or C₁-C₄ alkyl). In embodiments, R²⁴ is independently an unsubstituted alkyl (e.g., C₁-C₈ alkyl, C₁-C₆ alkyl, or C₁-C₄ alkyl).

In embodiments, R²⁴ is independently substituted or unsubstituted methyl. In embodiments, R²⁴ is independently substituted or unsubstituted C₂ alkyl. In embodiments, R²⁴ is independently substituted or unsubstituted C₃ alkyl. In embodiments, R²⁴ is independently substituted or unsubstituted C₄ alkyl. In embodiments, R²⁴ is independently substituted or unsubstituted C₅ alkyl. In embodiments, R²⁴ is independently substituted or unsubstituted C₆ alkyl. In embodiments, R²⁴ is independently substituted or unsubstituted C₇ alkyl. In embodiments, R²⁴ is independently substituted or unsubstituted C₈ alkyl. In embodiments, R²⁴ is independently substituted methyl. In embodiments, R²⁴ is independently substituted C₂ alkyl. In embodiments, R²⁴ is independently substituted C₃ alkyl. In embodiments, R²⁴ is independently substituted C₄ alkyl. In embodiments, R²⁴ is independently substituted C₅ alkyl. In embodiments, R²⁴ is independently substituted C₆ alkyl. In embodiments, R²⁴ is independently substituted C₇ alkyl. In embodiments, R²⁴ is independently substituted C₈ alkyl. In embodiments, R²⁴ is independently an unsubstituted methyl. In embodiments, R²⁴ is independently an unsubstituted C₂ alkyl. In embodiments, R² is independently an unsubstituted C₃ alkyl. In embodiments, R² is independently an unsubstituted C₄ alkyl. In embodiments, R²⁴ is independently an unsubstituted C₅ alkyl. In embodiments, R²⁴ is independently an unsubstituted C₆ alkyl. In embodiments, R²⁴ is independently an unsubstituted C₇ alkyl. In embodiments, R²⁴ is independently an unsubstituted C₈ alkyl.

R^{100A}, R^{100B}, R^{100C}, and R^{100D} are independently

hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{100A} and R^{100B} substituents bonded to the same nitrogen atom may optionally be joined to form a substituted or unsubstituted heterocycloalkyl or substituted or unsubstituted heteroaryl.

In embodiments, R^{100A}, R^{100B}, R^{100C}, and R^{100D} are independently

hydrogen, —CX₃, —CN, —COOH, —CONH₂, —CHX₂, —CH₂X, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{100A} and R^{100B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

In embodiments, R^{100A} is independently

hydrogen, —CX^{100A}₃, —CHX^{100A}₂, —CH₂X^{100A}, —CN, —COOH, —CONH₂, R^{101A}-substituted or unsubstituted alkyl (e.g., C₁-C₈, C₁-C₆, C₁-C₄, or C₁-C₂), R^{101A}-substituted or unsubstituted heteroalkyl (e.g., 2 to 8 membered, 2 to 6 membered, 4 to 6 membered, 2 to 3 membered, or 4 to 5 membered), R^{101A}-substituted or unsubstituted cycloalkyl (e.g., C₃-C₈, C₃-C₆, C₄-C₆, or C₅-C₆), R^{101A}-substituted or unsubstituted heterocycloalkyl (e.g., 3 to 8 membered, 3 to 6 membered, 4 to 6 membered, 4 to 5 membered, or 5 to 6 membered), R^{101A}-substituted or unsubstituted aryl (e.g., C₆-C₁₀ or phenyl), or R^{101A}-substituted or unsubstituted heteroaryl (e.g., 5 to 10 membered, 5 to 9 membered, or 5 to 6 membered). In embodiments, R^{100A} is independently
hydrogen, —CX^{100A}₃, —CHX^{100A}₂, —CH₂X^{100A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{100A} is independently —F, —Cl, —Br, or —I.

In embodiments, R^{100A} is independently hydrogen. In embodiments, R^{100A} is independently unsubstituted methyl. In embodiments, R^{100A} is independently unsubstituted ethyl.

In embodiments, R^{100A} and R^{100B} substituents bonded to the same nitrogen atom may optionally be joined to form a R^{101A}-substituted or unsubstituted heterocycloalkyl (e.g., 3 to 8 membered, 3 to 6 membered, 4 to 6 membered, 4 to 5 membered, or 5 to 6 membered) or R^{101A}-substituted or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form a R^{101A}-substituted or unsubstituted heterocycloalkyl (e.g., 3 to 8 membered, 3 to 6 membered, 4 to 6 membered, 4 to 5 membered, or 5 to 6 membered). In embodiments, R^{100A} and R^{100B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{100A} and R^{100B} substituents bonded to the same nitrogen atom may optionally be joined to form a R^{101A}-substituted or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form an unsubstituted heteroaryl (e.g., 5 to 10 membered, 5 to 9 membered, or 5 to 6 membered).

R^{101A} is independently oxo,

halogen, —CX^{101A}₃, —CHX^{101A}₂, —CH₂X^{101A}, —OCX^{101A}₃, —OCH₂X^{101A}, —OCHX^{101A}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{102A}-substituted or unsubstituted alkyl (e.g., C₁-C₈, C₁-C₆, C₁-C₄, or C₁-C₂), R^{102A}-substituted or unsubstituted heteroalkyl (e.g., 2 to 8 membered, 2 to 6 membered, 4 to 6 membered, 2 to 3 membered, or 4 to 5 membered), R^{102A}-substituted or unsubstituted cycloalkyl (e.g., C₃-C₈, C₃-C₆, C₄-C₆, or C₅-C₆), R^{102A}-substituted or unsubstituted heterocycloalkyl (e.g., 3 to 8 membered, 3 to 6 membered, 4 to 6 membered, 4 to 5 membered, or 5 to 6 membered), R^{102A}-substituted or unsubstituted aryl (e.g., C₆-C₁₀ or phenyl), or R^{102A}-substituted or unsubstituted heteroaryl (e.g., 5 to 10 membered, 5 to 9 membered, or 5 to 6 membered). In embodiments, R^{101A} is independently oxo,
halogen, —CX^{101A}₃, —CHX^{101A}₂, —CH₂X^{101A}, —OCX^{101A}₃, —OCH₂X^{101A}, —OCHX^{101A}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{101A} is independently —F, —Cl, —Br, or —I.

In embodiments, R^{101A} is independently unsubstituted methyl. In embodiments, R^{101A} is independently unsubstituted ethyl.

R^{102A} is independently oxo,

halogen, —CX^{102A}₃, —CHX^{102A}₂, —CH₂X^{102A}, —OCX^{102A}₃, —OCH₂X^{102A}, —OCHX^{102A}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{102A} is independently —F, —Cl, —Br, or —I.

In embodiments, R^{100B} is independently

hydrogen, —CX^{100B}₃, —CHX^{100B}₂, —CH₂X^{100B}, —CN, —COOH, —CONH₂, R^{101B}-substituted or unsubstituted alkyl (e.g., C₁-C₈, C₁-C₆, C₁-C₄, or C₁-C₂), R^{101B}-substituted or unsubstituted heteroalkyl (e.g., 2 to 8 membered, 2 to 6 membered, 4 to 6 membered, 2 to 3 membered, or 4 to 5 membered), R^{101B}-substituted or unsubstituted cycloalkyl (e.g., C₃-C₈, C₃-C₆, C₄-C₆, or C₅-C₆), R^{101B}-substituted or unsubstituted heterocycloalkyl (e.g., 3 to 8 membered, 3 to 6 membered, 4 to 6 membered, 4 to 5 membered, or 5 to 6 membered), R^{101B}-substituted or unsubstituted aryl (e.g., C₆-C₁₀ or phenyl), or R^{101B}-substituted or unsubstituted heteroaryl (e.g., 5 to 10 membered, 5 to 9 membered, or 5 to 6 membered). In embodiments, R^{100B} is independently
hydrogen, —CX^{100B}₃, —CHX^{100B}₂, —CH₂X^{100B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{100B} is independently —F, —Cl, —Br, or —I.

In embodiments, R^{100B} is independently hydrogen. In embodiments, R^{100B} is independently unsubstituted methyl. In embodiments, R^{100B} is independently unsubstituted ethyl.

In embodiments, R^{100A} and R^{100B} substituents bonded to the same nitrogen atom may optionally be joined to form a R^{101B}-substituted or unsubstituted heterocycloalkyl (e.g., 3 to 8 membered, 3 to 6 membered, 4 to 6 membered, 4 to 5 membered, or 5 to 6 membered) or R^{101B}-substituted or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form a R^{101B}-substituted or unsubstituted heterocycloalkyl (e.g., 3 to 8 membered, 3 to 6 membered, 4 to 6 membered, 4 to 5 membered, or 5 to 6 membered). In embodiments, R^{100A} and R^{100B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered). In embodiments, R^{100A} and R^{100B} substituents bonded to the same nitrogen atom may optionally be joined to form a R^{101B}-substituted or unsubstituted heteroaryl (e.g., 5 to 10 membered, 5 to 9 membered, or 5 to 6 membered). In embodiments, R^{100A} and R^{100B} substituents bonded to the same nitrogen atom may optionally be joined to form an unsubstituted heteroaryl (e.g., 5 to 10 membered, 5 to 9 membered, or 5 to 6 membered).

R^{101B} is independently oxo,

halogen, —CX^{101B}₃, —CHX^{101B}₂, —CH₂X^{101B}, —OCX^{101B}₃, —OCH₂X^{101B}, —OCHX^{101B}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{102B}-substituted or unsubstituted alkyl (e.g., C₁-C₈, C₁-C₆, C₁-C₄, or C₁-C₂), R^{102B}-substituted or unsubstituted heteroalkyl (e.g., 2 to 8 membered, 2 to 6 membered, 4 to 6 membered, 2 to 3 membered, or 4 to 5 membered), R^{102B}-substituted or unsubstituted cycloalkyl (e.g., C₃-C₈, C₃-C₆, C₄-C₆, or C₅-C₆), R^{102B}-substituted or unsubstituted heterocycloalkyl (e.g., 3 to 8 membered, 3 to 6 membered, 4 to 6 membered, 4 to 5 membered, or 5 to 6 membered), R^{102B}-substituted or unsubstituted aryl (e.g., C₆-C₁₀ or phenyl), or R^{102B}-substituted or unsubstituted heteroaryl (e.g., 5 to 10 membered, 5 to 9 membered, or 5 to 6 membered). In embodiments, R^{101B} is independently oxo,
halogen, —CX^{101B}₃, —CHX^{101B}₂, —CH₂X^{101B}, —OCX^{101B}₃, —OCH₂X^{101B}, —OCHX^{101B}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{101B} is independently —F, —Cl, —Br, or —I.

In embodiments, R^{101B} is independently unsubstituted methyl. In embodiments, R^{101B} is independently unsubstituted ethyl.

R^{102B} is independently oxo,

halogen, —CX^{102B}₃, —CHX^{102B}₂, —CH₂X^{102B}, —OCX^{102B}₃, —OCH₂X^{102B}, —OCHX^{102B}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{102B} is independently —F, —Cl, —Br, or —I.

In embodiments, R^{100C} is independently

hydrogen, —CX^{100C}₃, —CHX^{100C}₂, —CH₂X^{100C}, —CN, —COOH, —CONH₂, R^{101C}-substituted or unsubstituted alkyl (e.g., C₁-C₈, C₁-C₆, C₁-C₄, or C₁-C₂), R^{101C}-substituted or unsubstituted heteroalkyl (e.g., 2 to 8 membered, 2 to 6 membered, 4 to 6 membered, 2 to 3 membered, or 4 to 5 membered), R^{101C}-substituted or unsubstituted cycloalkyl (e.g., C₃-C₈, C₃-C₆, C₄-C₆, or C₅-C₆), R^{101C}-substituted or unsubstituted heterocycloalkyl (e.g., 3 to 8 membered, 3 to 6 membered, 4 to 6 membered, 4 to 5 membered, or 5 to 6 membered), R^{101C}-substituted or unsubstituted aryl (e.g., C₆-C₁₀ or phenyl), or R^{101C}-substituted or unsubstituted heteroaryl (e.g., 5 to 10 membered, 5 to 9 membered, or 5 to 6 membered). In embodiments, R^{100C} is independently
hydrogen, —CX^{100C}₃, —CHX^{100C}₂, —CH₂X^{100C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{100C} is independently —F, —Cl, —Br, or —I.

In embodiments, R^{100C} is independently hydrogen. In embodiments, R^{100C} is independently unsubstituted methyl. In embodiments, R^{100C} is independently unsubstituted ethyl.

R^{101C} is independently oxo,

halogen, —CX^{101C}₃, —CHX^{101C}₂, —CH₂X^{101C}, —OCX^{101C}₃, —OCH₂X^{101C}, —OCHX^{101C}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{102C}-substituted or unsubstituted alkyl (e.g., C₁-C₈, C₁-C₆, C₁-C₄, or C₁-C₂), R^{102C}-substituted or unsubstituted heteroalkyl (e.g., 2 to 8 membered, 2 to 6 membered, 4 to 6 membered, 2 to 3 membered, or 4 to 5 membered), R^{102C}-substituted or unsubstituted cycloalkyl (e.g., C₃-C₈, C₃-C₆, C₄-C₆, or C₅-C₆), R^{102C}-substituted or unsubstituted heterocycloalkyl (e.g., 3 to 8 membered, 3 to 6 membered, 4 to 6 membered, 4 to 5 membered, or 5 to 6 membered), R^{102C}-substituted or unsubstituted aryl (e.g., C₆-C₁₀ or phenyl), or R^{102C}-substituted or unsubstituted heteroaryl (e.g., 5 to 10 membered, 5 to 9 membered, or 5 to 6 membered). In embodiments, R^{101C} is independently oxo,
halogen, —CX^{101C}₃, —CHX^{101C}₂, —CH₂X^{101C}, —OCX^{101C}₃, —OCH₂X^{101C}, —OCHX^{101C}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{101C} is independently —F, —Cl, —Br, or —I.

In embodiments, R^{101C} is independently unsubstituted methyl. In embodiments, R^{101C} is independently unsubstituted ethyl.

R^{102C} is independently oxo,

halogen, —CX^{102C}₃, —CHX^{102C}₂, —CH₂X^{102C}, —OCX^{102C}₃, —OCH₂X^{102C}, —OCHX^{102C}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{102C} is independently —F, —Cl, —Br, or —I.

In embodiments, R^{100D} is independently

hydrogen, —CX^{100D}₃, —CHX^{100D}₂, —CH₂X^{100D}, —CN, —COOH, —CONH₂, R^{101D}-substituted or unsubstituted alkyl (e.g., C₁-C₈, C₁-C₆, C₁-C₄, or C₁-C₂), R^{101D}-substituted or unsubstituted heteroalkyl (e.g., 2 to 8 membered, 2 to 6 membered, 4 to 6 membered, 2 to 3 membered, or 4 to 5 membered), R^{101D}-substituted or unsubstituted cycloalkyl (e.g., C₃-C₈, C₃-C₆, C₄-C₆, or C₅-C₆), R^{101D}-substituted or unsubstituted heterocycloalkyl (e.g., 3 to 8 membered, 3 to 6 membered, 4 to 6 membered, 4 to 5 membered, or 5 to 6 membered), R^{101D}-substituted or unsubstituted aryl (e.g., C₆-C₁₀ or phenyl), or R^{101D}-substituted or unsubstituted heteroaryl (e.g., 5 to 10 membered, 5 to 9 membered, or 5 to 6 membered). In embodiments, R^{101D} is independently
hydrogen, —CX^{101D}₃, —CHX^{101D}₂, —CH₂X^{101D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{100D} is independently —F, —Cl, —Br, or —I.

In embodiments, R^{100D} is independently hydrogen. In embodiments, R^{100D} is independently unsubstituted methyl. In embodiments, R^{100D} is independently unsubstituted ethyl.

R^{101D} is independently oxo,

halogen, —CX^{101D}₃, —CHX^{101D}₂, —CH₂X^{101D}, —OCX^{101D}₃, —OCH₂X^{101D}, —OCHX^{101D}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{102D}-substituted or unsubstituted alkyl (e.g., C₁-C₈, C₁-C₆, C₁-C₄, or C₁-C₂), R^{102D}-substituted or unsubstituted heteroalkyl (e.g., 2 to 8 membered, 2 to 6 membered, 4 to 6 membered, 2 to 3 membered, or 4 to 5 membered), R^{102D}-substituted or unsubstituted cycloalkyl (e.g., C₃-C₈, C₃-C₆, C₄-C₆, or C₅-C₆), R^{102D}-substituted or unsubstituted heterocycloalkyl (e.g., 3 to 8 membered, 3 to 6 membered, 4 to 6 membered, 4 to 5 membered, or 5 to 6 membered), R^{102D}-substituted or unsubstituted aryl (e.g., C₆-C₁₀ or phenyl), or R^{102D}-substituted or unsubstituted heteroaryl (e.g., 5 to 10 membered, 5 to 9 membered, or 5 to 6 membered). In embodiments, R^{101D} is independently oxo,
halogen, —CX^{101D}₃, —CHX^{101D}₂, —CH₂X^{101D}, —OCX^{101D}₃, —OCH₂X^{101D}, —OCHX^{101D}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{101D} is independently —F, —Cl, —Br, or —I.

In embodiments, R^{101D} is independently unsubstituted methyl. In embodiments, R^{101D} is independently unsubstituted ethyl.

R^{102D} is independently oxo,

halogen, —CX^{102D}₃, —CHX^{102D}₂, —CH₂X^{102D}, —OCX^{102D}₃, —OCH₂X^{102D}, —OCHX^{102D}₂, —CN, —OH, —N H₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{102D} is independently —F, —Cl, —Br, or —I.

In embodiments, the compound has the formula:

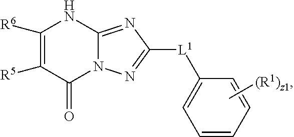

wherein R⁶, R³, L¹, R¹, and z1 are as described herein, including embodiments.

In embodiments, the compound has the formula:

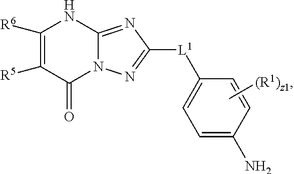

wherein R⁶, R⁵, L¹, R¹, and z1 are as described herein, including embodiments.

In embodiments, the compound has the formula:

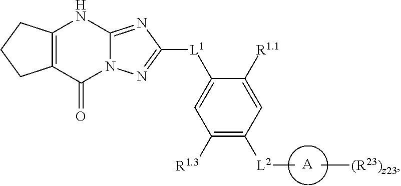

wherein L¹, L², Ring A, R²³, and z23 are as described herein, including embodiments. R^{1.1} and R^{1.3} are each hydrogen or R¹ at a fixed position on the attached ring. R^{1.1} and R^{1.3} may independently be hydrogen or any substituent of R¹ described herein, including in any aspect, embodiment, example, figure, or claim.

In embodiments, R^{1.1} and R^{1.3} are each independently substituted or unsubstituted alkyl (e.g., C₁-C₈ alkyl, C₁-C₆ alkyl, or C₁-C₄ alkyl). In embodiments, R^{1.1} and R^{1.3} are each independently substituted alkyl (e.g., C₁-C₈ alkyl, C₁-C₆ alkyl, or C₁-C₄ alkyl). In embodiments, R^{1.1} and R^{1.3} are each independently an unsubstituted alkyl (e.g., C₁-C₈ alkyl, C₁-C₆ alkyl, or C₁-C₄ alkyl). In embodiments, R^{1.1} and R^{1.3} are each independently substituted or unsubstituted methyl. In embodiments, R^{1.1} and R^{1.3} are each independently substituted or unsubstituted C₂ alkyl. In embodiments, R^{1.1} and R^{1.3} are each independently substituted or unsubstituted C₃ alkyl. In embodiments, R^{1.1} and R^{1.3} are each independently substituted or unsubstituted C₄ alkyl. In embodiments, R^{1.1} and R^{1.3} are each independently substituted or unsubstituted C₅ alkyl. In embodiments, R^{1.1} and R^{1.3} are each independently substituted or unsubstituted C₆ alkyl. In embodiments, R^{1.1} and R^{1.3} are each independently substituted or unsubstituted C₇ alkyl. In embodiments, R^{1.1} and R^{1.3} are each independently substituted or unsubstituted C₈ alkyl. In embodiments, R^{1.1} and R^{1.3} are each independently substituted methyl. In embodiments, R^{1.1} and R^{1.3} are each independently substituted C₂ alkyl. In embodiments, R^{1.1} and R^{1.3} are each independently substituted C₃ alkyl. In embodiments, R^{1.1} and R^{1.3} are each independently substituted C₄ alkyl. In embodiments, R^{1.1} and R^{1.3} are each independently substituted C₅ alkyl. In embodiments, R^{1.1} and R^{1.3} are each independently substituted C₆ alkyl. In embodiments, R^{1.1} and R^{1.3} are each independently substituted C₇ alkyl. In embodiments, R^{1.1} and R^{1.3} are each independently substituted C₈ alkyl. In embodiments, R^{1.1} and R^{1.3} are each independently an unsubstituted methyl. In embodiments, R^{1.1} and R^{1.3} are each independently an unsubstituted C₂ alkyl. In embodiments, R^{1.1} and R^{1.3} are each independently an unsubstituted C₃ alkyl. In embodiments, R^{1.1} and R^{1.3} are each independently an unsubstituted C₄ alkyl. In embodiments, R^{1.1} and R^{1.3} are each independently an unsubstituted C₅ alkyl. In embodiments, R^{1.1} and R^{1.3} are each independently an unsubstituted C₆ alkyl. In embodiments, R^{1.1} and R^{1.3} are each independently an unsubstituted C₇ alkyl. In embodiments, R^{1.1} and R^{1.3} are each independently an unsubstituted C₈ alkyl.

In embodiments, the compound has the formula:

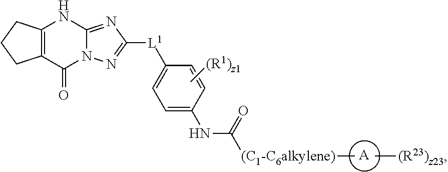

wherein L¹, Ring A, R²³, R¹, z1, and z23 are as described herein, including embodiments.

In embodiments, the compound has the formula:

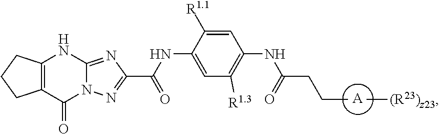

wherein R^{1.1}, R^{1.3}, Ring A, R²³, and z23 are as described herein, including embodiments.

In embodiments, the agent that inhibits MDA-9 is a compound having the formula:

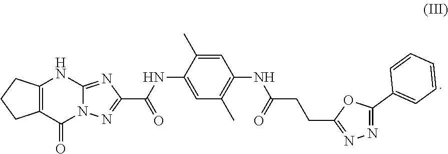 [SMILES: Cc1cc(NC(=O)c2nc3[nH]c4c(c(=O)n3n2)CCC4)c(C)cc1NC(=O)CCc1nnc(-c2ccccc2)o1]

Formula III is also referred to herein as 113B7, PDZ1in, and PDZ1i.

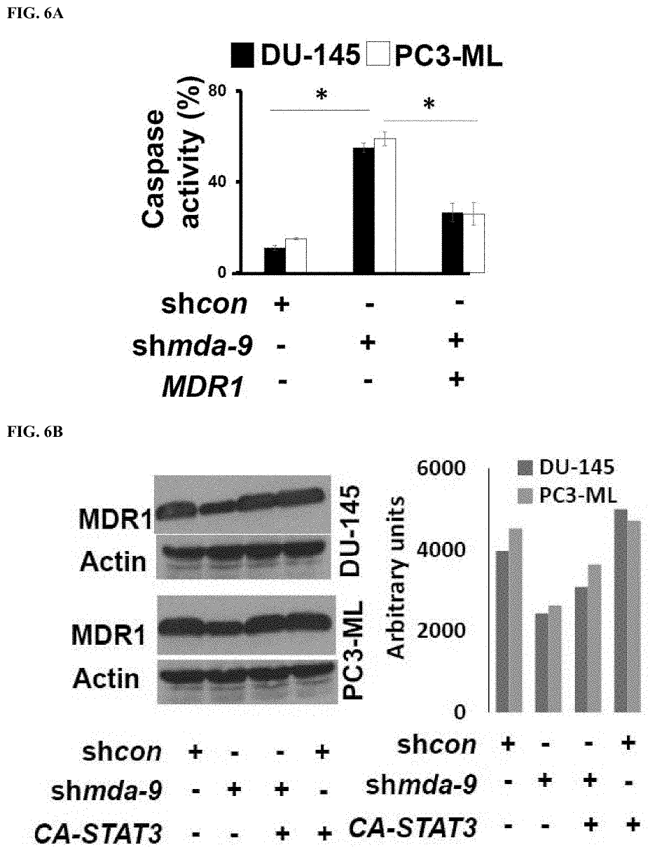

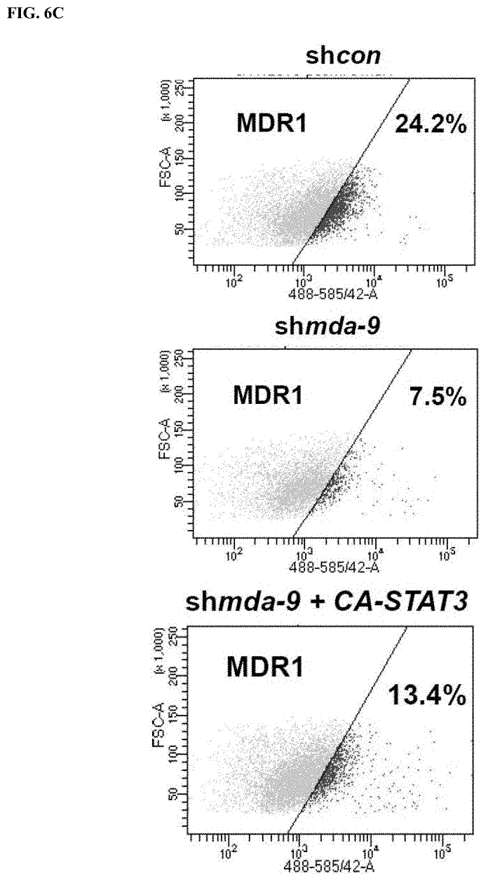

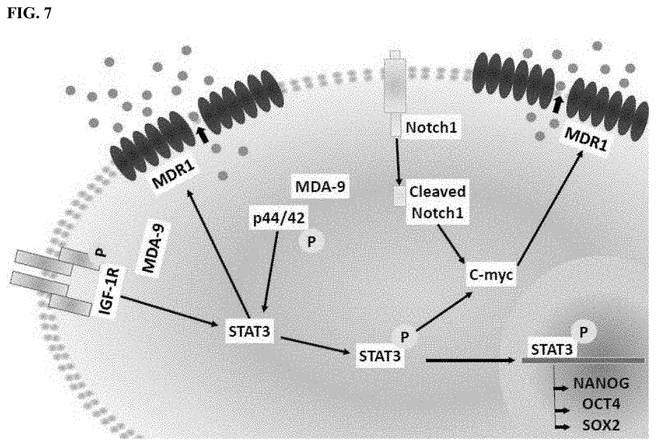

The compounds of Formula I, Formula II, Formula IIA, and Formula III can be synthesized according to procedures described herein, including in FIG. 6.

In some aspects or embodiments of the invention, the compounds described herein are provided as prodrugs. In this case, one or more than one functional group, and/or one or more than one type of functional group, is covalently attached to reactive group of the molecule, e.g. to an OH, an NH, a carboxyl, etc. In some aspects, the protecting group is removed by enzymatic and/or non-enzymatic reactions, e.g. by hydrolysis, at or near the site of action (e.g. at the site of a tumor). In other aspects, the protecting group remains attached to the drug at the site of action, but does not interfere with the activity of the drug, or at least does not interfere to an extent that make the drug ineffective. In yet other aspects or embodiments, the protecting groups are selected so as to be gradually removed non-enzymatically as the drug circulates, resulting in a slow release over time of an active drug. Exemplary protecting groups that may be used to make such prodrugs include but are not limited to: amidomethyl esters (for carboxyl groups); acyloxymethyl (for carboxyl groups); monomethoxytrityl (MMT) (for amino groups); carbamoyl moieties (carbamate esters of amino acids); pivaloyloxymethyl (POM, pivoxil, pivoxyl), benzoyl, acetyl, trimethylsilyl, triethylsilyl, or methoxymethyl groups (for hydroxy groups); etc. In some aspects or embodiments, carboxyl groups of the molecule are protected in this manner and the protecting group is removed in vivo, e.g. the protecting group is a cleavable alkyl of aryl ester at a C-terminal carboxylate.

In embodiments, n1 is 0. In embodiments, n1 is 1. In embodiments, n1 is 2. In embodiments, n1 is 3. In embodiments, n1 is 4. In embodiments, m1 is 1. In embodiments, m1 is 2. In embodiments, v1 is 1. In embodiments, v1 is 2.

In embodiments, z1 is 0. In embodiments, z1 is 1. In embodiments, z1 is 2. In embodiments, z1 is 3. In embodiments, z1 is 4. In embodiments, z1 is 5. In embodiments, the symbol z1 is an integer from 0 to 4. In embodiments, the symbol z1 is an integer from 0 to 2. In embodiments, z23 is 0. In embodiments, z23 is 1. In embodiments, z23 is 2. In embodiments, z23 is 3. In embodiments, z23 is 4. In embodiments, z23 is 5. In embodiments, the symbol z23 is an integer from 0 to 4.

In embodiments, n2 is 0. In embodiments, n2 is 1. In embodiments, n2 is 2. In embodiments, n2 is 3. In embodiments, n2 is 4. In embodiments, m2 is 1. In embodiments, m2 is 2. In embodiments, v2 is 1. In embodiments, v2 is 2.

In embodiments, n23 is 0. In embodiments, n23 is 1. In embodiments, n23 is 2. In embodiments, n23 is 3. In embodiments, n23 is 4. In embodiments, m23 is 1. In embodiments, m23 is 2. In embodiments, v23 is 1. In embodiments, v23 is 2.

In embodiments, the compound is a compound described herein, for example a compound in Table 1. In embodiments the compound is 113B12, 113B11, 113B9, 113B7, 112H9, 113B8, B112G11, 112G4, 112G3, 112G2, 112G1, 112F12, 112F11, 112F10, 112F1, 112E12, 112E7, 112D11, cmpd14, cmpd13, cmpd12, cmpd11, cmpd10, cmpd9, cmpd8, cmpd7, cmpd6, cmpd5, cmpd4, cmpd3, cmpd2, cmpd1, or 30A9 as identified in Table 1. In embodiments, the compound is 113B7.

TABLE 1

| | | Binding |
|---|---|---|
| | | affinity |
| | | ranking * |
| ID | Compound Structure | (K_{d} values) |
| 30A9 | 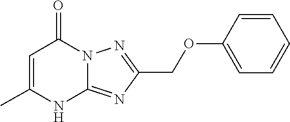 [SMILES: Cc1cc(=O)n2nc(COc3ccccc3)nc2[nH]1] | 4 (197.2 μM) |
| cmpd1 | 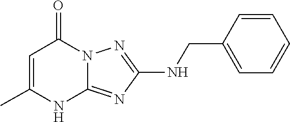 [SMILES: Cc1cc(=O)n2nc(NCc3ccccc3)nc2[nH]1] | 2 |
| cmpd2 | 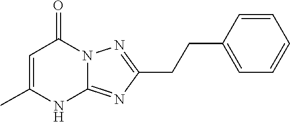 [SMILES: Cc1cc(=O)n2nc(CCc3ccccc3)nc2[nH]1] | 3 |
| cmpd3 | 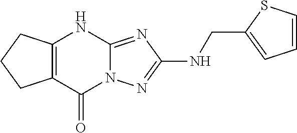 [SMILES: O=c1c2c([nH]c3nc(NCc4cccs4)nn13)CCC2] | 1 |
| cmpd4 | 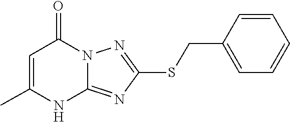 [SMILES: Cc1cc(=O)n2nc(SCc3ccccc3)nc2[nH]1] | 4 |
| cmpd5 | 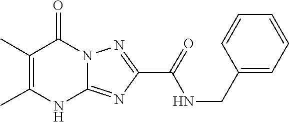 [SMILES: Cc1[nH]c2nc(C(=O)NCc3ccccc3)nn2c(=O)c1C] | 2 |
| cmpd6 | 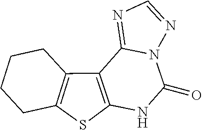 [SMILES: O=c1[nH]c2sc3c(c2c2ncnn12)CCCC3] | 1 |
| cmpd7 | 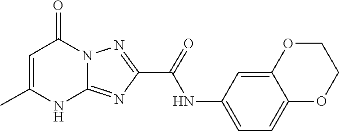 [SMILES: Cc1cc(=O)n2nc(C(=O)Nc3ccc4c(c3)OCCO4)nc2[nH]1] | 2 |
| cmpd8 | 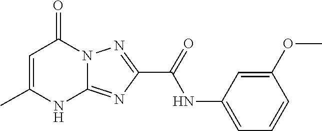 | 3 |
| cmpd9 | 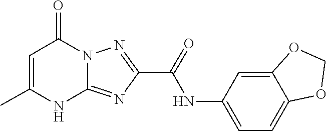 [SMILES: Cc1cc(=O)n2nc(C(=O)Nc3ccc4c(c3)OCO4)nc2[nH]1] | 3 |
| cmpd10 | 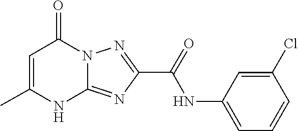 [SMILES: Cc1cc(=O)n2nc(C(=O)Nc3cccc(Cl)c3)nc2[nH]1] | 4 (151.4 μM) |
| cmpd11 | 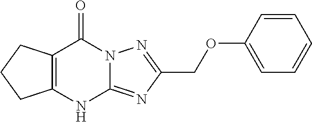 [SMILES: O=c1c2c([nH]c3nc(COc4ccccc4)nn13)CCC2] | 5 (170.6 μM) |
| cmpd12 | 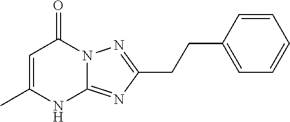 [SMILES: Cc1cc(=O)n2nc(CCc3ccccc3)nc2[nH]1] | 3 |
| cmpd13 | 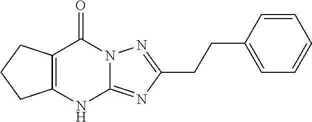 [SMILES: O=c1c2c([nH]c3nc(CCc4ccccc4)nn13)CCC2] | 3 |
| cmpd14 | 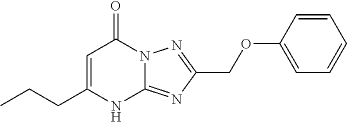 [SMILES: CCCc1cc(=O)n2nc(COc3ccccc3)nc2[nH]1] | 1 |
| 112D11 | 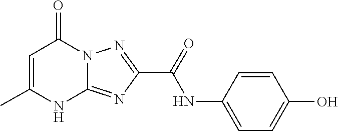 [SMILES: Cc1cc(=O)n2nc(C(=O)Nc3ccc(O)cc3)nc2[nH]1] | 2 (244.7 μM) |
| 112E7 | 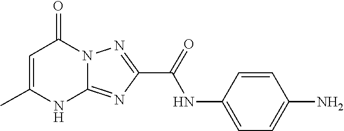 [SMILES: Cc1cc(=O)n2nc(C(=O)Nc3ccc(N)cc3)nc2[nH]1] | 1 (944.2 μM) |
| 112E12 | 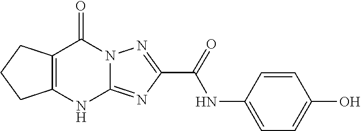 [SMILES: O=C(Nc1ccc(O)cc1)c1nc2[nH]c3c(c(=O)n2n1)CCC3] | 1 (1610 μM) |
| 112F1 | 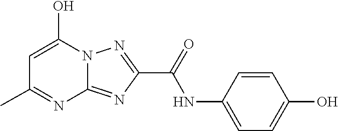 [SMILES: Cc1cc(O)n2nc(C(=O)Nc3ccc(O)cc3)nc2n1] | 1 |
| 112F10 | 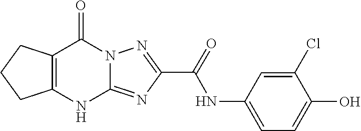 [SMILES: O=C(Nc1ccc(O)c(Cl)c1)c1nc2[nH]c3c(c(=O)n2n1)CCC3] | 4 |
| 112F11 | 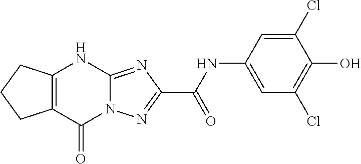 [SMILES: O=C(Nc1cc(Cl)c(O)c(Cl)c1)c1nc2[nH]c3c(c(=O)n2n1)CCC3] | 5 (71.76 μM) |
| 112F12 | 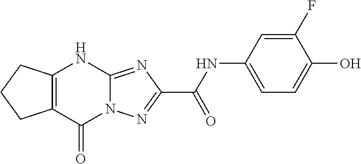 [SMILES: O=C(Nc1ccc(O)c(F)c1)c1nc2[nH]c3c(c(=O)n2n1)CCC3] | 2 (165.9 μM) |
| 112G1 | 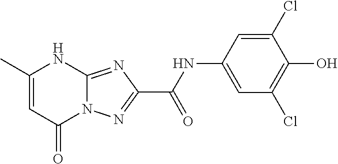 [SMILES: Cc1cc(=O)n2nc(C(=O)Nc3cc(Cl)c(O)c(Cl)c3)nc2[nH]1] | 6 (57.3 μM) |
| 112G2 | 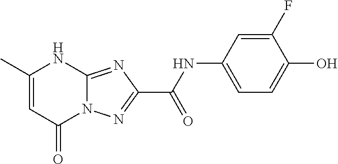 [SMILES: Cc1cc(=O)n2nc(C(=O)Nc3ccc(O)c(F)c3)nc2[nH]1] | 4 (146.8 μM) |
| 112G3 | 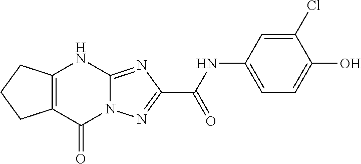 [SMILES: O=C(Nc1ccc(O)c(Cl)c1)c1nc2[nH]c3c(c(=O)n2n1)CCC3] | 4 (119.3 μM) |
| 112G4 | 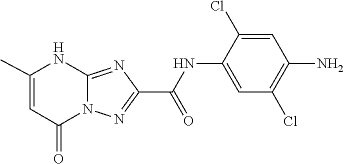 [SMILES: Cc1cc(=O)n2nc(C(=O)Nc3cc(Cl)c(N)cc3Cl)nc2[nH]1] | 6 (34.3 μM) |
| 112G11 | 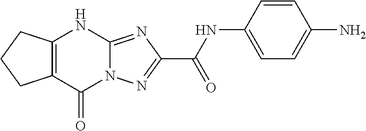 [SMILES: Nc1ccc(NC(=O)c2nc3[nH]c4c(c(=O)n3n2)CCC4)cc1] | 2 (263.1 μM) |
| 113B8 | 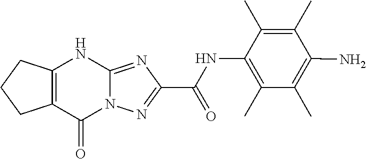 [SMILES: Cc1c(C)c(NC(=O)c2nc3[nH]c4c(c(=O)n3n2)CCC4)c(C)c(C)c1N] | 5 (151.1 μM) |
| 112H9 | 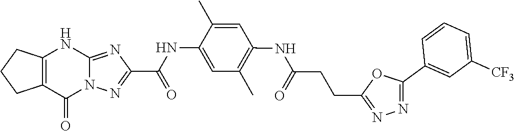 [SMILES: Cc1cc(NC(=O)c2nc3[nH]c4c(c(=O)n3n2)CCC4)c(C)cc1NC(=O)CCc1nnc(-c2cccc(C(F)(F)F)c2)o1] | 2 (38.33 μM) |
| 113B7 (PDZ1i) | 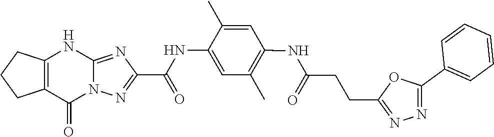 [SMILES: Cc1cc(NC(=O)c2nc3[nH]c4c(c(=O)n3n2)CCC4)c(C)cc1NC(=O)CCc1nnc(-c2ccccc2)o1] | 4 (21.37 μM) |
| 113B9 | 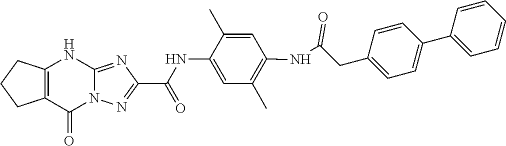 [SMILES: Cc1cc(NC(=O)c2nc3[nH]c4c(c(=O)n3n2)CCC4)c(C)cc1NC(=O)Cc1ccc(-c2ccccc2)cc1] | N.B. |
| 113B11 | 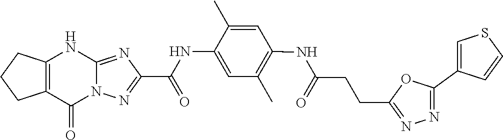 [SMILES: Cc1cc(NC(=O)c2nc3[nH]c4c(c(=O)n3n2)CCC4)c(C)cc1NC(=O)CCc1nnc(-c2ccsc2)o1] | 3 |
| 113B12 | 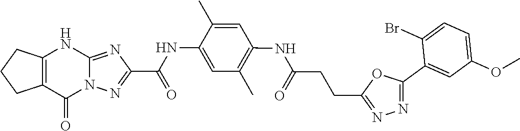 [SMILES: COc1ccc(Br)c(-c2nnc(CCC(=O)Nc3cc(C)c(NC(=O)c4nc5[nH]c6c(c(=O)n5n4)CCC6)cc3C)o2)c1] | 1 |

*Rank ordering of compound affinity for PDZ1 were estimated based on chemical shift changes (DPPM) of the backbone amide of residue 147 on the [¹⁵N,¹H] HSQC spectrum of 50 μM PDZ12 caused by compound at 1:1 molar ratio: 1 for DPPM 0.00-0.02; 2 for DPPM 0.02-0.04; 3 for DPPM 0.04-0.06; 4 for DPPM 0.06-0.08; 5 for DPPM 0.08-0.1; 6 for DPPM above 0.1; N.B. no appreciable binding detected. Kd values reported in parentheses were obtained by NMR titration experiments following the chemical shifts of the backbone amide of residue 147 at different ligand concentrations.

In an aspect is provided a pharmaceutical compositions including a compound as described herein, including embodiments thereof, and a pharmaceutically acceptable salt.

In an aspect is provided a pharmaceutical composition including a compound described herein, or a salt (e.g., pharmaceutically acceptable salt) thereof, and a pharmaceutically acceptable excipient. In embodiments, the compound as described herein is included in a therapeutically effective amount. In embodiments, the compound as described herein is included in an effective amount.

In embodiments of the pharmaceutical compositions, the compound, or salt (e.g., pharmaceutically acceptable salt) thereof, is included in a therapeutically effective amount. In embodiments of the pharmaceutical compositions, the compound, or salt (e.g., pharmaceutically acceptable salt) thereof, is a compound. In embodiments of the pharmaceutical compositions, the compound, or salt (e.g., pharmaceutically acceptable salt) thereof, is a salt (e.g., pharmaceutically acceptable salt) of the compound. In embodiments of the pharmaceutical compositions, the compound, or salt (e.g., pharmaceutically acceptable salt) thereof, is a pharmaceutically acceptable salt of the compound.

In embodiments, the agent that inhibits MDA-9 is an agent that decreases expression of MDA-9. In embodiments, the expression of MDA-9/Syntenin (SDCBP) is modulated with an agent, or with mutation, inactivation, knockdown or deletion of the gene of MDA-9/Syntenin (SDCBP). In one embodiment, the agent is a small interfering RNA (siRNA) or a short hairpin RNA (shRNA) comprising a sequence specific for the gene of MDA-9/Syntenin (SDCBP) or using CRSIPR/Cas9 or similar genome targeted editing approach. In another embodiment, the mutation, inactivation, knockdown or deletion of the gene of MDA-9/Syntenin (SDCBP) is achieved by CRSIPR/Cas9 or other genome targeted editing techniques.

In embodiments, the present disclosure provides anti-cancer agents for use in combination with an MDA-9 inhibiting agent as disclosed herein. In embodiments, the anti-cancer agent is a histone deacetylase (HDAC) inhibitor or a mitotic inhibitor.

In embodiments, the anti-cancer agent is an HDAC inhibitor. Examples of HDAC inhibitors include, but are not limited to, hydroxamic acids (or hydroxamates), such as trichostatin A, cyclic tetrapeptides (such as trapoxin B), and depsipeptides, benzamides, electrophilic ketones, and aliphatic acid compounds such as phenylbutyrate and valproic acid. In embodiments, the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide. In embodiments, the anti-cancer agent is trichostatin A (TSA). In embodiments, the anti-cancer agent is oxamflatin. In embodiments, the anti-cancer agent is apicidin. In embodiments, the anti-cancer agent is depsipeptide. In embodiments, the anti-cancer agent is depudecin. In embodiments, the anti-cancer agent is trapoxin. In embodiments, the anti-cancer agent is vorinostat. In embodiments, the anti-cancer agent is suberoylanilide hydroxamic acid. In embodiments, the anti-cancer agent is pyroxamide.

In embodiments, the anti-cancer agent is a mitotic inhibitor. In embodiments, the anti-cancer agent is an anti-microtubule agent. In embodiments, the anti-cancer agent is a taxane or a vinca alkaloid. In embodiments, the anti-cancer agent is a taxane selected from paclitaxel, docetaxel, cabazitaxel, and larotaxel. In embodiments, the anti-cancer agent is paclitaxel. In embodiments, the anti-cancer agent is docetaxel. In embodiments, the anti-cancer agent is cabazitaxel. In embodiments, the anti-cancer agent is larotaxel. In embodiments, the anti-cancer agent is a vinca alkaloid selected from vinblastine, vincristine, or vinorelbine. In embodiments, the anti-cancer agent is vinblastine. In embodiments, the anti-cancer agent is vincristine. In embodiments, the anti-cancer agent is vinorelbine.

Additional examples of agents that inhibit MDA-9 are disclosed in US20190017054A1 and US20190263824A1, which are incorporated herein by reference.

In embodiments, compounds and pharmaceutical compositions disclosed herein, and combinations thereof, are for use in a treating a subject, such as in accordance with methods disclosed herein. In embodiments, the compounds and pharmaceutical compositions disclosed herein, and combinations thereof, are for the preparation of a medicament for use in treating a subject, such as in accordance with methods disclosed herein. In embodiments, the agent that inhibits MDA-9 and the anti-cancer agent are in a combined effective amount. In embodiments, the combined effective amount is a combined synergistic amount.

In some aspects, the present disclosure provides kits. In embodiments, the kit includes one or more compounds or pharmaceutical compositions disclosed herein. In embodiments, the kit includes (a) an agent that inhibits MDA-9, and (b) an anti-cancer agent, wherein the anti-cancer agent is a mitotic inhibitor or a histone deacetylase (HDAC) inhibitor. In embodiments, active agents in the kit are packaged separately, such as in separate dosage forms. In embodiments, active agents are packaged together, such as in a single dosage form. A dosage form may be a unit dose, or a container with multiple doses. In embodiments, one or more active agents, or combinations thereof, are provided as a ready pharmaceutical composition. In embodiments, two or more active agents are formulated as separate, ready pharmaceutical compositions. In embodiments, one or more agents in a kit require reconstitution prior to use.

### III. Methods of Treating Cancer

Also provided herein are methods of treating cancer in a subject in need thereof. In embodiments, the method includes administering to the subject a combined effective amount of (a) an agent that inhibits MDA-9, and (b) an anti-cancer agent, wherein the anti-cancer agent is a mitotic inhibitor or a histone deacetylase (HDAC) inhibitor. In embodiments, is provided a method of treating cancer in a subject in need thereof, including administering to the subject a combined effective amount of (a) an agent that inhibits MDA-9, and (b) an anti-cancer agent, wherein the anti-cancer agent is a mitotic inhibitor. In embodiments, is provided a method of treating cancer in a subject in need thereof, including administering to the subject a combined effective amount of (a) an agent that inhibits MDA-9, and (b) an anti-cancer agent, wherein the anti-cancer agent is a histone deacetylase (HDAC) inhibitor. In embodiments, the agent that inhibits MDA-9 and the anticancer agent are selected from compounds disclosed herein, such with regard to any of the aspects disclosed herein.

In some aspects, the subject who is treated as described herein has, in fact, not been diagnosed with cancer. Rather, the subject is genetically prone to development of cancer (e.g. the subject may be a woman with a harmful mutation in the PALB2, BRCA1 and/or BRCA2, tumor suppressor genes; c-Kit, APC, mutated p53, PTEN deletion, Braf, are some common gene alterations that are currently tested for predicting cancer risk; activation of oncogenes including members of the Ras gene family, AEG-1 (MTDH), myc gene family (C-myc, N-myc, L-myc); deregulated cell cycle genes such as cyclin E1; etc. In these cases, the compounds of the invention are administered prophylactically and prevent or slow and/or lessen the extent of the cancer that develops and/or make the cancer more treatable when is does occur.

In yet other aspects, the compounds disclosed herein are used to treat subjects who are not diagnosed with cancer per se but rather with a pre-cancerous condition. For example, individuals with colon polyps that are benign, individuals with cervical dysplasia, individuals at high risk for cancer based on familial history, individuals exposed to a carcinogen potentially causing cancer (through the skin, respiratory track, gastrointestinal track, or other route of entry into the body), individuals who have been positively diagnosed using a genetic test for cancer (including containing potentially cancerous circulating tumor cells, presence of positive cancer associated biomarkers (proteins in plasma, miRNA in the blood) expressed in body fluids (blood, urine, saliva, etc.), etc.

In embodiments, the subject who is treated as described herein has been diagnosed with early stage cancer. In such cases, the compounds described herein may be used alone or in combination with other therapeutic modes to address the cancer and to prevent its spread or progression. For example, patients with early stage bladder or prostate cancer are treated to prevent invasion and development of metastatic lesions. Patients who underwent surgery to remove primary or metastatic tumor or have undergone treatment with chemotherapy/radiotherapy/immunotherapy for treating cancers. Potential applications of the current anti-invasive and anti-metastatic molecules is to prevent secondary tumor and metastasis development following conventional surgery or therapy (radiation, chemotherapy, immunotherapy) for cancer. Treatment would begin prior to surgery or other therapies (radiation, chemotherapy, immunotherapy) and continue after surgery or therapy.

In another aspect is provided a method of preventing or treating cancer in a subject in need thereof, including administering to the subject a therapeutically effective amount of aa compound described herein, including embodiments thereof, wherein the therapeutically effective amount is sufficient to prevent or treat the cancer.

In another aspect is provided a method of sensitizing cancer cells to killing by radiation, including contacting the cancer cells with an effective (e.g., therapeutically effective) amount of a compound described herein, including embodiments thereof, wherein the therapeutically effective amount is sufficient to sensitize the cancer cells to killing by radiation.

In another aspect is provided a method of slowing or preventing metastasis of cancer cells in a subject in need thereof, including administering to the subject an effective (e.g., therapeutically effective) amount of a compound described herein, including embodiments thereof, wherein the therapeutically effective amount is sufficient to slow or prevent the metastasis.

In another aspect is provided a method of treating a glioblastoma multiforme brain tumor in a subject in need thereof, including performing surgery on the subject to debulk the glioblastoma multiforme brain tumor; radiosensitizing remaining tumor cells by administering to the subject a therapeutically effective amount of at least one of the compounds of any of the invention, wherein the therapeutically effective amount is sufficient to sensitize the remaining tumor cells to killing by radiation; and providing radiation therapy to the subject.

In an aspect is provided a method of treating a glioblastoma multiforme brain tumor in a subject in need thereof. In embodiments, the method includes performing surgery on the subject to debulk the glioblastoma multiforme brain tumor. In embodiments, the method includes radiosensitizing the remaining tumor cells by administering to the subject a therapeutically effective amount of at least one of the compounds as described herein, including embodiments. In embodiments, the therapeutically effective amount is sufficient to sensitize the remaining tumor cells to killing by radiation. In embodiments, the method includes providing radiation therapy to the subject.

It is contemplated that inhibiting MDA-9 activity through binding of the MDA-9 PDZ1 domain by a PDZ1 domain binder is useful for the treatment of cancer (e.g., cancers having increased MDA-9 expression). Thus, in an aspect is provided a method of inhibiting MDA-9 protein activity, the method including contacting the MDA-9 protein with an effective amount of a PDZ1 domain binder, thereby inhibiting MDA-9 activity.

As mentioned above, a PDZ1 domain binder as referred to herein is a compound or pharmaceutical composition as described herein, including embodiments thereof, (e.g., small molecule, antibody, aptamer, ligand) that selectively binds to a PDZ1 domain of an MDA-9 protein. In embodiments, a PDZ1 domain of an MDA-9 protein includes the sequence of SEQ ID NO: 1. In embodiments, a PDZ1 domain of an MDA-9 protein is the sequence of SEQ ID NO: 1. In embodiments, the PDZ1 domain binder binds the PDZ1 domain, thereby inhibiting PDZ1 domain function. In embodiments, the PDZ1 domain binder binds a portion of the PDZ1 domain. In embodiments, the PDZ1 domain binder binds a portion of the PDZ1 domain and an amino acid interface region located between the PDZ1 domain and the PDZ2 domain. A portion of a PDZ1 domain and/or an amino acid interface region refers to less than all of the amino acids that make up the region. For example, in embodiments, PDZ1 domain binder binds to less than 100% of the amino acids included in the PDZ1 domain. In embodiments, PDZ1 domain binder binds to less than 90% of the amino acids included in the PDZ1 domain. In embodiments, PDZ1 domain binder binds to less than 80% of the amino acids included in the PDZ1 domain. In embodiments, PDZ1 domain binder binds to less than 70% of the amino acids included in the PDZ1 domain. In embodiments, PDZ1 domain binder binds to less than 60% of the amino acids included in the PDZ1 domain. In embodiments, PDZ1 domain binder binds to less than 50% of the amino acids included in the PDZ1 domain. In embodiments, PDZ1 domain binder binds to less than 40% of the amino acids included in the PDZ1 domain. In embodiments, PDZ1 domain binder binds to less than 30% of the amino acids included in the PDZ1 domain. In embodiments, PDZ1 domain binder binds to less than 20% of the amino acids included in the PDZ1 domain. In embodiments, PDZ1 domain binder binds to less than 15% of the amino acids included in the PDZ1 domain. In embodiments, PDZ1 domain binder binds to less than 10% of the amino acids included in the PDZ1 domain. In embodiments, PDZ1 domain binder binds to less than 9% of the amino acids included in the PDZ1 domain. In embodiments, PDZ1 domain binder binds to less than 8% of the amino acids included in the PDZ1 domain. In embodiments, PDZ1 domain binder binds to less than 7% of the amino acids included in the PDZ1 domain. In embodiments, PDZ1 domain binder binds to less than 6% of the amino acids included in the PDZ1 domain. In embodiments, PDZ1 domain binder binds to less than 5% of the amino acids included in the PDZ1 domain. In embodiments, PDZ1 domain binder binds to less than 4% of the amino acids included in the PDZ1 domain. In embodiments, PDZ1 domain binder binds to less than 3% of the amino acids included in the PDZ1 domain. In embodiments, PDZ1 domain binder binds to less than 2% of the amino acids included in the PDZ1 domain. In embodiments, PDZ1 domain binder binds to less than 1% of the amino acids included in the PDZ1 domain.

Similarly, in embodiments, PDZ1 domain binder binds to less than 100% of the amino acids included in the interface region. In embodiments, PDZ1 domain binder binds to less than 90% of the amino acids included in the interface region. In embodiments, PDZ1 domain binder binds to less than 80% of the amino acids included in the interface region. In embodiments, PDZ1 domain binder binds to less than 70% of the amino acids included in the interface region. In embodiments, PDZ1 domain binder binds to less than 60% of the amino acids included in the interface region. In embodiments, PDZ1 domain binder binds to less than 50% of the amino acids included in the interface region. In embodiments, PDZ1 domain binder binds to less than 40% of the amino acids included in the interface region. In embodiments, PDZ1 domain binder binds to less than 30% of the amino acids included in the interface region. In embodiments, PDZ1 domain binder binds to less than 20% of the amino acids included in the interface region. In embodiments, PDZ1 domain binder binds to less than 15% of the amino acids included in the interface region. In embodiments, PDZ1 domain binder binds to less than 10% of the amino acids included in the interface region. In embodiments, PDZ1 domain binder binds to less than 9% of the amino acids included in the interface region. In embodiments, PDZ1 domain binder binds to less than 8% of the amino acids included in the interface region. In embodiments, PDZ1 domain binder binds to less than 7% of the amino acids included in the interface region. In embodiments, PDZ1 domain binder binds to less than 6% of the amino acids included in the interface region. In embodiments, PDZ1 domain binder binds to less than 5% of the amino acids included in the interface region. In embodiments, PDZ1 domain binder binds to less than 4% of the amino acids included in the interface region. In embodiments, PDZ1 domain binder binds to less than 3% of the amino acids included in the interface region. In embodiments, PDZ1 domain binder binds to less than 2% of the amino acids included in the interface region. In embodiments, PDZ1 domain binder binds to less than 1% of the amino acids included in the interface region.

In embodiments, the PDZ1 domain binder occludes about 5% of the PDZ1 domain. In embodiments, the PDZ1 domain binder occludes about 10% of the PDZ1 domain. In embodiments, the PDZ1 domain binder occludes about 15% of the PDZ1 domain. In embodiments, the PDZ1 domain binder occludes about 20% of the PDZ1 domain. In embodiments, the PDZ1 domain binder occludes about 25% of the PDZ1 domain. In embodiments, the PDZ1 domain binder occludes about 30% of the PDZ1 domain. In embodiments, the PDZ1 domain binder occludes about 35% of the PDZ1 domain. In embodiments, the PDZ1 domain binder occludes about 40% of the PDZ1 domain. In embodiments, the PDZ1 domain binder occludes about 45% of the PDZ1 domain. In embodiments, the PDZ1 domain binder occludes about 50% of the PDZ1 domain. In embodiments, the PDZ1 domain binder occludes about 55% of the PDZ1 domain. In embodiments, the PDZ1 domain binder occludes about 60% of the PDZ1 domain. In embodiments, the PDZ1 domain binder occludes about 65% of the PDZ1 domain. In embodiments, the PDZ1 domain binder occludes about 70% of the PDZ1 domain. In embodiments, the PDZ1 domain binder occludes about 75% of the PDZ1 domain. In embodiments, the PDZ1 domain binder occludes about 80% of the PDZ1 domain. In embodiments, the PDZ1 domain binder occludes about 85% of the PDZ1 domain. In embodiments, the PDZ1 domain binder occludes about 90% of the PDZ1 domain. In embodiments, the PDZ1 domain binder occludes about 91% of the PDZ1 domain. In embodiments, the PDZ1 domain binder occludes about 92% of the PDZ1 domain. In embodiments, the PDZ1 domain binder occludes about 93% of the PDZ1 domain. In embodiments, the PDZ1 domain binder occludes about 94% of the PDZ1 domain. In embodiments, the PDZ1 domain binder occludes about 95% of the PDZ1 domain. In embodiments, the PDZ1 domain binder occludes about 96% of the PDZ1 domain. In embodiments, the PDZ1 domain binder occludes about 97% of the PDZ1 domain. In embodiments, the PDZ1 domain binder occludes about 98% of the PDZ1 domain. In embodiments, the PDZ1 domain binder occludes about 99% of the PDZ1 domain. In embodiments, the PDZ1 domain binder occludes 100% of the PDZ1 domain.

In embodiments, the PDZ1 domain binder occludes 5% of the PDZ1 domain. In embodiments, the PDZ1 domain binder occludes 10% of the PDZ1 domain. In embodiments, the PDZ1 domain binder occludes 15% of the PDZ1 domain. In embodiments, the PDZ1 domain binder occludes 20% of the PDZ1 domain. In embodiments, the PDZ1 domain binder occludes 25% of the PDZ1 domain. In embodiments, the PDZ1 domain binder occludes 30% of the PDZ1 domain. In embodiments, the PDZ1 domain binder occludes 35% of the PDZ1 domain. In embodiments, the PDZ1 domain binder occludes 40% of the PDZ1 domain. In embodiments, the PDZ1 domain binder occludes 45% of the PDZ1 domain. In embodiments, the PDZ1 domain binder occludes 50% of the PDZ1 domain. In embodiments, the PDZ1 domain binder occludes 55% of the PDZ1 domain. In embodiments, the PDZ1 domain binder occludes 60% of the PDZ1 domain. In embodiments, the PDZ1 domain binder occludes 65% of the PDZ1 domain. In embodiments, the PDZ1 domain binder occludes 70% of the PDZ1 domain. In embodiments, the PDZ1 domain binder occludes 75% of the PDZ1 domain. In embodiments, the PDZ1 domain binder occludes 80% of the PDZ1 domain. In embodiments, the PDZ1 domain binder occludes 85% of the PDZ1 domain. In embodiments, the PDZ1 domain binder occludes 90% of the PDZ1 domain. In embodiments, the PDZ1 domain binder occludes 91% of the PDZ1 domain. In embodiments, the PDZ1 domain binder occludes 92% of the PDZ1 domain. In embodiments, the PDZ1 domain binder occludes 93% of the PDZ1 domain. In embodiments, the PDZ1 domain binder occludes 94% of the PDZ1 domain. In embodiments, the PDZ1 domain binder occludes 95% of the PDZ1 domain. In embodiments, the PDZ1 domain binder occludes 96% of the PDZ1 domain. In embodiments, the PDZ1 domain binder occludes 97% of the PDZ1 domain. In embodiments, the PDZ1 domain binder occludes 98% of the PDZ1 domain. In embodiments, the PDZ1 domain binder occludes 99% of the PDZ1 domain. In embodiments, the PDZ1 domain binder occludes 100% of the PDZ1 domain.

In embodiments, the PDZ1 domain binder occludes at least 5% of the PDZ1 domain. In embodiments, the PDZ1 domain binder occludes at least 10% of the PDZ1 domain. In embodiments, the PDZ1 domain binder occludes at least 15% of the PDZ1 domain. In embodiments, the PDZ1 domain binder occludes at least 20% of the PDZ1 domain. In embodiments, the PDZ1 domain binder occludes at least 25% of the PDZ1 domain. In embodiments, the PDZ1 domain binder occludes at least 30% of the PDZ1 domain. In embodiments, the PDZ1 domain binder occludes at least 35% of the PDZ1 domain. In embodiments, the PDZ1 domain binder occludes at least 40% of the PDZ1 domain. In embodiments, the PDZ1 domain binder occludes at least 45% of the PDZ1 domain. In embodiments, the PDZ1 domain binder occludes at least 50% of the PDZ1 domain. In embodiments, the PDZ1 domain binder occludes at least 55% of the PDZ1 domain. In embodiments, the PDZ1 domain binder occludes at least 60% of the PDZ1 domain. In embodiments, the PDZ1 domain binder occludes at least 65% of the PDZ1 domain. In embodiments, the PDZ1 domain binder occludes at least 70% of the PDZ1 domain. In embodiments, the PDZ1 domain binder occludes at least 75% of the PDZ1 domain. In embodiments, the PDZ1 domain binder occludes at least 80% of the PDZ1 domain. In embodiments, the PDZ1 domain binder occludes at least 85% of the PDZ1 domain. In embodiments, the PDZ1 domain binder occludes at least 90% of the PDZ1 domain. In embodiments, the PDZ1 domain binder occludes at least 91% of the PDZ1 domain. In embodiments, the PDZ1 domain binder occludes at least 92% of the PDZ1 domain. In embodiments, the PDZ1 domain binder occludes at least 93% of the PDZ1 domain. In embodiments, the PDZ1 domain binder occludes at least 94% of the PDZ1 domain. In embodiments, the PDZ1 domain binder occludes at least 95% of the PDZ1 domain. In embodiments, the PDZ1 domain binder occludes at least 96% of the PDZ1 domain. In embodiments, the PDZ1 domain binder occludes at least 97% of the PDZ1 domain. In embodiments, the PDZ1 domain binder occludes at least 98% of the PDZ1 domain. In embodiments, the PDZ1 domain binder occludes at least 99% of the PDZ1 domain. In embodiments, the PDZ1 domain binder occludes at least 100% of the PDZ1 domain.

PDZ1 domain occlusion may be measured using methods known in the art. A Non-limiting example includes measuring the solvent accessible surface area in the presence and absence of the PDZ1 domain binder.

In embodiments, the PDZ1 domain binder is a small molecule (e.g., a compound described herein), an antibody, an aptamer, or a ligand. In embodiments, the PDZ1 domain binder is a small molecule (e.g., a compound as described herein). In embodiments, the PDZ1 domain binder is an antibody. In embodiments, the PDZ1 domain binder is an aptamer. In embodiments, the PDZ1 domain binder is a ligand.

In embodiments, the antibody may be linked (e.g., covalently, non-covalently) to a phosphorothioate nucleic acid, (e.g., wherein the phosphorothioate nucleic acid facilitates intracellular delivery of the antibody). In embodiments, the aptamer may be linked (e.g., covalently, non-covalently) to a phosphorothioate nucleic acid, (e.g., wherein the phosphorothioate nucleic acid facilitates intracellular delivery of the aptamer). In embodiments, the ligand may be linked (e.g., covalently, non-covalently) to a phosphorothioate nucleic acid, (e.g., wherein the phosphorothioate nucleic acid facilitates intracellular delivery of the ligand). Alternative, in embodiments, the antibody may be linked (e.g., covalently, non-covalently) to a ligand that binds a cell-surface receptor, (e.g., thereby promoting intracellular delivery of the antibody). In embodiments, the aptamer may be linked (e.g., covalently, non-covalently) to a ligand that binds a cell-surface receptor, (e.g., thereby promoting intracellular delivery of the aptamer). In embodiments, the ligand may be linked (e.g., covalently, non-covalently) to a ligand that binds a cell-surface receptor, (e.g., thereby promoting intracellular delivery of the ligand).

In embodiments, the small molecule is a compound as described herein, including embodiments thereof.

In embodiments, the PDZ1 domain binder (e.g., compound described herein) binds a PDZ1 domain with a Kd of less than 25 μM. In embodiments, the PDZ1 domain binder (e.g., compound described herein) binds a PDZ1 domain with a Kd of less than 24 μM. In embodiments, the PDZ1 domain binder (e.g., compound described herein) binds a PDZ1 domain with a Kd of less than 23 μM. In embodiments, the PDZ1 domain binder (e.g., compound described herein) binds a PDZ1 domain with a Kd of less than 22 μM. In embodiments, the PDZ1 domain binder (e.g., compound described herein) binds a PDZ1 domain with a Kd of less than 21 μM. In embodiments, the PDZ1 domain binder (e.g., compound described herein) binds a PDZ1 domain with a Kd of less than 20 μM. In embodiments, the PDZ1 domain binder (e.g., compound described herein) binds a PDZ1 domain with a Kd of less than 15 μM. In embodiments, the PDZ1 domain binder (e.g., compound described herein) binds a PDZ1 domain with a Kd of less than 10 μM. In embodiments, the PDZ1 domain binder (e.g., compound described herein) binds a PDZ1 domain with a Kd of less than 9 μM. In embodiments, the PDZ1 domain binder (e.g., compound described herein) binds a PDZ1 domain with a Kd of less than 8 μM. In embodiments, the PDZ1 domain binder (e.g., compound described herein) binds a PDZ1 domain with a Kd of less than 7 μM. In embodiments, the PDZ1 domain binder (e.g., compound described herein) binds a PDZ1 domain with a Kd of less than 6 μM. In embodiments, the PDZ1 domain binder (e.g., compound described herein) binds a PDZ1 domain with a Kd of less than 5 μM. In embodiments, the PDZ1 domain binder (e.g., compound described herein) binds a PDZ1 domain with a Kd of less than 4 μM. In embodiments, the PDZ1 domain binder (e.g., compound described herein) binds a PDZ1 domain with a Kd of less than 3 μM. In embodiments, the PDZ1 domain binder (e.g., compound described herein) binds a PDZ1 domain with a Kd of less than 2 μM. In embodiments, the PDZ1 domain binder (e.g., compound described herein) binds a PDZ1 domain with a Kd of less than 1 μM. In embodiments, the PDZ1 domain binder (e.g., compound described herein) binds a PDZ1 domain with a Kd of less than 500 nM. In embodiments, the PDZ1 domain binder (e.g., compound described herein) binds a PDZ1 domain with a Kd of less than 100 nM. In embodiments, the PDZ1 domain binder (e.g., compound described herein) binds a PDZ1 domain with a Kd of less than 50 nM. In embodiments, the PDZ1 domain binder (e.g., compound described herein) binds a PDZ1 domain with a Kd of less than 20 nM. In embodiments, the PDZ1 domain binder (e.g., compound described herein) binds a PDZ1 domain with a Kd of less than 10 nM. In embodiments, the PDZ1 domain binder (e.g., compound described herein) binds a PDZ1 domain with a Kd of less than 5 nM. In embodiments, the PDZ1 domain binder (e.g., compound described herein) binds a PDZ1 domain with a Kd of less than 1 nM. In embodiments, the PDZ1 domain binder (e.g., compound described herein) binds a PDZ1 domain with a Kd of less than 0.5 nM. In embodiments, the PDZ1 domain binder (e.g., compound described herein) binds a PDZ1 domain with a Kd of less than 0.1 nM. In embodiments, the PDZ1 domain binder (e.g., compound described herein) binds a PDZ1 domain with a Kd of less than 0.05 nM. In embodiments, the PDZ1 domain binder (e.g., compound described herein) binds a PDZ1 domain with a Kd of less than 0.025 nM.

In embodiments, the ligand is a natural ligand of a PDZ1 domain. In embodiments, the ligand is a natural ligand of a PDZ1 domain or a portion thereof. The term “natural ligand” refers to a naturally occurring ligand that binds a PDZ1 domain. Non-limiting examples of natural ligands include Merlin, neurexin, and interleukin-5 receptor alpha.

In an aspect is provided a method of treating cancer in a subject in need thereof, the method including administering to the subject an effective amount of a PDZ1 domain binder.

In embodiments, the PDZ1 domain binder is a compound described herein, including in embodiments, an aspect, an example, a figure, a claim, a scheme, or a table.

In embodiments, the method further includes administering to the subject an anti-cancer agent. In embodiments, the anti-cancer agent is radiation, 5-FU, sorafenib, taxol, temozolimide, or Mcl-1. In embodiments, the anti-cancer agent is radiation. In embodiments, the anti-cancer agent is 5-FU. In embodiments, the anti-cancer agent is sorafenib. In embodiments, the anti-cancer agent is taxol. In embodiments, the anti-cancer agent is temozolimide. In embodiments, the anti-cancer agent is Mcl-1.

In embodiments, the combined effective amount is a combined synergistic amount.

In embodiments, the agent that inhibits MDA-9 is a compound that binds to MDA-9 protein.

In embodiments, the agent that inhibits MDA-9 is a PDZ1 domain binder.

In embodiments, the agent that inhibits MDA-9 is a compound having the formula:

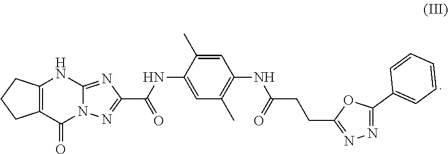 [SMILES: Cc1cc(NC(=O)c2nc3[nH]c4c(c(=O)n3n2)CCC4)c(C)cc1NC(=O)CCc1nnc(-c2ccccc2)o1]

In embodiments, the agent that inhibits MDA-9 is an agent that decreases expression of MDA-9.

In embodiments, the agent that inhibits MDA-9 is a small interfering RNA (siRNA) or a short hairpin RNA (shRNA) directed against MDA-9.

In embodiments, the anti-cancer agent is an anti-microtubule agent.

In embodiments, the anti-microtubule agent is a taxane or a vinca alkaloid. In embodiments, the anti-microtubule agent is a taxane. In embodiments, the anti-microtubule agent is a vinca alkaloid.

In embodiments, the anti-microtubule agent is a taxane selected from paclitaxel, docetaxel, cabazitaxel, and larotaxel. In embodiments, the anti-microtubule agent is paclitaxel. In embodiments, the anti-microtubule agent is cabazitaxel. In embodiments, the anti-microtubule agent is larotaxel.

In embodiments, the anti-microtubule agent is a vinca alkaloid selected from vinblastine, vincristine, or vinorelbine. In embodiments, the anti-cancer agent is vinblastine. In embodiments, the anti-cancer agent is vincristine. In embodiments, the anti-cancer agent is vinorelbine.

In embodiments, the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide. In embodiments, the anti-cancer agent is trichostatin A (TSA). In embodiments, the anti-cancer agent is oxamflatin. In embodiments, the anti-cancer agent is apicidin. In embodiments, the anti-cancer agent is depsipeptide. In embodiments, the anti-cancer agent is depudecin. In embodiments, the anti-cancer agent is trapoxin. In embodiments, the anti-cancer agent is vorinostat. In embodiments, the anti-cancer agent is suberoylanilide hydroxamic acid. In embodiments, the anti-cancer agent is pyroxamide.

In embodiments, the agent that inhibits MDA-9 and the anti-cancer agent are co-administered.

In embodiments, the agent that inhibits MDA-9 and the anti-cancer agent are effective to increase apoptotic cell death in cancer stem cells (CSCs), as compared to either agent alone.

In embodiments, the cancer is selected from the group consisting of prostate cancer, breast cancer, gastric cancer, lung cancer, brain cancer, colorectal cancer, pancreatic cancer, melanoma, and neuroblastoma. In embodiments, the cancer is prostate cancer. In embodiments, the cancer is breast cancer. In embodiments, the cancer is gastric cancer. In embodiments, the cancer is lung cancer. In embodiments, the cancer is brain cancer. In embodiments, the cancer is colorectal cancer. In embodiments, the cancer is pancreatic cancer. In embodiments, the cancer is melanoma. In embodiments, the cancer is neuroblastoma

In embodiments, the subject does not receive androgen ablation therapy.

In embodiments, the cancer is chemoresistant. In embodiments, the chemoresistance is MDR1-mediated.

In embodiments, the cancer is a recurrence of cancer.

In embodiments, the method further includes administering to the subject one or more additional therapies. In embodiments, the additional therapy is radiation. In embodiments, the additional therapy may be administered before the MDA-9 inhibitor (e.g., compound described herein). In embodiments, the second therapy may be administered after the MDA-9 inhibitor (e.g., compound described herein). In embodiments, the second therapy may be administered concurrently with the MDA-9 inhibitor (e.g., compound described herein). In embodiments, the additional therapy is radiation. In embodiments, the second therapy is administration of a additional therapeutic agent. In embodiments, the additional therapeutic agent is an anti-cancer agent. In embodiments, the additional therapeutic agent is a chemotherapeutic agent. In embodiments, the additional therapeutic agent is an agent for treating glioblastoma. In embodiments, the additional therapeutic agent is an agent for treating breast cancer. In embodiments, the additional therapeutic agent is an agent for treating urothelial cancer. In embodiments, the additional therapeutic agent is an agent for treating melanoma. In embodiments, the additional therapeutic agent is an agent for treating hepatocellular carcinoma. In embodiments, the additional therapeutic agent is an agent for treating colorectal cancer. In embodiments, the additional therapeutic agent is an agent for treating neuroblastoma. In embodiments, the additional therapeutic agent is an agent for treating colon cancer. In embodiments, the additional therapeutic agent is an agent for treating gastric cancer. In embodiments, the additional therapeutic agent is an agent for treating bladder cancer. In embodiments, the additional therapeutic agent is an agent for treating lung cancer. In embodiments, the additional therapeutic agent is an agent for treating pancreatic cancer. In embodiments, the additional therapeutic agent is an agent for treating head and neck cancer.

It is further contemplated that the compounds and pharmaceutical compositions provided herein, including embodiments thereof, are useful for preventing or reducing metastasis of cancer cells. The pharmaceutical compositions provided herein, including embodiments thereof, can accomplish prevention or reduction of metastasis of cancer cells by inhibiting cancer cell invasion and attachment of cancer cells and cancer-associated angiogenesis. Cancer cell invasion refers to the ability of cancer cells to become motile and infiltrate neighboring tissue or blood vessels. Cancer cell attachment refers the ability of cancer cells to adhere to blood vessel walls and extravasate, or to adhere to neighboring tissue, thereby forming metastases.

Therefore, in an aspect is provided a method of preventing metastasis of cancer cells in a subject in need thereof, the method including administering to the subject an effective amount of an MDA-9 inhibitor and an anti-cancer agent (e.g., mitotic inhibitor or an HDAC inhibitor), as disclosed herein.

In an aspect is provided a method of reducing metastasis of cancer cells in a subject in need thereof, the method including administering to the subject an effective amount of an MDA-9 inhibitor and an anti-cancer agent (e.g., mitotic inhibitor or an HDAC inhibitor), as disclosed herein. In embodiments, the method of reducing is measured relative to a control (e.g., the absence the MDA-9 inhibitor, the anti-cancer agent, or both).

In an aspect is provided a method of inhibiting cancer associated angiogenesis in a subject in need thereof, the method including administering to the subject an effective amount of an MDA-9 inhibitor and an anti-cancer agent (e.g., mitotic inhibitor or an HDAC inhibitor), as disclosed herein. In embodiments, the method of inhibiting is measured relative to a control (e.g., the absence the MDA-9 inhibitor, the anti-cancer agent, or both).

In embodiments, the cancer is associated with an increased MDA-9 gene expression. In embodiments, the cancer cells are associated with an increased MDA-9 gene expression. Detection of increased MDA-9 gene expression may be determined, for example, by comparing MDA-9 gene expression from a biological sample (e.g., tumor biopsy, blood) obtained from a patient against a control sample. The control sample may be a biological sample (e.g., healthy tissue) taken from the same patient. Alternatively, the control sample may be a biological sample (e.g., tissue, blood) obtained from a cancer-free subject. In embodiments, an increased MDA-9 gene expression level is at least 1.02, 1.03, 1.04, 1.05, 1.1, 1.2, 1.3, 1.4, 1.5, 1.6, 1.7, 1.8, 1.9, 2, 2.5, 3, 3.5, 4, 4.5, 5, 5.5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, or 300 times greater than the expression level observed in the control sample.

Detection of increased MDA-9 gene expression may also be determined by comparing the level of MDA-9 gene expression in a biological sample (e.g., tumor biopsy, blood) obtained from a patient against MDA-9 expression levels from biological samples obtained from a population of patients. Population data may be obtained from public genome-wide expression databases. In embodiments, an increased MDA-9 gene expression level is equal to or greater than the mean of the MDA-9 expression level of the patient population. In embodiments, an increased MDA-9 gene expression level is greater than the median of the MDA-9 expression level of the patient population. In embodiments, an increased expression level of MDA-9 may be a relative expression level of at least 1.02, 1.03, 1.04, 1.05, 1.1, 1.2, 1.3, 1.4, 1.5, 1.6, 1.7, 1.8, 1.9, 2, 2.5, 3, 3.5, 4, 4.5, 5, 5.5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, or 300. The relative expression level value (z) can be quantified by the following equation:

z=(Iₙ−Average Iₙₒᵣₘ)/standard deviationₙₒᵣₘ,

wherein n refers to every sample in the dataset (including tumors) and norm refers to normal samples only.

In embodiments, the cancer is prostate cancer, breast cancer, gastric cancer, lung cancer, brain cancer, colorectal cancer, pancreatic cancer, melanoma, or neuroblastoma. In embodiments, the cancer is prostate cancer. In embodiments, the cancer is breast cancer. In embodiments, the cancer is gastric cancer. In embodiments, the cancer is lung cancer. In embodiments, the cancer is brain cancer. In embodiments, the cancer is colorectal cancer. In embodiments, the cancer is pancreatic cancer. In embodiments, the cancer is melanoma. In embodiments, the cancer is neuroblastoma.

In embodiments, the compound (e.g., compound described herein) or method includes reducing the level of activity of NF-kB. In embodiments, the compound (e.g., compound described herein) or method includes reducing the level of activity of c-Src. In embodiments, the compound (e.g., compound described herein) or method includes reducing the level of activity of p38 MAPK. In embodiments, the compound (e.g., compound described herein) or method includes reducing the level of activity of a c-Src/p38 MAPK signaling pathway. In embodiments, the compound (e.g., compound described herein) or method includes reducing the level of activity of MMP2. In embodiments, the compound (e.g., compound described herein) or method includes reducing the level of activity of FAK. In embodiments, the compound (e.g., compound described herein) or method includes reducing the level of activity of EphA2. In embodiments, the compound (e.g., compound described herein) or method includes reducing the level of activity of EGFR. In embodiments, the compound (e.g., compound described herein) or method includes reducing the level of activity of EGFRvIII. In embodiments, the compound (e.g., compound described herein) or method includes reducing the level of activity of MMP9. In embodiments, the compound (e.g., compound described herein) or method includes reducing the level of activity of IGF-1R. In embodiments, the compound (e.g., compound described herein) or method includes reducing the level of activity of STAT3. In embodiments, the compound (e.g., compound described herein) or method includes reducing the level of activity of IGFBP-2. In embodiments, the compound (e.g., compound described herein) or method includes reducing the level of activity of RhoA. In embodiments, the compound (e.g., compound described herein) or method includes reducing the level of activity of Cdc42. In embodiments, the compound (e.g., compound described herein) or method includes reducing the level of activity of TGFβ1. In embodiments, the compound (e.g., compound described herein) or method includes reducing the level of activity of Slug. In embodiments, the compound (e.g., compound described herein) or method includes reducing the level of activity of Snail. In embodiments, the compound (e.g., compound described herein) or method includes reducing the level of activity of Zeb1. In embodiments, the compound (e.g., compound described herein) or method includes reducing the level of activity of N-cadherin. In embodiments, the compound (e.g., compound described herein) or method includes reducing the level of activity of IL-8. In embodiments, the compound (e.g., compound described herein) or method includes reducing the level of activity of cyclin D1. In embodiments, the compound (e.g., compound described herein) or method includes reducing the level of activity of CDK4. In embodiments, the compound (e.g., compound described herein) or method includes reducing the level of activity of PI3K. In embodiments, the compound (e.g., compound described herein) or method includes reducing the level of activity of CTNNB1. In embodiments, the compound (e.g., compound described herein) or method includes reducing the level of activity of integrin 01. In embodiments, the compound (e.g., compound described herein) or method includes reducing the level of activity of Akt. In embodiments, the compound (e.g., compound described herein) or method includes reducing the level of activity of HIF-1α. In embodiments, the compound (e.g., compound described herein) or method includes reducing the level of activity of VEGF-A. In embodiments, the compound (e.g., compound described herein) or method includes reducing the level of activity of AEG-1.

### V. Formulations

The compounds described herein are generally delivered (administered) as a pharmaceutical composition. Such pharmaceutical compositions generally include at least one of the disclosed compounds (e.g., in pure form, salt, or prodrugs), and more than one (a plurality) of different compounds (e.g. 2 or more such as 2, 3, 4, 5, 6, 7, 8, 9, 10 or more) may be included in a single formulation. Accordingly, the present invention encompasses such formulations and compositions. The pharmaceutical compositions generally include one or more substantially purified compounds as described herein, and a pharmacologically suitable (physiologically compatible) carrier, which may be aqueous or oil-based. In some embodiments, such pharmaceutical compositions are prepared as liquid solutions or suspensions. In other embodiments, the pharmaceutical compositions are prepared in solid forms such as tablets, pills, powders and the like. Solid forms suitable for solution, dissolution or suspension in liquids prior to administration are also contemplated (e.g. lyophilized forms of the compounds), as are emulsified preparations. In some embodiments, the liquid formulations are aqueous or oil-based suspensions or solutions. In some embodiments, the active ingredients are mixed with excipients which are pharmaceutically acceptable and compatible with the active ingredients, e.g. pharmaceutically acceptable salts. Suitable excipients include, for example, water, saline, dextrose, glycerol, cyclodextrin, ethanol and the like, or combinations thereof. In addition, the pharmaceutical composition may contain minor amounts of auxiliary substances such as wetting or emulsifying agents, pH buffering agents, preservatives, and the like. If it is desired to administer an oral form of the pharmaceutical composition, various thickeners, flavorings, diluents, emulsifiers, dispersing aids or binders and the like are added. The pharmaceutical composition of the present invention may contain any such additional ingredients so as to provide the pharmaceutical composition in a form suitable for administration. The final amount of compound in the formulations varies, but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which serve as pharmaceutically acceptable carriers include, but are not limited to, ion exchangers, alumina, aluminum stearate, lecithin, serum proteins (such as human serum albumin), buffer substances (such as Tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the pharmaceutical composition, according to the judgment of the formulator.

“Pharmaceutically acceptable salts” refers to the relatively non-toxic, inorganic and organic acid addition salts, and base addition salts, of compounds (e.g., compound described herein). These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

### VI. Administration

In embodiments, the formulation is administered in vivo by any suitable route including but not limited to: inoculation or injection (e.g. intravenous, intraperitoneal, intramuscular, subcutaneous, intra-aural, intraarticular, intramammary, intracranial, and the like), topical application (e.g. on any suitable skin or membrane surface), by absorption through epithelial or mucocutaneous linings (e.g., nasal, oral, vaginal, rectal, gastrointestinal mucosa, etc.) and the like. In embodiments, the compounds may be incorporated into implantable delivery means, e.g. drug permeated wafers, etc. Timed (sustained, extended, controlled) release formulations, e.g. in the form of pills, tablets, capsules, etc. are also contemplated, including various diffusion systems such as reservoir and matrix devices; osmotic, ion exchange, floating, bio-adhesive, and depot systems, etc. In embodiments, the mode of administration is intranasal, orally or parenteral, by intravenous, intraperitoneal, intramuscular, topical or subcutaneous routes, and usually is by intravenous injection.

In addition, in embodiments, the compounds may be administered in conjunction with other treatment modalities. Examples of such additional treatments include but are not limited to: administration of various anti-cancer agents, for example, cytotoxic chemotherapy agents; radiation; hormone therapy; T-cell therapy; various immune checkpoint inhibitors; various forms of targeted therapy (e.g. small molecules such as tyrosine-kinase inhibitors, folate receptor inhibitors, serine/threonine kinase inhibitors monoclonal antibodies, etc.).

In some aspects, the compounds as described herein, including embodiments, administered with or in conjunction with one or more anti-invasive and/or anti-attachment therapies. In some aspects, the compounds of the invention are administered with or in conjunction with one or more anti-cancer agents. Such combinations result in induction of apoptosis and/or toxic autophagy and death of cancer cells. For example, in embodiments, the present drugs can be combined with PDZ inhibitors, with radiation (e.g. for GBM) with sorafenib (for liver cancer), with taxol (for breast cancer), etc.

Administration of other therapies that are not specific for cancer, together with the compounds disclosed herein, is also contemplated, e.g. appetite stimulants, nutritional supplements; pain medication, anti-depressants, etc.

Administering a compound, antibody, aptamer, or ligand, as described herein, including embodiments, “with” or “in conjunction with” another agent may refer to administering a single pharmaceutical composition that includes both agents, but more typically refers to administering one or more compounds of the invention to the same subject during a course of treatment with the other agent, e.g. more or less consecutively, or on the same day but several hours apart, or several days (or even weeks) apart. In embodiments, the compound, antibody, aptamer, or ligand, as described herein, including embodiments, and the additional treatment (e.g., anti-cancer agent) are administered consecutively. In embodiments, compound, antibody, aptamer or ligand, as described herein, including embodiments, additional treatment (e.g., anti-cancer agent) are administered simultaneously. In embodiments, compound, antibody, aptamer or ligand, as described herein, including embodiments, are admixed with additional treatment (e.g., anti-cancer agent) prior to administration.

In embodiments, administration is carried out in a coordinated manner at time intervals which are spaced apart by minutes, hours, days or weeks, etc. and the use of the multiple agents is thus integrated into a treatment protocol in which the effects are at least additive (i.e., a combined additive amount), and may be synergistic (i.e., a combined synergistic amount).

A “combined additive amount” as used herein refers to the sum of a first amount of a first agent (e.g., compound as described herein) and a second amount of a second agent (e.g., an anti-cancer agent), that results in an additive effect (i.e. an effect equal to the sum of the effects). Therefore, the terms “additive”, “combined additive amount”, and “additive therapeutic effect” which are used herein interchangeably, refer to a measured effect of compounds administered in combination where the measured effect is equal to the sum of the individual effects of each of the compounds administered alone as a single agent.

A “combined synergistic amount” as used herein refers to the sum of a first amount of first agent (e.g., compound as described herein) and a second amount of a second agent (e.g., an anti-cancer agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a combined synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the first amount (e.g., MDA-9 inhibitor) when used separately from the second amount (e.g., anti-cancer agent). In embodiments, a combined synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the second amount (e.g., anti-cancer agent) when used separately from the first amount (e.g., PDZ1 domain binder).

Of particular interest is administration of the present agents together with radiation therapy, which are shown herein, results in a synergistic effect in that more cancer cells are killed by the combination than would be predicted if the effect was purely additive, when based on results obtained by administering each agent alone.

In some embodiments, the compounds of the invention are used to block and/or prevent metastatic seeding of cells in the circulatory system. Such methods are carried out by pretreating the patient with a compound (e.g., compound described herein), i.e. treating the patient before another cancer treatment is begun, and then performing at least one other cancer treatment. The second (“other”) cancer treatment may be, for example, surgical removal of the tumor, and may be performed in the presence of any of the compounds described herein. Thereafter, the method may include continuing to treat the patient for a suitable period of time after removing the tumor.

In yet other embodiments, the compounds of the invention are used to block and/or prevent metastatic seeding. In such methods, a patient with early stage cancer is treated with a compound (e.g., compound described herein), thereby preventing invasion and shedding of tumor cells into the bloodstream and preventing secondary seeding (colonization) of tumor cells in a distant site (metastasis).

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EMBODIMENTS

The present disclosure provides the following illustrative embodiments.

Embodiment 1. A method of treating cancer in a subject in need thereof, comprising administering to the subject a combined effective amount of (a) an agent that inhibits MDA-9, and (b) an anti-cancer agent, wherein the anti-cancer agent is a mitotic inhibitor or a histone deacetylase (HDAC) inhibitor.

Embodiment 2. The method of Embodiment 1, wherein the combined effective amount is a combined synergistic amount.

Embodiment 3. The method of Embodiment 1, wherein the agent that inhibits MDA-9 is a compound that binds to MDA-9 protein.

Embodiment 4. The method of Embodiment 3, wherein the agent that inhibits MDA-9 is a PDZ1 domain binder.

Embodiment 5. The method of Embodiment 4, wherein the agent that inhibits MDA-9 is a compound having the formula:

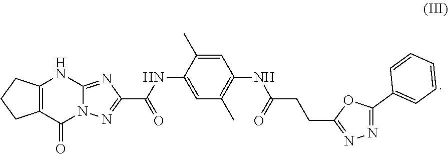 [SMILES: Cc1cc(NC(=O)c2nc3[nH]c4c(c(=O)n3n2)CCC4)c(C)cc1NC(=O)CCc1nnc(-c2ccccc2)o1]

Embodiment 6. The method of Embodiment 1, wherein the agent that inhibits MDA-9 is an agent that decreases expression of MDA-9.

Embodiment 7. The method of Embodiment 6, wherein the agent that inhibits MDA-9 is a small interfering RNA (siRNA) or a short hairpin RNA (shRNA) directed against MDA-9.

Embodiment 8. The method of any one of Embodiments 1-7, wherein the anti-cancer agent is an anti-microtubule agent.

Embodiment 9. The method of Embodiment 8, wherein the anti-microtubule agent is a taxane or a vinca alkaloid.

Embodiment 10. The method of Embodiment 9, wherein the anti-microtubule agent is a taxane selected from paclitaxel, docetaxel, cabazitaxel, and larotaxel.

Embodiment 11. The method of Embodiment 9, wherein the anti-microtubule agent is a vinca alkaloid selected from vinblastine, vincristine, or vinorelbine.

Embodiment 12. The method of Embodiment any one of Embodiments 1-7, wherein the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide.

Embodiment 13. The method of Embodiment 12, wherein the HDAC inhibitor is TSA.

Embodiment 14. The method of any one of Embodiments 1-13, wherein the agent that inhibits MDA-9 and the anti-cancer agent are co-administered.

Embodiment 15. The method of any one of Embodiments 1-14, wherein the agent that inhibits MDA-9 and the anti-cancer agent are effective to increase apoptotic cell death in cancer stem cells (CSCs), as compared to either agent alone.

Embodiment 16. The method of any one of Embodiments 1-15, wherein the cancer is selected from the group consisting of prostate cancer, breast cancer, gastric cancer, lung cancer, brain cancer, colorectal cancer, pancreatic cancer, melanoma, and neuroblastoma.

Embodiment 17. The method of Embodiment 16, wherein the cancer is prostate cancer.

Embodiment 18. The method of Embodiment 16 or 17, wherein the subject does not receive androgen ablation therapy.

Embodiment 19. The method of any one of Embodiments 1-18, wherein the cancer is chemoresistant.

Embodiment 20. The method of Embodiment 19, wherein the chemoresistance is MDR1-mediated.

Embodiment 21. The method of any one of Embodiments 1-20, wherein the cancer is a recurrence of cancer.

Embodiment 22. A pharmaceutical composition comprising (a) an agent that inhibits MDA-9, and (b) an anti-cancer agent, wherein the anti-cancer agent is a mitotic inhibitor or a histone deacetylase (HDAC) inhibitor.

Embodiment 23. The pharmaceutical composition of Embodiment 22, wherein the agent that inhibits MDA-9 is a compound that binds to MDA-9 protein.

Embodiment 24. The pharmaceutical composition of Embodiment 23, wherein the agent that inhibits MDA-9 is a PDZ1 domain binder.

Embodiment 25. The pharmaceutical composition of Embodiment 24, wherein the agent that inhibits MDA-9 is a compound having the formula:

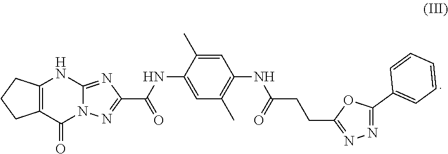 [SMILES: Cc1cc(NC(=O)c2nc3[nH]c4c(c(=O)n3n2)CCC4)c(C)cc1NC(=O)CCc1nnc(-c2ccccc2)o1]

Embodiment 26. The pharmaceutical composition of Embodiment 22, wherein the agent that inhibits MDA-9 is an agent that decreases expression of MDA-9.

Embodiment 27. The pharmaceutical composition of Embodiment 26, wherein the agent that inhibits MDA-9 is a small interfering RNA (siRNA) or a short hairpin RNA (shRNA) directed against MDA-9.

Embodiment 28. The pharmaceutical composition of any of Embodiments 22-27, wherein the anti-cancer agent is an anti-microtubule agent.

Embodiment 29. The pharmaceutical composition of Embodiment 28, wherein the anti-microtubule agent is a taxane or a vinca alkaloid.

Embodiment 30. The pharmaceutical composition of Embodiment 29, wherein the anti-microtubule agent is a taxane selected from paclitaxel, docetaxel, cabazitaxel, and larotaxel.

Embodiment 31. The pharmaceutical composition of Embodiment 29, wherein the anti-microtubule agent is a vinca alkaloid selected from vinblastine, vincristine, or vinorelbine.

Embodiment 32. The pharmaceutical composition of any one of Embodiments 22-27, wherein the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide.

Embodiment 33. The pharmaceutical composition of Embodiment 32, wherein the HDAC inhibitor is TSA.

Embodiment 34. The pharmaceutical composition of any one of Embodiments 22 to 33, further comprising a pharmaceutically acceptable carrier.

Embodiment 35. The pharmaceutical composition of any one of Embodiments 22 to 34, wherein the agent that inhibits MDA-9 and the anti-cancer agent are in a combined effective amount.

Embodiment 36. The pharmaceutical composition of Embodiment 35, wherein the combined effective amount is a combined synergistic amount.

Embodiment 37. Use of a pharmaceutical composition of any one of Embodiments 22 to 36 for the treatment of cancer.

Embodiment 38. The use of Embodiment 37, wherein the cancer is selected from the group consisting of prostate cancer, breast cancer, gastric cancer, lung cancer, brain cancer, colorectal cancer, pancreatic cancer, melanoma, and neuroblastoma.

Embodiment 39. The use of Embodiment 38, wherein the cancer is prostate cancer.

Embodiment 40. The use of any one of Embodiments 37-39, wherein the subject does not receive androgen ablation therapy.

Embodiment 41. The use of any one of Embodiments 37-40, wherein the cancer is chemoresistant.

Embodiment 42. The use of Embodiment 41, wherein the chemoresistance is MDR1-mediated.

Embodiment 43. The use of any one of Embodiments 37-42, wherein the cancer is a recurrence of cancer.

Embodiment 44. A kit comprising a pharmaceutical composition of any one of Embodiments 22 to 36.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### EXAMPLES

### Example 1. MDA-9 Expression is Elevated in the Unique Self-Renewing PCSC Subpopulation in Prostate Cancer

PCSCs from different prostate cancer cell lines (DU-145, ARCaP-M and PC3-ML) were sorted into putative CD44⁺ CD133⁺ PCSCs and CD44⁻ CD133⁻ non-stem cancer cells (NSCCs). mda-9 expression was analyzed in these putative stem and non-stem cancer cells by quantitative RT-PCR, and data were normalized to 18S and β-tubulin expression. We consistently observed elevated expression of mda-9 in all PCSC populations vs. NSCCs (Table 2). These PCSCs also expressed high levels of traditional stem-regulatory and self-renewal associated genes such as Nanog, Oct4, Sox2 and c-myc (Table 2).

TABLE 2
Expression of mda-9 and stemness genes in non-stem prostate
cancer cells and prostate cancer stem cells.

| | CELL LINE | | | | | |
|---|---|---|---|---|---|---|
| | DU-145 | | ARCaP-M | | PC3-ML | |
| | Non-stem | Cancer | Non-stem | Cancer | Non-stem | Cancer |
| GENES | cancer cell | stem cell | cancer cell | stem cell | cancer cell | stem cell |
| mda-9 | 1 ± 0.05 | 3.48 ± 0.10 | 1 ± 0.20 | 8.30 ± 0.07 | 1 ± 0.07 | 5.67 ± 0.25 |
| Stemness genes | | | | | | |
| Nanog | 1 ± 0.03 | 12.50 ± 0.02 | 1 ± 0.02 | 8.64 ± 0.11 | 1 ± 0.14 | 13.88 ± 2.0 |
| Sox2 | 1 ± 0.20 | 2.94 ± 0.04 | 1 ± 0.32 | 2.49 ± 0.06 | 1 ± 0.19 | 4.48 ± 1.93 |
| Oct4 | 1 ± 0.07 | 20.00 ± 0.01 | 1 ± 0.11 | 5.51 ± 0.01 | 1 ± 0.22 | 13.01 ± 0.45 |
| c-myc | 1 ± 0.15 | 2.44 ± 0.06 | 1 ± 0.09 | 2.39 ± 0.05 | 1 ± 0.03 | 3.93 ± 1.7 |

### Example 2 Mda-9 is Co-Expressed in PCSCs with Stem Cell Markers and Stemness-Regulating Genes

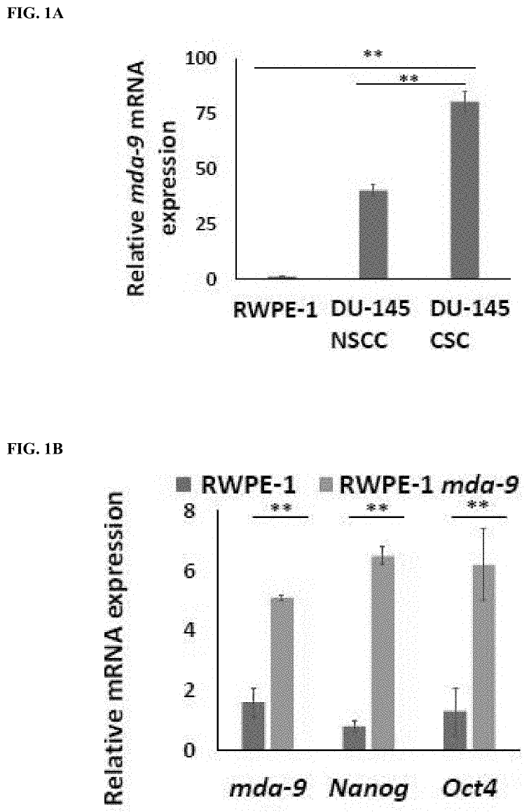

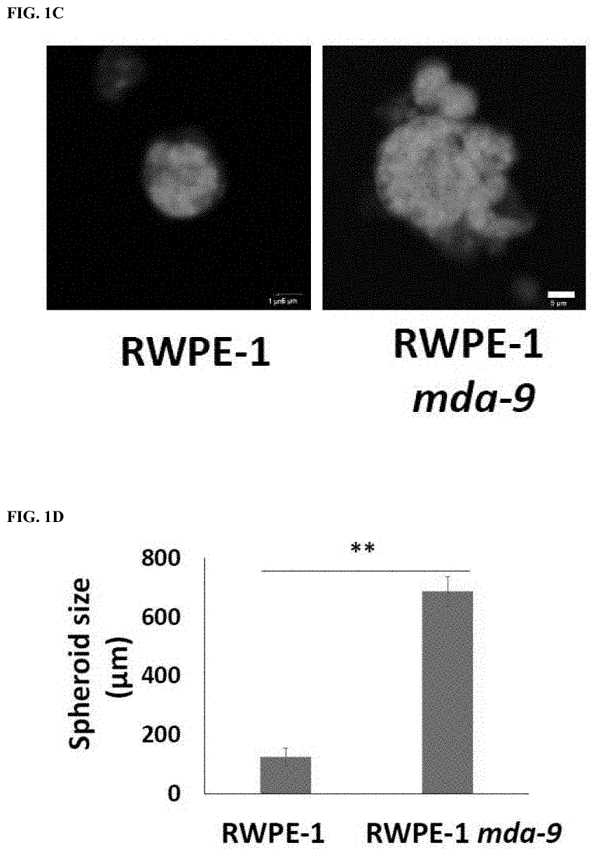

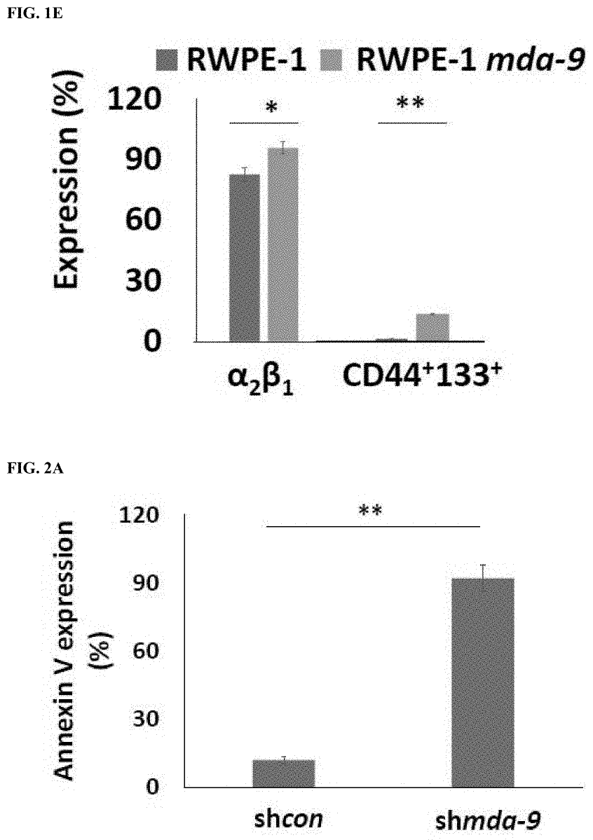

Normalized relative gene expression from PCSCs isolated from different cell lines was analyzed statistically by Pearson's correlation coefficient and ANOVA analysis for correlation and significance, respectively. A positive correlation was observed between mda-9 expression and sternness genes, including mda-9: Nanog, (Pearson's correlation coefficient R=0.7303), mda-9:Sox2 (R=0.6881), mda-9:Oct4 (R=0.4241), mda-9:c-myc (R=0.7279). The results were statistically significant (R²=0.7825, p<0.05) and the strongest correlation was observed between mda-9, Nanog and c-myc. mda-9 expression in DU-145 cancer cells was also several-fold higher than in normal prostate stem cells, with the highest expression being observed in cancer stem cells (FIG. 1A).

### Example 3. MDA-9 Over-Expression Leads to Expression of a Stem-Like Phenotype

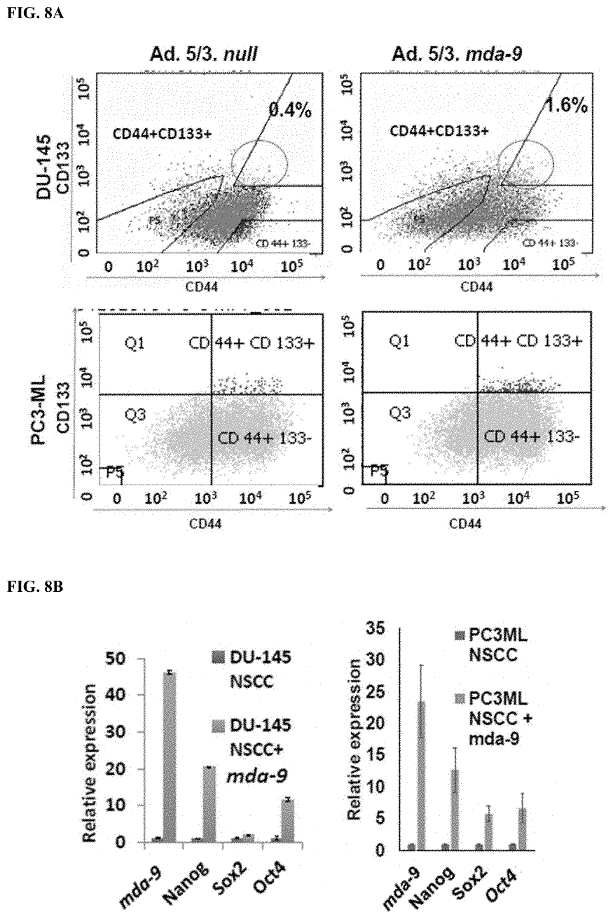

Forced expression of mda-9 in normal prostate non-stem cells lead to increased expression of self-renewal genes such as Nanog and Oct4 (˜6 fold) compared to that of parental cells (FIG. 1B). When the stem populations (stained with green fluorescent cell tracker) in the prostaspheres were studied, a significant increase in spheroid size, and number was observed (FIGS. 1C and 1D). mda-9 overexpression also increased stem populations, as demonstrated by a cell-surface marker-based flow cytometry analysis (FIGS. 1E and 8A). Overexpression of mda-9 in the non-stem cancer cells of DU-145 and PC3-ML also led to an approximately 2-4-fold increase in PCSCs as well as self-renewal associated genes (Nanog ˜13-22-fold, Sox2-2-6-fold, Oct4 ˜6.8-15-fold) (FIG. 8B). These results indicated that MDA-9 may have had a central role in the regulation of self-renewal in both normal and malignant prostate cells.

### Example 4. MDA-9 Activates Downstream Signaling, which Regulates Self-Renewal in PCSCs

To further ascertain the role of MDA-9 in regulating PCSC self-renewal and maintenance, we silenced mda-9 in PCSCs from DU-145, ARCaP-M and PC3-ML. Knocking down mda-9 in PCSCs significantly decreased the population of PCSCs (Table 3) as well as expression of self-renewal associated molecules at both RNA (Table 4) and protein levels (Table 5). Nanog was decreased by almost two-fold, ˜10-fold and ˜6.7-fold, Sox2 by ˜three-fold, ˜four-fold and ˜10-fold, Oct4 by ˜seven-fold, ˜10-fold and ˜33-fold, and c-myc by ˜six-fold, ˜20-fold and ˜11-fold, in DU-145, ARCaP-M and PC3-ML PCSCs, respectively, post mda-9 knock down (kd). These results suggested that mda-9 expression is important in maintaining expression of self-renewal associated genes in PCSCs.

TABLE 3
Effect of mda-9 expression on CSC populations
in prostate cancer cells.

| Cell line | CSC population (%) shcon | CSC population (%) shmda-9 |
|---|---|---|
| DU-145 | 40.3 ± 5.2 | 11 ± 5.5 |
| PC3-ML | 15.4 ± 3.5 | 3.5 ± 1.2 |
| ARCaP-M | 29.8 ± 3.7 | 9.9 ± 2.4 |

TABLE 4
Effect of knockdown of mda-9 (shmda-9) on the expression
of stemness genes in prostate cancer cell lines.

| | DU-145 | | ARCaP-M | | PC3-ML | |
|---|---|---|---|---|---|---|
| GENES | Shcon | Shmda-9 | Shcon | Shmda-9 | Shcon | Shmda-9 |
| mda-9 | 1 ± 0.07 | 0.2 ± 0.15 | 1 ± 0.05 | 0.3 ± 0.07 | 1 ± 0.06 | 0.1 ± 0.12 |
| Stemness genes | | | | | | |
| Nanog | 1 ± 0.02 | 0.55 ± 0.04 | 1 ± 0.03 | 0.10 ± 0.04 | 1 ± 0.02 | 0.15 ± 0.02 |
| Sox2 | 1 ± 0.06 | 0.32 ± 0.00 | 1 ± 0.01 | 0.26 ± 0.03 | 1 ± 0.08 | 0.10 ± 0.05 |
| Oct4 | 1 ± 0.11 | 0.14 ± 0.04 | 1 ± 0.25 | 0.10 ± 0.01 | 1 ± 0.05 | 0.03 ± 0.00 |
| c-myc | 1 ± 0.01 | 0.17 ± 0.03 | 1 ± 0.02 | 0.05 ± 0.01 | 1 ± 0.02 | 0.09 ± 0.00 |

TABLE 5
Effect of mda-9 knockdown (shmda-9) on gene
expression in prostate cancer cell lines.

| | % expression | | | | | |
|---|---|---|---|---|---|---|
| | DU-145 | DU-145 | ARCaP-M | ARCaP-M | PC3-ML | PC3-ML |
| Protein | shcon | shmda-9 | shcon | shmda-9 | shcon | shmda-9 |
| P-STAT3 | 62.3 ± 9.8 | 15.4 ± 4.1 | 6.5 ± 0.6 | 2.2 ± 0.3 | 41.3 ± 7.6 | 29.6 ± 3.1 |
| STAT3 | 85.1 ± 8.6 | 87.4 ± 2.7 | 24.6 ± 1.9 | 25.9 ± 5.2 | 62.7 ± 7.9 | 65.7 ± 4.6 |
| P-SRC | 68.2 ± 6.5 | 11.5 ± 5.3 | 37.9 ± 5.6 | 12.6 ± 3.8 | 53.9 ± 4.2 | 37.1 ± 2.7 |
| SRC | 77.4 ± 5.1 | 75.2 ± 4.5 | 39.4 ± 5.8 | 34.1 ± 3.5 | 69.9 ± 6.7 | 63.3 ± 5.1 |
| P-p44/42 | 18.8 ± 4.4 | 1.4 ± 0.07 | 21.2 ± 4.7 | 6.4 ± 0.6 | 6.4 ± 1.5 | 1.7 ± 0.2 |
| p44/42 | 21.4 ± 2.9 | 4.8 ± 2.9 | 21.6 ± 9.5 | 11.9 ± 2.9 | 16.2 ± 2.4 | 6.5 ± 1.2 |
| P-IGF1R | 20.2 ± 5.3 | 9.7 ± 2.5 | 18.5 ± 2.9 | 10.5 ± 3.1 | 7.2 ± 1.1 | 1.8 ± 0.4 |
| IGF1R | 35.7 ± 4.9 | 36.9 ± 7.2 | 34.8 ± 7.4 | 36.2 ± 4.4 | 25.6 ± 5.7 | 27.4 ± 3.2 |
| NOTCH1 | 94.3 ± 4.7 | 31.5 ± 8.5 | 49.7 ± 9.1 | 36.2 ± 2.6 | 74.7 ± 8.8 | 36.4 ± 4.6 |
| DLL1 | 41.8 ± 8.2 | 13.7 ± 2.6 | 44.5 ± 12.6 | 32.9 ± 3.3 | 49.4 ± 6.9 | 27.6 ± 2.6 |
| Numb | 22.3 ± 4.8 | 44.3 ± 5.9 | 0.5 ± 0.1 | 7.3 ± 1.7 | 9.81 ± 2.4 | 19.5 ± 3.8 |
| C-Myc | 53.8 ± 2.2 | 25.4 ± 3.5 | 60.6 ± 9.6 | 39.6 ± 7.6 | 53.0 ± 3.1 | 37.8 ± 1.5 |

Considering the significant role of STAT3 in prostate cancer [49] and PCSCs [34], we analyzed the effect of MDA-9 on STAT3 in PCSCs. mda-9 kd significantly decreased p-STAT3 (Tyr-705) expression by ˜1.5-3.5-fold in DU-145, ARCaP-M and PC3-ML PCSCs (Table 5). Active SRC was also known to positively regulate STAT3 [45] and we found significant decreased ranging from ˜1.5-6-fold, in SRC activation post mda-9 kd (Table 5). STAT3 is additionally regulated by p44/42 and IGF-1R [50-52], and considering this, we also examined the expression of these proteins in control and shmda-9 PCSCs. A significant decrease in p44/42 was observed (Table 5), and an even more profound decrease in phospho-p44/42 (Table 5). p-IGF-1R was also significantly decreased in the shmda-9 cells (Table 5). STAT3 is important for c-myc expression [53-55], which potentially adds another key regulatory element involved in PCSC maintenance.

### Example 5. MDA-9 Maintains PCSC-Mediated Survival and Tumorigenicity

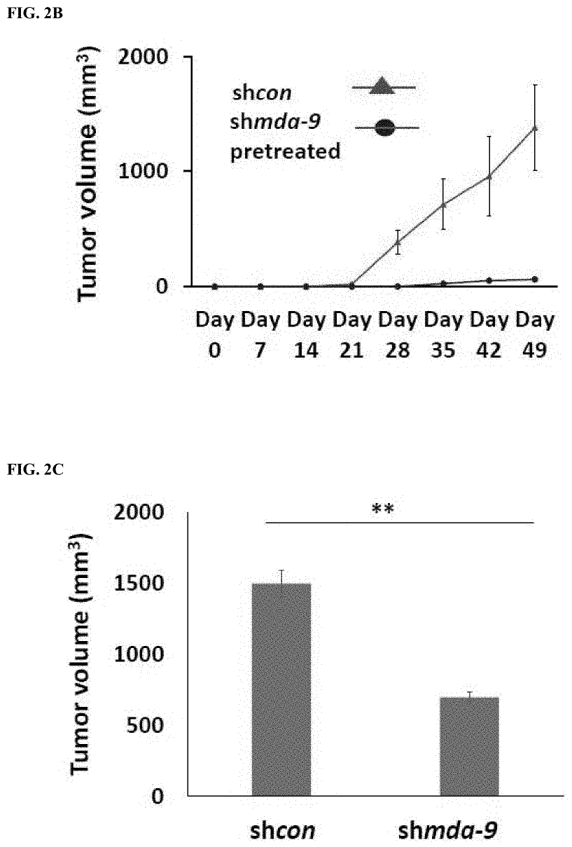

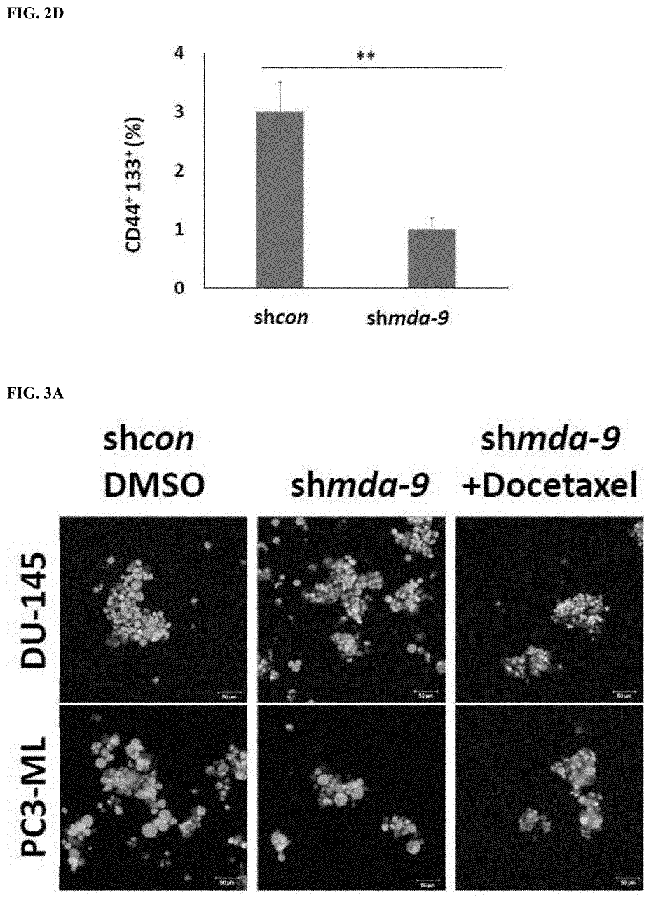

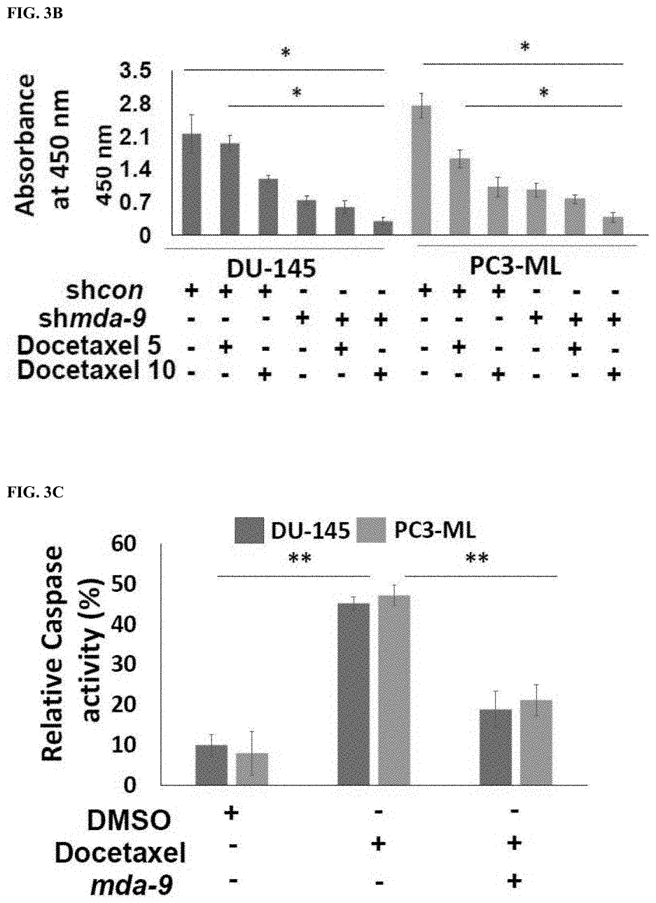

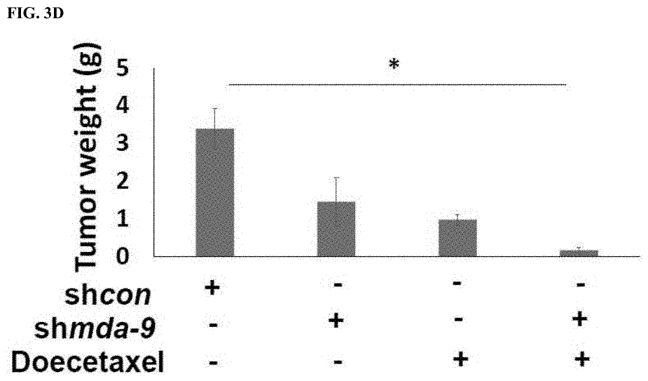

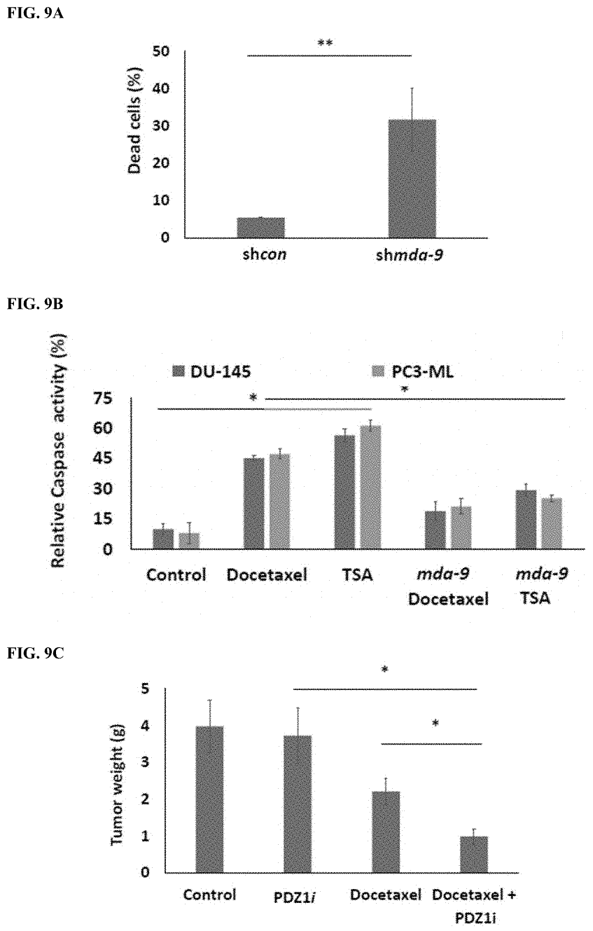

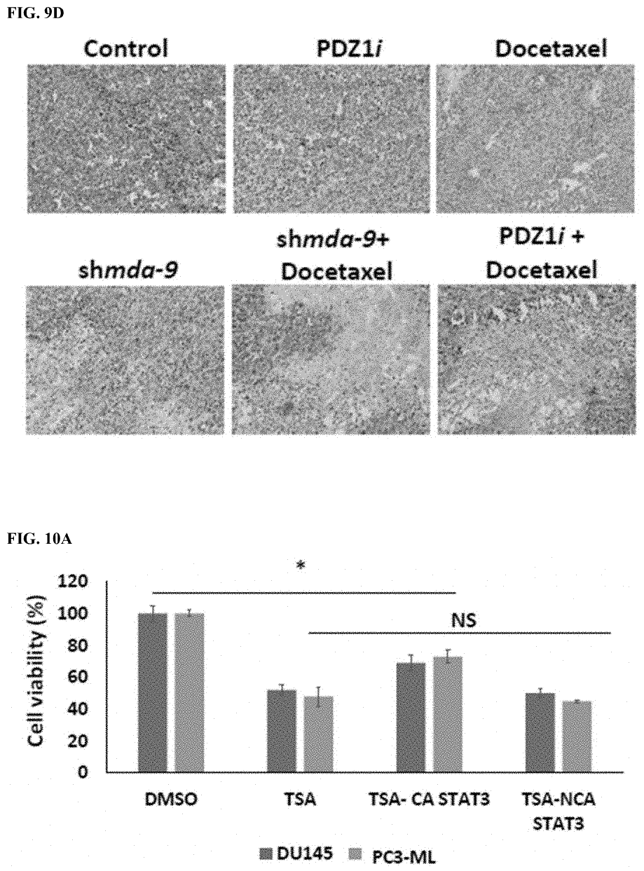

Apart from the loss of self-renewal (Table 3), mda-9 kd also significantly increased cell death and apoptosis in PCSCs from DU-145 cells, as early as 72 h post kd (FIGS. 2A and 9A). mda-9 kd in PCSCs decreased tumorigenicity. The pretreated shmda-9 cells were obtained by treating PCSCs with Ad.5/3.shmda-9 at 1000 v.p. per cell. When shcon and shmda-9 pre-treated DU-145 PCSCs were injected subcutaneously into male nude mice (n=10), the shcon group formed large tumors with a substantial population of PCSCs (FIG. 2B). However, the shmda-9 tumors were extremely small (FIG. 2B). mda-9 silencing with intra-tumoral injection of shmda-9 virus in subcutaneous DU-145 PCSC-derived tumors also resulted in smaller tumors, with decreased tumor growth kinetics and PCSC populations in the intra-tumoral treated groups (FIGS. 2C and 2D). The PCSCs are grown in anoikis conditions in vitro, and these cells are sensitive to anoikis post mda-9 kd [46]. This may have been the reason why pretreated PCSCs did not form tumors, but intra-tumoral mda-9 kd PCSCs were able to form small tumors. However, the results showed that MDA-9 was important for PCSC function, and the loss of MDA-9 lead to a sharp decrease in their ability to form tumors. MDA-9 was also important for PCSC survival and maintenance of the CSC-niche, as loss of MDA-9 lead to loss of spheroid integrity, and ultimately, loss of PCSC viability (FIG. 3A).

### Example 6. Suppression of MDA-9 Sensitizes PCSCs to Multiple Chemotherapeutic Drugs Via STAT3

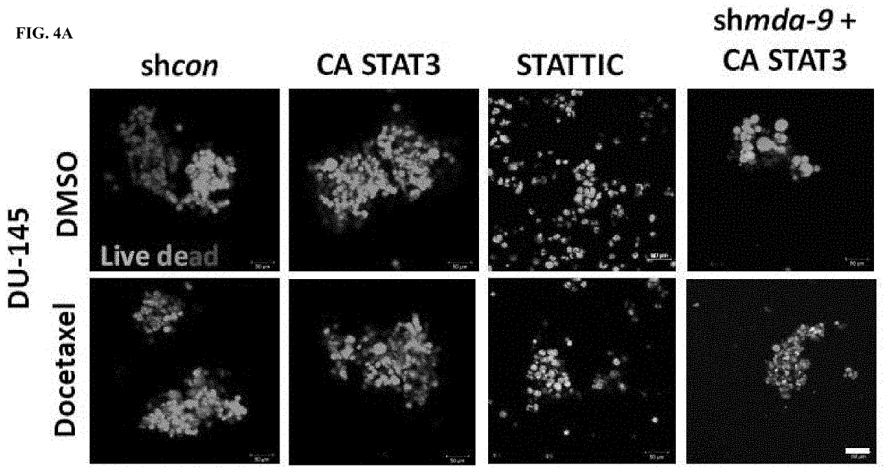

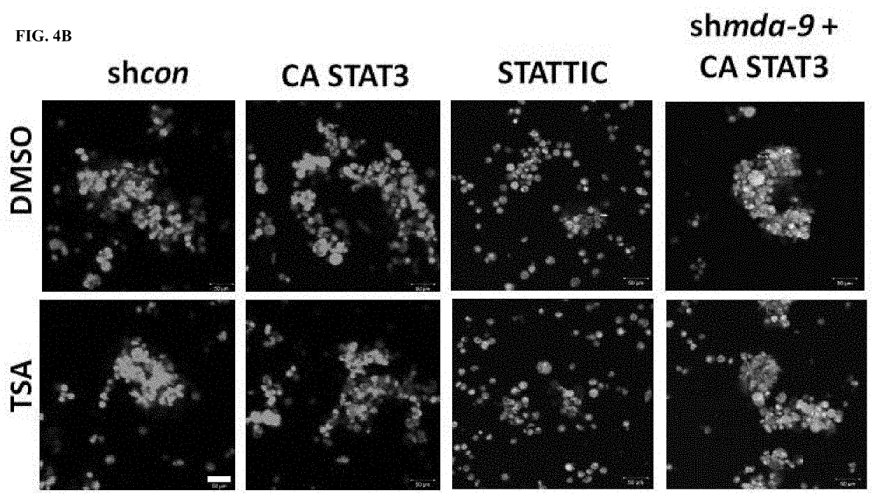

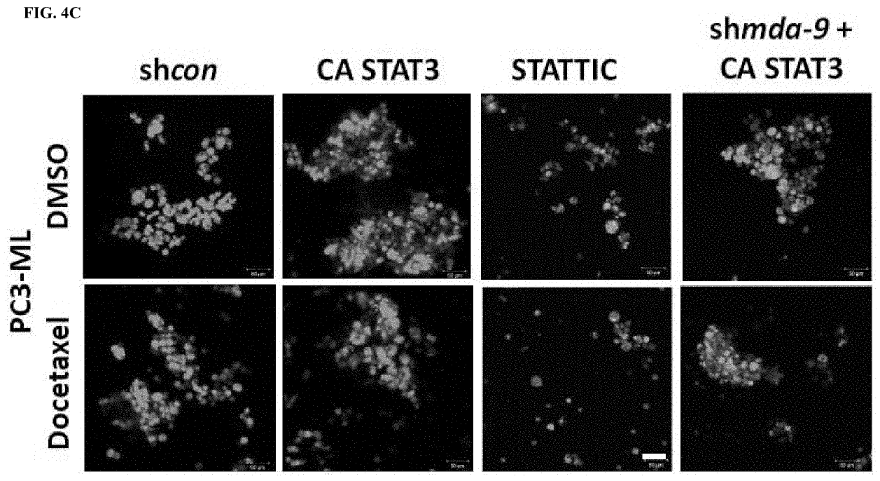

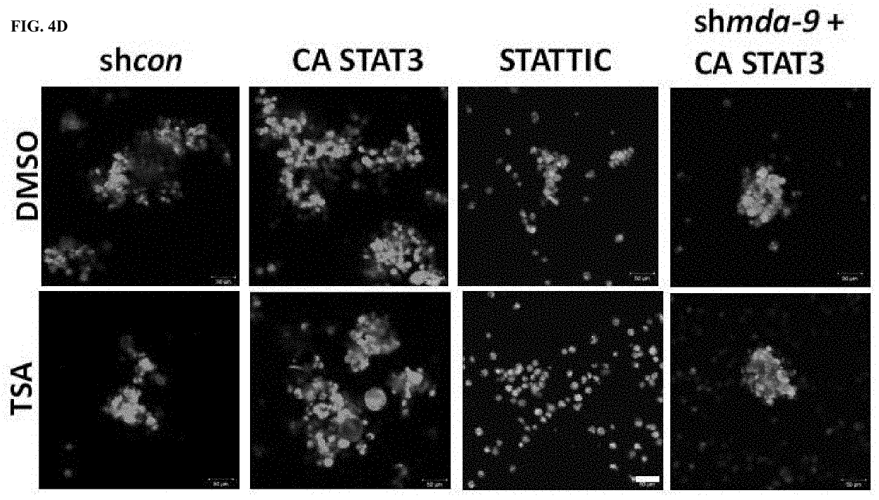

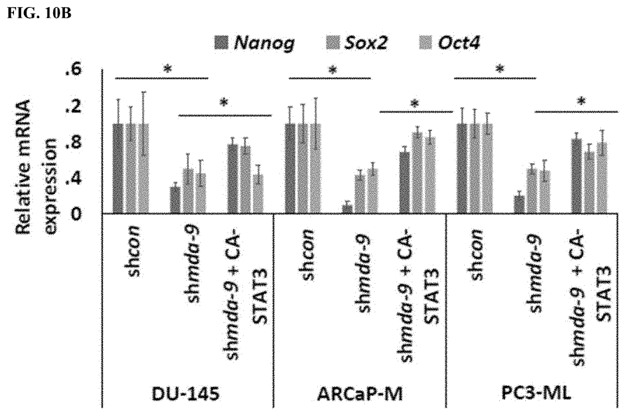

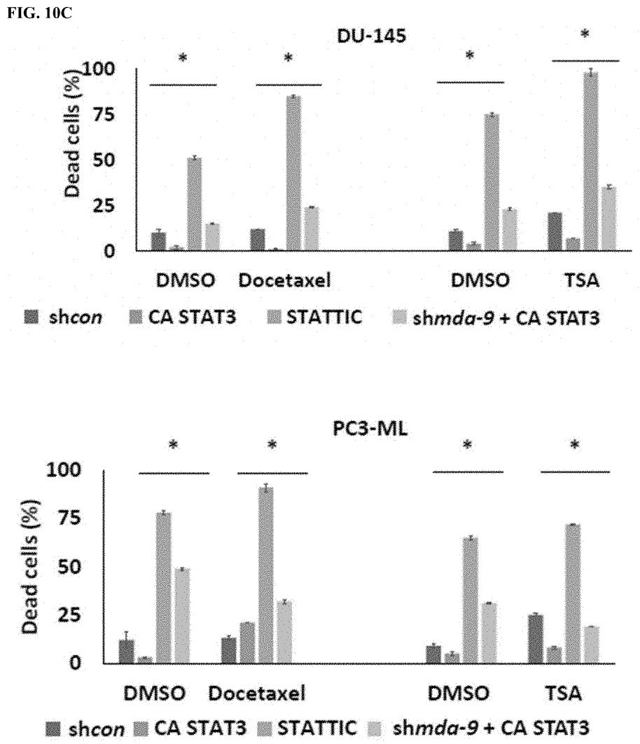

PCSCs are relatively resistant to docetaxel (FIGS. 3A-3B) resulting in a range of ˜65/6-70% cell proliferation vs. control post 10 and 5 nM treatment (FIG. 3B). However, the shmda-9 PCSCs show a range of ˜35%-40% and ˜20%-30% cell proliferation vs. control following 10 and 5 nM treatment, respectively (FIG. 3B). Overexpression of mda-9 led to a decrease in docetaxel and trichostatin-A-induced caspase activity (FIGS. 3C and 9B). These results indicated that the kd of MDA-9 results in sensitivity of PCSCs to docetaxel and trichostatin-A, and that the high expression of MDA-9 in the PCSCs may have conferred some chemoresistance to these unique populations of cells. In vivo xenograft studies also confirmed that loss of MDA-9 by both genetic and pharmacological techniques lead to sensitization to docetaxel treatment (FIGS. 3D, 9C and 9D). Further experiments suggested that MDA-9 regulates this resistance phenotype through STAT3 activation. This was confirmed through CA-STAT3 (constitutively active STAT3) overexpression and STAT3 inhibitor studies (STATTIC). We overexpressed a constitutively active STAT3 (A662C/N664C; CA-STAT3) in the shmda-9 PCSCs and observed a recovery of resistance to Docetaxel and Trichostatin A (FIGS. 4A-AD and 10A). Interestingly, when we used a non-constitutively active STAT3, this rescue effect was abrogated (FIG. 10A). CA-STAT3 was able to confer chemoresistance in shmda-9 PCSCs, whereas STATTIC caused chemosensitivity to both docetaxel and trichostatin-A (FIGS. 4A-4D and 10C). Additionally, overexpression of CA-STAT3 in the shmda-9 PCSCs abrogated the inhibitory effects of mda-9 kd on expression of sternness regulatory genes (FIG. 10B). These results showed that MDA-9 exerted a sternness and chemoresistance phenotype in PCSCs by regulating STAT3.

### Example 7. MDA-9 Mediates PCSC Chemoresistance Through the STAT3-MDR1 Axis

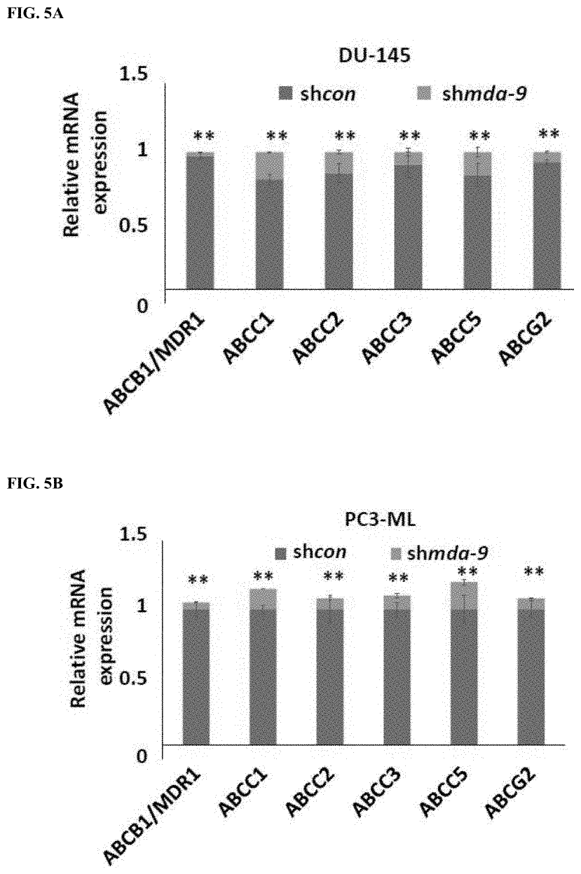

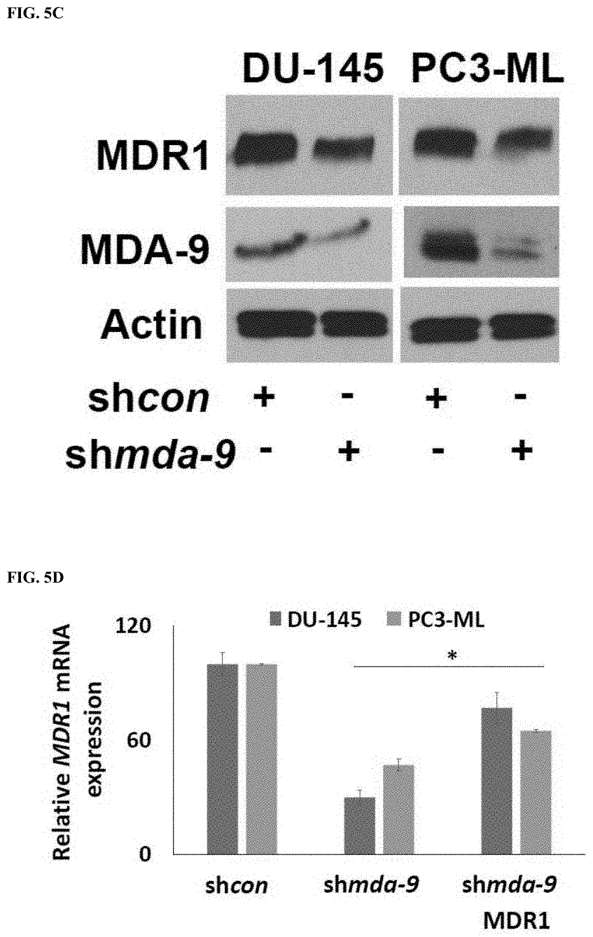

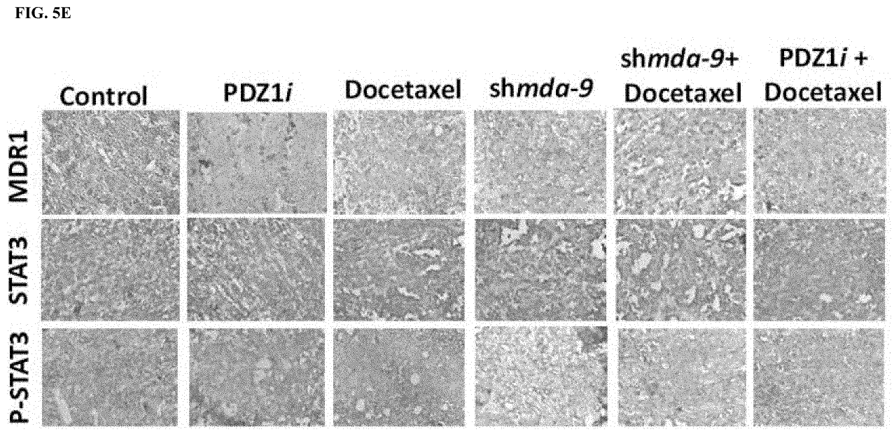

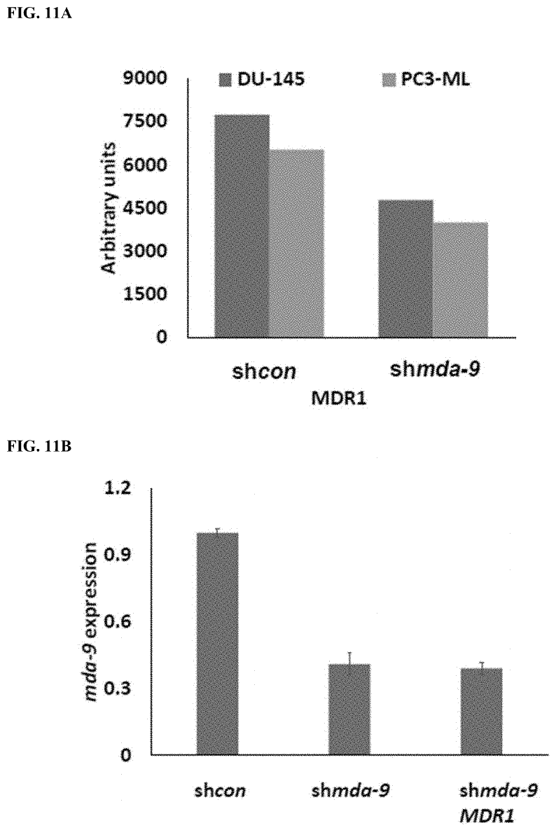

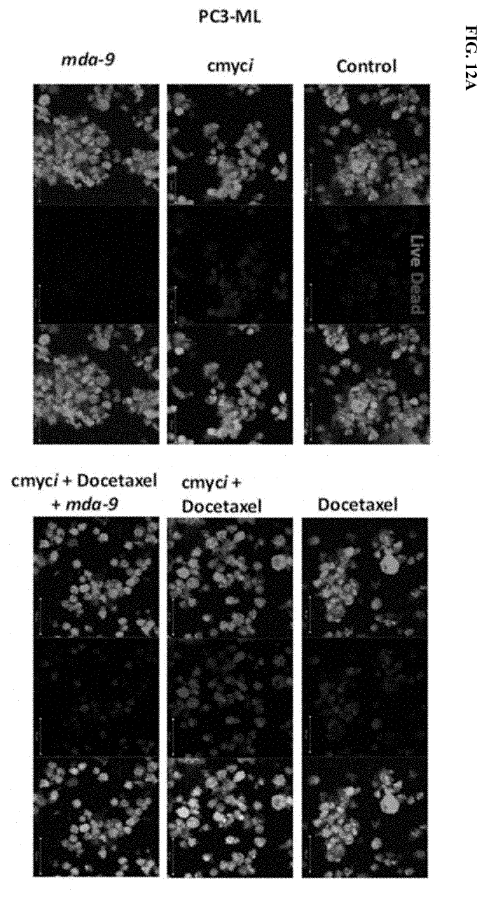

Since we observed a possible role of MDA-9 in PCSC chemoresistance, we analyzed the expression of the ABC family of genes in DU-145 and PC3-ML PCSCs (FIGS. 5A-5B). We observed that the ABC family of genes was highly expressed in both cell lines with ABCB1 or MDR1/P-glycoprotein being the most significantly affected by mda-9 kd (FIGS. 5A-5C). MDR1 mRNA expression decreased significantly in the shmda-9 group as compared to the shcon group (˜33-fold in DU-145 and ˜20-fold in PC3-ML, as shown in FIG. 5D). The protein levels of MDR1 also decreased significantly in the shmda-9 group as compared to the shcon group (DU-145-1.7-fold and PC3-ML ˜1.6-fold) as shown in FIGS. 5C and 11A. Protein levels in ARCaP-M shmda-9 PCSCs decreased by ˜three-fold, as shown in FIG. 6C. When we analyzed in vivo mice tumor xenograft histologies, these results were substantiated (FIG. 5E). mda-9 kd in PCSCs resulted in increased apoptotic cell death as evidenced by enhanced caspase 3 activity (FIG. 6A), which was rescued by MDR1 overexpression (FIGS. 5D, 6A and 11B). In addition, mda-9 kd-mediated MDR1 expression loss was significantly rescued by the overexpression of CA-STAT3 in DU-145, PC3-ML and ARCaP-M PCSCs (FIGS. 6B-6C). These results showed that MDR1 is important in lessening caspase-mediated cell death in mda-9 kd-treated cells (FIG. 6A). STAT3 appeared to play a very important role in MDA-9-mediated MDR1 expression, thereby contributing to chemoresistance to multiple drugs such as docetaxel and Trichostatin-A (TSA).

### Example 8. C-Myc Regulation by MDA-9 is Essential for Stem Cell Renewal, Maintenance, Survival and MDR1 Expression

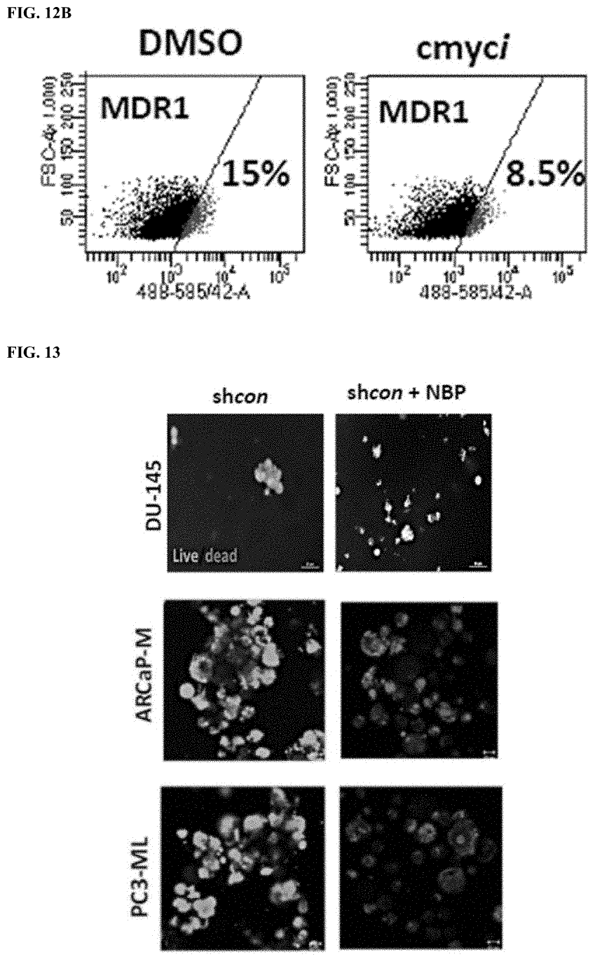

To confirm whether MDA-9 contributes to C-myc regulation in PCSCs, we analyzed its expression in shcon and shmda-9-treated PCSCs. Suppression of mda-9 by kd led to a significant decrease in C-myc expression at RNA and protein levels (RNA: ˜5.9 to ˜47.6-fold change Table 4; protein: ˜1.4 to two-fold change as compared to shcon (Table 5). Additionally, C-myc inhibition led to a decrease in MDR1 expression as well as chemoresistance to Docetaxel (FIGS. 12A-12B), which was recovered by overexpression of mda-9. C-myc expression is regulated by RBPJK via NOTCH1 signaling, and this is possibly the pathway by which MDA-9 mediates C-Myc regulation. NOTCH1 expression consistently decreased following mda-9 kd in DU-145, ARCaP-M and PC3-ML PCSCs (Table 5). This decrease in NOTCH1 expression in mda-9 kd PCSCs probably results from elevated NOTCH1 degradation due to the increased expression of NUMB in DU-145, ARCaP-M and PC3-ML PCSCs (Table 5). Utilizing a NOTCH-Blocking Peptide (NBP) to block NOTCH1 activation and downstream signaling also resulted in the same phenotype observed in mda-9 kd PCSCs (FIG. 13). These results indicated that MDA-9 regulated C-myc in PCSCs, through NOTCH1 signaling. Collectively, our findings suggested that MDA-9 regulated MDR1-mediated chemoresistance in PCSCs through C-myc, in addition to STAT3.

### Example 9. Material and Methods

### Cell Lines and Tissue Samples

Human prostate cancer cells DU-145 (ATCC, Manassas, Va., USA) and PC3-ML cells were cultured in DMEM supplemented with 10% fetal bovine serum and antibiotics. PC3-ML-Luc cells were obtained from Dr. Martin G. Pomper (Johns Hopkins Medical Institutions, Baltimore, Md., USA). ARCaP-M cells (Novicure Biotechnology©, Birmingham, Ala., USA) were cultured using MCAP medium supplemented with 10% fetal bovine serum and antibiotics (Novicure Biotechnology©). Isolated non-stem prostate cancer cells (NSCC, Seattle, Wash., USA), based on lack of expression of CD133 and CD44, were similarly cultured in monolayer culture. Normal immortal human prostate epithelial cells, RWPE-1, were obtained from ATCC and grown in Keratinocyte-SFM media supplemented with human recombinant Epidermal Growth Factor, Bovine Pituitary Extract (BPE) and antibiotics (Thermo Fisher Scientific©, Waltham, Mass., USA). The cumulative culture length of the cells was less than 6 months after being placed in culture. Early passage cells were used for all experiments. All the cell lines were routinely tested (every 6 months) for mycoplasma contamination using a mycoplasma detection kit from Sigma-Adlrich© (St. Louis, Mo., USA).

### Isolation and Culture of Putative Human PCSCs and NSCCs

Human PCSCs and NSCCs were isolated from 3 different prostate cancer cell lines: DU-145, ARCaP-M, and PC3-ML. DU-145 (ATCC) and PC3-ML cells were cultured in monolayers using complete DMEM media. PC3-ML-Luc cells were obtained from Dr. Martin G. Pomper (Johns Hopkins Medical Institutions, Baltimore, Md., USA). ARCaP-M cells (Novicure Biotechnology©) were cultured in monolayers using complete MCAP medium (Novicure Biotechnology©). The cells were next cultured in ultra-low attachment T25 or T75 culture flasks (Corning©, New York, N.Y., USA) in Essential 8 medium (Thermo Fisher Scientific©) to form PCSC-enriched prostaspheres and studied prior to 5 passages. Prostaspheres were disassociated and then labeled with CD44 and CD133 antibody (Miltenyi Biotech©, Auburn, Calif., USA) as PCSC markers [75]. Stained cells were sorted through a BD FACSAria™ II sorting station. Cells highly expressing both CD44 and CD133 were selected as PCSCs while, cells lacking both were selected as non-stem cancer cells [9,20,75,76]. CD44⁺ CD133⁺ PCSC and CD44⁻ CD133⁻ NSCC populations were counted and collected for further culturing. Xenografted human PCSCs were isolated from mice and analyzed for cell surface markers by flow cytometry. All PCSCs were grown in ultra-low attachment plates and flasks with Essential 8™ medium (Invitrogen™, Carlsbad, Calif., USA), and NSCCs were cultured in monolayer with complete DMEM medium, unless otherwise indicated.

### Isolation and Culture of Primary Prostate Epithelial Stem Cells

Primary immortal normal human prostate epithelial cells (RWPE-1, ATCC) were cultured initially in T175 flasks (Corning) in Keratinocyte-SFM media (Thermo Fisher Scientific©). The cells were grown in ultra-low attachment plates and flasks (Corning) using Essential 8 medium (Thermo Fisher Scientific©) to enrich the stem cell population. The cells were stained with CD44, CD133 and α₂β₁ antibodies as prostate stem cell markers [75], sorted and further cultured under ultra-low attachment conditions.

### Promoter Reporter Assays

2×10⁵ cells were infected with either Ad.5/3.shcon or Ad.5/3.shmda-9. The cells were transfected post-infection with an RBPJK-responsive luciferase reporter construct using Lipofectamine 2000 [46]. Cells were lysed and the resultant relative luciferase activity was measured using a Dual-Luciferase Reporter Assay system (Promega, Madison, Wis., USA) (manufacturer's instructions). Luciferase activity was normalized to Renilla activity, and the data show the average of triplicates±S.D.

### Reverse Transcription Polymerase Chain Reaction

PCSCs were pelleted by centrifugation, washed twice with PBS and then total RNA was extracted utilizing TRIzol™ (Invitrogen™) followed by purification of the resultant lysate using the RNeasy kit (Qiagen®, Frederick, Md., USA). First-strand cDNA was synthesized with SuperScript III reverse transcriptase (Invitrogen™). Quantitative PCR studies were performed using the TaqMan® Gene expression assays (Invitrogen™) using a ViiA 7 fast real-time PCR system (Applied Biosystems™, Foster City, Calif., USA). Relative gene expression was normalized to 18S expression (Invitrogen™).

### Western Blotting

Cells were washed with ice-cold PBS prior to lysis. All the samples were incubated on ice using lysis buffer (20 mM Tris-HCl (pH 7.5), 150 mM NaCl, 1 mM Na₂EDTA, 1 mM EGTA, 1% Triton-100, 2.5 mM Sodium pyrophosphate, 1 mM O-glycerophosphate, 1 mM Na₃VO₄, 1 μg/mL Leupeptin). Protein samples were prepared after determining protein concentration, and then loaded onto 8% SDS-PAGE. Membranes were stained with primary antibodies, followed by HRP-conjugated secondary antibodies. Individual bands were detected using ECL (Thermo Fisher Scientific©). The relative intensity values of all the proteins expressed in the samples were normalized against β-actin expression. For densitometry evaluation, X-ray films were scanned and evaluated with ImageJ software (National Institutes of Health [NIH], Bethesda, Md., USA). Antibody details are provided in the Supplemental Information.

### Immunofluorescent Staining and Confocal Microscopy

Cells were fixed with 4% paraformaldehyde for 20 min at room temperature, permeabilized with 0.1% Triton X-100 for 5 min and blocked with 5% bovine serum for 1 h. NANOG, SOX2 and OCT4 staining was performed according to the manufacturer's instructions (CST, Danvers, Mass., USA). Nuclei were visualized with 1 μg/mL DAPI. Cells were imaged using laser confocal microscopy (Leica, Buffalo Grove, Ill., USA). The images were analyzed by ZEISS© Zen software.

### Live Cell Imaging

Live cell images were obtained using a Zeiss (San Diego, Calif., USA) Cell Observer SD spinning disk confocal microscope furnished with a Yokogawa CSU-X1A spinning disk, two Photometrics Evolve 512 cameras (16-bit), a high-resolution piezoelectric driven Z-stage, an acousto-optic tunable filter, four lasers (405 nm, multiline argon 458/488/514 nm, 561 nm, 635 nm), and a PeCon A stage incubation system. The stage and chamber conditions were optimized to remain constantly at 37° C., with the CO₂ level at 5%. The CSCs were stained with Cell tracker green CMFDA (Invitrogen™). Sequential imaging was performed in green (488 nm excitation, 525/50 nm emission) channels.

### Live/Dead Cell Assay

Live/Dead cell imaging was performed to assess fluorescence based cell viability. Staining was performed as per the manufacturer's directions (Invitrogen™), which was followed by imaging utilizing laser confocal microscopy (Leica©). The images were analyzed by Zeiss© ZEN software.

### Cell Proliferation

Cells were seeded at 1×10⁵ cells/mL in 6-well ultra-low attachment plates. Next, MTT reagent (7.5 mg/mL) in phosphate-buffered saline was added (10 μL/well), and the cultures were incubated at 37° C. for 30 min. The reaction was stopped by the addition of acidified Triton buffer (0.1 M HCl, 10% (v/v) Triton X-100; 50 μL/well), and the tetrazolium crystals were dissolved by mixing on a plate shaker at room temperature for 20 min as well as pipetting. The samples were measured at 595 nm and a reference wavelength of 650 nm. The results represent the mean±S.E. of 5 wells from one experiment that is representative of experiments repeated at least three times.

### Flow Cytometry Sorting and Analysis

Cell surface markers CD44, CD133, DLL1, MDR1 and Annexin V were stained according to the manufacturer's instructions, followed by flow cytometry analysis using BD FACSDIVA™.

### Intracellular Flow Cytometry

Intracellular STAT3, p-STAT3, p44/42, p-p44/42, p-SRC, and Numb protein expression were analyzed by intra-cellular flow cytometry [61,62]. Cell fixation, permeabilization as well as primary and secondary antibody staining were executed according to the manufacturer's instructions. Flow cytometry was performed and analyzed using BD FACSDIVA™.

### Tumorigenicity Studies

All experiments and procedures using mice were approved by the Institutional Animal Care and Use Committee (IACUC) of Virginia Commonwealth University Richmond, Va., USA, protocol code: AM10183). For the subcutaneous xenograft model, athymic male NCr-nu/nu mice (National Cancer Institute-Bethesda, Md., USA) were used (n=10 per group).

1×10⁵ PCSCs were injected per mouse. Animals were closely monitored for tumor size, weight and volume, according to the VCU-IACUC approved protocol and the resultant data were evaluated. Once tumors in the control group reached 2000 mm³, the mice were euthanized and the tumors of all the groups were measured at the same time.

### Peptide Blocking Studies

Control and treated PCSCs (1×10⁵ cells) were cultured in 6-well ultra-low attachment plates. NOTCH1 blocking peptide (Biovision©, Exton, Pa., USA) were used at a concentration of 10 μg/mL and incubated with cells for 48 h. After incubation, the cells were stained and analyzed for viability, spheroid size and structure.

shRNA Knockdown

shRNA sequences were obtained from Qiagen® using the following sequences: 5′-TTGACTCTTAAGATTATGTAA-3′ (shmda-9 #3, SEQ ID NO: 2) and 5′-TGGGATGGTCTTAGAATATIT-3′ (shmda-9 #4, SEQ ID NO: 3). Ad.5/3.shmda-9 was constructed as previously described [46] utilizing the following primer sequences: forward: 5′-GCCTGCTITTATCTITGAACATATTATTAAGCGAATGAAGCCTAGTATAATGAAAAG CCTAATGGACCACACCATTCCTGAG-3′ (SEQ ID NO: 4), and reverse: 5′-CTCAGGAATGGTGTGGTCCATTAGGCTITTCATTATACTAGGCTTCATTCGCTTAAT AATATGTTCAAAGATAAAAGCAGGC-3′ (SEQ ID NO: 5).

### Chemotherapeutic Studies

Docetaxel, STATTIC and Trichostatin-A (TSA) were obtained from Sigma. The PCSCs were serum-starved for 24 h and then treated with the drugs for 48 h at a 10 μM concentration (unless mentioned otherwise) prior to assessing the sensitivity of PCSCs to these drugs. DMSO treated cells were used as control. In vitro PDZ1i treatments occurred at 25 μM concentration. In vivo PDZ1i treatments occurred at 25 mg/kg body weight of the mice, 3 times a week for a month.

### Statistical Analysis

All experiments performed in vitro, in vivo and ex vivo, were analyzed statistically using the Student's t test and ANOVA (Microsoft© Excel®, 15.37 Redmond, Wash., USA). Pearson's correlation coefficient (R) and coefficient of determination (R²) were calculated for correlation analysis. All statistical tests were two-sided, and p values ≤0.05 and ≤0.01 were considered to be significant and highly significant, respectively.

### Plasmids

Small hairpin (sh)RNA for mda-9/syntenin (shmda-9/syntenin) was created with pSilencer hygro expression vectors according to the manufacturer's protocol (Ambion®). The specific hairpin small interfering (si)RNA oligonucleotides sequences are:

sense
(SEQ ID NO: 6)
5′GATCCGCGGATGGCACCAAGCATTTTCAAGAGAAATGCTTGGTGCCA
TCCGCTTTTTTGGAAA-3′
and
antisense
(SEQ ID NO: 7)
5′AGCTTTTCCAAAAAAGCGGATGGCACCAAGCATTTCTCTTGAAAATG
CTTGGTGCCATCCGCG-3′.

The oligo nucleotides were annealed and ligated to pSilencer vector by T4 DNA ligase. Alternate siRNA sequences were obtained through Qiagen® with the following sequences: 5′-TTGACTCTTAAGATTATGTAA-3′ (simda-9 #3, SEQ ID NO: 2). shmda-9 resistant mda-9/syntenin plasmid was created using the following primer sequences:

forward
(SEQ ID NO: 8)
5′-GCCTGCTTTTATCTTTGAACATATTATTAAGCGAATGAAGCCTAGT
ATAATGAAAAGCCTAATGGACCACACCATTCCTGAG-3′
and
reverse:
(SEQ ID NO: 9)
5′-CTCAGGAATGGTGTGGTCCATTAGGCTTTTCATTATACTAGGCTTC
ATTCGCTTAATAATATGTTCAAAGATAAAAGCAGGC-3′.

These plasmids were cleaved and transfected into HEK-293 cells to obtain the corresponding Ad.5/3-based vectors. The viruses were then expanded using HEK-293 cells and purified by cesium chloride double ultracentrifugation (Beckman SW28 rotor) using standard protocols (OD260 Inc., Boise, Id.). The infectious viral particles were titered by plaque assay as described [46].

### RT-PCR Thermo Fisher Scientific© Taqman® Probes

The following RT-PCR Thermo Fisher Scientific© Taqman® probes were used:

mda-9 Hs01045460_g1

### myc Hs00153408_m1

### Nanog Hs0439%10_g1

### Sox2 Hs00415716_m1

### Oct4 Hs04260367_gH

### CD133 Hs01009250_m1

### Notch1 Hs01062014_m1

### 18S Hs99999901_s1

### ABCB1 Hs00184500_m1

### ABCC Hs01561483_m1

### ABCC2 Hs00960488_m1

### ABCC3 Hs00358656_m1

### ABCC5 Hs00981089_m1

### ABCG2 Hs01053790_m1

The interrogated sequence for each probe can be obtained at the Thermo Fisher Scientific© Gene expression assays (Taqman®) site.

### PCR Conditions

SuperScript™ Double-Stranded cDNA Synthesis Kit from Invitrogen™ was used to generate cDNA. We used the protocol as suggested by the company. 1 μg of Total RNA was used per 20 μl cDNA reaction. The tube was placed in the PCR machine programmed as follows: 25° C.-10 min, 50° C.-50 min, 85° C.-5 min. For RT-PCR, the cDNA reaction was diluted 1:10 with DEPC water and use 5p as template for a 20p0 RT-PCR reaction. RT-PCR was performed using ViiA 7 fast real-time PCR system, using the company protocol. These PCR conditions included polymerase activation at 95° C. for 4 min followed by 30 cycles at 95° C. for 30 sec, 63° C. for 30 sec, and 72° C. for 30 sec.

### Virus Infections

Viral infections were performed as described previously [46].

### Transfections

The cells were transfected with an constitutively active CA-STAT3 construct with Lipofectamine 2000 as described [45].

### Immunohistochemistry

The mice were monitored and euthanized according to an approved protocol, and the tumors were dissected. The tumor samples from all the groups of animals were fixed in phosphate-buffered formalin and paraffin sections were prepared using standard histology protocols. Paraffin-embedded sections were dewaxed and rehydrated through incubations in xylene and a gradient series of alcohol. Antigen retrieval was performed in 10 mM citric acid (pH 6.0) with microwave treatment for 20 min. Endogenous hydrogen peroxidase was quenched by 3% (vol/vol) H₂O₂ treatment for 20 min. Nonspecific binding sites were blocked with a solution of 5% (vol/vol) normal sera, and the sections were incubated with antibody overnight. The sections were incubated with biotinylated secondary antibodies and subsequently with avidin-biotin complex peroxidase (Vector Elite®; Vector Laboratories©). Colorimetric reactions were developed by DAB substrate (0.02% DAB (3,3′-diaminobenzidine), 0.005% hydrogen peroxide) treatment followed by 10% (vol/vol) Harris hematoxylin counterstaining. Hematoxylin & eosin staining was also conducted following a standard protocol as described earlier [46]. The images were analyzed under an Olympus BX41 microscope system equipped with DP25 digital camera and software.

### Antibodies

CD44, CD133 were obtained from Miltenyi Biotech. MDA-9 antibody was obtained from Alpha-Diagnostic International© (San Antonio, Tex.). IGFR, P-IGFR, P-STAT3, STAT3, Beta-actin, and MDR1 antibodies were obtained from Cell Signaling© (Beverly, Mass.).

### REFERENCES

1. Taitt, H. E. Global Trends and Prostate Cancer: A Review of Incidence, Detection, and Mortality as Influenced by Race, Ethnicity, and Geographic Location. Am. J. Mens. Health 2018, 12, 1807-1823.
2. Harris, W. P.; Mostaghel, E. A.; Nelson, P. S.; Montgomery, B. Androgen deprivation therapy: Progress in understanding mechanisms of resistance and optimizing androgen depletion. Nat. Clin. Pract. Urol. 2009, 6, 76-85.
3. Attard, G.; Parker, C.; Eeles, R. A.; Schrader, F.; Tomlins, S. A.; Tannock, I.; Drake, C. G.; de Bono, J. S. Prostate cancer. Lancet 2016, 387, 70-82.
4. Lang, S. H.; Frame, F. M.; Collins, A. T. Prostate cancer stem cells. J. Pathol. 2009, 217, 299-306.
5. Al-Hajj, M.; Wicha, M. S.; Benito-Hemandez, A.; Morrison, S. J.; Clarke, M. F. Prospective identification of tumorigenic breast cancer cells. Proc. Natl. Acad. Sci. USA 2003, 100, 3983-3988.
6. Singh, S. K.; Hawkins, C.; Clarke, I. D.; Squire, J. A.; Bayani, J.; Hide, T.; Henkelman, R. M.; Cusimano, M. D.; Dirks, P. B. Identification of human brain tumour initiating cells. Nature 2004, 432, 393-401.
7. Raff, M. Adult stem cell plasticity: Fact or artifact? Annu. Rev. Cell Dev. Biol. 2003, 19, 1-22.
8. Tang, D. G.; Patrawala, L.; Calhoun, T.; Bhatia, B.; Choy, G.; Schneider-Broussard, R.; Jeter, C. Prostate cancer stem/progenitor cells: Identification, characterization, and implications. Mol. Carcinog. 2007, 46, 1-14.
9. Collins, A. T.; Berry, P. A.; Hyde, C.; Stower, M. J.; Maitland, N.J. Prospective identification of tumorigenic prostate cancer stem cells. Cancer Res. 2005, 65, 10946-10951.
10. Collins, A. T.; Habib, F. K.; Maitland, N. J.; Neal, D. E. Identification and isolation of human prostate epithelial stem cells based on α2β1-integrin expression. J. Cell Sci. 2001, 114, 3865-3872.
11. Rybak, A. P.; Bristow, R. G.; Kapoor, A. Prostate cancer stem cells: Deciphering the origins and pathways involved in prostate tumorigenesis and aggression. Oncotarget 2015, 6, 1900-1919.
12. Leong, K. G.; Gao, W. Q. The Notch pathway in prostate development and cancer. Differentiation 2008, 76, 699-716.
13. Shen, M. M.; Abate-Shen, C. Molecular genetics of prostate cancer: New prospects for old challenges. Genes Dev. 2010, 24, 1967-2000.
14. Liu, C.; Lou, W.; Zhu, Y.; Nadiminty, N.; Schwartz, C. T.; Evans, C. P.; Gao, A. C. Niclosamide inhibits androgen receptor variants expression and overcomes enzalutamide resistance in castration-resistant prostate cancer. Clin. Cancer Res. 2014, 20, 3198-3210.
15. Yu, E. Y.; Miller, K.; Nelson, J.; Gleave, M.; Fizazi, K.; Moul, J. W.; Nathan, F. E.; Higano, C. S. Detection of previously unidentified metastatic disease as a leading cause of screening failure in a phase III trial of zibotentan versus placebo in patients with nonmetastatic, castration resistant prostate cancer. J. Urol. 2012, 188, 103-109.
16. Hellawell, G. O.; Turner, G. D.; Davies, D. R.; Poulsom, R.; Brewster, S. F.; Macaulay, V. M. Expression of the type 1 insulin-like growth factor receptor is up-regulated in primary prostate cancer and commonly persists in metastatic disease. Cancer Res. 2002, 62, 2942-2950.
17. Zhao, Y.; Bao, Q.; Renner, A.; Camaj, P.; Eichhom, M.; Ischenko, I.; Angele, M.; Kleespies, A.; Jauch, K. W.; Bruns, C. Cancer stem cells and angiogenesis. Int. J. Dev. Biol. 2011, 55, 477-482.
18. Bimie, R.; Bryce, S. D.; Roome, C.; Dussupt, V.; Droop, A.; Lang, S. H.; Berry, P. A.; Hyde, C. F.; Lewis, J. L.; Stower, M. J.; et al. Gene expression profiling of human prostate cancer stem cells reveals a pro-inflammatory phenotype and the importance of extracellular matrix interactions. Genome Biol. 2008, 9, R83.
19. Fodde, R.; Brabletz, T. Wnt/beta-catenin signaling in cancer sternness and malignant behavior. Curr. Opin. Cell Biol. 2007, 19, 150-158.
20. Gu, G.; Yuan, J.; Wills, M.; Kasper, S. Prostate cancer cells with stem cell characteristics reconstitute the original human tumor in vivo. Cancer Res. 2007, 67, 4807-4815.
21. Williams, K.; Motiani, K.; Giridhar, P. V.; Kasper, S. CD44 integrates signaling in normal stem cell, cancer stem cell and (pre)metastatic niches. Exp. Biol. Med. 2013, 238, 324-338.
22. Moltzahn, F.; Thalmann, G. N. Cancer stem cells in prostate cancer. Transl. Androl. Urol. 2013, 2, 242-253.
23. Patrawala, L.; Calhoun, T.; Schneider-Broussard, R.; Li, H.; Bhatia, B.; Tang, S.; Reilly, J. G.; Chandra, D.; Zhou, J.; Claypool, K.; et al. Highly purified CD44+ prostate cancer cells from xenograft human tumors are enriched in tumorigenic and metastatic progenitor cells. Oncogene 2006, 25, 1696-1708.
24. Grosse-Gehling, P.; Fargeas, C. A.; Dittfeld, C.; Garbe, Y.; Alison, M. R.; Corbeil, D.; Kunz-Schughart, L. A. CD133 as a biomarker for putative cancer stem cells in solid tumours: Limitations, problems and challenges. J. Pathol. 2013, 229, 355-378.
25. Baba, T.; Convery, P. A.; Matsumura, N.; Whitaker, R. S.; Kondoh, E.; Perry, T.; Huang, Z.; Bentley, R. C.; Mori, S.; Fujii, S.; et al. Epigenetic regulation of CD133 and tumorigenicity of CD133+ ovarian cancer cells. Oncogene 2009, 28, 209-218.
26. Dhir, R.; Ni, Z.; Lou, W.; DeMiguel, F.; Grandis, J. R.; Gao, A. C. Stat3 activation in prostatic carcinomas. Prostate 2002, 51, 241-246.
27. Ni, Z.; Lou, W.; Leman, E. S.; Gao, A. C. Inhibition of constitutively activated Stat3 signaling pathway suppresses growth of prostate cancer cells. Cancer Res. 2000, 60, 1225-1228.
28. Horinaga, M.; Okita, H.; Nakashima, J.; Kanao, K.; Sakamoto, M.; Murai, M. Clinical and pathologic significance of activation of signal transducer and activator of transcription 3 in prostate cancer. Urology 2005, 66, 671-675.
29. Liu, X.; He, Z.; Li, C. H.; Huang, G.; Ding, C.; Liu, H. Correlation analysis of JAK-STAT pathway components on prognosis of patients with prostate cancer. Pathol. Oncol. Res. 2012, 18, 17-23.
30. Tam, L.; McGlynn, L. M.; Traynor, P.; Mukherjee, R.; Bartlett, J. M.; Edwards, J. Expression levels of the JAK/STAT pathway in the transition from hormone-sensitive to hormone-refractory prostate cancer. Br. J. Cancer 2007, 97, 378-383.
31. Kroon, P.; Berry, P. A.; Stower, M. J.; Rodrigues, G.; Mann, V. M.; Simms, M.; Bhasin, D.; Chettiar, S.; Li, C.; Li, P. K.; et al. JAK-STAT blockade inhibits tumor initiation and clonogenic recovery of prostate cancer stem-like cells. Cancer Res. 2013, 73, 5288-5298.
32. Wang, S.; Garcia, A. J.; Wu, M.; Lawson, D. A.; Witte, O. N.; Wu, H. Pten deletion leads to the expansion of a prostatic stem/progenitor cell subpopulation and tumor initiation. Proc. Natl. Acad. Sci. USA 2006, 103, 1480-1485.
33. Qu, Y.; Oyan, A. M.; Liu, R.; Hua, Y.; Zhang, J.; Hovland, R.; Popa, M.; Liu, X.; Brokstad, K. A.; Simon, R.; et al. Generation of prostate tumor-initiating cells is associated with elevation of reactive oxygen species and IL-6/STAT3 signaling. Cancer Res. 2013, 73, 7090-7100.
34. Schroeder, A.; Herrmann, A.; Cherryholmes, G.; Kowolik, C.; Buettner, R.; Pal, S.; Yu, H.; Muller-Newen, G.; Jove, R. Loss of androgen receptor expression promotes a stem-like cell phenotype in prostate cancer through STAT3 signaling. Cancer Res. 2014, 74, 1227-1237.
35. Frame, F. M.; Pellacani, D.; Collins, A. T.; Simms, M. S.; Mann, V. M.; Jones, G. D.; Meuth, M.; Bristow, R. G.; Maitland, N.J. HDAC inhibitor confers radiosensitivity to prostate stem-like cells. Br. J. Cancer 2013, 109, 3023-3033.
36. Domingo-Domenech, J.; Vidal, S. J.; Rodriguez-Bravo, V.; Castillo-Martin, M.; Quinn, S. A.; Rodriguez-Barrueco, R.; Bonal, D. M.; Charytonowicz, E.; Gladoun, N.; de la Iglesia-Vicente, J.; et al. Suppression of acquired docetaxel resistance in prostate cancer through depletion of notch- and hedgehog-dependent tumor-initiating cells. Cancer cell 2012, 22, 373-388.
37. Moitra, K. Overcoming Multidrug Resistance in Cancer Stem Cells. Biomed. Res. Int. 2015, 2015, 635745.
38. Abdullah, L. N.; Chow, E. K. Mechanisms of chemoresistance in cancer stem cells. Clin. Transl. Med. 2013, 2, 3.
39. Noonan, K. E.; Beck, C.; Holzmayer, T. A.; Chin, J. E.; Wunder, J. S.; Andrulis, I. L.; Gazdar, A. F.; Willman, C. L.; Griffith, B.; Von Hoff, D. D.; et al. Quantitative analysis of MDR1 (multidrug resistance) gene expression in human tumors by polymerase chain reaction. Proc. Natl. Acad. Sci. USA 1990, 87, 7160-7164.
40. Vaidyanathan, A.; Sawers, L.; Gannon, A. L.; Chakravarty, P.; Scott, A. L.; Bray, S. E.; Ferguson, M. J.; Smith, G. ABCB1 (MDR1) induction defines a common resistance mechanism in paclitaxel- and olaparib-resistant ovarian cancer cells. Br. J. Cancer 2016, 115, 431-441.
41. Castellón, E. A.; Valenzuela, R.; Lillo, J.; Castillo, V.; Contreras, H. R.; Gallegos, I.; Mercado, A.; Huidobro, C. Molecular signature of cancer stem cells isolated from prostate carcinoma and expression of stem markers in different Gleason grades and metastasis. Biol. Res. 2012, 45, 297-305.
42. Kegelman, T. P.; Wu, B.; Das, S. K.; Talukdar, S.; Beckta, J. M.; Hu, B.; Emdad, L.; Valerie, K.; Sarkar, D.; Furnari, F. B.; et al. Inhibition of radiation-induced glioblastoma invasion by genetic and pharmacological targeting of MDA-9/Syntenin. Proc. Natl. Acad. Sci. USA 2017, 114, 370-375.
43. Sarkar, D.; Boukerche, H.; Su, Z. Z.; Fisher, P. B. mda-9/Syntenin: More than just a simple adapter protein when it comes to cancer metastasis. Cancer Res. 2008, 68, 3087-3093.
44. Talukdar, S.; Emdad, L.; Das, S. K.; Sarkar, D.; Fisher, P. B. Evolving Strategies for Therapeutically Targeting Cancer Stem Cells. Adv. Cancer Res. 2016, 131, 159-191.
45. Talukdar, S.; Das, S. K.; Pradhan, A. K; Emdad, L.; Shen, X. N.; Windle, J. J.; Sarkar, D.; Fisher, P. B. Novel function of MDA-9/Syntenin (SDCBP) as a regulator of survival and sternness in glioma stem cells. Oncotarget 2016, 7, 54102-54119.
46. Talukdar, S.; Pradhan, A K.; Bhoopathi, P.; Shen, X. N.; August, L. A.; Windle, J. J.; Sarkar, D.; Fumari, F. B.; Cavenee, W. K.; Das, S. K; et al. MDA-9/Syntenin regulates protective autophagy in anoikis-resistant glioma stem cells. Proc. Natl. Acad. Sci. USA 2018, 11, 5768-5773.
47. Das, S. K.; Bhutia, S.; Kegelman, T. P.; Peachy, L.; Oyesanya, R. A.; Dasgupta, S.; Sokhi, U. K.; Azab, B.; Dash, R; Quinn, B. A.; et al. MDA-9/syntenin: A positive gatekeeper of melanoma metastasis. Front. Biosci. Landmark Ed. 2012, 17, 1-15.
48. Qian, X. L.; Li, Y. Q.; Yu, B.; Gu, F.; Liu, F. F.; Li, W. D.; Zhang, X. M.; Fu, L. Syndecan binding protein (SDCBP) is overexpressed in estrogen receptor negative breast cancers, and is a potential promoter for tumor proliferation. PLoS ONE 2013, 8, e60046.
49. Abdulghani, J.; Gu, L.; Dagvadorj, A.; Lutz, J.; Leiby, B.; Bonuccelli, G.; Lisanti, M. P.; Zellweger, T.; Alanen, K.; Mirtti, T.; et al. Stat3 promotes metastatic progression of prostate cancer. Am. J. Pathol. 2008, 172, 1717-1728.
50. Plaza-Menacho, I.; van der Sluis, T.; Hollema, H.; Gimm, O.; Buys, C. H.; Magee, A. I.; Isacke, C. M.; Hofstra, R. M.; Eggen, B. J. Ras/ERK1/2-mediated STAT3 Ser727 phosphorylation by familial medullary thyroid carcinoma-associated RET mutants induces full activation of STAT3 and is required for c-fos promoter activation, cell mitogenicity, and transformation. J. Biol. Chem. 2007, 282, 6415-6424.
51. Zhang, W.; Zong, C. S.; Hermanto, U.; Lopez-Bergami, P.; Ronai, Z.; Wang, L. H. RACK1 recruits STAT3 specifically to insulin and insulin-like growth factor 1 receptors for activation, which is important for regulating anchorage-independent growth. Mol. Cell. Biol. 2006, 26, 413-424.
52. Tkach, M.; Rosemblit, C.; Rivas, M. A.; Proietti, C. J.; Diaz Flaque, M. C.; Mercogliano, M. F.; Beguelin, W.; Maronna, E.; Guzman, P.; Gercovich, F. G.; et al. p42/p44 MAPK-mediated Stat3Ser727 phosphorylation is required for progestin-induced full activation of Stat3 and breast cancer growth. Endocr. Relat. Cancer 2013, 20, 197-212.
53. Bowman, T.; Broome, M. A.; Sinibaldi, D.; Wharton, W.; Pledger, W. J.; Sedivy, J. M.; Irby, R.; Yeatman, T.; Courtneidge, S. A.; Jove, R. Stat3-mediated Myc expression is required for Src transformation and PDGF-induced mitogenesis. Proc. Natl. Acad. Sci. USA 2001, 98, 7319-7324.
54. Kiuchi, N.; Nakajima, K.; Ichiba, M.; Fukada, T.; Narimatsu. M.; Mizuno. K.; Hibi. M.; Hirano, T. STAT3 is required for the gp130-mediated full activation of the c-myc gene. J. Exp. Med. 1999, 189, 63-73.
55. Barre, B.; Vigneron, A.; Coqueret, O. The STAT3 transcription factor is a target for the Myc and riboblastoma proteins on the Cdc25A promoter. J. Biol. Chem. 2005, 280, 15673-15681.
56. Koh, C. M.; Bieberich, C. J.; Dang, C. V.; Nelson, W. G.; Yegnasubramanian, S.; De Marzo, A. M. MYC and Prostate Cancer. Genes Cancer 2010, 1, 617-628.
57. Wolfer, A.; Wittner, B. S.; Irimia, D.; Flavin, R. J.; Lupien, M.; Gunawardane, R. N.; Meyer, C. A.; Lightcap, E. S.; Tamayo, P.; Mesirov, J. P.; et al. MYC regulation of a “poor-prognosis” metastatic cancer cell state. Proc. Natl. Acad. Sci. USA 2010, 107, 3698-3703.
58. Murphy, M. J.; Wilson, A.; Trumpp, A. More than just proliferation: Myc function in stem cells. Trends Cell Biol. 2005, 15, 128-137.
59. Yoshida, G. J. Emerging roles of Myc in stem cell biology and novel tumor therapies. J. Exp. Clin. Cancer Res. 2018, 37, 173.
60. Yun, E. J.; Zhou, J.; Lin, C. J.; Hemandez, E.; Fazli, L.; Gleave, M.; Hsieh, J. T. Targeting Cancer Stem Cells in Castration-Resistant Prostate Cancer. Clin. Cancer Res. 2016, 22, 670-679.
61. Azare, J.; Leslie, K.; Al-Ahmadie, H.; Gerald, W.; Weinreb, P. H.; Violette, S. M.; Bromberg, J. Constitutively activated Stat3 induces tumorigenesis and enhances cell motility of prostate epithelial cells through integrin beta 6. Mol. Cell. Biol. 2007, 27, 4444-4453.
62. Lee, T. K.; Castilho, A.; Cheung, V. C.; Tang, K. H.; Ma, S.; Ng, I. O. CD24(+) liver tumor-initiating cells drive self-renewal and tumor initiation through STAT3-mediated NANOG regulation. Cell Stem Cell 2011, 9, 50-63.
63. Liu, K.; Jiang, M.; Lu, Y.; Chen, H.; Sun, J.; Wu, S.; Ku, W. Y.; Nakagawa, H.; Kita, Y.; Natsugoe, S.; et al. Sox2 cooperates with inflammation-mediated Stat3 activation in the malignant transformation of foregut basal progenitor cells. Cell Stem Cell 2013, 12, 304-315.
64. Boukerche, H.; Su, Z. Z.; Emdad, L.; Baril, P.; Balme, B.; Thomas, L.; Randolph, A.; Valerie, K.; Sarkar, D.; Fisher, P. B. mda-9/Syntenin: A positive regulator of melanoma metastasis. Cancer Res. 2005, 65, 10901-10911.
65. Kegelman, T. P.; Das, S. K.; Hu, B.; Bacolod, M. D.; Fuller, C. E.; Menezes, M. E.; Emdad, L.; Dasgupta, S.; Baldwin, A. S.; Bruce, J. N.; et al. MDA-9/syntenin is a key regulator of glioma pathogenesis. Neuro Oncol. 2014, 16, 50-61.
66. Das, S. K.; Bhutia, S. K.; Sokhi, U.K.; Azab, B.; Su, Z. Z.; Boukerche, H.; Anwar, T.; Moen, E. L.; Chatterjee, D.; Pellecchia, M.; et al. Raf kinase inhibitor RKIP inhibits MDA-9/syntenin-mediated metastasis in melanoma. Cancer Res. 2012, 72, 6217-6226.
67. Li, H.; Tang, D. G. Prostate cancer stem cells and their potential roles in metastasis. J. Surg. Oncol. 2011, 103, 558-562.
68. Das, S. K.; Pradhan, A. K.; Bhoopathi, P.; Talukdar, S.; Shen, X. N.; Sarkar, D.; Emdad, L.; Fisher, P. B. The MDA-9/Syntenin/IGF1R/STAT3 Axis Directs Prostate Cancer Invasion. Cancer Res. 2018, 78, 2852-2863.
69. Shen, D. W.; Fojo, A.; Chin, J. E.; Roninson, I. B.; Richert, N.; Pastan, I.; Gottesman, M. M. Human multidrug-resistant cell lines: Increased mdr1 expression can precede gene amplification. Science 1986, 232, 643-645.
70. Fojo, A. T.; Ueda, K.; Slamon, D. J.; Poplack, D. G.; Gottesman, M. M.; Pastan, I. Expression of a multidrug-resistance gene in human tumors and tissues. Proc. Natl. Acad. Sci. USA 1987, 84, 265-269.
71. Parekh, H.; Wiesen, K.; Simpkins, H. Acquisition of taxol resistance via P-glycoprotein- and non-P-glycoprotein-mediated mechanisms in human ovarian carcinoma cells. Biochem. Pharmacol. 1997, 53, 461-470.
72. Wang, Y. C.; Juric, D.; Francisco, B.; Yu, R. X.; Duran, G. E.; Chen, K. G.; Chen, X.; Sikic, B. I. Regional activation of chromosomal arm 7q with and without gene amplification in taxane-selected human ovarian cancer cell lines. Genes Chromosomes Cancer 2006, 45, 365-374.
73. Lawlor, D.; Martin, P.; Busschots, S.; Thery, J.; O'Leary, J. J.; Hennessy, B. T.; Stordal, B. PARP Inhibitors as P-glyoprotein Substrates. J. Pharm. Sci. 2014, 103, 1913-1920.
74. Zarrintaj, P.; Mostafapoor, F.; Milan, P. B.; Saeb, M. R. Iheranostic Platforms Proposed for Cancerous Stem Cells: A Review. Curr. Stem. Cell Res. Ther. 2019, 14, 137-145.
75. Miki, J.; Rhim, J. S. Prostate cell cultures as in vitro models for the study of normal stem cells and cancer stem cells. Prostate Cancer Prostatic Dis. 2008, 11, 32-39.
76. Liu, C.; Kelnar, K.; Liu, B.; Chen, X.; Calhoun-Davis, T.; Li, H.; Patrawala, L.; Yan, H.; Jeter, C.; Honorio, S.; et al. The microRNA miR-34a inhibits prostate cancer stem cells and metastasis by directly repressing CD44. Nat. Med. 2011, 17, 211-215.

INFORMAL SEQUENCE LISTING

SEQ ID NO: 1 (PDZ1 domain)
QGIREVILCKDQDGKIGLRLKSIDNGIFVQLVQANSPASLVGLRFGDQVLQINGENCAG
WSSDKAHKVLKQAFGEKITMTIRDR
SEQ ID NO: 2 (shmda-9 #3)
TTGACTCTTAAGATTATGTAA
SEQ ID NO: 3 (shmda-9 #4)
TGGGATGGTCTTAGAATATTT
SEQ ID NO: 4 (Ad.5/3.shmda-9, forward primer)
GCCTGCTTTTATCTTTGAACATATTATTAAGCGAATGAAGCCTAGTATAATGAAAAG
CCTAATGGACCACACCATTCCTGAG
SEQ ID NO: 5 (Ad.5/3.shmda-9, reverse primer)
CTCAGGAATGGTGTGGTCCATTAGGCTTTTCATTATACTAGGCTTCATTCGCTTAAT
AATATGTTCAAAGATAAAAGCAGGC
SEQ ID NO: 6 (mda-9/syntenin sense siRNA)
GATCCGCGGATGGCACCAAGCATTTTCAAGAGAAATGCTTGGTGCCATCCGCTTTTTTG
GAAA
SEQ ID NO: 7 (mda-9/syntenin antisense siRNA)
AGCTTTTCCAAAAAAGCGGATGGCACCAAGCATTTCTCTTGAAAATGCTTGGTGCCATC
CGCG
SEQ ID NO: 8 (alternate mda-9/syntenin sense siRNA)
GCCTGCTTTTATCTTTGAACATATTATTAAGCGAATGAAGCCTAGTATAATGAAAA
GCCTAATGGACCACACCATTCCTGAG
SEQ ID NO: 9 (alternate mda-9/syntenin antisense siRNA)
CTCAGGAATGGTGTGGTCCATTAGGCTTTTCATTATACTAGGCTTCATTCGCTTAATAAT
ATGTTCAAAGATAAAAGCAGGC

What is claimed is:

1. A method of treating cancer in a subject in need thereof, comprising administering to the subject a combined effective amount of (a) an agent that inhibits MDA-9, and (b) an anti-cancer agent, wherein the anti-cancer agent is a mitotic inhibitor or a histone deacetylase (HDAC) inhibitor.

2. The method of claim 1, wherein the combined effective amount is a combined synergistic amount.

3. The method of claim 1, wherein the agent that inhibits MDA-9 is a compound that binds to MDA-9 protein.

4. The method of claim 3, wherein the agent that inhibits MDA-9 is a PDZ1 domain binder.

5. The method of claim 4, wherein the agent that inhibits MDA-9 is a compound having the formula:

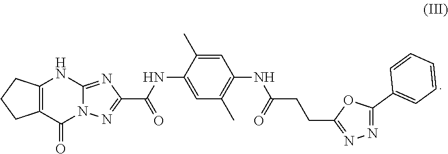 [SMILES: Cc1cc(NC(=O)c2nc3[nH]c4c(c(=O)n3n2)CCC4)c(C)cc1NC(=O)CCc1nnc(-c2ccccc2)o1]

6. The method of claim 1, wherein the agent that inhibits MDA-9 is an agent that decreases expression of MDA-9.

7. The method of claim 6, wherein the agent that inhibits MDA-9 is a small interfering RNA (siRNA) or a short hairpin RNA (shRNA) directed against MDA-9.

8. The method of any one of claims 1-7, wherein the anti-cancer agent is an anti-microtubule agent.

9. The method of claim 8, wherein the anti-microtubule agent is a taxane or a vinca alkaloid.

10. The method of claim 9, wherein the anti-microtubule agent is a taxane selected from paclitaxel, docetaxel, cabazitaxel, and larotaxel.

11. The method of claim 9, wherein the anti-microtubule agent is a vinca alkaloid selected from vinblastine, vincristine, or vinorelbine.

12. The method of claim any one of claims 1-7, wherein the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide.

13. The method of claim 12, wherein the HDAC inhibitor is TSA.

14. The method of any one of claims 1-7, wherein the agent that inhibits MDA-9 and the anti-cancer agent are co-administered.

15. The method of any one of claims 1-7, wherein the agent that inhibits MDA-9 and the anti-cancer agent are effective to increase apoptotic cell death in cancer stem cells (CSCs), as compared to either agent alone.

16. The method of any one of claims 1-7, wherein the cancer is selected from the group consisting of prostate cancer, breast cancer, gastric cancer, lung cancer, brain cancer, colorectal cancer, pancreatic cancer, melanoma, and neuroblastoma.

17. The method of claim 16, wherein the cancer is prostate cancer.

18. The method of claim 16, wherein the subject does not receive androgen ablation therapy.

19. The method of any one of claims 1-7, wherein the cancer is chemoresistant.

20. The method of claim 19, wherein the chemoresistance is MDR1-mediated.

21. The method of any one of claims 1-7, wherein the cancer is a recurrence of cancer.

22. A pharmaceutical composition comprising (a) an agent that inhibits MDA-9, and (b) an anti-cancer agent, wherein the anti-cancer agent is a mitotic inhibitor or a histone deacetylase (HDAC) inhibitor.

23. The pharmaceutical composition of claim 22, wherein the agent that inhibits MDA-9 is a compound that binds to MDA-9 protein.

24. The pharmaceutical composition of claim 23, wherein the agent that inhibits MDA-9 is a PDZ1 domain binder.

25. The pharmaceutical composition of claim 24, wherein the agent that inhibits MDA-9 is a compound having the formula:

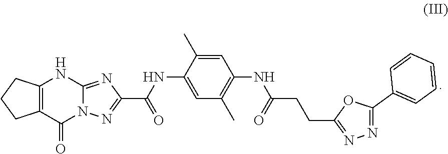 [SMILES: Cc1cc(NC(=O)c2nc3[nH]c4c(c(=O)n3n2)CCC4)c(C)cc1NC(=O)CCc1nnc(-c2ccccc2)o1]

26. The pharmaceutical composition of claim 22, wherein the agent that inhibits MDA-9 is an agent that decreases expression of MDA-9.

27. The pharmaceutical composition of claim 26, wherein the agent that inhibits MDA-9 is a small interfering RNA (siRNA) or a short hairpin RNA (shRNA) directed against MDA-9.

28. The pharmaceutical composition of any of claims 22-27, wherein the anti-cancer agent is an anti-microtubule agent.

29. The pharmaceutical composition of claim 28, wherein the anti-microtubule agent is a taxane or a vinca alkaloid.

30. The pharmaceutical composition of claim 29, wherein the anti-microtubule agent is a taxane selected from paclitaxel, docetaxel, cabazitaxel, and larotaxel.

31. The pharmaceutical composition of claim 29, wherein the anti-microtubule agent is a vinca alkaloid selected from vinblastine, vincristine, or vinorelbine.

32. The pharmaceutical composition of any one of claims 22-27, wherein the anti-cancer agent is an HDAC inhibitor selected from trichostatin A (TSA), oxamflatin, apicidin, depsipeptide, depudecin, trapoxin, vorinostat, suberoylanilide hydroxamic acid, and pyroxamide.

33. The pharmaceutical composition of claim 32, wherein the HDAC inhibitor is TSA.

34. The pharmaceutical composition of any one of claims 22-27, further comprising a pharmaceutically acceptable carrier.

35. The pharmaceutical composition of any one of claims 22-27, wherein the agent that inhibits MDA-9 and the anti-cancer agent are in a combined effective amount.

36. The pharmaceutical composition of claim 35, wherein the combined effective amount is a combined synergistic amount.

37. Use of a pharmaceutical composition of any one of claims 22-27 for the treatment of cancer.

38. The use of claim 37, wherein the cancer is selected from the group consisting of prostate cancer, breast cancer, gastric cancer, lung cancer, brain cancer, colorectal cancer, pancreatic cancer, melanoma, and neuroblastoma.

39. The use of claim 38, wherein the cancer is prostate cancer.

40. The use of claim 37, wherein the subject does not receive androgen ablation therapy.

41. The use of claim 37, wherein the cancer is chemoresistant.

42. The use of claim 41, wherein the chemoresistance is MDR1-mediated.

43. The use of claim 37, wherein the cancer is a recurrence of cancer.

44. A kit comprising a pharmaceutical composition of any one of claims 22-27.


SEQUENCE LISTING (9 sequences)

SEQ ID NO: 1 - protein, 84 aa, Artificial Sequence
  Synthetic construct
QGIREVILCKDQDGKIGLRLKSIDNGIFVQLVQANSPASLVGLRFGDQVLQINGENCAGWSSDKAHKVLKQAFGEKITMTIRDR

SEQ ID NO: 2 - DNA, 21 bp, Artificial Sequence
  Synthetic construct
ttgactcttaagattatgtaa

SEQ ID NO: 3 - DNA, 21 bp, Artificial Sequence
  Synthetic construct
tgggatggtcttagaatattt

SEQ ID NO: 4 - DNA, 82 bp, Artificial Sequence
  Synthetic construct
gcctgcttttatctttgaacatattattaagcgaatgaagcctagtataatgaaaagcctaatggaccacaccattcctgag

SEQ ID NO: 5 - DNA, 82 bp, Artificial Sequence
  Synthetic construct
ctcaggaatggtgtggtccattaggcttttcattatactaggcttcattcgcttaataatatgttcaaagataaaagcaggc

SEQ ID NO: 6 - DNA, 63 bp, Artificial Sequence
  Synthetic construct
gatccgcggatggcaccaagcattttcaagagaaatgcttggtgccatccgcttttttggaaa

SEQ ID NO: 7 - DNA, 63 bp, Artificial Sequence
  Synthetic construct
agcttttccaaaaaagcggatggcaccaagcatttctcttgaaaatgcttggtgccatccgcg

SEQ ID NO: 8 - DNA, 82 bp, Artificial Sequence; identical to SEQ ID NO: 4
  Synthetic construct

SEQ ID NO: 9 - DNA, 82 bp, Artificial Sequence; identical to SEQ ID NO: 5
  Synthetic construct